(19) United States
(12) Patent Application Publication
(10) Pub. No.: US 2009/0186907 A1
(43) Pub. Date: Jul. 23, 2009

(54) Pyrazinones As Cellular Proliferation Inhibitors

(75) Inventors: James Francis Bereznak, Newtown Square, PA (US); Thomas Martin Stevenson, Newark, DE (US); Andrew Edmund Taggi, Newark, DE (US); Paula Louise Sharpe, Middletown, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DuPont De Nemours and Company, Wilmington, DE (US)

(21) Appl. No.: 12/304,156

(22) PCT Filed: Jun. 19, 2007
(86) PCT No.: PCT/US07/14297

§ 371 Date: Dec. 10, 2008

Related U.S. Application Data
(60) Provisional application No. 60/815,359, filed on Jun. 21, 2006.

(51) Int. Cl. A61K 31/497; C12N 5/06; C07D 403/04

(52) U.S. Cl. 514/255.05; 435/375; 544/405

(57) ABSTRACT

This invention pertains to a method of inhibiting undesired animal cellular proliferation said method comprising contacting an animal cell with a compound of Formula 1 and all pharmaceutically acceptable, salts, N-oxides, hydrates, solvates, or geometric and stereoisomers thereof:

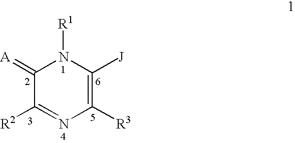

wherein
R¹ is NR⁴R⁵, N═CR¹⁹R²¹, OR⁶, G¹ or G²; or C₁-C₈ alkyl, C₂-C₈ alkenyl, each optionally substituted;
A is O, S or NR⁷;
R² is cyano, NR⁸—N═CR⁹R¹⁰ or NC(═O)R³⁰; or a 5- or 6-membered heteroaromatic ring, each ring or ring system optionally substituted;
R³ is H, halogen, cyano, C₁-C₆ alkyl;
J is C₁-C₈ alkyl or phenyl, optionally substituted; and
R⁴, R⁵, R⁶, R⁷, R⁸, R⁹, R¹⁰, R¹⁹, R²¹, R³⁰, G¹ and G² are as defined in the disclosure.

### FIELD OF THE INVENTION

The present invention is related to methods of inhibiting undesired cell proliferation by contacting said cells with novel heterocyclic compounds having antiproliferative and antimitotic activity.

### BACKGROUND OF THE INVENTION

There are many human and veterinary diseases that stem from processes of uncontrolled or abnormal cellular proliferation.

Accordingly, it is one object of the present invention to provide compounds which directly or indirectly are toxic to actively dividing cells and are useful in the treatment of conditions caused by undesired cellular proliferation. A further object of the present invention is to provide therapeutic compositions for treating said conditions.

Still further objects are to provide methods for inhibiting undesired cellular proliferation such as the proliferation of cancerous, infected, or epithelial cells, and treating all types of cancers, infections, inflammatory, and generally proliferative conditions. A further object is to provide methods for treating other medical conditions characterized by the presence of rapidly proliferating cells.

Other objects, features and advantages will become apparent to those skilled in the art from the following description and claims.

### SUMMARY OF THE INVENTION

This invention pertains to a method of inhibiting undesired proliferation of an animal cell, said method comprising contacting said cell or a tissue or organ in which proliferation of said cell is not desired with a compound of Formula 1, prodrugs thereof, and all pharmaceutically acceptable salts, N-oxides, hydrates, solvates, crystal forms or geometric and stereoisomers thereof:

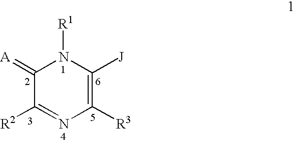

wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₅-C₁₀ alkylcycloalkylalkyl, C₇-C₁₄ alkylcycloalkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³, —NR⁸C(O)R²⁶, —NR⁸C(O)NR²⁷ or —NR⁸C(O)OR²⁸; or
R² is a 5- or 6-membered heteroaromatic ring or a 8-, 9- or 10-membered heteroaromatic bicyclic ring system, each ring or ring system optionally substituted with up to 5 substituents independently selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴; or
R² and R⁷ are taken together as —N═C(R¹⁶)—;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ is independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
each R⁸ is independently H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
R¹⁶ is H, halogen, cyano, C₁-C₆ alkyl, C₁-C₄ haloalkyl, C₃-C₆ cycloalkyl, C₃-C₆ halocycloalkyl, C₂-C₆ alkenyl, C₂-C₆ alkynyl, C₁-C₄ alkoxy, C₁-C₄ haloalkoxy, C₁-C₄ alkylthio, C₁-C₄ haloalkylthio or C₂-C₅ alkoxycarbonyl;
J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylsulfinyl, C₁-C₄ alkylsulfonyl, C₂-C₆ alkoxycarbonyl, C₂-C₆ alkylcarbonyl, C₁-C₄ alkylamino, C₂-C₆ dialkylamino and C₃-C₆ trialkylsilyl; or
J is a phenyl, benzyl, naphthalene, 5- or 6-membered heteroaromatic ring or 8-, 9- or 10-membered heteroaromatic bicyclic ring system, each ring or ring system optionally substituted with up to 5 substituents independently selected from R²⁹ and R³⁰;
R²⁹ is halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R³⁰ is —Y-X-Q;
Y is O, S(O)ₚ, NR³¹ or direct bond;
X is C₁-C₆ alkylene, C₂-C₆ alkenylene, C₃-C₆ alkynylene, C₃-C₆ cycloalkylene or C₃-C₆ cycloalkenylene, each optionally substituted with one or more substituents independently selected from the group consisting of halogen, cyano, nitro, hydroxy, (═O), C₁-C₆ alkoxy and C₁-C₆ haloalkoxy;
Q is NR³²R³³, OR³⁵ or S(O)ₚR³⁵;
R³¹ is H, C₁-C₆ alkyl, C₁-C₆ haloalkyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
each R³² and R³³ is independently H; or C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl; or R³² and R³³ when optionally taken together with the nitrogen atom to which each is attached form a heterocyclic ring of 3 to 6 ring atoms optionally substituted with R³⁴;
R³⁴ is halogen, C₁-C₆ alkyl, C₁-C₆ haloalkyl or C₁-C₆ alkoxy;
each R³⁵ is independently H, C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
p is 0, 1 or 2;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents independently selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ is independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or —CH₂CH(CH₃)OCH(CH₃)CH₂—;
each R²² and R²³ is independently H; or C₁-C₄ alkyl, C₁-C₄ alkoxy, C₃-C₈ cycloalkyl or C₄-C₈ cycloalkylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R²⁵ is H, C₁-C₄ alkyl or C₁-C₄ haloalkyl; and
R²⁶ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from R³⁶;
R³⁶ is C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy or C₁-C₄ haloalkoxy; and
each R²⁷ and R²⁸ is independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, halogen, cyano, nitro, C₁-C₄ alkoxy and C₁-C₄ haloalkoxy.

The invention also includes novel compounds of Formula 1 or salts thereof, wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₅-C₁₀ alkylcycloalkylalkyl, C₇-C₁₄ alkylcycloalkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³, —NR⁸C(O)R²⁶, —NR⁸C(O)NR²⁷ or —NR⁸C(O)OR²⁸; or
R² is a 5- or 6-membered heteroaromatic ring or a 8-, 9- or 10-membered heteroaromatic bicyclic ring system, each ring or ring system optionally substituted with up to 5 substituents independently selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴; or
R² and R⁷ are taken together as —N═C(R¹⁶)—;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ is independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
each R⁸ is independently H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
R¹⁶ is H, halogen, cyano, C₁-C₆ alkyl, C₁-C₄ haloalkyl, C₃-C₆ cycloalkyl, C₃-C₆ halocycloalkyl, C₂-C₆ alkenyl, C₂-C₆ alkynyl, C₁-C₄ alkoxy, C₁-C₄ haloalkoxy, C₁-C₄ alkylthio, C₁-C₄ haloalkylthio or C₂-C₅ alkoxycarbonyl;
J is a phenyl, benzyl, naphthalene, 5- or 6-membered heteroaromatic ring or 8-, 9- or 10-membered heteroaromatic bicyclic ring system, each ring or ring system substituted with 1 to 2 substituents independently selected from R³⁰ and optionally substituted up to 4 substituents independently selected from R²⁹;
R²⁹ is halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R³⁰ is —Y-X-Q;
Y is O, S(O)ₚ, NR³¹ or direct bond;
X is C₁-C₆ alkylene, C₂-C₆ alkenylene, C₃-C₆ alkynylene, C₃-C₆ cycloalkylene or C₃-C₆ cycloalkenylene, each optionally substituted with one or more substituents independently selected from the group consisting of halogen, cyano, nitro, hydroxy, (═O), C₁-C₆ alkoxy and C₁-C₆ haloalkoxy;
Q is NR³²R³³, OR³⁵ or S(O)ₚR³⁵;
R³¹ is H or C₁-C₆ alkyl, C₁-C₆ haloalkyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
each R³² and R³³ is independently H; or C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl; or R³² and R³³ when optionally taken together with the nitrogen atom to which each is attached form a heterocyclic ring of 3 to 6 ring atoms optionally substituted with R³⁴;
R³⁴ is halogen, C₁-C₆ alkyl, C₁-C₆ haloalkyl or C₁-C₆ alkoxy;
each R³⁵ is independently H, C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
p is 0, 1 or 2;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents independently selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ is independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or —CH₂CH(CH₃)OCH(CH₃)CH₂—;
each R²² and R²³ is independently H; or C₁-C₄ alkyl, C₁-C₄ alkoxy, C₃-C₈ cycloalkyl or C₄-C₈ cycloalkylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R²⁵ is H, C₁-C₄ alkyl or C₁-C₄ haloalkyl; and
R²⁶ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from R³⁶;
R³⁶ is C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy or C₁-C₄ haloalkoxy; and
each R²⁷ and R²⁸ is independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, halogen, cyano, nitro, C₁-C₄ alkoxy and C₁-C₄ haloalkoxy.

This invention pertains to a method of inhibiting animal derived microtubule function contacting said microtubules with a compound of Formula 1 including prodrugs thereof, and all pharmaceutically acceptable salts, N-oxides, hydrates, solvates, crystal forms or geometric and stereoisomers thereof.

The invention pertains to a method of inhibiting undesired animal cell proliferation said method comprising contacting said cells or a tissue or organ in which proliferation of said cell is not desired with a compound of Formula1 and wherein said compound inhibits microtubule function.

The invention also pertains to a method for treating a cellular hyperproliferation disorder in an individual comprising administering to the individual a therapeutically effective amount of a compound of Formula 1 including all prodrugs thereof, pharmaceutically acceptable salts, N-oxides, hydrates, solvates, crystal forms or geometric and stereoisomers thereof.

The invention also pertains to a method of treating cancer in an individual comprising administering to the individual a therapeutically effective amount of a compound of Formula1 including all prodrugs thereof, pharmaceutically acceptable salts, N-oxides, hydrates, solvates, crystal forms or geometric and stereoisomers thereof.

The invention also pertains to the use of a compound of Formula 1 as a treatment for a cellular hyperproliferation disorder in an individual.

The invention also pertains to the use of a Compound of Formula 1 in the manufacture of a medicament for the therapeutic and/or prophylactic treatment of a cellular hyperproliferation disorder in an individual.

### DETAILS OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and Both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular. For example, a composition of the present invention comprises a biologically effective amount of “a” compound of Formula 1 which should be read that the composition includes one or at least one compound of Formula 1.

“Inhibiting microtubule function” means disrupting the dynamic process of tubulin polymerization and depolymerization by any mechanism of action including the inhibition of polymerization, causing depolymerization of oligomeric or higher forms of tubulin aggregates, or the stabilization of polymerized tubulin or microtubular structures.

An “individual” or “animal in need of treatment” can be a human in need of treatment, but can also be another animal in need of treatment, e.g. companion animals (such as dogs, cats and the like), farm animals (such as cows, pigs, horses, chickens and the like) and laboratory animals (such as rats, mice, guinea pigs and the like). Therefore, in addition to individuals such as humans, a variety of other mammals including other primates can be treated according to the methods of the present invention. For instance, mammals including, but not limited to, cows, sheep, goats, horses, dogs, cats, guinea pigs, rats or other bovine, ovine, equine, canine, feline, rodent or murine species can be treated. Furthermore, the methods can also be practiced in other species, such as avian species (e.g., chickens).

An “animal cell” therefore is a cell found in or derived from an animal including a human including those exemplified above. The animals may be mammals or non-mammals including avian species as noted above.

A “therapeutically effective amount” is the quantity of compound which results in an improved clinical outcome as a result of the treatment compared with a typical clinical outcome in the absence of the treatment. An “improved clinical outcome” includes a longer life expectancy or relief of unwanted symptoms for the individual receiving treatment. It can also include slowing or arresting the rate of growth of a tumor, causing shrinkage in the size of the tumor, a decreased rate of metastasis, and/or a decreased rate of abnormal or undesired proliferation and/or angiogenesis. It can also include inhibition of microtubule function.

An “effective amount” or “amount sufficient” refers to an amount of compound or composition effective to depress, suppress or regress the undesired activity.

The terms “administration of” and “administering a” compound should be understood to mean providing a compound of the invention to the individual in need of treatment.

The term “composition” as used herein is intended to encompass a product comprising the specified ingredients in the specified amounts, as well as any product which results, directly or indirectly, from combination of the specified ingredients in the specified amounts.

By “pharmaceutically acceptable” or “physiologically acceptable” it is meant the salts, N-oxides, hydrates, solvates, crystal forms, geometric and stereoisomers of the compounds or a carrier, diluent or excipient must be compatible with the other ingredients of the formulation and not generally deleterious to animal cellular systems.

A “cellular hyperproliferation disorder” as used herein is intended to mean any disease state in an individual characterized by the presence of undesired proliferating cells wherein the cellular proliferation is causative of the disease state.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Dialkoxyalkyl” denotes dialkoxy substitution on alkyl. Examples of “dialkoxyalkyl” include (CH₃O)₂CH₂, (CH₃O)₂CH₂CH₂, (CH₃CH₂O)₂CH₂ and (CH₃CH₂O)₂CH₂CH₂. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O), CH₃CH₂S(O), CH₃CH₂CH₂S(O), (CH₃)₂CHS(O) and the different butylsulfinyl, pentylsulfinyl and hexylsulfinyl isomers. Examples of “alkylsulfonyl” include CH₃S(O)₂, CH₃CH₂S(O)₂, CH₃CH₂CH₂S(O)₂, (CH₃)₂CHS(O)₂ and the different butylsulfonyl, pentylsulfonyl and hexylsulfonyl isomers. “Alkylamino”, “dialkylamino”, and the like, are defined analogously to the above examples. “Alkylcycloalkylamino” denotes alkyl and cycloalkyl groups substituted with one amino group. Examples of “alkylcycloalkylamino” include methylcyclopropylamino and methylcyclohexylamino. “Cycloalkyl” includes, for example, cyclopropyl, cyclobutyl, cyclopentyl, and cyclohexyl. “Cycloalkenyl” includes groups such as cyclopentenyl and cyclohexenyl as well as groups with more than one double bond such as 1,3- and 1,4-cyclohexadienyl. Examples of “cycloalkylalkyl” include cyclopropylmethyl, cyclopentylethyl, and other cycloalkyl moieties bonded to straight-chain or branched alkyl groups. “Alkylcycloalkyl” denotes alkyl substitution on a cycloalkyl moiety. Examples include 4-methylcyclohexyl and 3-ethylcyclopentyl. “Alkylcycloalkylalkyl” denotes alkyl substitution on a cycloalkylalkyl moiety. Examples include 4-methylcyclohexylmethyl and 3-ethylcyclopentylmethyl. “Alkylcycloalkylcycloalkyl” denotes alkylcycloalkyl substitution on a cycloalkyl moiety. Examples include 4-methyl-4-cyclohexylcyclohexyl and 2-methyl-2-cyclopropylcyclopropyl. The term “carbocyclic ring” denotes a ring wherein the atoms forming the ring backbone and selected only from carbon. The term “aromatic ring system” denotes fully unsaturated carbocycles and heterocycles in which the polycyclic ring system is aromatic. Aromatic indicates that each of ring atoms is essentially in the same plane and has a p-orbital perpendicular to the ring plane, and in which (4n+2) π electrons, when n is 0 or a positive integer, are associated with the ring to comply with Hickel's rule. The term “nonaromatic carbocyclic ring system” denotes fully saturated carbocycles as well as partially or fully unsaturated carbocycles wherein none of the rings in the ring system are aromatic. The term “nonaromatic heterocyclic ring system” denotes fully saturated heterocycles as well as partially or fully unsaturated heterocycles wherein none of the rings in the ring system are aromatic. The heterocyclic ring systems can be attached through any available carbon or nitrogen by replacement of a hydrogen on said carbon or nitrogen. The term “heteroaromatic ring” denotes a fully aromatic heterocyclic ring in which at least one ring atom is not carbon and which comprises 1 to 4 heteroatoms independently selected from the group consisting of nitrogen, oxygen and sulfur, provided that each heterocyclic ring includes no more than 4-nitrogens, no more than 2 oxygens and no more than 2 sulfurs. The term “heteroaromatic bicyclic ring system” denotes a bicyclic ring which contains at least one heteroatom and in which at least one ring of the bicyclic ring system is aromatic. The heteroaromatic rings or heterobicyclic ring systems can be attached through any available carbon or nitrogen by replacement of a hydrogen on said carbon or nitrogen. One skilled in the art will appreciate that not all nitrogen containing heterocycles can form N-oxides since the nitrogen requires an available lone pair of electrons for oxidation to the oxide; one skilled in the art will recognize those nitrogen containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethydroxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “halocycloalkyl”, “haloalkoxy”, “haloalkylthio”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(O), CCl₃S(O), CF₃CH₂S(O) and CF₃CF₂S(O). Examples of “haloalkylsulfonyl” include CF₃S(O)₂, CCl₃S(O)₂, CF₃CH₂S(O)₂ and CF₃CF₂S(O)₂. “Trialkylsilyl” includes 3 branched and/or straight-chain alkyl radicals attached to and linked through a silicon atom such as trimethylsilyl, triethylsilyl and t-butyldimethylsilyl.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₄ cycloalkylalkyl designates cyclopropylmethyl; C₅ cycloalkylalkyl designates, for example, cyclopropylethyl or cyclobutylmethyl; and C₆ cycloalkylalkyl designates the various ring size of a cycloalkyl group substituted with an alkyl group containing a total of sixcarbon atoms, examples including cyclopentylmethyl, 1-cyclobutylethyl, 2-cyclobutylethyl and 2-cyclopropylpropyl. Examples of “alkylcarbonyl” include C(O)CH₃, C(O)CH₂CH₂CH₃ and C(O)CH(CH₃)₂. Examples of “alkoxycarbonyl” include CH₃C(═O), CH₃CH₂C(═O), CH₃CH₂CH₂C(═O), (CH₃)₂CHOC(═O) and the different butoxy- or pentoxycarbonyl isomers. Examples of “alkylaminocarbonyl” include CH₃NHC(═O)—, CH₃CH₂NHC(═O)—, CH₃CH₂CH₂NHC(═O)—, (CH₃)₂CHNHC(═O)— and the different butylamino- or pentylaminocarbonyl isomers. Examples of “dialkylaminocarbonyl” include (CH₃)₂NC(═O)—, (CH₃CH₂)₂NC(═O)—, CH₃CH₂(CH₃)NC(═O)—, (CH₃)₂CHN(CH₃)C(═O)— and CH₃CH₂CH₂(CH₃)NC(═O)—. In the above recitations, when a compound of Formula1 is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents is greater than 1, said substituents are independently selected from the group of defined substituents. Further, when the subscript indicates a range, e.g. (R)ᵢ₋ⱼ, then the number of substituents may be selected from the integers between i and j inclusive.

When a group contains a substituent which can be hydrogen, for example R³, R⁴, R⁵, R⁶, R⁷, R¹⁰, R¹¹, R¹², R¹³, R¹⁴, R¹⁶, R²², R²³, R²⁵, R²⁶, R³¹, R³² or R³³ then, when this substituent is taken as hydrogen, it is recognized that this is equivalent to said group being unsubstituted. When R² and R⁷ are taken together as —N═C(R¹⁶)—, the left-hand bond is connected as R² and the right-hand bond is connected as R⁷. The term “optionally substituted” in connection with groups listed for R¹, R², R⁴, R⁵, R⁶, R²², R²³, R³⁰, R³¹, R³², J, G¹ and G² refers to groups that are unsubstituted or have at least 1 non-hydrogen substituent. These groups may be substituted with as many optional substituents as can be accommodated by replacing a hydrogen atom with a non-hydrogen substituent on any available carbon or nitrogen atom. Commonly, the number of optional substituents (when present) ranges from 1 to 5. Examples of 5- or 6-membered heteroaromatic rings optionally substituted with up to 5 substituents described for R² include the rings H-1 through H-24 illustrated in Exhibit 1 wherein each R²⁰ is independently R²⁴ and r is an integer from 0 to 5 and the ring U-62 illustrated in Exhibit 3 wherein the N in the ring is unsubstituted. Examples of 5- or 6-membered heteroaromatic rings optionally substituted with up to 5 substituents described for J include the rings H-1 through H-24 illustrated in Exhibit1 wherein each R²⁰ is independently R²⁹ and r is an integer from 0 to 5. Examples of 5- or 6-membered heteroaromatic rings optionally substituted with up to 4 substituents described for G² include the rings H-1 through H-24 illustrated in Exhibit1 wherein each R²⁰ is independently R¹⁸ and r is an integer from 0 to 4. Examples of 5- or 6-membered heteroaromatic rings optionally substituted with up to 4 substituents described for R²⁶ include the rings H-1 through H-24 illustrated in Exhibit 1 wherein each R²⁰ is independently R³⁶ and r is an integer from 0 to 4. Examples of 8-, 9- or 10-membered heteroaromatic bicyclic rings optionally substituted with from 1 to 5 substituents described for R² include the rings B-1 through B-39 illustrated in Exhibit2 wherein each R²⁰ is independently R²⁴ and r is an integer from 0 to 5. Examples of 8-, 9- or 10-membered heteroaromatic bicyclic rings optionally substituted with from 1 to 5 substituents described for J include the rings B-1 through B-39 illustrated in Exhibit2 wherein each R²⁰ is independently R²⁹ and r is an integer from 0 to 5. Examples of 5- or 6-membered saturated or partially saturated heterocyclic rings, each optionally substituted with up to 5 substituents described for R² include the rings U-20 through U-68 illustrated in Exhibit3 wherein each R²⁰ is independently R²⁴ and r is an integer from 0 to 5. Examples of 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents described for G¹ include the rings U-1 through U-77 illustrated in Exhibit3 wherein R²⁰ is R¹⁷, and r is an integer from 0 to 4. Although R²⁰ groups are shown in the structures showed in Exhibit 1, Exhibit 2 and Exhibit 3, it is noted that they do not need to be present since they are optional substituents. The nitrogen atoms that require substitution to fill their valence are substituted with H or R²⁰. Note that when the nitrogen of the ring of U-54 or U-62 illustrated in Exhibit 3 is unsubstituted, U-54 or U-62 has 6-membered aromatic ring structure and belongs to the groups illustrated in Exhibit 1. Note that some H groups in Exhibit 1 can only be substituted with less than 4 R²⁰ groups as described for G² (e.g. H-1 through H-24). Note that some B groups in Exhibit 2 can only be substituted with less than 5 R²⁰ groups (e.g. B-5 through B-9, B-21 through B-23, B-25 through B-27 and B-37 through B-39). Note that some U groups in Exhibit 3 can only be substituted with less than 5 R²⁰ groups (e.g. U-1, U-6, U-10, U-11, U-16 through U-19, U-24 through U-40, U-54, U-56 through U-60, U-62 through U-64 and U-66 through U-68). Note that when the attachment point between (R²⁰)ᵣ and the H, B or U group is illustrated as floating, (R²⁰)ᵣ can be attached to any available carbon atom or nitrogen atom of the H, B or U group. Note that when the attachment point of the H, B or U group is illustrated as floating, the H, B or U group can be attached to the remainder of Formula 1 through any available carbon atom or nitrogen atom of the H, B or U group by replacement of a hydrogen atom.

### Exhibit 1

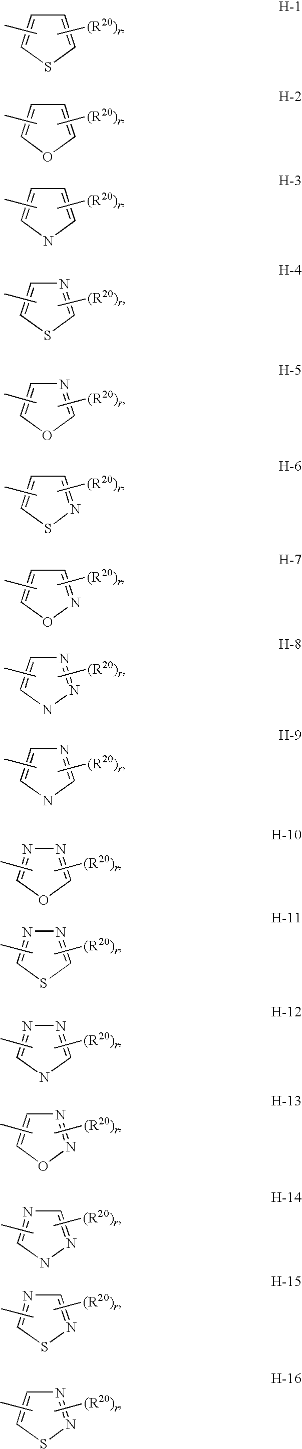

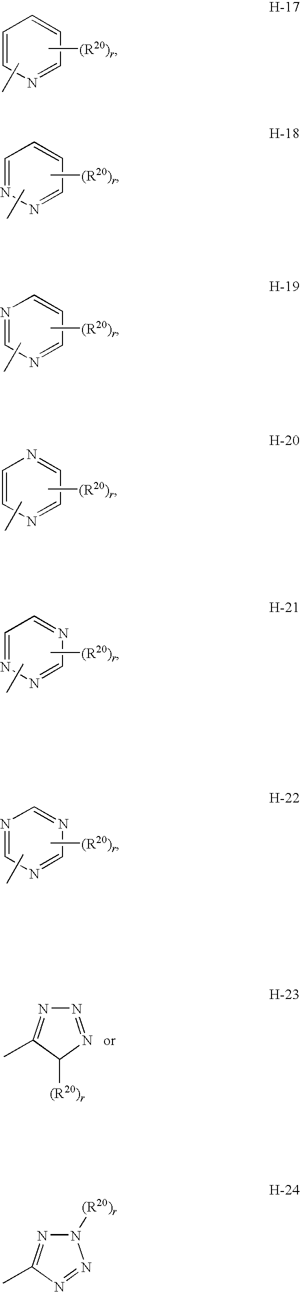

### Exhibit 2

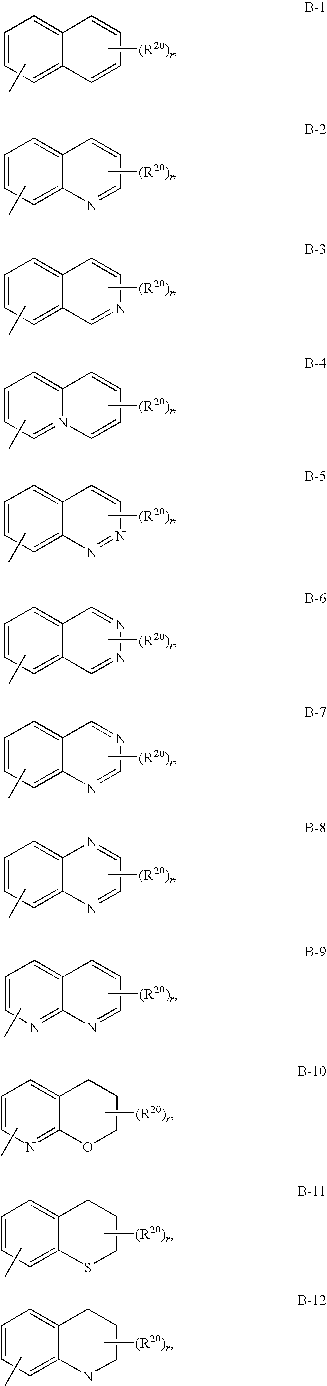

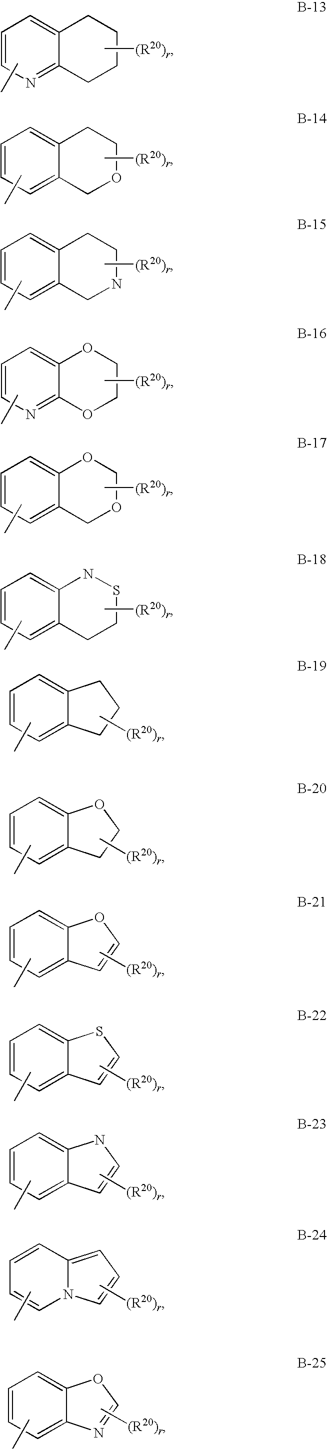

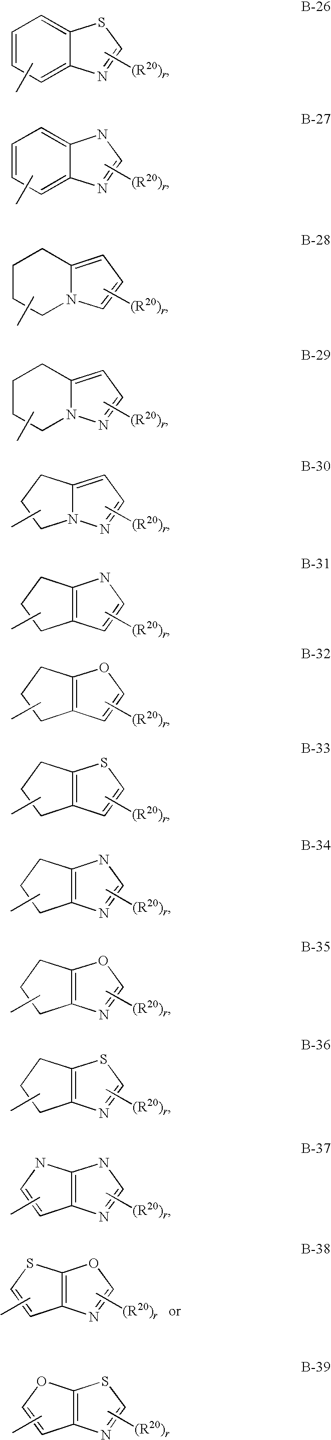

### Exhibit 3

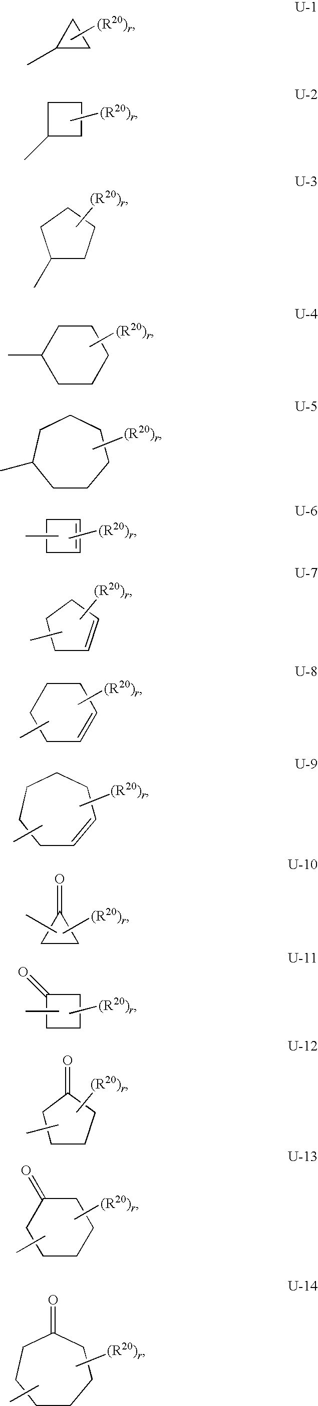

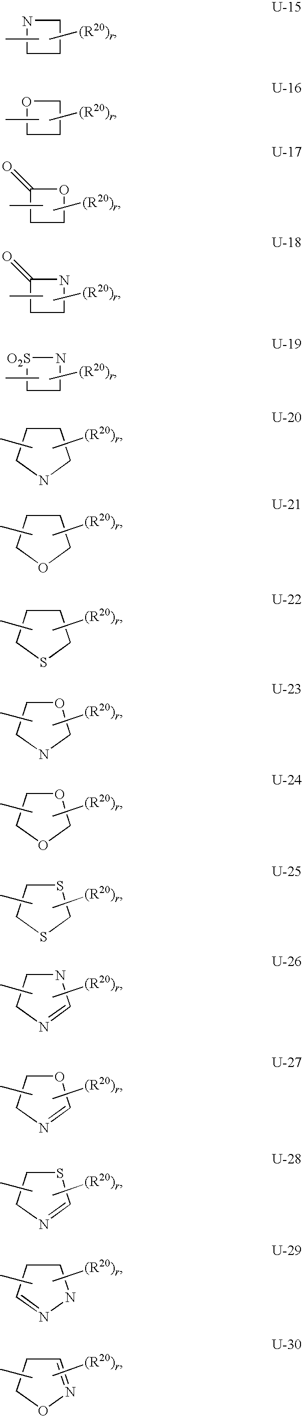

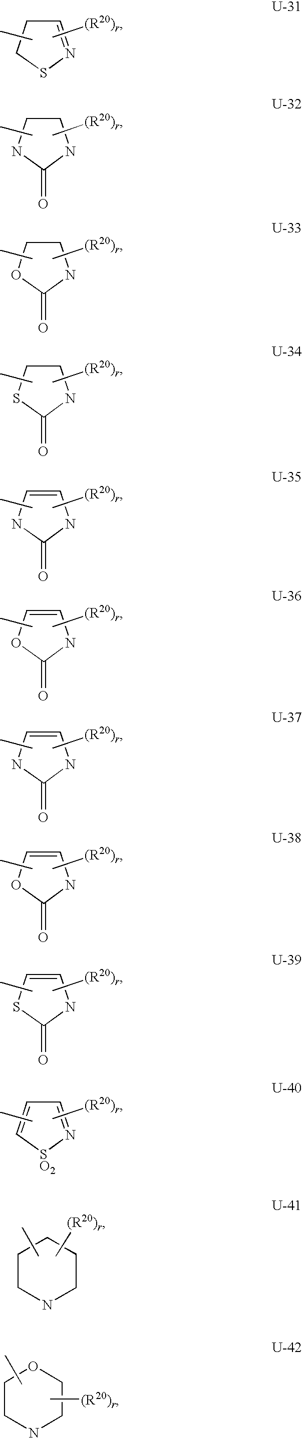

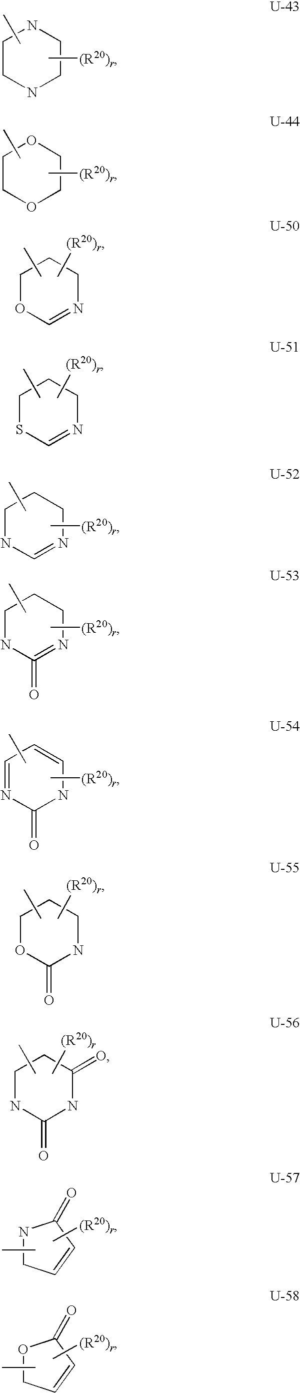

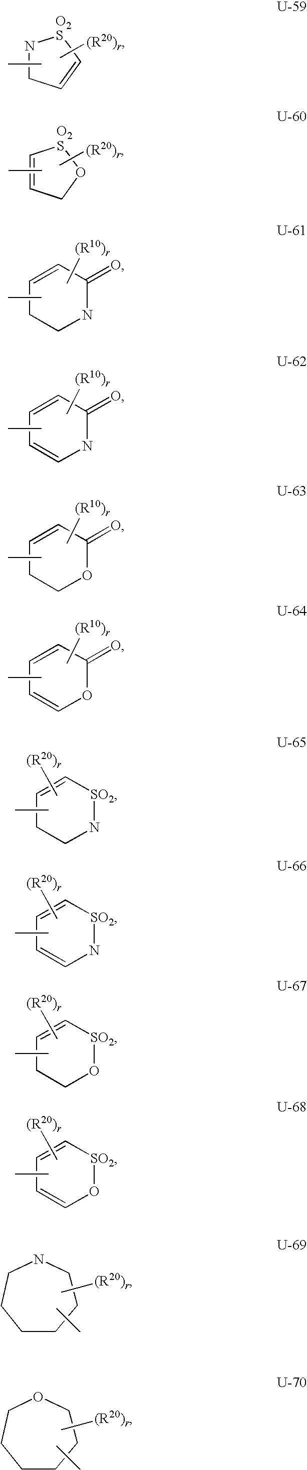

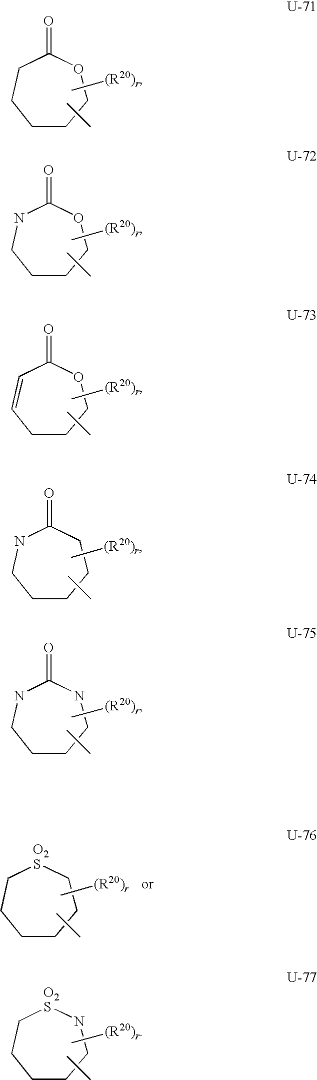

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the present invention comprises compounds selected from Formula1, N-oxides and pharmaceutically suitable salts thereof. The compounds of the invention may be present as a mixture of stereoisomers, individual stereoisomers, or as an optically active form. For example, when R¹ is 2-methylbutyl group, Formula 1 possesses a chiral center at the carbon atom identified with the asterisk (*). This invention comprises racemic mixtures, and also includes with compounds that are enriched compared to the racemic mixture with an enantiomer of Formula 1.

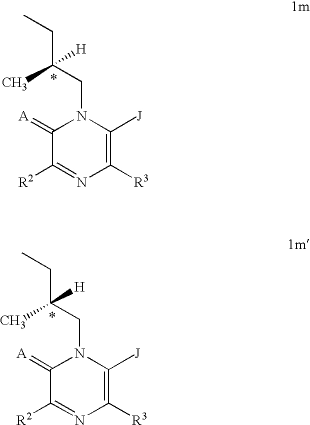

Included are the essentially pure enantiomers of compounds of Formula 1, for example, Formula 1m and Formula 1m′ (Formula 1 wherein R¹ is 2-methylbutyl group).

When a compound is enantiomerically enriched, one enantiomer is present in greater amounts than the other, and the extent of enrichment can be specified by an expression of enantiomeric excess (“ee”), which is defined as (2x−1)100%, where x is the mole fraction of the dominant enantiomer in the mixture (e.g., an ee of 20% corresponds to a 60:40 ratio of enantiomers).

For the compounds of Formula 1 where R¹ is 2-methylbutyl group, the more active enantiomer is believed to be the enantiomer in which the hydrogen atom attached to the carbon atom identified with an asterisk (*) lies below the plane defined by the 3 non-hydrogen atoms attached to the carbon atom identified with the asterisk (*), as is shown in Formula 1m. The carbon atom identified with an asterisk (*) in Formula 1m has the S configuration.

Preferably the compositions of this invention have at least a 50% enantiomeric excess; more preferably at least a 75% enantiomeric excess; still more preferably at least a 90% enantiomeric excess; and most preferably at least a 94% enantiomeric excess of the more active isomer. Of particular note are enantiomerically pure embodiments of the more active isomer.

In particular, when J is a phenyl ring substituted with R²⁹ at the ortho position of the ring, or an analogous naphthalene, 5- or 6-membered heteroaromatic ring or 8-, 9- or 10-membered heteroaromatic bicyclic ring system, wherein R²⁹ is as described for J ring or ring system substituents in the Summary of the Invention, then Formula 1 possesses an axis of chirality differentiating two atropisomers (chiral rotational isomers). The atropisomers of Formula 1 can be separated because rotation about the single bond connecting J is prevented or greatly retarded. This invention comprises racemic mixtures of such atropisomer. And also includes compounds that are enriched compared to the racemic mixture with an atropisomer of Formula 1n or 1n′.

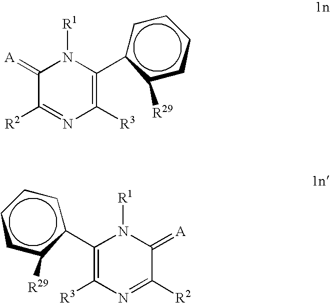

The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, succinic, 4-toluenesulfonic or valeric acids when the compound contains a basic group such as an amine.

The salts of the compounds of the invention also include those formed with organic bases (e.g., pyridine, ammonia, or triethylamine) or inorganic bases (e.g., hydrides, hydroxides, or carbonates of sodium, potassium, lithium, calcium, magnesium or barium) when the compound contains an acidic group such as a carboxylic acid or phenol.
Embodiments of the present invention also include:
Embodiment A. A method of inhibiting undesired proliferation of an animal cell, said method comprising contacting said cell or a tissue or organ in which proliferation of said cell is not desired with a compound of Formula 1 wherein R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G².
Embodiment A1. A method of Embodiment A wherein R¹ is C₁-C₈ alkyl, C₁-C₈ haloalkyl, C₃-C₈ cycloalkyl, C₄-C₈ cycloalkylalkyl, NR⁴R⁵, —N═CR¹⁹R²¹, G¹ or G².
Embodiment A2. A method of Embodiment A1 wherein R¹ is C₂-C₆ alkyl, C₂-C₆ haloalkyl, C₄-C₈ cycloalkylalkyl, NR⁴R⁵, G¹ or G².
Embodiment A3. A method of Embodiment A2 wherein R¹ is C₂-C₆ alkyl, C₂-C₆ haloalkyl or C₄-C₈ cycloalkylalkyl.
Embodiment A4. A method of Embodiment A3 wherein R¹ is C₃-C₆ alkyl, C₃-C₆ haloalkyl or C₄-C₆ cyclopropylalkyl.
Embodiment A5. A method of Embodiment A2 wherein R¹ is NR⁴R⁵.
Embodiment A6. A method of Embodiment A2 wherein R¹ is G¹.
Embodiment A7. A method of Embodiment A2 wherein R¹ is G².
Embodiment A8. A method of Embodiment A5 wherein each R⁴ and R⁵ is independently H, C₁-C₈ alkyl or C₁-C₈ haloalkyl.
Embodiment A9. A method of Embodiment A8 wherein each R⁴ and R⁵ is independently H, C₃-C₆ alkyl or C₃-C₆ haloalkyl.
Embodiment A10. A method of Embodiment A6 wherein G¹ is a 5- to 6-membered nonaromatic carbocyclic or heterocyclic ring, optionally including 1 or 2 ring members selected from the group consisting of C(═O), C(═S), S(O) and S(O)₂.

Embodiment A11. A method of Embodiment A10 wherein G¹ is a 5- to 6-membered nonaromatic carbocyclic or heterocyclic ring, optionally including 1 or 2 ring members selected from the group consisting of C(═O).
Embodiment A12. A method of Embodiment A7 wherein G² is a phenyl ring, optionally substituted with from 1 to 4 substituents independently selected from R¹⁸.
Embodiment A13. A method of Embodiment A7 wherein G² is a 5- or 6-membered heteroaromatic ring, each ring or ring system optionally substituted with from 1 to 4 substituents independently selected from R¹⁸.
Embodiment A14. A method of inhibiting undesired animal cellular proliferation said method comprising contacting said animal cells or a tissue or organ in which proliferation of said cell is not desired with a compound of Formula 1 wherein A is O or S.
Embodiment A15. A method of Embodiment A14 wherein A is O.
Embodiment A16. A method administering the compound of Formula 1 wherein R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³ or —NR⁸C(═O)R²⁶.
Embodiment A17. A method of Embodiment A16 wherein R² is cyano, —R⁸N═CR⁹R¹⁰, —CR¹³═NOR¹⁴, —CR¹³═NNR¹¹R¹², —C(W)NR²²R²³ or —NR⁸C(═O)R²⁶.
Embodiment A18. A method of Embodiment A17 wherein R² is cyano, —C(W)NR²²R²³ or —NR⁸C(═O)R²⁶.
Embodiment A19. A method of Embodiment A18 wherein R² is cyano, —CONH₂ or —NHC(═O)CH₃.
Embodiment A20. A method of Embodiment A18 wherein W is O.
Embodiment A21. A method of Embodiment A18 wherein each R²² and R²³ is independently H or C₁-C₄ alkyl.
Embodiment A22. A method of inhibiting undesired animal cellular proliferation said method comprising contacting said animal cells or a tissue or organ in which proliferation of said cell is not desired with a compound of Formula1 wherein R² is a 5- or 6-membered heteroaromatic ring, each ring optionally substituted with up to 5 substituents selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴.
Embodiment A23. A method of Embodiment A22 wherein R² is a 5- or 6-membered heteroaromatic ring, each ring optionally substituted with up to 4 substituents selected from R²⁴; or 5- or 6-membered saturated or partially saturated heterocyclic ring, optionally including 1-3 ring members selected from the group consisting of C(═O), optionally substituted with up to 5 substituents independently selected from R²⁴.
Embodiment A24. A method of Embodiment A23 wherein R² is a 5- or 6-membered heteroaromatic ring, each ring optionally substituted with up to 3 substituents selected from R²⁴; or 5- or 6-membered saturated or partially saturated heterocyclic ring, optionally including 1-2 ring members selected from the group consisting of C(═O), optionally substituted with up to 3 substituents independently selected from R²⁴.
Embodiment A25. A method of Embodiment A24 wherein R² is a 5- or 6-membered heteroaromatic ring, each ring optionally substituted with up to 3 substituents independently selected from R²⁴.
Embodiment A26. A method of Embodiment A25 wherein R² is a 5-membered heteroaromatic ring, each ring optionally substituted with up to 3 substituents independently selected from R²⁴.
Embodiment A27. A method of Embodiment A25 wherein R² is a 6-membered heteroaromatic ring, each ring optionally substituted with up to 3 substituents independently selected from R²⁴.
Embodiment A28. A method of Embodiment A25 wherein R² is 1H-pyrazol-1-yl, 1H-1,2,4-triazol-1-yl, 1H-pyrazol-3-yl or 2-pyridinyl, each optionally substituted with up to 3 substituents independently selected from R²⁴.
Embodiment A29. A method of Embodiment A28 wherein R² is 1H-pyrazol-1-yl, 1H-1,2,4-triazol-1-yl, or 2-pyridinyl, each optionally substituted with up to 3 substituents independently selected from R²⁴.
Embodiment A30. A method of Embodiment A28 wherein R² is 1H-pyrazol-1-yl or 1H-1,2,4-triazol-1-yl.
Embodiment A31. A method of Embodiment A28 wherein R² is 2-pyridinyl.
Embodiment A32. A method of Embodiment A22 wherein each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio or C₃-C₆ trialkylsilyl.
Embodiment A33. A method of Embodiment A32 wherein each R²⁴ is independently halogen, C₁-C₆ alkyl, C₁-C₆ haloalkyl, cyano, nitro, C₁-C₆ alkoxy or C₁-C₆ haloalkoxy.
Embodiment A34. A method of Embodiment A33 wherein each R²⁴ is independently halogen, C₁-C₆ alkyl, C₁-C₆ haloalkyl or cyano.
Embodiment A35. A method of Embodiment A34 wherein each R²⁴ is independently halogen, C₁-C₄ alkyl, C₁-C₄ haloalkyl or cyano.
Embodiment A36. A method of Embodiment A28 wherein R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, C₁-C₆ alkyl, C₁-C₆ haloalkyl or cyano.
Embodiment A37 A method of Embodiment A28 wherein R² is 1H-pyrazol-1-yl, 1H-1,2,4-triazol-1-yl, or 2-pyridinyl, each optionally substituted with from 1 to 3 substituents independently selected from halogen, C₁-C₆ alkyl, C₁-C₆ haloalkyl or cyano.
Embodiment A38. A method of inhibiting undesired animal cellular proliferation said method comprising contacting said animal cells or a tissue or organ in which proliferation of said cell is not desired with a compound of Formula1 wherein R³ is halogen, cyano, C₁-C₆ alkyl, C₁-C₄ haloalkyl, C₃-C₆ cycloalkyl, C₃-C₆ halocycloalkyl, or —CHO.
Embodiment A39. A method of Embodiment A36 wherein R³ is halogen, cyano, C₁-C₆ alkyl or C₁-C₄ haloalkyl.
Embodiment A40. A method of Embodiment A37 wherein R³ is halogen, cyano or C₁-C₆ alkyl.
Embodiment A41. A method of Embodiment A38 wherein R³ is halogen, cyano or C₁-C₃ alkyl.
Embodiment A42. A method of Embodiment A39 wherein R³ is chloro, fluoro, bromo or methyl.
Embodiment A43. A method of inhibiting undesired animal cellular proliferation said method comprising contacting said animal cells or a tissue or organ in which proliferation of said cell is not desired with a compound of Formula 1 wherein J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, each optionally substituted with one or more substituents independently selected from the group consisting of halogen, cyano, nitro, hydroxy, C₁-C₄ alkoxy, C₁-C₄ haloalkoxy, C₂-C₆ alkoxycarbonyl, C₂-C₆ alkylcarbonyl, C₁-C₄ alkylamino and C₂-C₆ dialkylamino; or phenyl, benzyl, naphthalene, 5- or 6-membered heteroaromatic ring, each ring optionally substituted with up to 5 substituents independently selected from R²⁹ and R³⁰.
Embodiment A44. A method of Embodiment A43 wherein J is C₁-C₆ alkyl, C₂-C₆ alkenyl, C₃-C₆ alkynyl, C₃-C₆ cycloalkyl, C₃-C₆ cycloalkenyl, C₄-C₆ cycloalkylalkyl, each optionally substituted with one or more substituents independently selected from the group consisting of halogen, cyano, nitro, hydroxy, C₁-C₄ alkoxy, C₁-C₄ haloalkoxy, C₁-C₄ alkylamino and C₂-C₆ dialkylamino; or phenyl, benzyl, 5- or 6-membered heteroaromatic ring, each ring optionally substituted with up to 4 substituents independently selected from R²⁹ and R³⁰.
Embodiment A45. A method of Embodiment A44 wherein J is phenyl, benzyl, 5- or 6-membered heteroaromatic ring, each ring optionally substituted with up to 4 substituents independently selected from substituents independently selected from R²⁹ and R³⁰.
Embodiment A46. A method of Embodiment A45 wherein J is phenyl, benzyl, 5- or 6-membered heteroaromatic ring, each ring optionally substituted with up to 4 substituents independently selected from halogen, C₁-C₆ alkyl, C₃-C₆ cycloalkyl, C₁-C₆ haloalkyl, cyano, nitro, C₁-C₆ alkoxy, C₁-C₆ haloalkoxy, C₁-C₆ alkylamino, C₂-C₆ dialkylamino and R³⁰.
Embodiment A47. A method of Embodiment A46 wherein J is phenyl, optionally substituted at the 2, 4 and 6 positions with substituents independently selected from halogen, C₁-C₆ alkyl, C₁-C₆ alkoxy and R³⁰.
Embodiment A48. A method of Embodiment A47 wherein J is phenyl, optionally substituted at the 2, 4 and 6 positions with substituents independently selected from chloro, fluoro, methyl, methoxy and R³⁰.
Embodiment A49. A method administering the compound of Formula 1 wherein Y is O or NR³¹.
Embodiment A50. A method of Embodiment A49 wherein Y is O or NH.
Embodiment A51. A method of Embodiment A50 wherein Y is O.
Embodiment A52. A method administering the compound of Formula 1 wherein X is C₁-C₆ alkylene, C₂-C₆ alkenylene or C₃-C₆ cycloalkylene.
Embodiment A53. A method of Embodiment A52 wherein X is C₁-C₆ alkylene or C₂-C₆ alkenylene.
Embodiment A54. A method of Embodiment A53 wherein X is C₂-C₄ alkylene or C₂-C₄ alkenylene.
Embodiment A55. A method of Embodiment A54 wherein X is C₃-C₄ alkylene.
Embodiment A56. A method of inhibiting undesired animal cellular proliferation said method comprising contacting said animal cells or a tissue or organ in which proliferation of said cell is not desired with a compound of Formula 1 wherein Q is NR³²R³³ or OR³⁵.
Embodiment A57. A method of Embodiment A56 wherein Q is NR³²R³³.
Embodiment A58. A method of Embodiment A57 wherein each R³² and R³³ is independently H or C₁-C₆ alkyl, C₁-C₆ haloalkyl, C₃-C₆ cycloalkyl, C₃-C₆ halocycloalkyl, C₂-C₆ alkenyl or C₃-C₆ alkynyl; or R³² and R³³ when optionally taken together with the nitrogen atom to which each R³² and R³³ is attached form a heterocyclic ring of 4 to 6 ring atoms optionally substituted with R³⁴.
Embodiment A59. A method of Embodiment A58 wherein each R³² and R³³ is independently H or C₁-C₆ alkyl, C₁-C₆ haloalkyl, C₃-C₆ cycloalkyl or C₃-C₆ halocycloalkyl; or R³² and R³³ when optionally taken together with the nitrogen atom to which each R³² and R³³ is attached form a heterocyclic ring of 4 to 6 ring atoms optionally substituted with R³⁴.
Embodiment A60. A method of Embodiment A59 wherein each R³² and R³³ is independently H or C₂-C₆ alkyl or C₂-C₆ haloalkyl.
Embodiment A61. A method of Embodiment A60 wherein each R³² and R³³ is independently H or C₂-C₆ alkyl.
Embodiment A62. A method of Embodiment A58 wherein R³⁴ is halogen or C₂-C₆ alkyl.
Embodiment A63. A method of Embodiment A56 wherein R³⁵ is H, C₁-C₆ alkyl, C₁-C₆ haloalkyl, C₃-C₆ cycloalkyl or C₃-C₆ halocycloalkyl.
Embodiment A64. A method of Embodiment A63 wherein R³⁵ is H, C₁-C₆ alkyl or C₁-C₆ haloalkyl.
Embodiment A65. A method of Embodiment A64 wherein R³⁵ is H or C₁-C₆ alkyl.
Embodiment A66. A method of any of Embodiments A1-A65 wherein the compound of Formula 1 inhibits microtubule function.
Embodiment A67. A method of any of Embodiments A1-A66 wherein said undesired cellular proliferation occurs in an individual and wherein said contacting is accomplished by administering to said individual a therapeutically effective amount of the compound of Formula 1.
Embodiment A68. The method of Embodiment A67 wherein the undesired cellular proliferation results in the growth of a neoplasm.
Embodiment A69. The method of Embodiment A68 wherein the neoplasm is selected from the group consisting of mammary, small-cell lung, non-small-cell lung, colorectal, leukemia, lymphoma, melanoma, pancreatic, renal, liver, myeloma, multiple mycloma, mesothelioma, central nervous system, ovarian, prostate, sarcoma of soft tissue or bone, head and neck, esophageal, stomach, bladder, retinoblastoma, squamous cell, testicular, vaginal, and neuroendocrine-related neoplasms
Embodiment A70. The method of Embodiment A69 wherein the neoplasm is cancerous.
The invention includes combinations of Embodiments A1-A65. Combinations of Embodiments A1-A65 are illustrated by:
Embodiment B1. A method of inhibiting undesired cellular proliferation said method comprising contacting said cells or a tissue or organ in which proliferation of said cell is not desired with a compound of Formula 1 wherein
   A is O or S;
   R¹ is C₂-C₆ alkyl, C₂-C₆ haloalkyl, C₃-C₈ cycloalkyl, C₄-C₈ cycloalkylalkyl, NR⁴R⁵, G¹ or G²;
   R² is cyano, —C(W)NR²²R²³ or —NR⁸C(═O)R²⁶; or a 5- or 6-membered heteroaromatic ring; or a 5- or 6-membered saturated or partially saturated heterocyclic ring, optionally including 1-3 ring members selected from the group consisting of C(═O);
   W is O or S;
   R³ is halogen, cyano or C₁-C₆ alkyl;
   X is C₁-C₆ alkylene or C₂-C₆ alkenylene;
   R⁴ and R⁵ are independently H, C₁-C₈ alkyl or C₁-C₈ haloalkyl; and
   J is phenyl optionally substituted with substituents independently selected from halogen, C₁-C₆ alkyl, C₁-C₆ haloalkyl and R³⁰.
Embodiment B2. A method of Embodiment B1 wherein
   A is O;
   R¹ is C₂-C₆ alkyl, C₂-C₆ haloalkyl, C₄-C₈ cycloalkylalkyl, G¹ or G²;
   R² is 5- or 6-membered heteroaromatic ring, cyano, —CONH₂ or —NHC(═O)CH₃;
   R³ is halogen, cyano or C₁-C₃ alkyl;
   X is C₃-C₄ alkylene or C₂-C₄ alkenylene; and
   J is phenyl, optionally substituted at the 2, 3, 4 and 6 positions with substituents independently selected from halogen, C₁-C₆ alkyl, C₁-C₆ haloalkyl and R³⁰.
Embodiment B3. A method of Embodiment B2 wherein
   R¹ is C₃-C₆ alkyl, C₃-C₆ haloalkyl, C₄-C₈ cycloalkylalkyl, or phenyl, optionally substituted with from 1 to 4 substituents independently selected from R¹⁸;
   R² is 5- or 6-membered heteroaromatic ring, each ring optionally substituted with up to 3 substituents independently selected from R²⁴; or —CONH₂ or —NHC(═O)CH₃;
   R³ is fluoro, chloro, bromo or methyl;
   X is C₃-C₄ alkylene; and
   J is phenyl optionally substituted at the 2, 3, 4 and 6 positions with substituents independently selected from chloro and fluoro, methyl, and R³⁰.
Embodiment B4. A method of Embodiment B3 wherein
   R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₆ alkyl or C₁-C₄ haloalkyl; or —CONH₂;
   Y is O or NR³¹; and
   Q is NR³²R³³ or OR³⁵.
Embodiment B5. A method of Embodiment B4 wherein
   R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₄ alkyl or C₁-C₃ haloalkyl; or —CONH₂;
   Y is O or NH; and
   each R³², R³³ and R³⁵ is independently H or C₁-C₄ alkyl or C₁-C₃ haloalkyl.
Embodiment B6. A method of inhibiting undesired cellular proliferation said method comprising contacting said cells or a tissue or organ in which proliferation of said cell is not desired with a compound of Formula1 is selected from:
5-chloro-6-[4-[3-(dimethylamino)propoxy]-2,6-difluorophenyl]-1-[(2S)-2-methylbutyl]-3-(1H-pyrazol-1-yl)-2(1H)-pyrazinone (Compound 482),
5-chloro-1-cyclopropylmethyl-6-[4-[3-(dimethylamino)propoxy]-2,6-difluorophenyl]-3-(1H-pyrazol-1-yl)-2(1H)-pyrazinone (Compound 481),
5-chloro-6-[2,6-difluoro-4-[3-(methylamino)propoxy]phenyl]-1-[(2S)-2-methylbutyl]-3-(1H-pyrazol-1-yl)-2(1H)-pyrazinone,
6-chloro-5-[4-[3-(dimethylamino)propoxy]-2,6-difluorophenyl]-3,4-dihydro-4-[(2S)-2-methylbutyl]-3-oxopyrazinecarboxamide (Compound 486),
6-chloro-5-[2,6-difluoro-4-[3-(methylamino)propoxy]phenyl]-3,4-dihydro-4-[(2S)-2-methylbutyl]-3-oxopyrazinecarboxamide,
6-chloro-5-[4-[3-(dimethylamino)propoxy]-2,6-difluorophenyl]-3,4-dihydro-3-oxo-4-(3,3,3-trifluoro-2-methylpropyl)pyrazinecarboxamide,
6-chloro-5-[2,6-difluoro-4-[3-(methylamino)propoxy]phenyl]-3,4-dihydro-3-oxo-4-(3,3,3-trifluoro-2-methylpropyl)pyrazinecarboxamide,
5-chloro-6-[4-[3-(dimethylamino)propoxy]-2,6-difluorophenyl]-1-(3-fluorophenyl)-3-(1H-pyrazol-1-yl)-2(1H)-pyrazinone,
5-chloro-6-[2,6-difluoro-4-[3-(methylamino)propoxy]phenyl]-1-(3-fluorophenyl)-3-(1H-pyrazol-1-yl)-2(1H)-pyrazinone,
5-chloro-6-[4-[3-(dimethylamino)propoxy]-2,6-difluorophenyl]-3-(1H-pyrazol-1-yl)-1-(3,3,3-trifluoro-2-methylpropyl)-2(1H)-pyrazinone (Compound 485),
5-chloro-6-[2,6-difluoro-4-[3-(methylamino)propoxy]phenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-1-[(2S)-2-methylbutyl]-3-(3-methyl-1H-pyrazol-1-yl)-2(1H)-pyrazinone (Compound 494),
5-chloro-6-[4-[3-(dimethylamino)propoxy]-2,6-difluorophenyl]-1-[(2S)-2-methylbutyl]-3-(3-methyl-1H-pyrazol-1-yl)-2(1H)-pyrazinone (Compound 498),
5-chloro-6-[2-chloro-6-fluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(1H-pyrazol-1-yl)-2(1H)-pyrazinone (Compound 493),
5-chloro-6-[2,6-difluoro-4-[3-(methylamino)propoxy]phenyl]-1-[(2S)-2-methylbutyl]-3-(1-methyl-1H-pyrazol-3-yl)-2(1H)-pyrazinone (Compound 502),
5-Chloro-1-[(2S)-2-methylbutyl)-3-(1H-pyrazol-1-yl)-6-(2,4,6-trifluorophenyl)-2(1H)-pyrazinone (Compound 155),
5-Chloro-1-[(2S)-2-methylbutyl)-3-(1H-pyrazol-1-yl)-6-(2,6-difluoro-4-methoxyphenyl)-2(1H)-pyrazinone (Compound 457), and
5-Chloro-1-[(2S)-2-methylbutyl)-3-(1H-3-methyl-pyrazol-1-yl)-6-(2,6-difluoro-4-methoxyphenyl)-2(1H)-pyrazinone (Compound 490).
   Embodiment B7. A method of any of Embodiments B1-B6 wherein the compound of Formula 1 inhibits microtubule function.
   Embodiment B8. A method of any of Embodiments B1-B6 wherein said undesired cellular proliferation occurs in an individual and wherein said contacting is accomplished by administering to said individual a therapeutically effective amount of the compound of Formula 1.
   Embodiment B9. The method of Embodiment B8 wherein the undesired cellular proliferation results in the growth of a neoplasm
   Embodiment B10. The method of Embodiment B9 wherein the neoplasm is selected from the group consisting of mammary, small-cell lung, non-small-cell lung, colorectal, leukemia, lymphoma, melanoma, pancreatic, renal, liver, myeloma, multiple mycloma, mesothelioma, central nervous system, ovarian, prostate, sarcoma of soft tissue or bone, head and neck, esophageal, stomach, bladder, retinoblastoma, squamous cell, testicular, vaginal, and neuroendocrine-related neoplasms
   Embodiment B11. The method of Embodiment B10 wherein the neoplasm is cancerous.
   Embodiment C1. A compound of Formula 1 or a salt thereof wherein
      R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₅-C₁₀ alkylcycloalkylalkyl, C₇-C₁₄ alkylcycloalkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
      A is O, S or NR⁷;
      R⁷ is H, C₁-C₄ alkyl, C₁-C₄ haloalkyl, C₂-C₆ alkylcarbonyl or C₂-C₆ alkoxycarbonyl;
      R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³, —NR⁸C(O)R²⁶, —NR⁸C(O)NR²⁷ or —NR⁸C(O)OR²⁸; or
      R² is a 5- or 6-membered heteroaromatic ring or a 8-, 9- or 10-membered heteroaromatic bicyclic ring system, each ring or ring system optionally substituted with up to 5 substituents independently selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴; or
      R² and R⁷ are taken together as —N═C(R¹⁶)—;
      W is O, S or ═NR²⁵;
      R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
      each R⁴ and R⁵ is independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
      R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
      R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
      each R⁸ is independently H, C₁-C₄ alkyl or C₁-C₄ haloalkyl;
      R⁹ is C₁-C₄ alkyl or C₁-C₄ haloalkyl;
      R¹⁰ is H, C₁-C₄ alkyl or C₁-C₄ haloalkyl; or
      R⁹ and R¹⁰ are taken together as —(CH₂)₃—, —(CH₂)₄—, —(CH₂)₅— or —(CH₂)₆—;
      R¹¹ is H, C₁-C₄ alkyl or C₁-C₄ haloalkyl;
      R¹² is H, C₁-C₄ alkyl, C₁-C₄ haloalkyl, C₂-C₃ alkylcarbonyl or C₂-C₃ alkoxycarbonyl; or
      R¹¹ and R¹² are taken together as —(CH₂)₄—, —(CH₂)₅, —CH₂CH₂OCH₂CH₂— or —CH₂CH(CH₃)OCH(CH₃)CH₂—;
      R¹³ is H, NH₂, C₁-C₄ alkyl or C₁-C₄ haloalkyl;
      R¹⁴ is H, C₁-C₄ alkyl or C₁-C₄ haloalkyl;
      R¹⁶ is H, halogen, cyano, C₁-C₆ alkyl, C₁-C₄ haloalkyl, C₃-C₆ cycloalkyl, C₃-C₆ halocycloalkyl, C₂-C₆ alkenyl, C₂-C₆ alkynyl, C₁-C₄ alkoxy, C₁-C₄ haloalkoxy, C₁-C₄ alkylthio, C₁-C₄ haloalkylthio or C₂-C₅ alkoxycarbonyl;
      J is a phenyl, benzyl, naphthalene, 5- or 6-membered heteroaromatic ring or 8-, 9- or 10-membered heteroaromatic bicyclic ring system, each ring or ring system substituted with 1 to 2 substituents independently selected from R³⁰ and optionally substituted up to 4 substituents independently selected from R²⁹;
      R²⁹ is halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
      R³⁰ is —Y-X-Q;
      Y is O, S(O)ₚ, NR³¹ or direct bond;
      X is C₁-C₆ alkylene, C₂-C₆ alkenylene, C₃-C₆ alkynylene, C₃-C₆ cycloalkylene or C₃-C₆ cycloalkenylene, each optionally substituted with one or more substituents independently selected from the group consisting of halogen, cyano, nitro, hydroxy, (═O), C₁-C₆ alkoxy and C₁-C₆ haloalkoxy;
      Q is NR³²R³³, OR³⁵ or S(O)ₚR³⁵;
      R³¹ is H or C₁-C₆ alkyl, C₁-C₆ haloalkyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
      each R³² and R³³ is independently H; or C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl; or R³² and R³³ when optionally taken together with the nitrogen atom to which each is attached form a heterocyclic ring of 3 to 6 ring atoms optionally substituted with R³⁴;
      R³⁴ is halogen, C₁-C₆ alkyl, C₁-C₆ haloalkyl or C₁-C₆ alkoxy;
      each R³⁵ is independently H, C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
      p is 0, 1 or 2;
      G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents independently selected from R¹⁷;
      G² is a phenyl ring, 5- or 6-membered heteroaromatic ring, each ring or ring system optionally substituted with from 1 to 4 substituents independently selected from R¹⁸;
      each R¹⁷ is independently C₁-C₂ alkyl, C₁-C₂ haloalkyl, halogen, cyano, nitro or
      C₁-C₂ alkoxy;
      each R¹⁸ is independently C₁-C₄ alkyl, C₂-C₄ alkenyl, C₂-C₄ alkynyl, C₃-C₆
      cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
      each R¹⁹ and R²¹ is independently H, C₁-C₄ alkyl, C₁-C₄ haloalkyl or C₃-C₈ cycloalkyl; or
      R¹⁹ and R²¹ are taken together as —(CH₂)₄—, —(CH₂)₅, —CH₂CH₂OCH₂CH₂— or —CH₂CH(CH₃)OCH(CH₃)CH₂—;
      each R²² and R²³ is independently H; or C₁-C₄ alkyl, C₁-C₄ alkoxy, C₃-C₈ cycloalkyl or C₄-C₈ cycloalkylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
      R²² and R²³ are taken together as —(CH₂)₄—, —(CH₂)₅, —CH₂CH₂OCH₂CH₂— or —CH₂CH(CH₃)OCH(CH₃)CH₂—;
      each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
      R²⁵ is H, C₁-C₄ alkyl or C₁-C₄ haloalkyl; and
      R²⁶ is H, C₁-C₆ alkyl, C₁-C₄ haloalkyl, C₃-C₆ cycloalkyl, C₃-C₆ halocycloalkyl,
      C₂-C₆ alkenyl or C₃-C₆ alkynyl; or phenyl ring, 5- or 6-membered heteroaromatic ring, each ring or ring system optionally substituted with from 1 to 4 substituents independently selected from R³⁶;
      R³⁶ is C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy or C₁-C₄ haloalkoxy; and
      each R²⁷ and R²⁸ is independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, halogen, cyano, nitro, C₁-C₄ alkoxy and C₁-C₄ haloalkoxy.
   Embodiment C2. A compound of Embodiment Cl wherein R¹ is C₁-C₈ alkyl, C₁-C₈ haloalkyl, C₃-C₈ cycloalkyl, C₄-C₈ cycloalkylalkyl, NR⁴R⁵, —N═CR¹⁹R²¹, G¹ or G².
   Embodiment C3. A compound of Embodiment C2 wherein R¹ is C₂-C₆ alkyl, C₂-C₆ haloalkyl, C₄-C₈ cycloalkylalkyl, NR⁴R⁵, G¹ or G².
   Embodiment C4. A compound of Embodiment C3 wherein R¹ is C₂-C₆ alkyl, C₂-C₆ haloalkyl or C₄-C₈ cycloalkylalkyl.
   Embodiment C5. A compound of Embodiment C4 wherein R¹ is C₃-C₆ alkyl, C₃-C₆ haloalkyl or C₄-C₆ cyclopropylalkyl.
   Embodiment C6. A compound of Embodiment C5 wherein R¹ is NR⁴R⁵.
   Embodiment C7. A compound of Embodiment C2 wherein R¹ is G¹.
   Embodiment C8. A compound of Embodiment C2 wherein R¹ is G².
   Embodiment C9. A compound of Embodiment C3 wherein each R⁴ and R⁵ is independently H, C₁-C₈ alkyl or C₁-C₈ haloalkyl.
   Embodiment C10. A compound of Embodiment C9 wherein each R⁴ and R⁵ is independently H, C₃-C₆ alkyl or C₃-C₆ haloalkyl.
   Embodiment C11. A compound of Embodiment C7 wherein G¹ is a 5- to 6-membered nonaromatic carbocyclic or heterocyclic ring, optionally including 1 or 2 ring members selected from the group consisting of C(═O), C(═S), S(O) and S(O)₂.
   Embodiment C12. A compound of Embodiment C11 wherein G¹ is a 5- to 6-membered nonaromatic carbocyclic or heterocyclic ring, optionally including 1 or 2 ring members selected from the group consisting of C(═O).
   Embodiment C13. A compound of Embodiment C8 wherein G² is a phenyl ring, optionally substituted with from 1 to 4 substituents independently selected from R¹⁸.
   Embodiment C14. A compound of Embodiment C8 wherein G² is a 5- or 6-membered heteroaromatic ring, each ring or ring system optionally substituted with from 1 to 4 substituents independently selected from R¹⁸.
   Embodiment C15. A compound of Embodiment C1 wherein A is O or S.
   Embodiment C16. A compound of Embodiment C15 wherein A is O.
   Embodiment C 17. A compound of Embodiment C1 wherein R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³ or —NR⁸C(═O)R²⁶.
   Embodiment C18. A compound of Embodiment C17 wherein R² is cyano, —NR⁸N═CR⁹R¹⁰, —CR¹³═NOR¹⁴, —CR¹³═NNR¹¹R¹², —C(W)NR²²R²³ or —NR⁸C(═O)R²⁶.
   Embodiment C19. A compound of Embodiment C18 wherein R² is cyano, —C(W)NR²²R²³ or —NR⁸C(═O)R²⁶.
   Embodiment C20. A compound of Embodiment C19 wherein R² is cyano, —CONH₂ or —NHC(═O)CH₃.
   Embodiment C21. A compound of Embodiment C19 wherein W is O.
   Embodiment C22. A compound of Embodiment C19 wherein each R²² and R²³ is independently H or C₁-C₄ alkyl.
   Embodiment C23. A compound of Embodiment C1 wherein R² is a 5- or 6-membered heteroaromatic ring, each ring optionally substituted with up to 5 substituents selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴.
   Embodiment C24. A compound of Embodiment C23 wherein R² is a 5- or 6-membered heteroaromatic ring, each ring optionally substituted with up to 4 substituents selected from R²⁴; or 5- or 6-membered saturated or partially saturated heterocyclic ring, optionally including 1-3 ring members selected from the group consisting of C(═O), optionally substituted with up to 5 substituents independently selected from R²⁴.
   Embodiment C25. A compound of Embodiment C24 wherein R² is a 5- or 6-membered heteroaromatic ring, each ring optionally substituted with up to 3 substituents selected from R²⁴; or 5- or 6-membered saturated or partially saturated heterocyclic ring, optionally including 1-2 ring members selected from the group consisting of C(═O), optionally substituted with up to 3 substituents independently selected from R²⁴.
   Embodiment C26. A compound of Embodiment C25 wherein R² is a 5- or 6-membered heteroaromatic ring, each ring optionally substituted with up to 3 substituents independently selected from R²⁴.
   Embodiment C27. A compound of Embodiment C26 wherein R² is a 5-membered heteroaromatic ring, each ring optionally substituted with up to 3 substituents independently selected from R²⁴.
   Embodiment C28. A compound of Embodiment C26 wherein R² is a 6-membered heteroaromatic ring, each ring optionally substituted with up to 3 substituents independently selected from R²⁴.
   Embodiment C29. A compound of Embodiment C26 wherein R² is 1H-pyrazol-1-yl, 1H-1,2,4-triazol-1-yl, 1H-pyrazol-3-yl or 2-pyridinyl, each optionally substituted with up to 3 substituents independently selected from R²⁴.
   Embodiment C29a. A compound of Embodiment C29 wherein R² is 1H-pyrazol-1-yl, 1H-1,2,4-triazol-1-yl or 2-pyridinyl, each optionally substituted with up to 3 substituents independently selected from R²⁴.
   Embodiment C30. A compound of Embodiment C29 wherein R² is 1H-pyrazol-1-yl or 1H-1,2,4-triazol-1-yl.
   Embodiment C31. A compound of Embodiment C29 wherein R² is 2-pyridinyl.
   Embodiment C32. A compound of Embodiment C23 wherein each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio or C₃-C₆ trialkylsilyl.
   Embodiment C33. A compound of Embodiment C32 wherein each R²⁴ is independently halogen, C₁-C₆ alkyl, C₁-C₆ haloalkyl, cyano, nitro, C₁-C₆ alkoxy or C₁-C₆ haloalkoxy.
   Embodiment C34. A compound of Embodiment C33 wherein each R²⁴ is independently halogen, C₁-C₆ alkyl, C₁-C₆ haloalkyl or cyano.
   Embodiment C35. A compound of Embodiment C34 wherein each R²⁴ is independently halogen, C₁-C₄ alkyl, C₁-C₄ haloalkyl or cyano.
   Embodiment C36. A compound of Embodiment C29 wherein R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, C₁-C₆ alkyl, C₁-C₆ haloalkyl or cyano.
   Embodiment C36a. A compound of Embodiment C36 wherein R² is 1H-pyrazol-1-yl, 1H-1,2,4-triazol-1-yl or 2-pyridinyl, each optionally substituted with from 1 to 3 substituents independently selected from halogen, C₁-C₆ alkyl, C₁-C₆ haloalkyl or cyano.
   Embodiment C37. A compound of Embodiment C1 wherein R³ is halogen, cyano, C₁-C₆ alkyl, C₁-C₄ haloalkyl, C₃-C₆ cycloalkyl, C₃-C₆ halocycloalkyl, or —CHO.
   Embodiment C38. A compound of Embodiment C37 wherein R³ is halogen, cyano, C₁-C₆ alkyl or C₁-C₄ haloalkyl.
   Embodiment C39. A compound of Embodiment C38 wherein R³ is halogen, cyano or C₁-C₆ alkyl.
   Embodiment C40. A compound of Embodiment C39 wherein R³ is halogen, cyano or C₁-C₃ alkyl.
   Embodiment C41. A compound of Embodiment C40 wherein R³ is chloro, fluoro, bromo or methyl.
   Embodiment C42. A compound of Embodiment C1 wherein J is a phenyl, benzyl, naphthalene, 5- or 6-membered heteroaromatic ring or 8-, 9- or 10-membered heteroaromatic bicyclic ring system, each ring or ring system substituted with one substituent selected from R³⁰ and optionally substituted up to 4 substituents independently selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl and C₃-C₆ trialkylsilyl.
   Embodiment C43. A compound of Embodiment C42 wherein J is phenyl, benzyl, 5- or 6-membered heteroaromatic ring, each ring or ring system substituted with one substituent selected from R³⁰ and optionally substituted up to 4 substituents independently selected from halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylamino, C₂-C₆ dialkylamino, C₂-C₆ alkylcarbonyl, C₂-C₆ alkoxycarbonyl, C₂-C₆ alkylaminocarbonyl and C₃-C₆ dialkylaminocarbonyl.
   Embodiment C44. A compound of Embodiment C43 wherein J is phenyl, benzyl, 5- or 6-membered heteroaromatic ring, each ring or ring system substituted with one substituent selected from R³⁰ and optionally substituted up to 4 substituents independently selected from halogen, C₁-C₆ alkyl, C₃-C₆ cycloalkyl, C₁-C₆ haloalkyl, cyano, nitro, C₁-C₆ alkoxy, C₁-C₆ haloalkoxy, C₁-C₆ alkylamino and C₂-C₆ dialkylamino.
   Embodiment C45. A compound of Embodiment C44 wherein J is phenyl substituted with one substituent selected from R³⁰ and optionally substituted up to 4 substituents independently selected from halogen, C₁-C₆ alkyl, C₃-C₆ cycloalkyl, C₁-C₆ haloalkyl, cyano, nitro, C₁-C₆ alkoxy, C₁-C₆ haloalkoxy, C₁-C₆ alkylamino and C₂-C₆ dialkylamino.
   Embodiment C46. A compound of Embodiment C45 wherein J is phenyl substituted at the 4 position with one substituent selected from R³⁰ and optionally substituted up to 4 substituents independently selected from halogen, C₁-C₆ alkyl, C₃-C₆ cycloalkyl, C₁-C₆ haloalkyl, cyano, nitro, C₁-C₆ alkoxy, C₁-C₆ haloalkoxy, C₁-C₆ alkylamino and C₂-C₆ dialkylamino.
   Embodiment C47. A compound of Embodiment C46 wherein J is phenyl substituted at the 4 position with one substituent selected from R³⁰.
   Embodiment C48. A compound of Embodiment C1 wherein Y is O or NR³¹.
   Embodiment C49. A compound of Embodiment C48 wherein Y is O or NH.
   Embodiment C50. A compound of Embodiment C49 wherein Y is O.
   Embodiment C51. A compound of Embodiment C1 wherein X is C₁-C₆ alkylene, C₂-C₆ alkenylene or C₃-C₆ cycloalkylene.
   Embodiment C52. A compound of Embodiment C51 wherein X is C₁-C₆ alkylene or C₂-C₆ alkenylene.
   Embodiment C53. A compound of Embodiment C52 wherein X is C₂-C₄ alkylene or C₂-C₄ alkenylene.
   Embodiment C54. A compound of Embodiment C53 wherein X is C₃-C₄ alkylene.
   Embodiment C55. A compound of Embodiment Cl wherein Q is NR³²R³³ or OR³⁵.
   Embodiment C56. A compound of Embodiment C55 wherein Q is NR³²R³³.
   Embodiment C57. A compound of Embodiment C56 wherein each R³² and R³³ is independently H or C₁-C₆ alkyl, C₁-C₆ haloalkyl, C₃-C₆ cycloalkyl, C₃-C₆ halocycloalkyl, C₂-C₆ alkenyl or C₃-C₆ alkynyl; or R³² and R³³ when optionally taken together with the nitrogen atom to which each R³² and R³³ is attached form a heterocyclic ring of 4 to 6 ring atoms optionally substituted with R³⁴.
   Embodiment C58. A compound of Embodiment C57 wherein each R³² and R³³ is independently H or C₁-C₆ alkyl, C₁-C₆ haloalkyl, C₃-C₆ cycloalkyl or C₃-C₆ halocycloalkyl; or R³² and R³³ when optionally taken together with the nitrogen atom to which each is attached form a heterocyclic ring of 4 to 6 ring atoms optionally substituted with R³⁴.
   Embodiment C59. A compound of Embodiment C58 wherein each R³² and R³³ is independently H or C₂-C₆ alkyl or C₂-C₆ haloalkyl.
   Embodiment C60. A compound of Embodiment C59 wherein each R³² and R³³ is independently H or C₂-C₆ alkyl.
   Embodiment C61. A compound of Embodiment C57 wherein R³⁴ is halogen or C₂-C₆ alkyl.
   Embodiment C62. A compound of Embodiment C55 wherein R³⁵ is H, C₁-C₆ alkyl, C₁-C₆ haloalkyl, C₃-C₆ cycloalkyl or C₃-C₆ halocycloalkyl.
   Embodiment C63. A compound of Embodiment C62 wherein R³⁵ is H, C₁-C₆ alkyl or C₁-C₆ haloalkyl.
   Embodiment C64. A compound of Embodiment C63 wherein R³⁵ is H or C₁-C₆ alkyl.
   The invention includes combinations of Embodiments C1-C64. Combinations of Embodiments C1-C64 are illustrated by:
   Embodiment D1. A compound of Embodiment C1 wherein
      A is O or S;
      R¹ is C₂-C₆ alkyl, C₂-C₆ haloalkyl, C₃-C₈ cycloalkyl, C₄-C₈ cycloalkylalkyl, NR⁴R⁵, G¹ or G²;
      R² is cyano, —C(W)NR²²R²³ or —NR⁸C(═O)R²⁶; or a 5- or 6-membered heteroaromatic ring; or a 5- or 6-membered saturated or partially saturated heterocyclic ring, optionally including 1-3 ring members selected from the group consisting of C(═O);
      W is O or S;
      R³ is halogen, cyano or C₁-C₆ alkyl;
      X is C₁-C₆ alkylene or C₂-C₆ alkenylene;
      R⁴ and R⁵ are independently H, C₁-C₈ alkyl or C₁-C₈ haloalkyl; and
      J is phenyl substituted with R³⁰.
   Embodiment D2. A compound of Embodiment D1 wherein
      A is O;
      R¹ is C₂-C₆ alkyl, C₂-C₆ haloalkyl, C₄-C₈ cycloalkylalkyl, G¹ or G²;
      R² is 5- or 6-membered heteroaromatic ring, cyano, —CONH₂ or —NHC(═O)CH₃;
      R³ is halogen, cyano or C₁-C₃ alkyl;
      X is C₃-C₄ alkylene or C₂-C₄ alkenylene; and
      J is phenyl substituted at the 4 position with R³⁰.
   Embodiment D3. A compound of Embodiment D2 wherein
      R¹ is C₃-C₆ alkyl, C₃-C₆ haloalkyl, C₄-C₈ cycloalkylalkyl, or phenyl, optionally substituted with from 1 to 4 substituents independently selected from R¹⁸;
      R² is 5- or 6-membered heteroaromatic ring, each ring optionally substituted with up to 3 substituents independently selected from R²⁴; or —CONH₂ or —NHC(═O)CH₃;
      R³ is fluoro, chloro, bromo or methyl;
      Y is O or NH;
      X is C₃-C₄ alkylene;
      Q is NR³²R³³ or OR³⁵;
      each R³² and R³³ is independently H or C₂-C₆ alkyl or C₂-C₆ haloalkyl; and
      R³⁵ is H, C₁-C₆ alkyl or C₁-C₆ haloalkyl.
   Embodiment D4. A compound of Embodiment D3 wherein
      R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₆ alkyl or C₁-C₄ haloalkyl; or —CONH₂;
      Y is NH; and
      Q is NR³²R³³.
   Embodiment D5. A compound of Embodiment D4 wherein
      R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₄ alkyl or C₁-C₃ haloalkyl; or —CONH₂; and
      each R³², R³³ and R³⁵ is independently H or C₁-C₄ alkyl or C₁-C₃ haloalkyl.
   Embodiment D6. A compound of Embodiment C1 selected from the group consisting of:
5-chloro-6-[4-[3-(dimethylamino)propoxy]-2,6-difluorophenyl]-1-[(2S)-2-methylbutyl]-3-(1H-pyrazol-1-yl)-2(1H)-pyrazinone (Compound 482),
5-chloro-1-cyclopropylmethyl-6-[4-[3-(dimethylamino)propoxy]-2,6-difluorophenyl]-3-(1H-pyrazol-1-yl)-2(1H)-pyrazinone (Compound 481),
5-chloro-6-[2,6-difluoro-4-[3-(methylamino)propoxy]phenyl]-1-[(2S)-2-methylbutyl]-3-(1H-pyrazol-1-yl)-2(1H)-pyrazinone,
6-chloro-5-[4-[3-(dimethylamino)propoxy]-2,6-difluorophenyl]-3,4-dihydro-4-[(2S)-2-methylbutyl]-3-oxopyrazinecarboxamide (Compound 486),
6-chloro-5-[2,6-difluoro-4-[3-(methylamino)propoxy]phenyl]-3,4-dihydro-4-[(2S)-2-methylbutyl]-3-oxopyrazinecarboxamide,
6-chloro-5-[4-[3-(dimethylamino)propoxy]-2,6-difluorophenyl]-3,4-dihydro-3-oxo-4-(3,3,3-trifluoro-2-methylpropyl)pyrazinecarboxamide,
6-chloro-5-[2,6-difluoro-4-[3-(methylamino)propoxy]phenyl]-3,4-dihydro-3-oxo-4-(3,3,3-trifluoro-2-methylpropyl)pyrazinecarboxamide,
5-chloro-6-[4-[3-(dimethylamino)propoxy]-2,6-difluorophenyl]-1-(3-fluorophenyl)-3-(1H-pyrazol-1-yl)-2(1H)-pyrazinone,
5-chloro-6-[2,6-difluoro-4-[3-(methylamino)propoxy]phenyl]-1-(3-fluorophenyl)-3-(1H-pyrazol-1-yl)-2(1H)-pyrazinone,
5-chloro-6-[4-[3-(dimethylamino)propoxy]-2,6-difluorophenyl]-3-(1H-pyrazol-1-yl)-1-(3,3,3-trifluoro-2-methylpropyl)-2(1H)-pyrazinone (Compound 485),
5-chloro-6-[2,6-difluoro-4-[3-(methylamino)propoxy]phenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-1-[(2S)-2-methylbutyl]-3-(3-methyl-1H-pyrazol-1-yl)-2(1H)-pyrazinone (Compound 494),
5-chloro-6-[4-[3-(dimethylamino)propoxy]-2,6-difluorophenyl]-1[(2S)-2-methylbutyl]-3-(3-methyl-1H-pyrazol-1-yl)-2(1H)-pyrazinone (Compound 498),
5-chloro-6-[2-chloro-6-fluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(1H-pyrazol-1-yl)-2(1H)-pyrazinone (Compound 493),
5-chloro-6-[2,6-difluoro-4-[3-(methylamino)propoxy]phenyl]-1-[(2S)-2-methylbutyl]-3-(1-methyl-1H-pyrazol-3-yl)-2(1H)-pyrazinone (Compound 502),
5-Chloro-1-[(2S)-2-methylbutyl)-3-(1H-pyrazol-1-yl)-6-(2,4,6-trifluorophenyl)-2(1H)-pyrazinone (Compound 155),
5-Chloro-1-[(2S)-2-methylbutyl)-3-(1H-pyrazol-1-yl)-6-(2,6-difluoro-4-methoxyphenyl)-2(1H)-pyrazinone (Compound 457), and
5-Chloro-1-[(2S)-2-methylbutyl)-3-(1H-3-methyl-pyrazol-1-yl)-6-(2,6-difluoro-4-methoxyphenyl)-2(1H)-pyrazinone (Compound 490).

Also of note is a method of inhibiting undesired animal cellular proliferation, said method comprising contacting said animal cells or a tissue or organ in which proliferation of said cell is not desired with a compound of Formula1 wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G².

Also of note are compounds of Formula 1 or salts thereof, wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G².

Of note is a composition which comprises a compound of any one of Embodiments C1 through C64 and D1 through D6 or a pharmaceutically acceptable salt thereof optionally with a physiologically acceptable carrier.

Of note is a method of inhibiting undesired animal cellular proliferation said method comprising contacting an animal cell with the compound or composition comprising the compound of any of Embodiments C1 through C64 and D1 through D6.

Of further note is a method as noted above wherein said animal cell is comprised within a tissue or organ in which proliferation of said cell is not desired.

Of further note is a method as noted above wherein the compound of Formula 1 inhibits microtubule function.

Of further note is a method as noted above wherein polymerization is inhibited.

Of further note is a method as noted above wherein polymerized tubulin or microtubule structures are stabilized.

The compounds of Formula 1 can be prepared by one or more of the following methods and variations as described in Schemes 1-20. The definitions of R¹, R², R³, R¹¹, R¹², R¹³, R¹⁴, R¹⁹, R²¹, R²², R²³, A and J in the compounds of Formulae1-32 below are as defined above in the Summary of the Invention. Compounds of Formulae1a-1t are various subsets of the compounds of Formula1.

Compounds of Formula 1 wherein R² is a heterocycle linked through N can be made as shown in Scheme 1. Reaction of an heterocycle comprising NH of Formula 3 with a compound of Formula 2 wherein X¹ is a suitable leaving group such as halogen (e.g., Cl, Br, I), OS(O)₂CH₃ (methanesulfonate), OS(O)₂CF₃, OS(O)₂Ph-p-CH₃ (p-toluenesulfonate) or other nucleofuge as outlined in Scheme 1 in the presence of an acid acceptor gives the compounds of Formula 1 in which R² is a N-linked heterocycle. Suitable acid acceptors for the reaction include inorganic bases, such as alkali or alkaline earth metal (such as lithium, sodium, potassium, cesium) hydrides, alkoxides, carbonates, phosphates and hydroxides, and organic bases, such as triethylamine, pyrazole, N,N-diisopropylethylamine and 1,8-diazabicyclo[5.4.0]undec-7-ene. Preferred acid acceptors are potassium carbonate and potassium hydroxide. A wide variety of solvents are suitable for the reaction, including, for example but not limitation, N,N-dimethylformamide, N,N-dimethylacetamide, N-methylpyrrolidinone, acetonitrile and acetone, as well as mixtures of these solvents. This reaction can be conducted between about 0 and 200° C., and preferably between about 20 and 80° C.

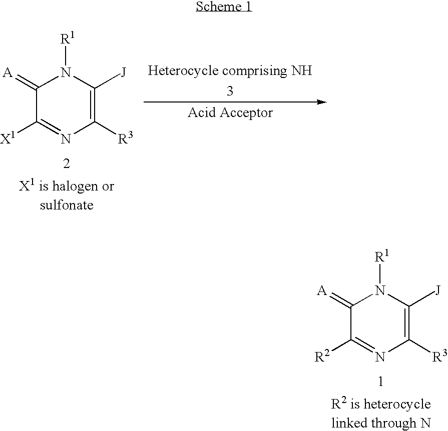

As shown in Scheme 2, compounds of Formula 1 in which R² is a hydrazone, oxime, hydrazine derivative or hydroxylamine derivative can be synthesized by a reaction of the appropriate nucleophile of Formula 4 with a compound of Formula 2 in the presence of an acid acceptor. Preferred solvents include N,N-dimethylformamide, N,N-dimethylacetamide, N-methylpyrrolidinone, acetonitrile and acetone. Acid acceptors such as tertiary amines, alkali carbonates, alkali hydroxides and alkali hydrides may be used in this reaction. Potassium carbonate and tertiary amines such as triethylamine are preferred acid acceptors for hydrazones and hydrazines. Alkali metal hydrides such as sodium hydride are preferred acid acceptors for the oximes and hydroxylamines.

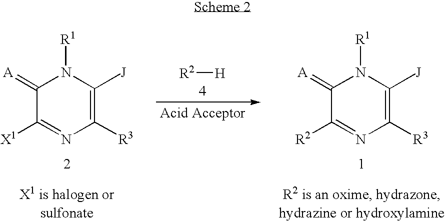

Compounds of Formula 1a and Formula 1b can be synthesized as shown in Scheme 3. Reaction of compounds of Formula 2 with a cyanide salt gives the products of Formula 1a. The reaction may be carried out in protic or aprotic solvents. Preferred solvents are N,N-dimethylformamide, lower alcohols and mixtures of these solvents with water. The reaction may be successfully carried out at temperatures from 0 to 200° C., with temperatures of 60-120° C. preferred. Compounds of Formula 1b may be obtained from the reaction of compounds of Formula 1a with hydrogen sulfide or other sulfide source. This reaction may be carried out in a variety of solvents and temperatures. Reaction in mixtures of lower alcohols and water is preferred. For a convenient procedure using ammonium as the sulfide source see Bagley et. al., Synlett, 2004, 2615-2617.

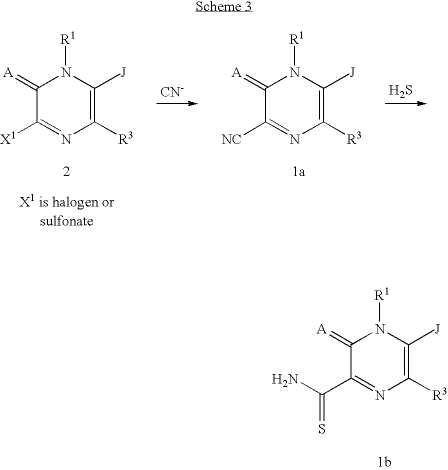

As shown in Scheme 4, compounds of Formula 1 wherein R² is a C-linked heterocycle can be obtained by transition metal-catalyzed reactions of compounds of Formula 2 wherein X¹ is halogen with compounds of Formula 5. Transition metal catalyzed cross coupling reactions of halopyrazinones are known from the work of Hoornaert et al., Tetrahedron, 1991, 47, 9259-9268 and Tetrahedron Letters, 2004, 45, 1885-1888. Reaction of various organometallic heterocycles of Formula 5 under palladium or nickel catalysis is possible. For synthesis of organometallic heterocycles suitable for use in this reaction see, Gribble and Li, “Palladium in Heterocyclic Chemistry”, Pergamon Press, Amsterdam, 2000, page 411. This book also describes a wide variety of catalysts and reaction conditions suitable for carrying out the cross coupling reactions described in Scheme 4. When the metal is magnesium, the coupling does not necessarily require added transition metal catalyst.

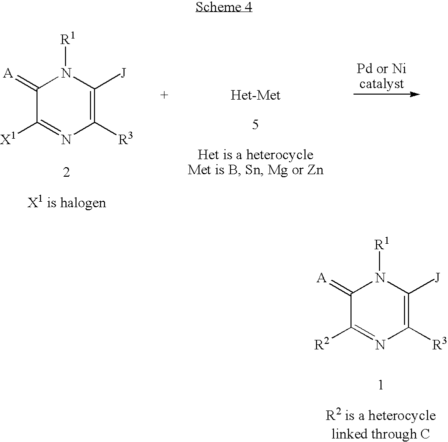

Compounds of Formula 1 wherein R² is a C-linked heterocycle can also be obtained by the conversion of a halogen substituted pyrazinone of Formula 2 into an organometallic derivative followed by a cross coupling reaction as shown in Scheme 5. Most preferably the organometallic pyrazinone is made by the reaction of a bimetallic reagent such as hexamethylditin with compounds of Formula 2 under palladium catalysis. Other reagents such as pinacolatodiborane may also be used. The resulting tin compound of Formula 6 can be transformed to compounds of Formula 1 by palladium-catalyzed coupling with haloheterocycles of Formula 7. Examples of this reaction to make heterocyclic tin compounds may be found in Majeed et al., Tetrahedron, 1989, 45, 993-1006.

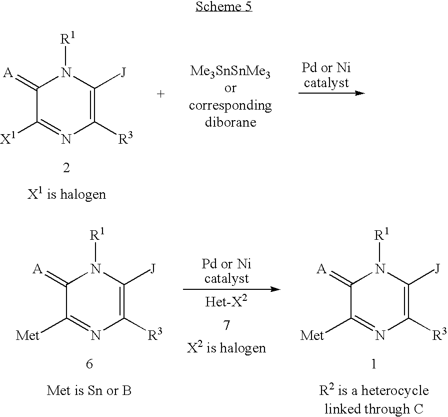

Compounds of Formula 1d (i.e. Formula 1 wherein R³ is alkoxy, thioalkyl or cyano) can be synthesized by the reaction of a halopyrazinone of Formula 1c with the appropriate nucleophile as shown in Scheme 6. The compound of Formula 1c is treated in an aprotic solvent with the appropriate nucleophile at temperatures between about 0 and 160° C. In the case of cyanide and thioalkyl nucleophiles the reaction is best carried out in solvents such as N,N-dimethylformamide and N-methylpyrrolidinone. In the case of alkoxides, the reaction is best carried out in the alcohol from which the alkoxide is generated. Among appropriate acid acceptors are alkali metals such as sodium hydride. In the case of cyanide an acid acceptor is not necessary.

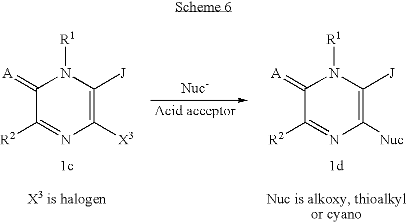

In compounds of Formula 1 R³ is an alkyl, alkenyl, alkynyl or cycloalkyl group may be introduced by means of transition metal-catalyzed reactions involving compounds of Formula 1c as shown in Scheme 7. The alkyl, alkenyl, alkynyl or cycloalkyl metal species may be derived from B, Sn, Si, Mg, Al or Zn. Conditions for the couplings are as described previously in Scheme 4, and description of conditions for these transformations is found in Gribble and Li (“Palladium in Heterocyclic Chemistry”, Pergamon Press, Amsterdam, 2000). Typical procedures for other palladium-catalyzed reactions of pyrazinones can be found in Tetrahedron, 2005, 61, 3953-3962. For alkynyl compounds the Sonogashira reaction is most useful. For alkenyl substrates the Heck and Stille reactions are most useful. For alkyl and cycloalkyl the Kumada and Suzuki couplings are very useful.

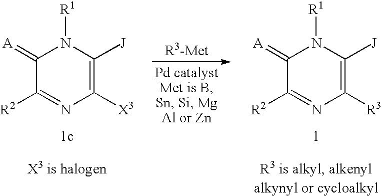

Compounds of Formula 9 (subset of Formula 2 above) wherein X⁴ are halogens can be made by the reaction of cyanoamines of Formula 8 with oxalyl halides as shown in Scheme 8. The reaction is carried out with an excess of an oxalyl halide. The reaction is best carried out in an inert solvent such as 1,2-dichlorobenzene, toluene, chlorobenzene or xylenes at elevated temperatures between about 60 and 150° C. In some cases, the reaction can be carried out at lower temperatures from about 20 to about 60° C. if N,N-dimethylformamide is added to the mixture after the addition of the oxalyl halide. The addition of a halide source such as tetraalkylammonium halides or trialkylammonium halides can sometimes also result in higher yields of product and/or lower reaction temperatures. This type of cyclization can be found in J. Heterocyclic Chemistry, 1983, 20, 919-923, Bull Soc. Chim. Belg. 1994, 103, 583-589, J. Med. Chem., 2005, 48, 1910-1918, and Tetrahedron, 2004, 60, 11597-11612, and references cited therein.

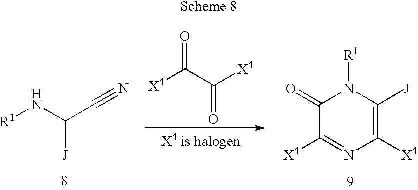

Scheme 9 shows how compounds of Formula 8 can be made by means of the Strecker reaction. This well known reaction involves the reaction of an aldehyde of Formula 10 and an amine of Formula 11 with a cyanide source. The free aldehyde of Formula 10 may be used or it can also be treated with sodium bisulfite prior to the addition to form a bisulfite adduct. The amine of Formula 11 may be in the form of a free base or as an acid addition salt. A variety of solvents and cyanide sources can be employed. For cases in which R¹ is aryl the presence of a Lewis acid such as indium(III) chloride can be advantageous. (For example, see, Ranu et. al., Tetrahedron, 2002, 58, 2529-2532 for typical conditions). This reaction has been the subject of a number of reviews. For conditions and variations of this reaction see the following references and references cited therein: D. T. Mowry, Chemical Reviews, 1948, 42, 236, H. Groeger, Chemical Reviews, 2003, 103, 2795-2827, and M. North in “Comprehensive Organic Functional Group Transformations” A. R. Katritsky, O. Meth-Cohn and C. W. Rees Editors., Volume 3, 615-617; Pergamon, Oxford, 1995.

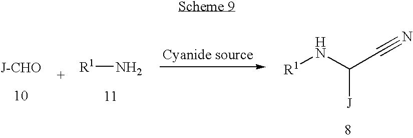

As seen in Scheme 10, compounds of Formula 1e can be made by reaction of compounds of Formula 1a with organometallic reagents of Formula 12 to form ketones of Formula 13, followed by reaction with hydroxylamines and hydrazines of Formula 14. The reaction of Formula 1a with organometallic reagents, preferably Grignard and lithium derivatives, can be carried out at temperatures from −100 to 25° C. Preferably the reaction is carried out in ether or tetrahydrofuran, beginning at −50 to −78° C. and then allowing the reaction mixture to warm to 20 to 25° C. The ketones of Formula 13 can be converted to the compounds of Formula 1e by reaction with the reagents of Formula 14 in a variety of solvents and temperatures. Preferred solvents for this transformation include lower alcohols, tetrahydrofuran and dioxane optionally mixed with water. Most preferred is the use of ethanol. The reaction can be carried out at temperatures from 0 to 120° C. and is most commonly done at the reflux temperature of the solvent used.

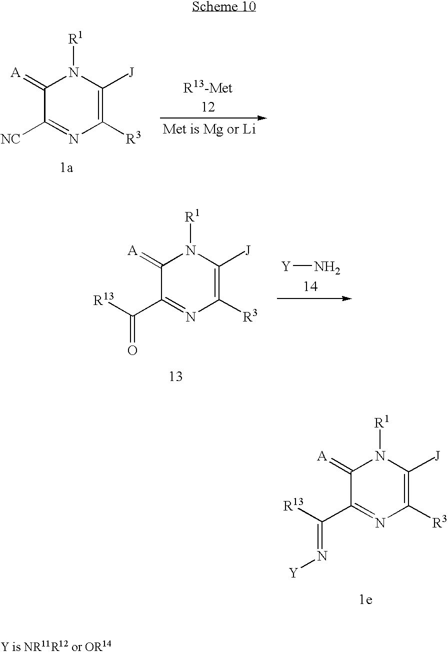

As shown in Scheme 11, various amides of Formula 1f can be made by the reaction of compounds of Formula 2 with a compound of Formula 15 followed by reaction with an oxidizing agent and an amine of Formula 16. The compound of Formula 15 is treated with a strong base such as sodium hexamethyldisilazide, sodium hydride, or 1,8-diazabicyclo-[5.4.0]undec-7-ene and added to a compound of Formula 2. This mixture is further treated with an oxidant such as peracetic acid, t-butyl hydroperoxide, sodium hypochlorite, m-chloroperbenzoic acid, nickel peroxide or other oxidizing agent. Finally an amine of Formula 16 is added to give the compound of Formula if. Reaction temperatures between −20 C and 80° C. are preferred with a temperature of 20 to 30° C. being most preferred. A variety of solvents may be employed with tetrahydrofuran being preferred. For a survey of the use of this amide formation technique with a variety of heterocyclic halides, see Zhang, Synlett, 2004, 2323-2326.

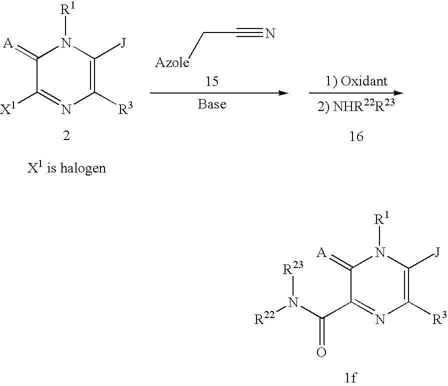

As shown in Scheme 12, compounds of Formula 1g can be converted to a compound of Formula 1j by the following reactions. A compound of Formula 1g can be converted to a compound of Formula 17 by treatment with strong acid. A variety of acids may be successfully employed. Trifluoroacetic acid is a preferred acid for this transformation. The reaction is generally carried out at about 20 to 30° C. in an inert solvent such as dichloromethane. A variety of reagents can convert compounds of Formula 17 to compounds of Formula 1h. Many amination reagents are known in the literature and have been discussed in some detail in Vedejs, Org. Lett., 2003, 7, 4187-4190 and references cited within. A preferred reagent is O-di(p-methoxyphenyl)phosphinylhydroxylamine. The presence of a base such as sodium hydride is preferred. Reaction of compounds of Formula 1h with aldehydes and ketones of Formula 18 give compounds of Formula 1i. The reaction can be carried in the presence of an acid with or without a solvent. Appropriate solvents include tetrahydrofuran, dichloromethane or lower alcohols. Compounds of Formula 1i can be reduced to compounds of Formula 1j by standard reduction techniques. Generally these reactions are conducted by reaction of a boron-based reducing agent such as sodium borohydride or sodium triacetoxyborohydride with the compound of Formula 1i in a solvent such as lower alcohols or tetrahydrofuran. Other reduction techniques known to those skilled in the art may also be employed. A compendium of methods and techniques of reduction of imine type bonds can be found in Organic Reactions, (New York) 2002, 59, 1-714.

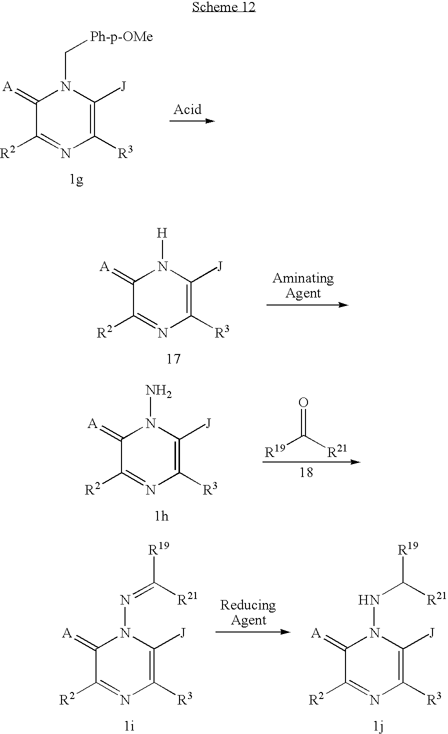

Compounds of Formula 1k in wherein A is NH and R² is a nitrile can be synthesized from compounds of enamines of Formula 19 by a two-step procedure as shown in Scheme 13. The enamines are reacted with [[[(4-methylphenyl)sulfonyl]oxy]imino]propanedinitrile in the presence of a base such as pyridine or triethylamine in a variety of solvents to afford compounds of Formula 20. Preferred solvents include chloroform, dichloromethane and N,N-dimethylformamide. In a second step the compounds of Formula 20 are reacted with an amine of Formula 11 to afford the desired compounds of Formula 1k. Examples of these procedures can be found in Lang et al., Helv. Chem. Acta., 1986, 69, 1025-1033.

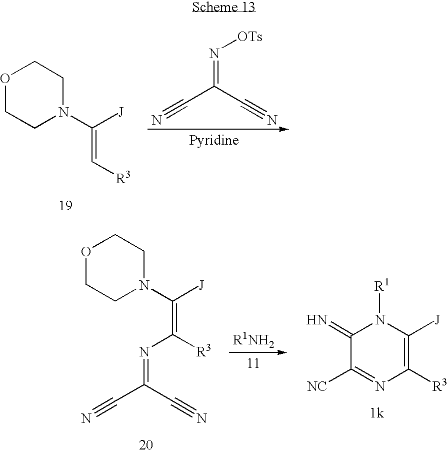

The synthesis of enamines of Formula 19 is well known in the art. For a review of preparative methods see for example Hickmott, et al., Tetrahedron, 1982, 38, 1975-2050 and Tetrahedron, 1982, 38, 3363-3446.

Compounds of Formula 1l in wherein A is NH and R² is CONH₂ can be synthesized from compounds of Formula 1k in wherein A is NH and R² is a nitrile by acidic hydrolysis as shown in Scheme 14. Reagents such as trifluoroacetic acid and trifluoroacetic acid/sulfuric acid mixtures can be employed. This reaction can be conducted between about 0 and 200° C., and preferably between about 20 and 80° C.

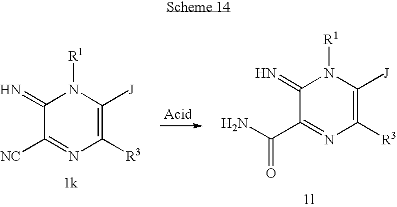

As shown in Scheme 15, compounds of Formula 1m can be prepared by the reaction of compounds of Formula 22 with compounds of Formula 21 wherein Z¹ is a suitable leaving group such as halogen (e.g., F, Cl, Br, I), OS(O)₂CH₃ (methanesulfone), OS(O)₂CF₃, OS(O)₂Ph-p-CH₃ (p-toluenesulfone) and the like, and preferably fluoride. This reaction is carried out in the presence of a strong base such as metal hydride, alkali metal hydroxide or alkali metal carbonate in the presence or absence of a suitable aprotic solvent such as N,N-dimethylformamide and dimethylsulfoxide. A suitable temperature range for this reaction is between about 0 and 150° C. This reaction works particularly well when Z¹ is in the 4-position of the phenyl ring of Formula 21 and at least two of the substituents R^{20a} are electron withdrawing groups such as fluoride.

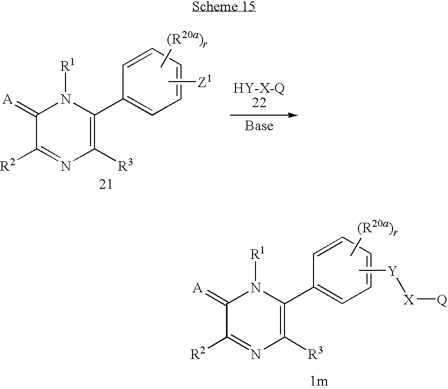

wherein each R^{20a} is independently R²⁹ as defined above in the Summary of the Invention, r is an integer from 0 to 4, and Y, X and Q are defined above in the Summary of the Invention.

As shown in Scheme 16, compounds of Formula 1m can also be prepared from compounds of Formula 1n wherein Y is a heteroatom such as O or N and G¹ is a suitable protecting group such as alkyl group, preferably Y is oxygen and G¹ is CH₃. In this preferred case, compounds of Formula 1n are deprotected with a suitable deprotecting agent to form compounds of Formula 23. Suitable deprotecting agents such as BBr₃, AlCl₃ and HBr in acetic acid can be used in the presence or absence of solvents such as dichloromethane and dichloroethane in a temperature range of about −80 to 120° C. (see: Greene T. W. et al. in “Protective Groups in Organic Synthesis”).

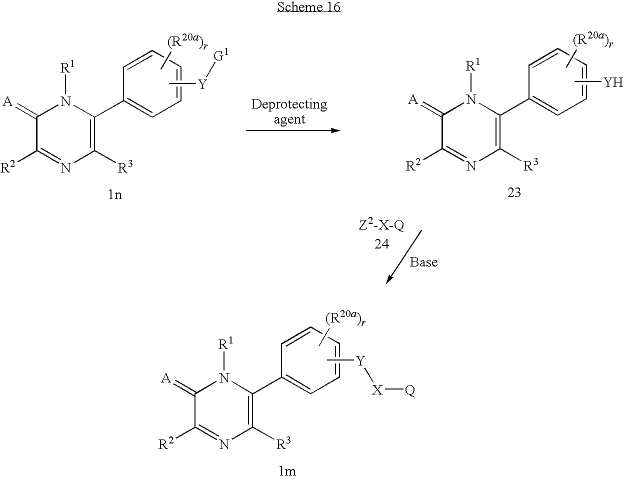

wherein Z² is a suitable leaving group such as halogen (e.g., Cl, Br, I), OS(O)₂CH₃ (methanesulfone), OS(O)₂CF₃, OS(O)₂Ph-p-CH₃ (p-toluenesulfone) and the like. Compounds of Formula 23 are then reacted with alkylating agents 24 in conjunction with a base such as a metal hydride, alkali metal hydroxide or alkali metal carbonate in the presence or absence of a suitable aprotic solvent such as N,N-dimethylformamide or dimethylsulfoxide between 0° C. and 120° C. A particularly noteworthy procedure employs Ca₂CO₃ in the presence of N,N-dimethylformamide at 70° C.

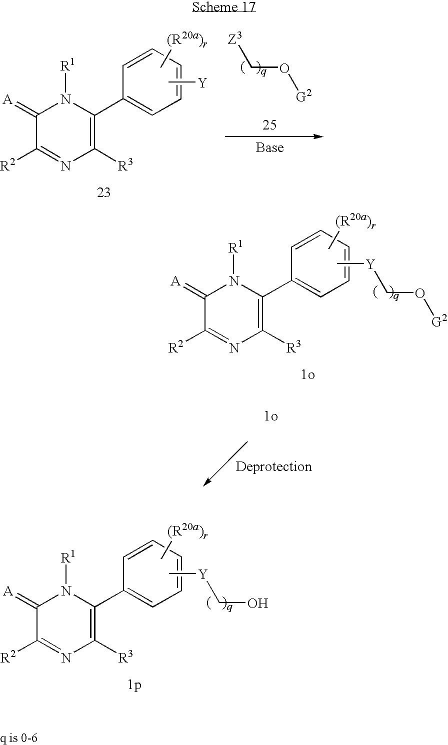

Scheme 17 outlines the case where an alkylating agent 25, wherein G² is a protecting group and Z³ is a leaving group such as halogen (e.g., Cl, Br, I), OS(O)₂CH₃ (methanesulfone), OS(O)₂CF₃, OS(O)₂Ph-p-CH₃ (p-toluenesulfone) and like, has been utilized with the compounds of Formula 23 resulting in compounds of Formula 1o. Most preferably, the protecting group G² is benzyl but other groups such as trialkyl silanes and esters can be used. In the case where benzyl is used, deprotection occurs using palladium-catalyzed hydrogenation (see: Greene T. W. et al. in “Protective Groups in Organic Synthesis”) resulting in compounds of Formula 1p.

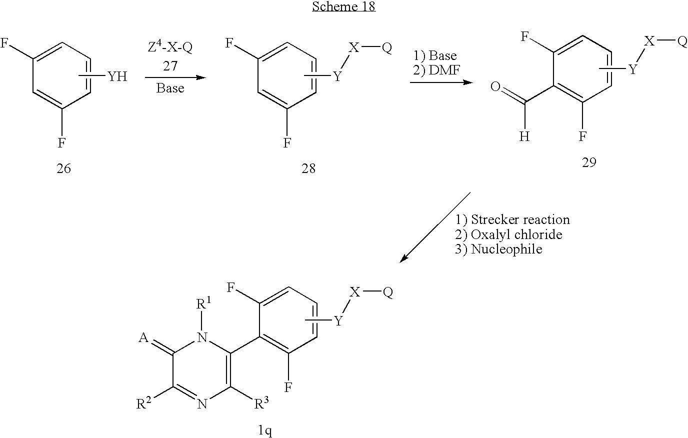

As shown in Scheme 18, compounds of type 1q can be made starting from compounds of Formula 26, wherein Y is O, S, or HNR, which is reacted with compounds of formula 27, wherein Z⁴ is a suitable leaving group such as halogen (e.g., Cl, Br, I), OS(O)₂CH₃ (methanesulfone), OS(O)₂CF₃, OS(O)₂Ph-p-CH₃ (p-toluenesulfone) and like, in the presence of a base such as NaH, Cs₂CO₃ or triethylamine in an aprotic solvent such as N,N-dimethylformamide at a temperature between about −10 and 50° C. The resultant compounds of Formula 28 are then treated with a strong base such as n-BuLi in a suitable aprotic solvent such as tetrahydrofuran or diethyl ether at a temperature between about −80 and 0° C. followed by addition of N,N-dimethylformamide to yield aldehydes of Formula 29, which are then subjected to the aforementioned procedures to yield compounds of Formula 1q.

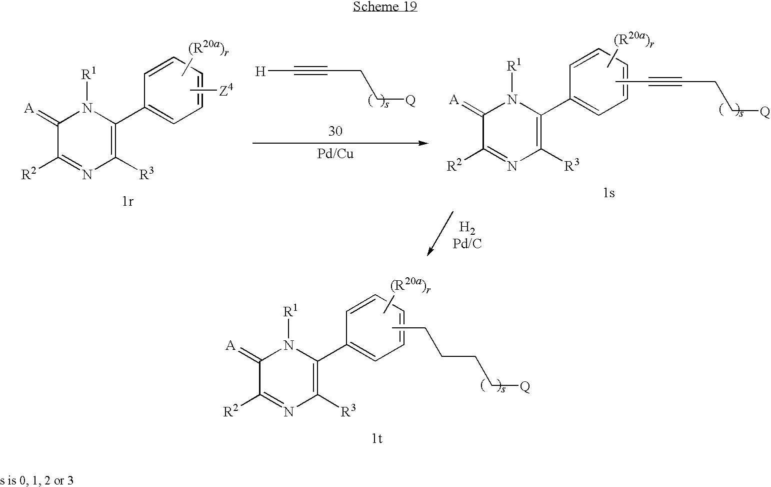

Compounds of Formula 1t wherein Z⁴ is a leaving group such as halogen (e.g., F, Cl, Br, I), OS(O)₂CH₃ (methanesulfone), OS(O)₂CF₃, OS(O)₂Ph-p-CH₃ (p-toluenesulfone) and like can be synthesized from compounds 1r using various coupling reagents in conjunction with a palladium catalyzed coupling reaction. In particular, Scheme 19 illustrates that compounds of Formula 1r can be subjected to a Sonogashira reaction (see: Sonogashira, K. In Metal-Catalyzed Cross-Coupling Reactions; Diederich, F., Stang, P. J., Eds.; Wiley-VCH: New York, 1998; Chapter 5) with compounds of Formula 30 in the presence of Pd and Cu catalysts and a base, such as triethylamine at a temperature between about 20 and 150° C. to result in compounds of Formula 1s. Reduction of compounds of Formula 1s with Pd catalysts in the presence of hydrogen gas according to common procedures produces compounds of Formula 1t.

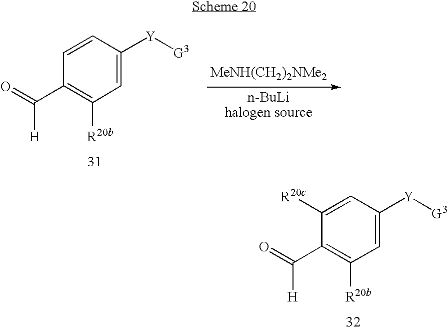

wherein R^{20c} is halogen such as F, Cl, Br or I

Halogenation of the ortho position of benzaldehyde can be prepared by directed metallation. Certain compounds of Formula 32 wherein R^{20b} is a substituents such as proton, halogen or an alkyl group, R^{20c} is a halogen, Y is O, and G³ is an alkyl group can be prepared by reaction of the parent compound of Formula 31 and a halogen source as shown in Scheme 20. In one example, a substituted diaminoethane such as N,N,N′-trimethylethylenediamine in conjunction with an excess of an alkyllithium such as n-butyllithium or s-butyllithium in an aprotic solvent such as tetrahydrofuran or diethyl ether at a temperature between −100° C. and 0° C. is reacted with an aldehyde of Formula 31. The further addition of a halogen source as a suitable electrophile such as N-chlorosuccinimide, hexachloroethane, SelectFluo# or iodomethane results in a compound of Formula 32. Examples of this procedure can be found in Comins, D. L. and Brown, J. D., J. Org. Chem., 1984, 49, 1078-1083.

It is recognized that some reagents and reaction conditions described above for preparing compounds of Formula1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. MPLC means medium pressure chromatography on silica gel. HPLC means high performance liquid chromatography. ¹HNMR spectra are reported in ppm downfield from tetramethylsilane; “s” means singlet, “d” means doublet, “t” means triplet, “m” means multiplet, “dd” means doublet of doublets, “ddd” means doublet of doublet of doublets, “br s” means broad singlet.

### Example 1

### Preparation of 5-Chloro-6-(2,6-difluorophenyl)-1-(2-methylpropyl)-3-(1H-pyrazol-1-yl)-2(1H)-pyrazinone (Compound 1)

### Step A: Preparation of 2,6-Difluoro-α-[(2-methylpropyl)amino]benzeneacetonitrile

To a solution of isobutylamine (2.92 g, 40 mmol) and sodium cyanide (1.94 g, 40 mmol) in water (40 mL) was added a solution of 2,6-difluorobenzaldehyde (5.7 g, 40 mmol) in methanol (40 mL). The addition was done at such a rate so that the temperature remained below 35° C. The reaction mixture was stirred at room temperature for 18 h. The mixture was partitioned between water (150 mL) and dichloromethane (150 mL). The organic layer was washed with water (2×50 mL). The organic layer was dried (MgSO₄) and evaporated under reduced pressure to give an oil. Flash chromatographic purification on silica gel with hexanes as eluant and pooling of appropriate fractions gave 4.92 g of the title compound as an oil.

¹H NMR (CDCl₃) δ 8.4 (br s, 1H), 7.3-7.2 (m, 1H), 6.9 (m, 2H), 3.5 (m, 2H), 2.0 (m, 1H), 0.9 (m, 6H).

### Step B: Preparation of 3,5-Dichloro-6-(2,6-difluorophenyl)-1-(2-methylpropyl)-2(1H)-pyrazinone

A solution of oxalyl chloride (3.34 g, 26 mmol) in chlorobenzene (35 mL) was stirred at 25° C. and 2.46 g (80% pure, 9 mmol) of 2,6-difluoro-α-[(2-methylpropyl)amino]-benzeneacetonitrile (i.e. the product of Example 1 step A) was added via an addition funnel. The resulting reaction mixture was heated at 70° C. for 18 h and at 90° C. for 24 h. The solvent was evaporated under reduced pressure to leave an oil. This residue was subjected to silica gel chromatographic purification using a gradient of ethyl acetate/hexanes (1:9 to 2:3), and the appropriate fractions were pooled to give 1.2 g of the title compound as an oil which solidified on standing. This product was of sufficient purity to use in subsequent reactions.

¹H NMR (CDCl₃) δ 7.6 (m, 1H), 7.1 (m, 1H), 7.0 (m, 1H), 3.7 (m, 2H), 1.9 (m, 1H), 0.9 (m, 3H), 0.7 (d, 3H).

### Step C: Preparation of 5-Chloro-6-(2,6-difluorophenyl)-1-(2-methylpropyl)-3-(1H-pyrazol-1-yl)-2(1H)-pyrazinone (Compound 1)

A mixture of 3,5-dichloro-6-(2,6-difluorophenyl)-1-(2-methylpropyl)-2(1H)-pyrazinone (i.e. the product of Example 1 step B) (200 mg, 0.6 mmol), pyrazole (45 mg, 0.66 mmol) and potassium carbonate (166 mg, 1.2 mmol) dissolved in N,N-dimethylformamide (2 mL) was heated at 60° C. for 18 h. The mixture was partitioned between ethyl acetate (20 mL) and water (10 mL). The organic layer was washed with water (3×10 mL). The residue after evaporation was subjected to silica gel chromatographic purification using a gradient of hexanes/ethyl acetate (1:9 to 2:3) as eluant to give 60 mg of the title product, a compound of the present invention as an oil which later solidified, melting at 118-119° C.

¹H NMR (CDCl₃) δ 9.1 (m, 1H), 7.9 (m, 1H), 7.5 (m, 1H), 7.1 (m, 2H), 6.5 (m, 1H), 3.8 (d, 2H), 2.0 (m, 1H), 0.8 (d, 6H).

### Example 2

### Preparation of 5-Chloro-6-(2,6-difluorophenyl)-1-(2-methylpropyl)-3-(2-pyridinyl)-2(1H)-pyrazinone (Compound 2)

A mixture of 3,5-dichloro-6-(2,6-difluorophenyl)-1-(2-methylpropyl)-2(1H)-pyrazinone (i.e. the product of Example 1 step B) (200 mg, 0.6 mmol), tributylstannylpyridine (Lancaster Synthesis, 240 mg, 0.63 mmol) and bis(triphenylphoshino)palladium(II) chloride (20 mg, 0.03 mmol) was heated in toluene at 110° C. for 18 h. The mixture was filtered through a pad of Celite® diatomaceous filter aid, and rinsed with ethyl acetate. The solvent was evaporated under reduced pressure. The residue after evaporation was subjected to silica gel chromatographic purification using a gradient of ethyl acetate/hexanes (1:9 to 2:3), and the appropriate fractions were pooled to give 56 mg of the title product, a compound of the present invention, as an oil.

¹H NMR (CDCl₃) δ 8.86 (m, 1H), 8.43 (m, 1H), 7.83 (m, 1H), 7.59 (m, 1H), 7.38 (m, 1H), 7.12 (m, 2H), 3.79 (d, 2H), 2.00 (m, 1H), 0.79 (d, 6H).

### Example 3

### Preparation of 6-(2,6-Difluorophenyl)-1-(2-methylpropyl)-3-(1H-pyrazol-1-yl)-2(1H)-pyrazinone (Compound 342)

A mixture of 5-chloro-6-(2,6-difluorophenyl)-1-(2-methylpropyl)-3-(1H-pyrazol-1-yl)-2(1H)-pyrazinone (i.e. the product of Example 1 step C) (0.70 g, 1.92 mmol), triethylamine (0.40 mL, 2.88 mmol) and 10% palladium on carbon (50 mg, 0.471 mmol) in ethyl acetate (10 mL) was shaked under 50 psi (345 kPa) pressure of hydrogen overnight. The reaction mixture was filtered through Celite® diatomaceous filter aid. The solvent was removed with a rotary evaporator. The residue was taken up in ethyl acetate and was washed with water. The organic layer was dried, and the solvent was removed with a rotary evaporator. The residue was purified by silica gel flash chromatography (1 to 33% ethyl acetate in hexanes as eluant) to give 110 mg of the title product, a compound of the present invention, as an oil which later solidified, melting at 91-92° C.

¹H NMR (CDCl₃) δ 9.10 (s, 1H), 7.86 (s, 1H), 7.54 (m, 1H), 7.31 (s, 1H), 7.09 (m, 2H), 6.50 (s, 1H), 3.80 (d, 2H), 2.04 (m, 1H), 0.78 (d, 6H).

### Example 4

### Preparation of 5-Chloro-6-(2,6-difluorophenyl)-1-[(4-methoxyphenyl)methyl]-3-(1H-pyrazol-1-yl)-2(1H)-pyrazinone (Compound 271), 1-Amino-5-chloro-6-(2,6-difluorophenyl)-3-(1H-pyrazol-1-)-2(1H)-pyrazinone (Compound 400) and 5-Chloro-6-(2,6-difluorophenyl)-1-[(1-methylethylidene)amino]-3-(1H-pyrazol-1-yl)-2(1H)-pyrazinone (Compound 392)

### Step A: Preparation of 2,6-Difluoro-α-[[(4-methoxyphenyl)methyl]amino]benzene-acetonitrile

To a solution of sodium hydrogensulfite (19.9 g, 0.191 mol) in water (180 mL) and methanol (18 mL) was added 2,6-difluorobenzaldehyde (25.95 g, 0.182 mol). The reaction mixture was stirred at room temperature for 15 minutes. A mild exotherm to 30° C. was observed. Then sodium cyanide (8.93 g, 0.182 mol) was added, and the reaction mixture was stirred for 25 minutes. The reaction mixture was cooled to 10° C. and 4-methoxybenzylamine (24.99 g, 0.182 mol) was added dropwise. The reaction mixture was heated to 65° C. for 5 h and allowed to cool to room temperature overnight. The reaction mixture was diluted with diethyl ether (200 mL) and washed with brine (2×100 mL). The aqueous layer was extracted once with diethyl ether. The organic layers were combined, dried (MgSO₄), filtered and concentrated under reduced pressure to give 51.26 g of the title compound as an oil.

¹H NMR (CDCl₃) δ 7.37-7.28 (m, 3H), 6.96 (t, 2H), 6.88 (d, 2H), 4.94 (s, 1H), 4.05 (d, 1H), 3.89 (d, 1H), 3.81 (s, 3H), 2.27 (s, 1H).

### Step B: Preparation of 3,5-Dichloro-6-(2,6-difluorophenyl)-1-[(4-methoxyphenyl)-methyl]-2(1H)-pyrazinone

To a solution of 2,6-difluoro-α-[[(4-methoxyphenyl)methyl]amino]benzene-acetonitrile (i.e. the product of Example 4 step A) (48.8 g, 0.169 mol) in chlorobenzene (550 mL) was added oxalyl chloride (64.45 g, 0.507 mol) dropwise keeping temperature below 15° C. The reaction mixture was then warmed to room temperature and stirred for 30 minutes. Then triethylamine hydrochloride (46.6 g, 0.338 mol) was added and reaction mixture was heated to 80° C. for 2 h. The reaction mixture was allowed to stir at room temperature overnight. The resulting mixture was then concentrated under reduced pressure, and purified by silica gel flash chromatography (25% ethyl acetate in hexanes as eluant) to afford 31.2 g of the title compound as an oil.

¹H NMR (CDCl₃) δ 7.55 (s, 1H), 7.02 (dd, 2H), 6.77-6.67 (m, 4H), 5.04 (s, 2H), 3.75 (s, 3H).

### Step C: Preparation of 5-Chloro-6-(2,6-difluorophenyl)-1-[(4-methoxyphenyl)-methyl]-3-(1H-pyrazol-1-yl)-2(1H)-pyrazinone (Compounds 271)

To a solution of 3,5-dichloro-6-(2,6-difluorophenyl)-1-[(4-methoxyphenyl)-methyl]-2(1H)-pyrazinone (i.e. the product of Example 4 step B) (20 g, 50.0 mmol) in acetonitrile (250 mL) was added pyrazole (3.43 g, 60.0 mmol) and potassium bicarbonate (20.74 g, 150 mmol), and stirred at 60° C. for 3 h. The reaction mixture was then cooled to room temperature and poured into ice water (500 mL). After stirring for 10 minutes, resulting precipitate was filtered, rinsed with cold water, and dried to afford 21.17 g of the title product, a compound of the present invention as an off-white solid.

¹H NMR (CDCl₃) δ 9.13 (d, 1H), 7.90 (d, 1H), 7.54 (s, 1H), 7.05-6.97 (m, 2H), 6.83-6.75 (m, 2H), 6.74-6.68 (m, 2H), 6.52 (dd, 1H), 5.13 (s, 2H), 3.75 (s, 3H).

### Step D: Preparation of 5-Chloro-6-(2,6-difluorophenyl)-3-(1H-pyrazol-1-yl)-2(1H)-pyrazinone

A solution of 5-chloro-6-(2,6-difluorophenyl)-1-[(4-methoxyphenyl)-methyl]-3-(1H-pyrazol-1-yl)-2(1H)-pyrazinone (i.e. the product of Example 4 step C) (21.17 g, 49.0 mmol) in trifluoroacetic acid (37 mL, 493 mmol) was stirred under reflux for 6 h and allowed to cool to room temperature overnight. The reaction mixture was concentrated under reduced pressure and the resulting crude oil was purified by silica gel flash chromatography using 100% dichloromethane as eluant. It was the recrystallized from methanol to give 6.07 g of the title compound as an oil.

¹H NMR (CDCl₃) δ 12.74 (s, 1H), 8.63 (d, 1H), 7.84 (s, 1H), 7.44 (ddd, 1H), 7.02 (t, 2H), 6.64 (s, 1H).

### Step E: Preparation of 1-Amino-5-chloro-6-(2,6-difluorophenyl)-3-(1H-pyrazol-1-yl)-2(1H)-pyrazinone (Compound 400)

To a slurry of sodium hydride (55% of oil dispersion, 42.5 mg, 0.974 mmol) in tetrahydrofuran (8 mL) at approximately −78° C. was added a solution of 1-amino-5-chloro-6-(2,6-difluorophenyl)-3-(1H-pyrazol-1-yl)-2(1H)-pyrazinone (i.e. the product of Example 4 step D) (250 mg, 0.812 mmol) in tetrahydrofuran (11 mL). The reaction mixture was stirred at −78° C. for 15 minutes and then at 0° C. for 15 additional minutes. Then 1,1-dimethylethyl [[bis(4-methoxyphenyl)phosphinyl]oxy]carbamate (262 mg, 8.93 mmol) was added, and the reaction mixture was allowed to warm to room temperature overnight. The reaction mixture was then concentrated under reduced pressure and purified by MPLC (0 to 100% ethyl acetate in hexanes as eluant) to afford 36 mg of the title product, a compound of the present invention, as an oil.

¹H NMR (CDCl₃) δ 9.12-9.03 (m, 1H), 7.91 (s, 1H), 7.64-7.49 (m, 1H), 7.17-7.05 (m, 2H), 6.54 (s, 1H), 5.43 (s, 2H).

### Step F: Preparation of 5-Chloro-6-(2,6-difluorophenyl)-1-[(1-methylethylidene)-amino]-3-(1H-pyrazol-1-yl)-2(1H)-pyrazinone (Compound 392)

To a solution of 1-amino-5-chloro-6-(2,6-difluorophenyl)-3-(1H-pyrazol-1-yl)-2(1H)-pyrazinone (i.e. the product of Example 4 step E) (36 mg, 0.111 mmol) in acetone (10 mL) was added a solution of 2 M hydrogen chloride in diethyl ether (2 mL) and 4 Å molecular sieves. The reaction mixture was then stirred at room temperature overnight. The resulting mixture was concentrated under reduced pressure to give 40 mg of the title product, a compound of the present invention.

¹H NMR (CDCl₃) δ 9.10 (s, 1H), 7.90 (s, 1H), 7.54-7.45 (m, 1H), 7.12-7.03 (m, 1H), 7.03-6.95 (m, 1H), 6.51 (s, 1H), 2.10 (s, 3H), 1.94 (s, 3H).

### Example 5

### Preparation of 5-Chloro-6-(1-methylpropyl)-1-(2-methylpropyl)-3-(1H-pyrazol-1-yl)-2(1H)-pyrazinone (Compound 424)

### Step A: Preparation of 3-Methyl-2-[(2-methylpropyl)amino]pentanenitrile

To a solution of sodium hydrogensulfite (2.31 g, 22.2 mmol) in water (20 mL) and methanol (2 mL) was added 2-methylbutyraldehyde (1.82 g, 21.1 mmol) at room temperature. The reaction mixture was then stirred for 15 minutes, and sodium cyanide (1.09 g, 22.2 mmol) was added. The reaction mixture was stirred for an additional 20 minutes. The reaction mixture was then cooled in an ice water bath, and a solution of isobutylamine (1.70 g, 23.2 mmol) in methanol (4 mL) was added over an approximately 2 minute period. The reaction mixture was stirred at 0° C. for 15 minutes and then heated to 35° C. for 2 h. The reaction mixture was then extracted with ethyl acetate (2×20 mL) and the combined organic layers were washed with brine, dried (MgSO₄), and concentrated to give 3.1 g of the title compound as a yellow oil.

¹H NMR (CDCl₃) δ 3.41-3.33 (m, 1H), 2.71-2.65 (m, 1H), 2.44-2.36 (m, 1H), 1.79-1.66 (m, 2H), 1.66-1.54 (m, 1H), 1.39-1.29 (m, 1H), 1.10-1.03 (m, 3H), 0.97-0.89 (m, 9H).

### Step B: Preparation of 3,5-Dichloro-6-(1-methylpropyl)-1-(2-methylpropyl)-2(1H)-pyrazinone

A solution of 3-methyl-2-[(2-methylpropyl)amino]pentanenitrile (i.e. the product of Example 5 step A) (3.1 g, 18.4 mmol) in chlorobenzene (12 mL) was added over 20 minutes to a solution of oxalyl chloride (11.7 g, 92.1 mmol) in chlorobenzene (43 mL) at room temperature. Then N,N-dimethylformamide (3 mL) was added dropwise. The reaction mixture was then heated to 95° C. overnight. The reaction mixture was concentrated under reduced pressure and the residue was purified by MPLC (0 to 100% gradient of ethyl acetate in hexanes as eluant) to afford 3.7 g of the title compound, as a solid.

¹H NMR (CDCl₃) δ 4.22-4.08 (m, 1H), 4.02-3.92 (m, 1H), 3.02-2.88 (m, 1H), 2.09-1.98 (m, 2H), 1.97-1.87 (m, 1H), 1.45 (d, 3H), 1.02-0.91 (m, 9H).

### Step C: Preparation of 5-Chloro-6-(1-methylpropyl)-1-(2-methylpropyl)-3-(1H-pyrazol-1-yl)-2(1H)-pyrazinone (Compound 424)

A mixture of 3,5-dichloro-6-(1-methylpropyl)-1-(2-methylpropyl)-2(1H)-pyrazinone (i.e. the product of Example 5 step B) (0.30 g, 1.09 mmol), pyrazole (0.081 g, 1.20 mmol) and potassium carbonate (0.30 g, 2.17 mmol) in N,N-dimethylformamide (4 mL) was heated at 60° C. overnight. The reaction mixture was then concentrated under reduced pressure. The residue was purified by MPLC (0 to 100% gradient of ethyl acetate in hexanes as eluant) to give 0.22 g of the title product, a compound of the present invention.

¹H NMR (CDCl₃) δ 8.96 (br s, 1H), 7.83 (br s, 1H), 6.45 (br s, 1H), 4.40-4.15 (m, 1H), 4.16-3.97 (m, 1H), 3.12-2.92 (m, 1H), 2.16-2.01 (m, 2H), 2.02-1.88 (m, 1H), 1.49 (d, 3H), 1.05-0.98 (m, 6H), 0.98-0.92 (m, 3H).

### Example 6

### Preparation of 5-Chloro-6-(2-chloro-4-fluorophenyl)-1-(2-methylpropyl)-3-(1H-pyrazol-1-yl)-2(1H)-pyrazinone (Compound 53)

### Step A: Preparation of 2-Chloro-4-fluoro-α-[(2-methylpropyl)amino]benzene-acetonitrile

To a solution of sodium hydrogensulfite (1.53 g, 14.8 mmol) in a mixture of deionized water (14 mL) and methanol (1.3 mL) was added 2-chloro-4-fluorobenzaldehyde (2.23 g, 14.1 mmol) at room temperature. The reaction mixture was stirred for 15 minutes, and sodium cyanide (0.724 g, 14.8 mmol) was added. The reaction mixture was stirred for an additional 20 minutes. The reaction mixture was cooled using an ice water bath, and a solution of isobutylamine (1.13 g, 15.5 mmol) in methanol (2.67 mL) was added over approximately 2 minutes. The reaction mixture was stirred at 0° C. for 15 minutes and then heated to 35° C. for 2 h. The resulting mixture was then extracted with ethyl acetate (2×20 mL), and the combined organic layers were washed with brine, dried (MgSO₄) and concentrated to give 3.09 g of the title compound as a yellow oil.

¹H NMR (CDCl₃) δ 7.65-7.61 (m, 1H), 7.22-7.18 (m, 1H), 7.10-7.04 (m, 1H), 5.01 (s, 1H), 2.70-2.64 (m, 1H), 2.58-2.51 (m, 1H), 1.81-1.71 (m, 1H), 0.97-0.92 (m, 6H).

### Step B: Preparation of 3,5-Dichloro-6-(2-chloro-4-fluorophenyl)-1-(2-methylpropyl)-2(1H)-pyrazinone

A solution of 2-chloro-4-fluoro-α-[(2-methylpropyl)amino]benzeneacetonitrile (i.e. the product of Example 6 step A) (3.09 g, 12.8 mmol) dissolved in chlorobenzene (8 mL) was added dropwise over 20 minutes to a solution of oxalyl chloride (8.15 g, 64.2 mmol) in chlorobenzene (30 mL) at room temperature. The reaction mixture was then heated to 100° C. overnight. The solvent was removed under reduced pressure, and the residue was purified by MPLC (0 to 100% ethyl acetate in hexanes as eluant) to give 2.13 g of the title compound as a solid.

¹H NMR (CDCl₃) δ 7.38-7.31 (m, 2H), 7.23-7.17 (m, 1H), 4.02-3.95 (m, 1H), 3.38-3.30 (m, 1H), 2.01-1.90 (m, 1H), 0.82 (d, 3H), 0.72 (d, 3H).

### Step C: Preparation of 5-Chloro-6-(2-chloro-4-fluorophenyl)-1-(2-methylpropyl)-3-(1H-pyrazol-1-yl)-2(1H)-pyrazinone (Compound 53)

A mixture of 3,5-dichloro-6-(2-chloro-4-fluorophenyl)-1-(2-methylpropyl)-2(1H)-pyrazinone (i.e. the product of Example 6 step B) (0.350 g, 1.00 mmol), pyrazole (0.075 g, 1.10 mmol) and potassium carbonate (0.276 g, 2.00 mmol) in N,N-dimethylformamide (4 mL) was heated to 60° C. overnight. The reaction mixture was concentrated under reduced pressure, and the residue was purified by MPLC (0 to 100% ethyl acetate in hexanes as eluant) to give 0.256 g of the title product, a compound of the present invention, as a solid melting at 137-139° C.

¹H NMR (CDCl₃) δ 9.10 (d, 1H), 7.89 (d, 1H), 7.48-7.38 (m, 1H), 7.37-7.30 (m, 1H), 7.27-7.14 (m, 1H), 6.56-6.46 (m, 1H), 4.16-4.03 (m, 1H), 3.48-3.36 (m, 1H), 2.08-1.91 (m, 1H), 0.84 (d, 3H), 0.75 (d, 3H).

### Example 7

### Separation of the Atropisomers of 5-Chloro-6-(2-chloro-4-fluorophenyl)-1-(2-methylpropyl)-3-(1H-pyrazol-1-yl)-2(1H)-pyrazinone: (Compound 302) and (Compound 303)

5-Chloro-6-(2-chloro-4-fluorophenyl)-1-(2-methylpropyl)-3-(1H-pyrazol-1-yl)-2(1H)-pyrazinone (i.e. the product of Example 6 step C) (40 mg, 0.10 mmol) was purified on a ChiralCel® OJ, analytical HPLC column by Daicel Chemical Industries, LTD., (0.1% formic acid in a mixture of 49.9% methanol and 50% acetonitrile as eluant, 1 mL/min) to afford 16 mg of the second title product, Compound 303 of the present invention at the retention time of 18.9 minutes, and 16.5 mg of the first title product, Compound 302 of the present invention, at the retention time of 22.6 minutes.

¹H NMR (CDCl₃) of 5-Chloro-6-(2-chloro-4-fluorophenyl)-1-(2-methylpropyl)-3-(1H-pyrazol-1-yl)-2(1H)-pyrazinone (Compound 302): δ 9.10 (br s, 1H), 7.89 (br s, 1H), 7.42-7.37 (m, 1H), 7.36-7.31 (m, 1H), 7.24-7.16 (m, 1H), 6.51 (br s, 1H), 4.17-4.04 (m, 1H), 3.46-3.34 (m, 1H), 2.09-1.93 (m, 1H), 0.85 (d, 3H), 0.75 (d, 3H).

¹H NMR (CDCl₃) of 5-Chloro-6-(2-chloro-4-fluorophenyl)-1-(2-methylpropyl)-3-(1H-pyrazol-1-yl)-2(1H)-pyrazinone (Compound 303): δ 9.09 (br s, 1H), 7.89 (br s, 1H), 7.42-7.36 (m, 1H), 7.36-7.31 (m, 1H), 7.23-7.17 (m, 1H), 6.52 (br s, 1H), 4.16-4.04 (m, 1H), 3.45-3.34 (m, 1H), 2.09-1.93 (m, 1H), 0.84 (d, 3H), 0.75 (d, 3H).

### Example 8

### Preparation of 6-Chloro-4-(3-fluorophenyl)-3,4-dihydro-3-oxo-5-(2,4,6-trifluorophenyl)pyrazinecarboxamide (Compound 414)

### Step A: Preparation of 2,4,6-Trifluoro-α-[(3-fluorophenyl)amino]benzeneacetonitrile

To a solution of 2,4,6-trifluorobenzaldehyde (3.20 g, 20.0 mmol) in tetrahydrofuran (25 mL) was added 3-fluorophenylaniline (2.02 g, 18.2 mmol), potassium cyanide (4.74 g, 72.7 mmol) and indium(III) chloride (4.02 g, 18.2 mmol) in sequence at room temperature. Then the reaction mixture was stirred overnight. The reaction mixture was diluted with water and extracted with ethyl acetate (2×100 mL). The organic extracts were dried (MgSO₄), filtered, and concentrated to afford 5.33 g of the title compound as an oil.

¹H NMR (CDCl₃) δ 7.25 (m, 1H), 6.81 (m, 2H), 6.62 (m, 1H), 6.53 (m, 2H), 5.64 (d, 1H), 4.42 (d, 1H).

### Step B: Preparation of 3,5-Dichloro-1-(3-fluorophenyl)-6-(2,4,6-trifluorophenyl)-2(1H)-pyrazinone

A solution of 2,4,6-trifluoro-α-[(3-fluorophenyl)amino]benzeneacetonitrile (i.e. the product of Example 8 step A) (5.33 g, 19.0 mmol) in chlorobenzene (20 mL) was treated dropwise with oxalyl chloride (8.30 mL, 95.2 mmol) at room temperature. The resulting mixture was heated to 100° C. for 2.5 h. One drop of N,N-dimethylformamide was then added, and heating was continued overnight. The reaction mixture was cooled to room temperature, and concentrated under reduced pressure. The residue was purified by silica gel flash chromatography (15 to 30% ethyl acetate in hexanes as eluant) to afford 6.49 g of the title compound as an oil.

¹H NMR (CDCl₃) δ 7.35 (m, 1H), 6.94 (m, 2H), 6.64 (m, 2H).

### Step C: Preparation of 6-Chloro-4-(3-fluorophenyl)-3,4-dihydro-3-oxo-5-(2,4,6-trifluorophenyl)pyrazinecarboxamide (Compound 414)

To a solution of 3,5-dichloro-1-(3-fluorophenyl)-6-(2,4,6-trifluorophenyl)-2(1H)-pyrazinone (i.e. the product of Example 8 step B) (0.39 g, 1.00 mmol) in tetrahydrofuran (5 mL) was added 1H-benzotriazole-1-acetonitrile (0.24 g, 1.50 mmol) and lithium bis(trimethylsilyl)amide (1.0 M solution in tetrahydrofuran, 2.5 mL, 2.50 mmol). The reaction mixture was stirred at room temperature for 1.5 h. Then a solution of ammonia in dioxane (0.5 M, 6 mL, 3.0 mmol) was added and the reaction mixture was stirred an additional 10 minutes. Peracetic acid (32 wt. % solution in acetic acid, 0.84 mL) was added dropwise to the reaction mixture, and the resulting mixture was stirred at room temperature for 3 h. Saturated aqueous sodium hydrogensulfite was then added (50 mL), and the reaction mixture was extracted with ethyl acetate (2×50 mL). The combined organic extracts were dried (MgSO₄), filtered, and concentrated under reduced pressure. The residue was purified by silica gel flash chromatography (50 to 80% ethyl acetate in hexanes as eluant) to afford 0.15 g of the title product, a compound of the present invention, as an oil.

¹H NMR (CDCl₃) δ 8.98 (s, 2H), 7.63 (m, 2H), 7.40 (m, 1H), 7.12 (m, 2H), 6.24 (s, 1H).

### Example 9

### Preparation of 5-Bromo-6-(2,6-difluorophenyl)-1-(2-methylpropyl)-3-(1H-pyrazol-1-yl)-2(1H)-pyrazinone (Compound 99) and 5-Methyl-6-(2,6-difluorophenyl)-1-(2-methylpropyl)-3-(1H-pyrazol-1-yl)-2(1H)-pyrazinone (Compound 149)

### Step A: Preparation of 3,5-Dibromo-6-(2,6-difluorophenyl)-1-(2-methylpropyl)-2(1H)-pyrazinone

To a solution of oxalyl bromide (8.66 g, 40.1 mmol) in chlorobenzene (40 mL) was added a solution of 2,6-difluoro-α-[(2-methylpropyl)amino]benzeneacetonitrile (i.e. the product of Example 1 step A) (3.0 g, 13.3 mmol) in chlorobenzene (20 mL) at a temperature below 30° C. The reaction mixture was stirred at room temperature for 45 minutes. Then a catalytic amount of N,N-dimethylformamide was added, and the reaction mixture was heated at 100° C. for 18 h. The solvent was removed with a rotary evaporator. The residue was purified by silica gel flash chromatography (5% ethyl acetate in hexanes as eluant) to afford 2 g of the title compound as a solid melting at 125-126° C.

¹H NMR (CDCl₃) δ 7.6 (m, 1H), 7.1 (m, 2H), 3.7 (d, 2H), 1.9 (m, 1H), 0.7 (d, 6H).

### Step B: Preparation of 5-Bromo-6-(2,6-difluorophenyl)-1-(2-methylpropyl)-3-(1H-pyrazol-1-yl)-2(1H)-pyrazinone (Compound 99)

A mixture of 3,5-dibromo-6-(2,6-difluorophenyl)-1-(2-methylpropyl)-2(1H)-pyrazinone (i.e. the product of Example 9 step A) (1.4 g, 3.3 mmol), pyrazole (248 mg, 3.6 mmol) and potassium carbonate (1.3 g, 9.9 mmol) in acetonitrile (10 mL) was the reaction mixture was heated at 80° C. for 2 h, then 60° C. overnight. Then additional pyrazole (100 mg) was added, and heated at 80° C. for 2 h. The reaction mixture was diluted with water, and the resulting solid was filtered. The filtered solid was dissolved with dichloromethane, passed through a ChemElute®, diatomaceous earth column (Varian) and concentrated under reduced pressure to leave an oil. The residue was triturated with a mixture of hexanes and diethyl ether to give 1.05 g of the title product, a compound of the present invention, as a white solid melting at 111-112° C.

¹H NMR (CDCl₃) δ 9.0 (d, 1H), 7.8 (s, 1H), 7.6 (m, 1H), 7.1 (m, 2H), 6.5 (d, 1H), 3.8 (d, 2H), 1.9 (m, 1H), 0.7 (d, 6H).

### Step C: Preparation of 5-Methyl-6-(2,6-difluorophenyl)-1-(2-methylpropyl)-3-(1H-pyrazol-1-yl)-2(1H)-pyrazinone (Compound 149)

To a solution of 5-bromo-6-(2,6-difluorophenyl)-1-(2-methylpropyl)-3-(1H-pyrazol-1-yl)-2(1H)-pyrazinone (i.e. the product of Example 9 step B) (200 mg, 0.48 mmol) and tetrakis(triphenylphosphine)palladium (16 mg, 0.015 mmol) in 1,2-dimethoxyethane (5 mL) at a temperature below 10° C. under nitrogen atmosphere was added dropwise a solution of 2 M trimethylaluminum in hexanes (0.26 mL, 0.51 mmol). The reaction mixture was warmed to room temperature and then heated at 80° C. for about 90 minutes. The resulting mixture was cooled with an ice-water bath and quenched with saturated aqueous ammonium chloride solution (10 mL). The reaction mixture was diluted with ethyl acetate, and the separated organic layer was washed with brine. The resulting organic layer was passed through a ChemElute®, diatomaceous earth column (Varian) and concentrated under reduced pressure to give an oil. This residue was purified by silica gel flash chromatography (5 to 40% ethyl acetate in hexanes as eluant) to afford 44 mg of the title product, a compound of the present invention, as a white solid melting at 105-106° C.

¹H NMR (CDCl₃) δ 9.12 (s, 1H), 7.86 (s, 1H), 7.58 (m, 1H), 7.10 (m, 2H), 6.48 (s, 1H), 3.77 (d, 2H), 2.17 (s, 3H), 1.95 (m, 1H), 0.75 (d, 6H).

### Example 10

### Preparation of 6-Chloro-5-(2,6-difluorophenyl)-3,4-dihydro-4-(2-methylpropyl)-3-oxopyrazinecarbonitrile (Compound 5)

A mixture of 3,5-dichloro-6-(2,6-difluorophenyl)-1-(2-methylpropyl)-2(1H)-pyrazinone (i.e. the product of Example 1 step B) (200 mg, 0.6 mmol) and sodium cyanide (31 mg, 0.63 mmol) in N,N-dimethylformamide (2 mL) was heated at 60° C. overnight. The reaction mixture was diluted with water and extracted with diethyl ether. The organic layer was separated and washed with water, passed through a ChemElute® diatomaceous earth column (Varian) and concentrated under reduced pressure to give an oil. This residue was purified by silica gel flash chromatography (10 to 20% ethyl acetate in hexanes as eluant) to afford 70 mg of the title product, a compound of the present invention, as a white solid melting at 100-102° C.

¹H NMR (CDCl₃) δ 7.6 (m, 1H), 7.1 (m, 2H), 3.7 (d, 2H), 1.9 (m, 1H), 0.7 (m, 6H).

### Example 11

### Preparation of 5-Chloro-6-(2,6-difluorophenyl)-1-(2-methylpropyl)-3-(1-methyl-1H-imidazol-4-yl)-2(1H)-pyrazinone (Compound 85)

To a solution of 4-iodo-1-methyl-1H-imidazole (0.31 g, 1.50 mmol) in dichloromethane (5 mL) was added ethylmagnesium bromide (3.0 M solution in tetrahydrofuran, 0.50 mL, 1.50 mmol). The reaction mixture was stirred at room temperature for 15 minutes, and a solution of 3,5-dichloro-6-(2,6-difluorophenyl)-1-(2-methylpropyl)-2(1H)-pyrazinone (i.e. the product of Example 10 step A) (0.50 g, 1.50 mmol) in dichloromethane (5 mL) was added. The reaction mixture was stirred at room temperature overnight, and then quenched with saturated aqueous ammonium chloride solution (1 mL). The resulting mixture was passed through a ChemElute®, diatomaceous earth column (Varian) and concentrated under reduced pressure to give an oil. This residue was purified by silica gel flash chromatography (5% methanol in ethyl acetate as eluant) to afford 150 mg of the title product, a compound of the present invention.

¹H NMR (CDCl₃) δ 8.35 (s, 1H), 7.59 (s, 1H), 7.58-7.51 (m, 1H), 7.08 (t, 2H), 3.78-3.74 (m, 5H), 2.01-1.92 (m, 1H), 0.76 (d, 6H).

### Example 12

### Preparation of 5-Chloro-6-(2,6-difluorophenyl)-1-(2-methylpropyl)-3-(5-methyl-2-pyridinyl)-2(1H)-pyrazinone (Compound 209)

### Step A: Preparation of 5-Chloro-6-(2,6-difluorophenyl)-3-iodo-1-(2-methylpropyl)-2(1H)-pyrazinone

To a solution of 3,5-dichloro-6-(2,6-difluorophenyl)-1-(2-methylpropyl)-2(1H)-pyrazinone (i.e. the product of Example 10 step A) (0.50 g, 1.50 mmol) in acetonitrile (10 mL) was added sodium iodide (0.34 g, 2.25 mmol), hydroiodic acid (10 drops), and acetone (1 mL). The resulting mixture was heated at reflux for 2 h and allowed to cool to room temperature. The reaction mixture was diluted with diethyl ether, filtered, and concentrated in vacuo. The residue was passed through a ChemElute®, diatomaceous earth column (Varian) washed with dichloromethane, and concentrated under reduced pressure to give an oil. This residue was purified using a Bond Elut® SI, silica gel column (Varian) and dichloromethane as eluant to afford 620 mg of the title compound.

¹H NMR (CDCl₃) δ 7.62-7.55 (m, 1H), 7.10 (t, 2H), 3.68 (d, 2H), 1.93 (s, 1H), 0.77-0.73 (m, 6H).

### Step B: Preparation of 5-Chloro-6-(2,6-difluorophenyl)-1-(2-methylpropyl)-3-(5-methyl-2-pyridinyl)-2(1H)-pyrazinone (Compound 209)

To a solution of 5-chloro-6-(2,6-difluorophenyl)-3-iodo-1-(2-methylpropyl)-2(1H)-pyrazinone (i.e. the product of Example 12 step A) (0.50 g, 1.18 mmol) in tetrahydrofuran (20 mL) was added tetrakis(triphenylphosphine)palladium(0) (0.13 g, 0.12 mmol) and 4-methyl-2-pyridinylzinc bromide (Aldrich, 0.5 M solution in tetrahydrofuran, 3.54 mL, 1.77 mmol). The resulting mixture was heated at 80° C. overnight and concentrated in vacuo. The residue was purified by silica gel flash chromatography (20% ethyl acetate in dichloromethane eluant) to provide 380 mg of the title product, a compound of the present invention.

¹H NMR (CDCl₃) δ 8.68 (s, 1H), 8.40 (s, 1H), 7.65-7.57 (m, 2H), 7.12 (t, 2H), 3.79 (d, 2H), 2.44 (s, 3H), 2.04-1.99 (m, 1H), 0.78 (d, 6H).

### Example 13

### Preparation of 5-Chloro-6-(2,6-difluorophenyl)-3-formamido-1-(2-methylpropyl)-2(1H)-pyrazinone (Compound 422)

To a solution of 3,5-dichloro-6-(2,6-difluorophenyl)-1-(2-methylpropyl)-2(1H)-pyrazinone (i.e. the product of Example 10 step A) (500 mg, 1.5 mmol) and 4 Å molecular sieves (8.0 g) in N,N-dimethylformamide (6 mL) was added sodium hydride (55% dispersion in mineral oil, 0.297 g, 3.75 mmol) at room temperature. The reaction mixture was stirred for 15 minutes, and formamide (0.203 g, 4.5 mmol) was added. The reaction mixture was stirred for 3 h at 60° C. and then filtered through a sintered glass frit and concentrated under reduced pressure. The residue was purified by MPLC (20 to 100% ethyl acetate in hexanes as eluant) to afford 258 mg of the title product, a compound of the present invention, as an oil.

¹H NMR (CDCl₃) δ 9.41 (d, 1H), 9.15-9.08 (m, 1H), 7.62-7.53 (m, 1H), 7.14-7.07 (m, 2H), 3.71 (d, 2H), 1.94-1.84 (m, 1H), 0.76 (d, 6H).

### Example 14

### Preparation of 5-(2,4-Difluorophenyl)-3,4-dihydro-3-imino-6-methyl-4-(2-methylbutyl)pyrazinecarbonitrile (Compound 471) and N-[3-Cyano-6-(2,4-difluorophenyl)-5-methyl-1-(2-methylbutyl)-2(1H)-pyrazinylidene]acetamide (Compound 475)

### Step A: Preparation of 4-[1-(2,4-Difluorophenyl)-1-propenyl]morpholine

To a solution of 1-(2,4-difluorophenyl)-1-propanone (17 g, 100 mmol) and morpholine (35 mL, 400 mmol) in toluene (350 mL) was added dropwise a 1 M solution of titanium(IV) chloride in toluene (50 mL, 50 mmol) at such a rate as to maintain a temperature below −10° C. After the addition was complete the reaction mixture was allowed to warm to room temperature and stirred overnight. It was then filtered through Celite® diatomaceous filter aid. The solvent was removed with a rotary evaporator to afford 16 g of the title compound as an oil.

¹H NMR (CDCl₃) δ 7.28 (m, 1H), 6.89 (dd, 1H), 6.82 (dd, 1H), 4.82 (q, 1H), 3.68 (m, 4H), 2.72 (m, 4H), 1.46 (d, 3H).

### Step B: Preparation of [[2-(2,4-Difluorophenyl)-1-methyl-2-(4-morpholinyl)ethenyl]-imino]propanedinitrile

To a solution of 4-[1-(2,4-difluorophenyl)-1-propenyl]morpholine (i.e. the product of Example 14 Step A) (8.0 g, 34 mmol) and [[[(4-methylphenyl)sulfonyl]oxy]imino]-propanedinitrile (8.3 g, 34 mmol) in diethyl ether (250 mL) at 0° C. was added dropwise a solution of pyridine (3.0 mL, 37 mmol) in diethyl ether (50 mL). After the addition was complete the reaction mixture was allowed to warm to room temperature and stirred for three days. The reaction mixture was diluted with hexanes, and a solid was filtered off. The solvent was removed from the filtrate using a rotary evaporator. The residue was triturated with chlorobutane and then water. The solid obtained was dried in a vacuum oven to afford 7.1 g of the title compound.

¹H NMR (CDCl₃) δ 7.24 (m, 1H), 7.05 (dd, 1H), 6.99 (dd, 1H), 3.74 (m, 4H), 2.99 (m, 4H), 2.45 (s, 3H).

### Step C: Preparation of 5-(2,4-Difluorophenyl)-3,4-dihydro-3-imino-6-methyl-4-(2-methylbutyl)pyrazinecarbonitrile (Compound 471)

To a solution of [[2-(2,4-difluorophenyl)-1-methyl-2-(4-morpholinyl)ethenyl]imino]-propanedinitrile (i.e. the product of Example 14 Step B) (2.0 g, 6.3 mmol) in chloroform (20 mL) at room temperature was added 2-methylbutylamine (0.87 mL, 7.6 mmol). The reaction mixture was allowed to stand overnight. The solvent was removed with a rotary evaporator. The residue was purified by MPLC (15 to 30% ethyl acetate in hexanes as eluant) to afford an impure sample of the title compound (0.87 g). This material was purified further by MPLC (20 to 30% ethyl acetate in hexanes as eluant) to afford 0.4 g of the title product, a compound of the present invention, as a red oil.

¹H NMR (CDCl₃) δ 7.25 (m, 1H), 7.08 (dd, 1H), 7.02 (dd, 1H), 3.76 (br s, 1H), 3.60 (br s, 1H), 1.92 (m, 1H), 1.90 (s, 3H), 0.72 (m, 6H).

### Step D: Preparation of N-[3-Cyano-6-(2,4-difluorophenyl)-5-methyl-1-(2-methyl-butyl)-2(1H)-pyrazinylidene]acetamide (Compound 475)

5-(2,4-Difluorophenyl)-3,4-dihydro-3-imino-6-methyl-4-(2-methylbutyl)pyrazine-carbonitrile (i.e. the product of Example 14 Step C) (0.13 g, 0.41 mmol) was dissolved in acetic anhydride (2 mL). The reaction mixture was stirred at room temperature overnight and then concentrated with a rotary evaporator. Diethyl ether was added, and the organic layer was washed with 1 N sodium hydroxide aqueous solution. It was dried (NaSO₄) and concentrated with a rotary evaporator. The residue was purified by MPLC (30 to 50% ethyl acetate in hexanes as eluant) to afford 90 mg of the title product, a compound of the present invention, as a viscous oil.

¹H NMR (CDCl₃) δ 7.25 (m, 1H), 7.14 (dd, 1H), 7.07 (dd, 1H), 3.96 (br s, 1H), 3.84 (br s, 1H), 2.31 (s, 3H), 2.09 (s, 3H), 1.82 (m, 1H), 1.17 (m, 1H), 1.01 (m, 1H), 0.72 (m, 6H).

### Example 15

### Preparation of 5-Chloro-6-(2,6-difluoro-4-methoxyphenyl)-1-(2-methylbutyl)-3-(1H-pyrazol-1-yl)-2(1H)-pyrazinone (Compound 451), 5-Chloro-6-(2,6-difluoro-4-hydroxyphenyl)-1-(2-methylbutyl)-3-(1H-pyrazol-1-yl)-2(1H)-pyrazinone (Compound 453) and 5-Chloro-6-[4-[2-(dimethylamino)ethoxy]-2,6-difluorophenyl]-1-(2-methylbutyl)-3-(1H-pyrazol-1-yl)-2(1H)-pyrazinone (Compound 479)

### Step A: Preparation of 2,6-Difluoro-4-methoxybenzaldehyde

3,5-Difluoroanisole (5 g, 34.7 mmol) was dissolved in tetrahydrofuran (73 mL) and cooled to −78° C. A solution of n-butyl lithium (2.5 M solution in tetrahydrofuran, 2.5 mL, 2.50 mmol) was slowly added, and the reaction mixture was stirred at −78° C. for 1.5 h. At this point, N,N-dimethylformamide (10 mL) was added, and the reaction was stirred for 10 minutes at −78° C. and then another 10 minutes at 0° C. The reaction mixture was then quenched with 50 mL of 1M HCl. The reaction mixture was extracted with ethyl acetate (3×50 mL), the organic layers combined, dried over MgSO₄, concentrated and the crude oil was purified by MPLC (0 to 20% gradient of ethyl acetate in hexanes as eluant) to yield 5.45 g of the title product as a fluffy yellow solid.

¹H NMR (CDCl₃) δ 10.20 (s, 1H), 6.49 (d, 2H), 3.87 (s, 3H).

### Step B: Preparation of 2,6-Difluoro-4-methoxy-α-[(2-methoxybutyl)-amino]benzeneacetonitrile

To a solution of sodium hydrogensulfite (1.03 g, 9.9 mmol) in a mixture of deionized water (20 mL) and methanol (2.0 mL) at room temperature was added 2,6-difluoro-4-methoxybenzaldehyde (i.e. the product of Example 15 Step A) (1.62 g, 9.4 mmol). The reaction mixture was stirred for 15 minutes, and sodium cyanide (0.49 g, 9.9 mmol) was added. The reaction mixture was stirred for an additional 20 minutes and cooled using an ice water bath. A solution of methylbutylamine (0.90 g, 10.4 mmol) in methanol (4.0 mL) was added over approximately 2 minutes, and the resulting reaction mixture was stirred at 0° C. for 15 minutes and then heated to 35° C. for 2 h. The resulting mixture was then extracted with ethyl acetate (2×40 mL), and the combined organic layers were washed with brine, dried (MgSO₄) and concentrated to give 2.51 g of the title product as an oil.

¹H NMR (CDCl₃) δ 6.51 (m, 2H), 4.83 (br s, 1H), 3.80 (s, 3H), 2.76 (m, 1H), 2.52 (m, 1H), 1.49 (m, 2H), 1.17 (m, 1H), 0.90 (m, 6H).

### Step C: Preparation of 3,5-Dichloro-6-(2,6-difluoro-4-methoxyphenyl)-1-(2-methylbutyl)-2(1H)-pyrazinone

A solution of 2,6-difluoro-4-methoxy-α-[(2-methoxybutyl)-amino]benzeneacetonitrile (i.e. the product of Example 15 Step B) (2.51 g, 9.4 mmol) in chlorobenzene (10 mL) was added dropwise over 20 minutes to a solution of oxalyl chloride (5.94 g, 46.8 mmol) in chlorobenzene (25 mL) at room temperature. The reaction mixture was then heated to 100° C. overnight. N,N-Dimethylformamide (0.5 mL) was then added, and the reaction mixture was heated for an additional 2 h. The reaction mixture was then concentrated under reduced pressure and the resulting residue was purified by MPLC (0 to 100% gradient of ethyl acetate in hexanes as eluant) to give 2.88 g of the title product as an oil.

¹H NMR (CDCl₃) δ 6.61 (m, 2H), 3.90 (s, 3H), 3.76 (m, 2H), 1.70 (m, 1H), 1.20 (m, 1H), 1.03 (m, 1H), 0.74 (m, 6H).

### Step D: Preparation of 5-Chloro-6-(2,6-difluoro-4-methoxyphenyl)-1-(2-methylbutyl)-3-(1H-pyrazol-1-yl)-2(1H)-pyrazinone (Compound 451)

A mixture of 3,5-dichloro-6-(2,6-difluoro-4-methoxyphenyl)-1-(2-methylbutyl)-2(1H)-pyrazinone (i.e. the product of Example 15 Step C) (1.0 g, 2.65 mmol), pyrazole (0.20 g, 2.92 mmol) and potassium carbonate (0.73 g, 5.30 mmol) in N,N-dimethylformamide (12 mL) was heated to 60° C. overnight. The reaction mixture was concentrated under reduced pressure and the residue was purified by MPLC (0 to 100% gradient of ethyl acetate in hexanes as eluant) to give 0.676 g of the title product, a compound of the present invention.

¹H NMR (CDCl₃) δ 9.09 (m, 1H), 7.88 (m, 1H), 6.63 (m, 2H), 6.50 (m, 1H), 3.87 (m, 5H), 1.75 (m, 1H), 1.25 (m, 1H), 1.05 (m, 1H), 0.74 (m, 6H).

### Step E: Preparation of 5-Chloro-6-(2,6-difluoro-4-hydroxyphenyl)-1-(2-methylbutyl)-3-(1H-pyrazol-1-yl)-2(1H)-pyrazinone (Compound 453)

To a solution of 5-chloro-6-(2,6-difluoro-4-methoxyphenyl)-1-(2-methylbutyl)-3-(1H-pyrazol-1-yl)-2(1H)-pyrazinone (i.e. the product of Example 15 Step D) (0.676 g, 1.65 mmol) in dichloromethane (15 mL) at −78° C. was slowly added a solution of boron tribromide (1 M solution in dichloromethane, 6.61 mL, 6.61 mmol). The reaction mixture was allowed to warm to room temperature overnight. Then the reaction mixture was cooled to 0° C. and quenched with saturated aqueous ammonium chloride solution. The reaction mixture was extracted with dichloromethane (2×40 mL) and ethyl acetate (2×30 mL). The organic layers were combined, dried over MgSO₄ and concentrated. The crude residue was purified by MPLC (0 to 100% gradient of ethyl acetate in hexanes as eluant) to yield 0.344 g of the title product, a compound of the present invention.

¹H NMR (CDCl₃) δ 10.53 (br s, 1H), 9.24 (d, 1H), 7.94 (d, 1H), 6.74 (d, 2H), 6.57 (m, 1H), 3.90 (d, 2H), 1.76 (m, 1H), 1.26 (m, 1H), 1.05 (m, 1H), 0.77 (m, 6H).

### Step F: Preparation of 5-Chloro-6-[4-[2-(dimethylamino)ethoxy]-2,6-difluorophenyl]-1-(2-methylbutyl)-3-(1H-pyrazol-1-yl)-2(1H)-pyrazinone (Compound 479)

To a solution of 5-chloro-6-(2,6-difluoro-4-hydroxyphenyl)-1-(2-methylbutyl)-3-(1H-pyrazol-1-yl)-2(1H)-pyrazinone (i.e. the product of Example 15 Step E) (0.314 g, 0.80 mmol) in N,N-dimethylformamide (10 mL) was added cesium carbonate (1.30 g, 3.98 mmol). The reaction mixture was heated to 70° C. for 10 minutes, and then solid 2-chloro-N,N-dimethylethylamine hydrochloride (0.344 g, 2.39 mmol) was added. The reaction mixture was heated for an additional 2.25 h. The solids were then filtered off, and the reaction mixture was concentrated. The crude residue was purified by MPLC (0 to 20% gradient of methanol in dichloromethane as eluant) to yield 0.123 g of the title product, a compound of the present invention.

¹H NMR (CDCl₃) δ 9.09 (m, 1H), 7.88 (m, 1H), 6.65 (m, 2H), 6.50 (m, 1H), 4.12 (m, 2H), 3.85 (m, 2H), 2.77 (m, 2H), 2.36 (s, 6H), 1.73 (m, 1H), 1.24 (m, 1H), 1.04 (m, 1H), 0.75 (m, 6H).

### Example 16

### Preparation of 5-Chloro-1-(2,2,3,3,3-pentafluoropropyl)-3-(1H-pyrazol-1-yl)-6-(2,4,6-trifluorophenyl)-2(1H)-pyrazinone (Compound 468)

### Step A: Preparation of 2,2,3,3,3-Pentafluoro-N-[(2,4,6-trifluorophenyl)methylene]-1-propanamine

A mixture of 2,4,6-trifluorobenzaldehyde (4.51 g, 28.00 mmol) and 2,2,3,3,3-pentafluoropropylamine (4.20 g, 28.17 mmol) in toluene (30 mL) was heated at reflux overnight using Dean-Stark apparatus. The reaction mixture was allowed to cool to room temperature and concentrated in vacuo to provide 6.55 g of the title product. This compound was of sufficient purity to use in subsequent reactions.

¹H NMR (CDCl₃) δ 8.50 (s, 1H), 6.80-6.72 (m, 2H), 4.23-4.16 (m, 2H).

### Step B: Preparation of 2,4,6-Trifluoro-α-[(2,2,3,3,3-pentafluoropropyl)-amino]benzeneacetonitrile

A mixture of 2,2,3,3,3-pentafluoro-N-[(2,4,6-trifluorophenyl)methylene]-1-propanamine (i.e. the product of Example 16 Step A) (6.55 g, 22.50 mmol), zinc iodide (7.18 g, 22.50 mmol), and 5 Å molecular sieves (22.5 g) in dichloromethane (25 mL) was treated with trimethylsilyl cyanide (18.0 mL, 135.1 mmol) and the reaction mixture was heated to reflux overnight. After cooling to room temperature, the reaction mixture was filtered through Celite® diatomaceous filter aid and concentrated in vacuo. The reaction residue was treated with methanol (100 mL) and 10% aqueous sodium bicarbonate solution (20 mL), and the resulting mixture was extracted with diethyl ether (2×50 mL). The ether phase was separated, dried over MgSO₄, and concentrated in vacuo. The resulting crude residue was purified via silica gel flash chromatography (5 to 10% gradient of ethyl acetate in hexane as eluant) to provide 1.0 g of the title product.

¹H NMR (CDCl₃) δ 6.86-6.74 (m, 2H), 5.04 (d, 1H), 3.55-3.30 (m, 2H), 2.27-2.21 (m, 1H).

### Step C: Preparation of 3,5-Dichloro-1-(2,2,3,3,3-pentafluoropropyl)-6-(2,4,6-trifluorophenyl)-2(1H)-pyrazinone

Oxalyl chloride (4.33 mL, 49.65 mmol) was added dropwise to a mixture of 2,4,6-trifluoro-α-[(2,2,3,3,3-pentafluoropropyl)amino]benzeneacetonitrile (i.e. the product of Example 16 Step B) (3.16 g, 9.93 mmol) in chlorobenzene (20 mL) at room temperature. The resulting mixture was heated to 100° C. for 3 h, and then allowed to cool to room temperature. One drop of N,N-dimethylformamide was then added. The reaction mixture was reheated to 100° C. overnight. Then the reaction mixture was again allowed to cool to room temperature and concentrated in vacuo to provide a crude residue, which was purified via silica gel flash chromatography (10% ethyl acetate in hexane as eluant) to provide 0.47 g of the title product.

¹H NMR (CDCl₃) δ 6.94-6.89 (m, 2H), 4.65-4.45 (m, 2H).

### Step D: Preparation of 5-Chloro-1-(2,2,3,3,3-pentafluoropropyl)-3-(1H-pyrazol-1-yl)-6-(2,4,6-trifluorophenyl)-2(1H)-pyrazinone (Compound 468)

A mixture of 3,5-dichloro-1-(2,2,3,3,3-pentafluoropropyl)-6-(2,4,6-trifluorophenyl)-2(1H)-pyrazinone (i.e. the product of Example 16 Step C) (0.47 g, 1.10 mmol) and pyrazole (0.15 g, 2.20 mmol) in N,N-dimethylformamide (5 mL) was heated to 60° C. overnight. The reaction mixture was allowed to cool to room temperature and concentrated in vacuo. The resulting residue was subjected to silica gel flash chromatography (10% to 20% gradient of ethyl acetate in hexane as eluant) to provide partially purified material. Trituration of this material with a mixture of hexane and n-butyl chloride provided 0.30 g of the title product, a compound of the present invention, as a white solid melting at 147-149° C.

¹H NMR (CDCl₃) δ 9.05 (d, 1H), 7.93 (d, 1H), 6.94-6.88 (m, 2H), 6.55 (s, 1H), 4.75-4.50 (m, 2H).

### Example 17

### Preparation of 5-chloro-6-[2-chloro-6-fluoro-4-[3-(methylamino)propoxy]phenyl]-1-[(2S)-2-methylbutyl]-3-(1H-pyrazol-1-yl)-2(1H)-pyrazinone (Compound 493)

### Step A: Preparation of Phenylmethyl N-(3-chloropropyl)-N-methylcarbamate

A mixture of N-methyl-3-chloropropylamine hydrochloride (1.11 g, 7.7 mmol), benzyl chloroformate (1.45 g, 8.5 mmol) and N,N-diisopropylethylamine (2.24 g, 17.3 mmol) were dissolved in dichloromethane (25 mL) at 0° C. The reaction mixture was allowed to warm to room temperature overnight. The reaction mixture was then concentrated under reduced pressure and purified by MPLC (0 to 100% gradient of ethyl acetate in hexanes as eluant) to provide 1.57 g of the title product.

¹H NMR (CDCl₃) δ 7.36 (m, 4H), 7.33 (m, 1H), 5.13 (s, 2H), 3.54 (m, 2H), 3.44 (t, 2H), 2.96 (s, 3H), 2.04 (m, 2H).

### Step B: Preparation of Phenylmethyl N-[3-[4-[3-chloro-1,6-dihydro-1-[(2S)-2-methylbutyl]-6-oxo-5-(1H-pyrazol-1-yl)-2-pyrazinyl]-3,5-difluorophenoxy]propyl]-N-methylcarbamate

To a solution of 5-chloro-6-(2,6-difluoro-4-hydroxyphenyl)-1-[(2S)-2-methylbutyl]-3-(1H-pyrazol-1-yl)-2(1H)-pyrazinone (prepared in the same manner as Example 15 Step E using (S)-(−)-2-methylbutylamine) (0.35 g, 0.89 mmol) in N,N-dimethylformamide (4 mL) was added dry activated 4 Å molecular sieves (3.0 g). The reaction mixture was stirred for 3 h at room temperature. Tetrabutylammonium iodide (0.065 g, 0.18 mmol) and phenylmethyl N-(3-chloropropyl)-N-methylcarbamate (i.e. the product of Example 17 Step A) (0.641 g, 2.66 mmol) in N,N-dimethylformamide (1 mL), were added and the reaction mixture was stirred for 15 minutes at room temperature. Then cesium carbonate (0.867 g, 2.66 mmol) was added and stirring was continued for another 15 minutes. The reaction mixture was then heated to 75° C. for 2 h and then cooled to room temperature. After the molecular sieves and cesium carbonate were removed by filtering through Celite®, diatomaceous filter aid, the reaction mixture was concentrated under reduced pressure. The crude oil was purified by MPLC (0 to 100% gradient of ethyl acetate in hexanes as eluant) to provide 0.442 g of the title product.

¹H NMR (CDCl₃) δ 9.09 (d, 1H), 7.88 (d, 1H), 7.33 (m, 5H), 6.59 (m, 1H), 6.50 (m, 2H), 5.11 (s, 2H), 4.00 (m, 2H), 3.85 (m, 2H), 3.51 (t, 2H), 2.98 (s, 3H), 2.10 (m, 2H), 1.72 (m, 1H), 1.20 (m, 1H), 1.03 (m, 1H), 0.74 (m, 6H).

### Step C: Preparation of 5-chloro-6-[2-chloro-6-fluoro-4-[3-(methylamino)propoxy]-phenyl]-1-[(2S)-2-methylbutyl]-3-(1H-pyrazol-1-yl)-2(1H)-pyrazinone

Phenylmethyl N-[3-[4-[3-chloro-1,6-dihydro-1-[(2S)-2-methylbutyl]-6-oxo-5-(1H-pyrazol-1-yl)-2-pyrazinyl]-3,5-difluorophenoxy]propyl]-N-methylcarbamate (i.e. the product of Example 17 Step B) (0.44 g, 7.36 mmol) was dissolved in methanol (50 mL) and flushed with nitrogen. Hydrogen chloride (1M solution in diethyl ether, 4 mL) was added followed by palladium on carbon (10% wt/wt, 0.117 g, 0.110 mmol) and flushing with nitrogen was continued. A balloon containing hydrogen gas was attached to the reaction mixture and the reaction mixture was stirred at room temperature for 2 h. The reaction mixture was filtered through Celite®, diatomaceous filter aid, and concentrated under reduced pressure. The reaction mixture was redissolved in methanol, filtered, and then concentrated to give 0.35 g of the title product, a compound of the present invention.

¹H NMR (methanol-d₄) δ 9.08 (d, 1H), 8.23 (m, 1H), 7.89 (d, 1H), 6.91 (m, 2H), 6.59 (s, 1H), 4.22 (t, 2H), 3.87 (m, 2H), 3.23 (m, 2H), 2.75 (s, 3H), 2.21 (m, 2H), 1.73 (m, 1H), 1.24 (m, 1H), 1.06 (m, 1H), 0.73 (m, 6H).

### Example 18

### Preparation of 5-chloro-6-(2,6-difluoro-4-methoxyphenyl)-1-[(2S)-2-methylbutyl]-3-(1-methyl-1H-pyrazol-3-yl)-2(1H)-pyrazinone (Compound 490)

A mixture of 3,5-dichloro-6-(2,6-difluoro-4-methoxyphenyl)-1-[2(S)-methylbutyl]-2(1H)-pyrazinone (prepared according to the procedure of the compound of Example 15 Step C) (0.5 g, 1.33 mmol), (1-methyl-1H-pyrazol-3-yl)tributylstannane (0.447 g, 1.20 mmol) and trans-dichlorobis(triphenylphosphine)palladium (II) (0.042 g, 0.06 mmol) in toluene (10 mL) were heated to reflux overnight. The reaction mixture was concentrated under reduced pressure and the resulting residue was purified by MPLC (0 to 100% gradient of ethyl acetate in hexanes as eluant) to give 0.37 g of the title product, a compound of the present invention.

¹H NMR (CDCl₃) δ 7.44 (d, 1H), 7.40 (d, 1H), 6.61 (m, 2H), 4.05 (s, 3H), 3.88 (s, 3H), 3.81 (m, 2H), 1.77 (m, 1H), 1.25 (m, 1H), 1.01 (m, 1H), 0.74 (m, 6H).

By the procedures described herein together with methods known in the art, the following compounds of Tables 1 to 7 can be prepared. The following abbreviations are used in the Tables which follow: t means tertiary, s means secondary, n means normal, i means iso, c means cyclo, Me means methyl, Et means ethyl, Pr means propyl, i-Pr means isopropyl, Bu means butyl, Hex means hexyl, Ph means phenyl, OMe meansmethoxy, OEtmeansethoxy, SMe means methylthio, S(O) means sulfinyl, S(O)₂ means sulfonyl, CN means cyano, NO₂ means nitro, and 2-Cl-4-F means 2-chloro-4-fluoro, and other substituent abbreviations are defined analogously.

TABLE 1a

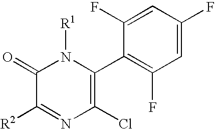

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-l-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| CH₂CH₂CH₂F | 1H-1,2,4-triazol-1-yl |
| CH₂CF₂CF₃ | 1H-1,2,4-triazol-1-yl |
| CH₂CH₂CF₃ | 1H-1,2,4-triazol-1-yl |
| CH₂CH(Me)CF₃ | 1H-1,2,4-triazol-1-yl |
| (S)-CH₂CH(Me)CF₃ | 1H-1,2,4-triazol-1-yl |
| CH₂CH₂CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3 -Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 1-methyl-1H-pyrazol-3-yl |
| Et | 1-methyl-1H-pyrazol-3-yl |
| i-Pr | 1-methyl-1H-pyrazol-3-yl |
| n-Pr | 1-methyl-1H-pyrazol-3-yl |
| i-Bu | 1-methyl-1H-pyrazol-3-yl |
| n-Bu | 1-methyl-1H-pyrazol-3-yl |
| s-Bu | 1-methyl-1H-pyrazol-3-yl |
| 3-Me-Bu | 1-methyl-1H-pyrazol-3-yl |
| n-pentyl | 1-methyl-1H-pyrazol-3-yl |
| n-Hex | 1-methyl-1H-pyrazol-3-yl |
| 2-propenyl | 1-methyl-1H-pyrazol-3-yl |
| 2-Me-2-propenyl | 1-methyl-1H-pyrazol-3-yl |
| 3-butenyl | 1-methyl-1H-pyrazol-3-yl |
| 3-pentenyl | 1-methyl-1H-pyrazol-3-yl |
| 2-propynyl | 1-methyl-1H-pyrazol-3-yl |
| 3-butynyl | 1-methyl-1H-pyrazol-3-yl |
| 4-butynyl | 1-methyl-1H-pyrazol-3-yl |
| c-Pr | 1-methyl-1H-pyrazol-3-yl |
| c-pentyl | 1-methyl-1H-pyrazol-3-yl |
| 2-cyclohexenyl | 1-methyl-1H-pyrazol-3-yl |
| 3-cyclohexenyl | 1-methyl-1H-pyrazol-3-yl |
| CH₂-c-Pr | 1-methyl-1H-pyrazol-3-yl |
| CH₂-c-Hex | 1-methyl-1H-pyrazol-3-yl |
| CH₂-2-cyclohexenyl | 1-methyl-1H-pyrazol-3-yl |
| 4-tetrahydropyranyl | 1-methyl-1H-pyrazol-3-yl |
| 3-tetrahydropyranyl | 1-methyl-1H-pyrazol-3-yl |
| 3-tetrahydrofuranyl | 1-methyl-1H-pyrazol-3-yl |
| 2-pyridinyl | 1-methyl-1H-pyrazol-3-yl |
| 2-pyrimidyl | 1-methyl-1H-pyrazol-3-yl |
| 2-pyrazinyl | 1-methyl-1H-pyrazol-3-yl |
| 2-thiazolyl | 1-methyl-1H-pyrazol-3-yl |
| 2-oxazolyl | 1-methyl-1H-pyrazol-3-yl |
| CF₃ | 1-methyl-1H-pyrazol-3-yl |
| CF₂CF₃ | 1-methyl-1H-pyrazol-3-yl |
| CH₂CF₃ | 1-methyl-1H-pyrazol-3-yl |
| CH(Me)CF₃ | 1-methyl-1H-pyrazol-3-yl |
| CH₂CH₂F | 1-methyl-1H-pyrazol-3-yl |
| CH₂CH₂CH₂F | 1-methyl-1H-pyrazol-3-yl |
| CH₂CF₂CF₃ | 1-methyl-1H-pyrazol-3-yl |
| CH₂CH₂CF₃ | 1-methyl-1H-pyrazol-3-yl |
| CH₂CH(Me)CF₃ | 1-methyl-1H-pyrazol-3-yl |
| (S)-CH₂CH(Me)CF₃ | 1-methyl-1H-pyrazol-3-yl |
| CH₂CH₂CH₂CH₂F | 1-methyl-1H-pyrazol-3-yl |
| 2-chloro-2-propenyl | 1-methyl-1H-pyrazol-3-yl |
| 3,3-dichloro-2-propenyl | 1-methyl-1H-pyrazol-3-yl |
| CH₂-2-tetrahydrofuranyl | 1-methyl-1H-pyrazol-3-yl |
| CH₂-2-tetrahydropyranyl | 1-methyl-1H-pyrazol-3-yl |
| CH₂CN | 1-methyl-1H-pyrazol-3-yl |
| CH₂NO₂ | 1-methyl-1H-pyrazol-3-yl |
| CH₂CH₂OH | 1-methyl-1H-pyrazol-3-yl |
| CH₂CH₂OMe | 1-methyl-1H-pyrazol-3-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3 -Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |
| Me | CONH₂ |
| Et | CONH₂ |
| i-Pr | CONH |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH2 |
| CH₂CH₂CH₂F | CONH₂ |
| CH₂CF₂CF₃ | CONH₂ |
| CH₂CH₂CF₃ | CONH₂ |
| CH₂CH(Me)CF₃ | CONH₂ |
| (S)-CH₂CH(Me)CF₃ | CONH₂ |
| CH₂CH₂CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3 -pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |
| CH(Me)CH₂OMe | 1-methyl-1H-pyrazol-3-yl |
| CH(Me)CH(OMe)₂ | 1-methyl-1H-pyrazol-3-yl |
| CH₂-2-dioxolanyl | 1-methyl-1H-pyrazol-3-yl |
| CH₂CH₂OCF₃ | 1-methyl- 1H-pyrazol-3-yl |
| CH₂CH₂SMe | 1-methyl-1H-pyrazol-3-yl |
| CH₂CH(Me)SMe | 1-methyl-1H-pyrazol-3-yl |
| CH₂CH₂S(O)Me | 1-methyl-1H-pyrazol-3-yl |
| CH₂CH₂S(O)₂Me | 1-methyl-1H-pyrazol-3-yl |
| CH₂CO₂Me | 1-methyl-1H-pyrazol-3-yl |
| CH₂CO₂-i-Pr | 1-methyl-1H-pyrazol-3-yl |
| CH(Me)CO₂Me | 1-methyl-1H-pyrazol-3-yl |
| CH₂C(O)Me | 1-methyl-1H-pyrazol-3-yl |
| CH₂CH₂C(O)Me | 1-methyl-1H-pyrazol-3-yl |
| CH₂SiMe₃ | 1-methyl-1H-pyrazol-3-yl |
| CH₂CH₂SiMe₃ | 1-methyl-1H-pyrazol-3-yl |
| CH₂OPh | 1-methyl-1H-pyrazol-3-yl |
| CH₂Ph | 1-methyl-1H-pyrazol-3-yl |
| CH₂CH₂Ph | 1-methyl-1H-pyrazol-3-yl |
| CH(Me)Ph | 1-methyl-1H-pyrazol-3-yl |
| CH₂-2-Cl-Ph | 1-methyl-1H-pyrazol-3-yl |
| CH₂-3-Cl-Ph | 1-methyl-1H-pyrazol-3-yl |
| CH₂-4-Cl-Ph | 1-methyl-1H-pyrazol-3-yl |
| CH₂-2-thienyl | 1-methyl-1H-pyrazol-3-yl |
| CH₂-2-pyridinyl | 1-methyl-1H-pyrazol-3-yl |
| CH2-3-pyridinyl | 1-methyl-1H-pyrazol-3-yl |
| CH(Et)₂ | 1-methyl-1H-pyrazol-3-yl |
| CH₂CH(Et)₂ | 1-methyl-1H-pyrazol-3-yl |
| CH₂CH(n-Pr)Me | 1-methyl-1H-pyrazol-3-yl |
| CH(Me)Et | 1-methyl-1H-pyrazol-3-yl |
| CH(Me)-n-Pr | 1-methyl-1H-pyrazol-3-yl |
| CH(CF₃)Et | 1-methyl-1H-pyrazol-3-yl |
| CH(Et)-n-Pr | 1-methyl-1H-pyrazol-3-yl |
| CH(Me)-n-Bu | 1-methyl-1H-pyrazol-3-yl |
| 2,2-dimethylpropyl | 1-methyl-1H-pyrazol-3-yl |
| CH₂CH₂CH(Me)₂ | 1-methyl-1H-pyrazol-3-yl |
| CH₂-2-F-Ph | 1-methyl-1H-pyrazol-3-yl |
| CH₂-3-F-Ph | 1-methyl-1H-pyrazol-3-yl |
| CH₂-4-F-Ph | 1-methyl-1H-pyrazol-3-yl |
| CH₂-2-Me-Ph | 1-methyl-1H-pyrazol-3-yl |
| CH₂-3-Me-Ph | 1-methyl-1H-pyrazol-3-yl |
| CH₂-4-Me-Ph | 1-methyl-1H-pyrazol-3-yl |
| CH₂-2-OMe-Ph | 1-methyl-1H-pyrazol-3-yl |
| CH₂-3-OMe-Ph | 1-methyl-1H-pyrazol-3-yl |
| CH₂-4-OMe-Ph | 1-methyl-1H-pyrazol-3-yl |
| cis-2-Me-c-Hex | 1-methyl-1H-pyrazol-3-yl |
| trans-2-Me-c-Hex | 1-methyl-1H-pyrazol-3-yl |
| cis-3-Me-c-Hex | 1-methyl-1H-pyrazol-3-yl |
| trans-3-Me-c-Hex | 1-methyl-1H-pyrazol-3-yl |
| cis-4-Me-c-Hex | 1-methyl-1H-pyrazol-3-yl |
| trans-4-Me-c-Hex | 1-methyl-1H-pyrazol-3-yl |
| CH₂CH(Me)OMe | 1-methyl-1H-pyrazol-3-yl |

TABLE 1b

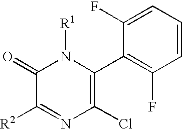

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-C1-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-C1-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |

TABLE 1c

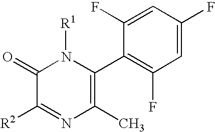

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-C1-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-C1-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |

TABLE 1d

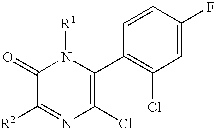

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3-dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-C1-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| CH₂-2-F-Ph | 1H-pyrazol-1-yl |
| CH₂-3-F-Ph | 1H-pyrazol-1-yl |
| CH₂-4-F-Ph | 1H-pyrazol-1-yl |
| CH₂-2-Me-Ph | 1H-pyrazol-1-yl |
| CH₂-3-Me-Ph | 1H-pyrazol-1-yl |
| CH₂-4-Me-Ph | 1H-pyrazol-1-yl |
| CH₂-2-OMe-Ph | 1H-pyrazol-1-yl |
| CH₂-3-OMe-Ph | 1H-pyrazol-1-yl |
| CH₂-4-OMe-Ph | 1H-pyrazol-1-yl |
| cis-2-Me-c-Hex | 1H-pyrazol-1-yl |
| trans-2-Me-c-Hex | 1H-pyrazol-1-yl |
| cis-3-Me-c-Hex | 1H-pyrazol-1-yl |
| trans-3-Me-c-Hex | 1H-pyrazol-1-yl |
| cis-4-Me-c-Hex | 1H-pyrazol-1-yl |
| trans-4-Me-c-Hex | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| CH₂CH₂CH₂F | 1H-1,2,4-triazol-1-yl |
| CH₂CF₂CF₃ | 1H-1,2,4-triazol-1-yl |
| CH₂CH₂CF₃ | 1H-1,2,4-triazol-1-yl |
| CH₂CH(Me)CF₃ | 1H-1,2,4-triazol-1-yl |
| (S)-CH₂CH(Me)CF₃ | 1H-1,2,4-triazol-1-yl |
| CH₂CH₂CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH₂-2-dioxolanyl | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-C1-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethylpropyl | 1H-1,2,4-triazol-1-yl |
| CH₂CH₂CH(Me)₂ | 1H-1,2,4-triazol-1-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3-dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-C1-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| CH₂-2-F-Ph | 2-pyridinyl |
| CH₂-3-F-Ph | 2-pyridinyl |
| CH₂-4-F-Ph | 2-pyridinyl |
| CH₂-2-Me-Ph | 2-pyridinyl |
| CH₂-3-Me-Ph | 2-pyridinyl |
| CH₂-4-Me-Ph | 2-pyridinyl |
| CH₂-2-OMe-Ph | 2-pyridinyl |
| CH₂-3-OMe-Ph | 2-pyridinyl |
| CH₂-4-OMe-Ph | 2-pyridinyl |
| cis-2-Me-c-Hex | 2-pyridinyl |
| trans-2-Me-c-Hex | 2-pyridinyl |
| cis-3-Me-c-Hex | 2-pyridinyl |
| trans-3-Me-c-Hex | 2-pyridinyl |
| cis-4-Me-c-Hex | 2-pyridinyl |
| trans-4-Me-c-Hex | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| CH₂CH₂CH₂F | CONH₂ |
| CH₂CF₂CF₃ | CONH₂ |
| CH₂CH₂CF₃ | CONH₂ |
| CH₂CH(Me)CF₃ | CONH₂ |
| (S)-CH₂CH(Me)CF₃ | CONH₂ |
| CH₂CH₂CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH₂-2-dioxolanyl | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-C1-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethylpropyl | CONH₂ |
| CH₂CH₂CH(Me)₂ | CONH₂ |

TABLE 1e

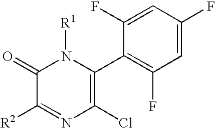

| R¹ | R² |
|---|---|
| 2-F-Ph | 1H-pyrazol-1-yl |
| 3-F-Ph | 1H-pyrazol-1-yl |
| 4-F-Ph | 1H-pyrazol-1-yl |
| 2,3-di-F-Ph | 1H-pyrazol-1-yl |
| 2,4-di-F-Ph | 1H-pyrazol-1-yl |
| 2,5-di-F-Ph | 1H-pyrazol-1-yl |
| 2,6-di-F-Ph | 1H-pyrazol-1-yl |
| 3,4-di-F-Ph | 1H-pyrazol-1-yl |
| 3,5-di-F-Ph | 1H-pyrazol-1-yl |
| 2,3-di-Cl-Ph | 1H-pyrazol-1-yl |
| 2,4-di-Cl-Ph | 1H-pyrazol-1-yl |
| 2,5-di-Cl-Ph | 1H-pyrazol-1-yl |
| 2,6-di-Cl-Ph | 1H-pyrazol-1-yl |
| 3,4-di-Cl-Ph | 1H-pyrazol-1-yl |
| 3,5-di-Cl-Ph | 1H-pyrazol-1-yl |
| 2-OMe-Ph | 1H-pyrazol-1-yl |
| 3-OMe-Ph | 1H-pyrazol-1-yl |
| 4-OMe-Ph | 1H-pyrazol-1-yl |
| 2-Me-Ph | 1H-pyrazol-1-yl |
| 3-Me-Ph | 1H-pyrazol-1-yl |
| 4-Me-Ph | 1H-pyrazol-1-yl |
| 2-CF₃-Ph | 1H-pyrazol-1-yl |
| 3-CF₃-Ph | 1H-pyrazol-1-yl |
| 4-CF₃-Ph | 1H-pyrazol-1-yl |
| 2-CN-Ph | 1H-pyrazol-1-yl |
| 3-CN-Ph | 1H-pyrazol-1-yl |
| 4-CN-Ph | 1H-pyrazol-1-yl |
| 2-NO₂-Ph | 1H-pyrazol-1-yl |
| 3-NO₂-Ph | 1H-pyrazol-1-yl |
| 4-NO₂-Ph | 1H-pyrazol-1-yl |
| 3-(CH═CH₂)-Ph | 1H-pyrazol-1-yl |
| 3-(CCH)-Ph | 1H-pyrazol-1-yl |
| 4-c-Pr-Ph | 1H-pyrazol-1-yl |
| 3-(CH═CCl₂)-Ph | 1H-pyrazol-1-yl |
| 3-(CCCl)-Ph | 1H-pyrazol-1-yl |
| 3-(2,2-di-Cl-c-Pr)-Ph | 1H-pyrazol-1-yl |
| 2-OCF₃-Ph | 1H-pyrazol-1-yl |
| 3-OCF₃-Ph | 1H-pyrazol-1-yl |
| 4-OCF₃-Ph | 1H-pyrazol-1-yl |
| 3-SMe-Ph | 1H-pyrazol-1-yl |
| 3-S(O)Me-Ph | 1H-pyrazol-1-yl |
| 3-SO2Me-Ph | 1H-pyrazol-1-yl |
| 3-NHMe-Ph | 1H-pyrazol-1-yl |
| 3-NMe₂-Ph | 1H-pyrazol-1-yl |
| 3-NH-c-Pr-Ph | 1H-pyrazol-1-yl |
| 3-COMe-Ph | 1H-pyrazol-1-yl |
| 3-CO₂Me-Ph | 1H-pyrazol-1-yl |
| 3-CONHMe-Ph | 1H-pyrazol-1-yl |
| 3-CONMe₂-Ph | 1H-pyrazol-1-yl |
| 3-SiMe₃-Ph | 1H-pyrazol-1-yl |
| 2,3-di-Me-Ph | 1H-pyrazol-1-yl |
| 2-F-Ph | 1H-1,2,4-triazol-1-yl |
| 3-F-Ph | 1H-1,2,4-triazol-1-yl |
| 4-F-Ph | 1H-1,2,4-triazol-1-yl |
| 2,3-di-F-Ph | 1H-1,2,4-triazol-1-yl |
| 2,4-di-F-Ph | 1H-1,2,4-triazol-1-yl |
| 2,5-di-F-Ph | 1H-1,2,4-triazol-1-yl |
| 2,6-di-F-Ph | 1H-1,2,4-triazol-1-yl |
| 3,4-di-F-Ph | 1H-1,2,4-triazol-1-yl |
| 3,5-di-F-Ph | 1H-1,2,4-triazol-1-yl |
| 2,3-di-Cl-Ph | 1H-1,2,4-triazol-1-yl |
| 2,4-di-Cl-Ph | 1H-1,2,4-triazol-1-yl |
| 2,5-di-Cl-Ph | 1H-1,2,4-triazol-1-yl |
| 2,6-di-Cl-Ph | 1H-1,2,4-triazol-1-yl |
| 3,4-di-Cl-Ph | 1H-1,2,4-triazol-1-yl |
| 3,5-di-Cl-Ph | 1H-1,2,4-triazol-1-yl |
| 2-OMe-Ph | 1H-1,2,4-triazol-1-yl |
| 3-OMe-Ph | 1H-1,2,4-triazol-1-yl |
| 4-OMe-Ph | 1H-1,2,4-triazol-1-yl |
| 2-Me-Ph | 1H-1,2,4-triazol-1-yl |
| 3-Me-Ph | 1H-1,2,4-triazol-1-yl |
| 4-Me-Ph | 1H-1,2,4-triazol-1-yl |
| 2-CF₃-Ph | 1H-1,2,4-triazol-1-yl |
| 3-CF₃-Ph | 1H-1,2,4-triazol-1-yl |
| 4-CF₃-Ph | 1H-1,2,4-triazol-1-yl |
| 2-CN-Ph | 1H-1,2,4-triazol-1-yl |
| 3-CN-Ph | 1H-1,2,4-triazol-1-yl |
| 4-CN-Ph | 1H-1,2,4-triazol-1-yl |
| 2-NO₂-Ph | 1H-1,2,4-triazol-1-yl |
| 3-NO₂-Ph | 1H-1,2,4-triazol-1-yl |
| 4-NO₂-Ph | 1H-1,2,4-triazol-1-yl |
| 3-(CH═CH₂)-Ph | 1H-1,2,4-triazol-1-yl |
| 3-(CCH)-Ph | 1H-1,2,4-triazol-1-yl |
| 4-c-Pr-Ph | 1H-1,2,4-triazol-1-yl |
| 3-(CH═CCl)-Ph | 1H-1,2,4-triazol-1-yl |
| 3-(CCCl)-Ph | 1H-1,2,4-triazol-1-yl |
| 3-(2,2-di-Cl-c-Pr)-Ph | 1H-1,2,4-triazol-1-yl |
| 2-OCF₃-Ph | 1H-1,2,4-triazol-1-yl |
| 3-OCF₃-Ph | 1H-1,2,4-triazol-1-yl |
| 4-OCF₃-Ph | 1H-1,2,4-triazol-1-yl |
| 3-SMe-Ph | 1H-1,2,4-triazol-1-yl |
| 3-S(O)Me-Ph | 1H-1,2,4-triazol-1-yl |
| 3-SO₂Me-Ph | 1H-1,2,4-triazol-1-yl |
| 3-NHMe-Ph | 1H-1,2,4-triazol-1-yl |
| 3-NMe₂-Ph | 1H-1,2,4-triazol-1-yl |
| 3-NH-c-Pr-Ph | 1H-1,2,4-triazol-1-yl |
| 3-COMe-Ph | 1H-1,2,4-triazol-1-yl |
| 3-CO₂Me-Ph | 1H-1,2,4-triazol-1-yl |
| 3-CONHMe-Ph | 1H-1,2,4-triazol-1-yl |
| 3-CONMe₂-Ph | 1H-1,2,4-triazol-1-yl |
| 3-SiMe₃-Ph | 1H-1,2,4-triazol-1-yl |
| 2,3-di-Me-Ph | 1H-1,2,4-triazol-1-yl |
| 2-F-Ph | 1-methyl-1H-pyrazol-3-yl |
| 3-F-Ph | 1-methyl-1H-pyrazol-3-yl |
| 4-F-Ph | 1-methyl-1H-pyrazol-3-yl |
| 2,3-di-F-Ph | 1-methyl-1H-pyrazol-3-yl |
| 2,4-di-F-Ph | 1-methyl-1H-pyrazol-3-yl |
| 2,5-di-F-Ph | 1-methyl-1H-pyrazol-3-yl |
| 2,6-di-F-Ph | 1-methyl-1H-pyrazol-3-yl |
| 3,4-di-F-Ph | 1-methyl-1H-pyrazol-3-yl |
| 3,5-di-F-Ph | 1-methyl-1H-pyrazol-3-yl |
| 2,3-di-Cl-Ph | 1-methyl-1H-pyrazol-3-yl |
| 2,4-di-Cl-Ph | 1-methyl-1H-pyrazol-3-yl |
| 2,5-di-Cl-Ph | 1-methyl-1H-pyrazol-3-yl |
| 2,6-di-Cl-Ph | 1-methyl-1H-pyrazol-3-yl |
| 3,4-di-Cl-Ph | 1-methyl-1H-pyrazol-3-yl |
| 3,5-di-Cl-Ph | 1-methyl-1H-pyrazol-3-yl |
| 2-OMe-Ph | 1-methyl-1H-pyrazol-3-yl |
| 3-OMe-Ph | 1-methyl-1H-pyrazol-3-yl |
| 4-OMe-Ph | 1-methyl-1H-pyrazol-3-yl |
| 2-Me-Ph | 1-methyl-1H-pyrazol-3-yl |
| 3-Me-Ph | 1-methyl-1H-pyrazol-3-yl |
| 4-Me-Ph | 1-methyl-1H-pyrazol-3-yl |
| 2-CF₃-Ph | 1-methyl-1H-pyrazol-3-yl |
| 3-CF₃-Ph | 1-methyl-1H-pyrazol-3-yl |
| 4-CF₃-Ph | 1-methyl-1H-pyrazol-3-yl |
| 2-CN-Ph | 1-methyl-1H-pyrazol-3-yl |
| 3-CN-Ph | 1-methyl-1H-pyrazol-3-yl |
| 2-F-Ph | 2-pyridinyl |
| 3-F-Ph | 2-pyridinyl |
| 4-F-Ph | 2-pyridinyl |
| 2,3-di-F-Ph | 2-pyridinyl |
| 2,4-di-F-Ph | 2-pyridinyl |
| 2,5-di-F-Ph | 2-pyridinyl |
| 2,6-di-F-Ph | 2-pyridinyl |
| 3,4-di-F-Ph | 2-pyridinyl |
| 3,5-di-F-Ph | 2-pyridinyl |
| 2,3-di-Cl-Ph | 2-pyridinyl |
| 2,4-di-Cl-Ph | 2-pyridinyl |
| 2,5-di-Cl-Ph | 2-pyridinyl |
| 2,6-di-Cl-Ph | 2-pyridinyl |
| 3,4-di-Cl-Ph | 2-pyridinyl |
| 3,5-di-Cl-Ph | 2-pyridinyl |
| 2-OMe-Ph | 2-pyridinyl |
| 3-OMe-Ph | 2-pyridinyl |
| 4-OMe-Ph | 2-pyridinyl |
| 2-Me-Ph | 2-pyridinyl |
| 3-Me-Ph | 2-pyridinyl |
| 4-Me-Ph | 2-pyridinyl |
| 2-CF₃-Ph | 2-pyridinyl |
| 3-CF₃-Ph | 2-pyridinyl |
| 4-CF₃-Ph | 2-pyridinyl |
| 2-CN-Ph | 2-pyridinyl |
| 3-CN-Ph | 2-pyridinyl |
| 4-CN-Ph | 2-pyridinyl |
| 2-NO₂-Ph | 2-pyridinyl |
| 3-NO₂-Ph | 2-pyridinyl |
| 4-NO₂-Ph | 2-pyridinyl |
| 3-(CH═CH₂)-Ph | 2-pyridinyl |
| 3-(CCH)-Ph | 2-pyridinyl |
| 4-c-Pr-Ph | 2-pyridinyl |
| 3-(CH═CCl₂)-Ph | 2-pyridinyl |
| 3-(CCCl)-Ph | 2-pyridinyl |
| 3-(2,2-di-Cl-c-Pr)-Ph | 2-pyridinyl |
| 2-OCF₃-Ph | 2-pyridinyl |
| 3-OCF₃-Ph | 2-pyridinyl |
| 4-OCF₃-Ph | 2-pyridinyl |
| 3-SMe-Ph | 2-pyridinyl |
| 3-S(O)Me-Ph | 2-pyridinyl |
| 3-SO2Me-Ph | 2-pyridinyl |
| 3-NHMe-Ph | 2-pyridinyl |
| 3-NMe₂-Ph | 2-pyridinyl |
| 3-NH-c-Pr-Ph | 2-pyridinyl |
| 3-COMe-Ph | 2-pyridinyl |
| 3-CO₂Me-Ph | 2-pyridinyl |
| 3-CONHMe-Ph | 2-pyridinyl |
| 3-CONMe₂-Ph | 2-pyridinyl |
| 3-SiMe₃-Ph | 2-pyridinyl |
| 2,3-di-Me-Ph | 2-pyridinyl |
| 2-F-Ph | CONH₂ |
| 3-F-Ph | CONH₂ |
| 4-F-Ph | CONH₂ |
| 2,3-di-F-Ph | CONH₂ |
| 2,4-di-F-Ph | CONH₂ |
| 2,5-di-F-Ph | CONH₂ |
| 2,6-di-F-Ph | CONH₂ |
| 3,4-di-F-Ph | CONH₂ |
| 3,5-di-F-Ph | CONH₂ |
| 2,3-di-Cl-Ph | CONH₂ |
| 2,4-di-Cl-Ph | CONH₂ |
| 2,5-di-Cl-Ph | CONH₂ |
| 2,6-di-Cl-Ph | CONH₂ |
| 3,4-di-Cl-Ph | CONH₂ |
| 3,5-di-Cl-Ph | CONH₂ |
| 2-OMe-Ph | CONH₂ |
| 3-OMe-Ph | CONH₂ |
| 4-OMe-Ph | CONH₂ |
| 2-Me-Ph | CONH₂ |
| 3-Me-Ph | CONH₂ |
| 4-Me-Ph | CONH₂ |
| 2-CF₃-Ph | CONH₂ |
| 3-CF₃-Ph | CONH₂ |
| 4-CF₃-Ph | CONH₂ |
| 2-CN-Ph | CONH₂ |
| 3-CN-Ph | CONH₂ |
| 4-CN-Ph | CONH₂ |
| 2-NO₂-Ph | CONH₂ |
| 3-NO₂-Ph | CONH₂ |
| 4-NO₂-Ph | CONH₂ |
| 3-(CH═CH₂)-Ph | CONH₂ |
| 3-(CCH)-Ph | CONH₂ |
| 4-c-Pr-Ph | CONH₂ |
| 3-(CH═CCl)-Ph | CONH₂ |
| 3-(CCCl)-Ph | CONH₂ |
| 3-(2,2-di-Cl-c-Pr)-Ph | CONH₂ |
| 2-OCF₃-Ph | CONH₂ |
| 3-OCF₃-Ph | CONH₂ |
| 4-OCF₃-Ph | CONH₂ |
| 3-SMe-Ph | CONH₂ |
| 3-S(O)Me-Ph | CONH₂ |
| 3-SO₂Me-Ph | CONH₂ |
| 3-NHMe-Ph | CONH₂ |
| 3-NMe₂-Ph | CONH₂ |
| 3-NH-c-Pr-Ph | CONH₂ |
| 3-COMe-Ph | CONH₂ |
| 3-CO₂Me-Ph | CONH₂ |
| 3-CONHMe-Ph | CONH₂ |
| 3-CONMe₂-Ph | CONH₂ |
| 3-SiMe₃-Ph | CONH₂ |
| 2,3-di-Me-Ph | CONH₂ |
| 4-CN-Ph | 1-methyl-1H-pyrazol-3-yl |
| 2-NO₂-Ph | 1-methyl-1H-pyrazol-3-yl |
| 3-NO₂-Ph | 1-methyl-1H-pyrazol-3-yl |
| 4-NO₂-Ph | 1-methyl-1H-pyrazol-3-yl |
| 3-(CH ═CH₂)-Ph | 1-methyl-1H-pyrazol-3-yl |
| 3-(CCH)-Ph | 1-methyl-1H-pyrazol-3-yl |
| 4-c-Pr-Ph | 1-methyl-1H-pyrazol-3-yl |
| 3-(CH═CCl₂)-Ph | 1-methyl-1H-pyrazol-3-yl |
| 3-(CCCl)-Ph | 1-methyl-1H-pyrazol-3-yl |
| 3-(2,2-di-Cl-c-Pr)-Ph | 1-methyl-1H-pyrazol-3-yl |
| 2-OCF₃-Ph | 1-methyl-1H-pyrazol-3-yl |
| 3-OCF₃-Ph | 1-methyl-1H-pyrazol-3-yl |
| 4-OCF₃-Ph | 1-methyl-1H-pyrazol-3-yl |
| 3-SMe-Ph | 1-methyl-1H-pyrazol-3-yl |
| 3-S(O)Me-Ph | 1-methyl-1H-pyrazol-3-yl |
| 3-SO₂Me-Ph | 1-methyl-1H-pyrazol-3-yl |
| 3-NHMe-Ph | 1-methyl-1H-pyrazol-3-yl |
| 3-NMe₂-Ph | 1-methyl-1H-pyrazol-3-yl |
| 3-NH-c-Pr-Ph | 1-methyl-1H-pyrazol-3-yl |
| 3-COMe-Ph | 1-methyl-1H-pyrazol-3-yl |
| 3-CO₂Me-Ph | 1-methyl-1H-pyrazol-3-yl |
| 3-CONHMe-Ph | 1-methyl-1H-pyrazol-3-yl |
| 3-CONMe₂-Ph | 1-methyl-1H-pyrazol-3-yl |
| 3-SiMe₃-Ph | 1-methyl-1H-pyrazol-3-yl |
| 2,3-di-Me-Ph | 1-methyl-1H-pyrazol-3-yl |

TABLE 1f

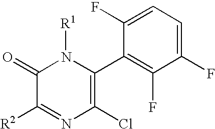

| R¹ | R² |
|---|---|
| 2-F-Ph | 1H-pyrazol-1-yl |
| 3-F-Ph | 1H-pyrazol-1-yl |
| 4-F-Ph | 1H-pyrazol-1-yl |
| 2,3-di-F-Ph | 1H-pyrazol-1-yl |
| 2,4-di-F-Ph | 1H-pyrazol-1-yl |
| 2,5-di-F-Ph | 1H-pyrazol-1-yl |
| 2,6-di-F-Ph | 1H-pyrazol-1-yl |
| 3,4-di-F-Ph | 1H-pyrazol-1-yl |
| 3,5-di-F-Ph | 1H-pyrazol-1-yl |
| 2,3-di-Cl-Ph | 1H-pyrazol-1-yl |
| 2,4-di-Cl-Ph | 1H-pyrazol-1-yl |
| 2,5-di-Cl-Ph | 1H-pyrazol-1-yl |
| 2,6-di-Cl-Ph | 1H-pyrazol-1-yl |
| 3,4-di-Cl-Ph | 1H-pyrazol-1-yl |
| 3,5-di-Cl-Ph | 1H-pyrazol-1-yl |
| 2-OMe-Ph | 1H-pyrazol-1-yl |
| 3-OMe-Ph | 1H-pyrazol-1-yl |
| 4-OMe-Ph | 1H-pyrazol-1-yl |
| 2-Me-Ph | 1H-pyrazol-1-yl |
| 3-Me-Ph | 1H-pyrazol-1-yl |
| 4-Me-Ph | 1H-pyrazol-1-yl |
| 2-CF₃-Ph | 1H-pyrazol-1-yl |
| 3-CF₃-Ph | 1H-pyrazol-1-yl |
| 4-CF₃-Ph | 1H-pyrazol-1-yl |
| 2-CN-Ph | 1H-pyrazol-1-yl |
| 3-CN-Ph | 1H-pyrazol-1-yl |
| 4-CN-Ph | 1H-pyrazol-1-yl |
| 2-NO₂-Ph | 1H-pyrazol-1-yl |
| 3-NO₂-Ph | 1H-pyrazol-1-yl |
| 4-NO₂-Ph | 1H-pyrazol-1-yl |
| 3-(CH═CH₂)-Ph | 1H-pyrazol-1-yl |
| 3-(CCH)-Ph | 1H-pyrazol-1-yl |
| 4-c-Pr-Ph | 1H-pyrazol-1-yl |
| 3-(CH═CCl₂)-Ph | 1H-pyrazol-1-yl |
| 3-(CCCl)-Ph | 1H-pyrazol-1-yl |
| 3-(2,2-di-Cl-c-Pr)-Ph | 1H-pyrazol-1-yl |
| 2-OCF₃-Ph | 1H-pyrazol-1-yl |
| 3-OCF₃-Ph | 1H-pyrazol-1-yl |
| 4-OCF₃-Ph | 1H-pyrazol-1-yl |
| 3-SMe-Ph | 1H-pyrazol-1-yl |
| 3-S(O)Me-Ph | 1H-pyrazol-1-yl |
| 3-SO2Me-Ph | 1H-pyrazol-1-yl |
| 3-NHMe-Ph | 1H-pyrazol-1-yl |
| 3-NMe₂-Ph | 1H-pyrazol-1-yl |
| 3-NH-c-Pr-Ph | 1H-pyrazol-1-yl |
| 3-COMe-Ph | 1H-pyrazol-1-yl |
| 3-CO₂Me-Ph | 1H-pyrazol-1-yl |
| 3-CONHMe-Ph | 1H-pyrazol-1-yl |
| 3-CONMe₂-Ph | 1H-pyrazol-1-yl |
| 3-SiMe₃-Ph | 1H-pyrazol-1-yl |
| 2,3-di-Me-Ph | 1H-pyrazol-1-yl |
| 2-F-Ph | 1H-1,2,4-triazol-1-yl |
| 3-F-Ph | 1H-1,2,4-triazol-1-yl |
| 4-F-Ph | 1H-1,2,4-triazol-1-yl |
| 2,3-di-F-Ph | 1H-1,2,4-triazol-1-yl |
| 2,4-di-F-Ph | 1H-1,2,4-triazol-1-yl |
| 2,5-di-F-Ph | 1H-1,2,4-triazol-1-yl |
| 2,6-di-F-Ph | 1H-1,2,4-triazol-1-yl |
| 3,4-di-F-Ph | 1H-1,2,4-triazol-1-yl |
| 3,5-di-F-Ph | 1H-1,2,4-triazol-1-yl |
| 2,3-di-Cl-Ph | 1H-1,2,4-triazol-1-yl |
| 2,4-di-Cl-Ph | 1H-1,2,4-triazol-1-yl |
| 2,5-di-Cl-Ph | 1H-1,2,4-triazol-1-yl |
| 2,6-di-Cl-Ph | 1H-1,2,4-triazol-1-yl |
| 3,4-di-Cl-Ph | 1H-1,2,4-triazol-1-yl |
| 3,5-di-Cl-Ph | 1H-1,2,4-triazol-1-yl |
| 2-OMe-Ph | 1H-1,2,4-triazol-1-yl |
| 3-OMe-Ph | 1H-1,2,4-triazol-1-yl |
| 4-OMe-Ph | 1H-1,2,4-triazol-1-yl |
| 2-Me-Ph | 1H-1,2,4-triazol-1-yl |
| 3-Me-Ph | 1H-1,2,4-triazol-1-yl |
| 4-Me-Ph | 1H-1,2,4-triazol-1-yl |
| 2-CF₃-Ph | 1H-1,2,4-triazol-1-yl |
| 3-CF₃-Ph | 1H-1,2,4-triazol-1-yl |
| 4-CF₃-Ph | 1H-1,2,4-triazol-1-yl |
| 2-CN-Ph | 1H-1,2,4-triazol-1-yl |
| 3-CN-Ph | 1H-1,2,4-triazol-1-yl |
| 4-CN-Ph | 1H-1,2,4-triazol-1-yl |
| 2-NO₂-Ph | 1H-1,2,4-triazol-1-yl |
| 3-NO₂-Ph | 1H-1,2,4-triazol-1-yl |
| 4-NO₂-Ph | 1H-1,2,4-triazol-1-yl |
| 3-(CH═CH₂)-Ph | 1H-1,2,4-triazol-1-yl |
| 3-(CCH)-Ph | 1H-1,2,4-triazol-1-yl |
| 4-c-Pr-Ph | 1H-1,2,4-triazol-1-yl |
| 3-(CH═CCl)-Ph | 1H-1,2,4-triazol-1-yl |
| 3-(CCCl)-Ph | 1H-1,2,4-triazol-1-yl |
| 3-(2,2-di-Cl-c-Pr)-Ph | 1H-1,2,4-triazol-1-yl |
| 2-OCF₃-Ph | 1H-1,2,4-triazol-1-yl |
| 3-OCF₃-Ph | 1H-1,2,4-triazol-1-yl |
| 4-OCF₃-Ph | 1H-1,2,4-triazol-1-yl |
| 3-SMe-Ph | 1H-1,2,4-triazol-1-yl |
| 3-S(O)Me-Ph | 1H-1,2,4-triazol-1-yl |
| 3-SO₂Me-Ph | 1H-1,2,4-triazol-1-yl |
| 3-NHMe-Ph | 1H-1,2,4-triazol-1-yl |
| 3-NMe₂-Ph | 1H-1,2,4-triazol-1-yl |
| 3-NH-c-Pr-Ph | 1H-1,2,4-triazol-1-yl |
| 3-COMe-Ph | 1H-1,2,4-triazol-1-yl |
| 3-CO₂Me-Ph | 1H-1,2,4-triazol-1-yl |
| 3-CONHMe-Ph | 1H-1,2,4-triazol-1-yl |
| 3-CONMe₂-Ph | 1H-1,2,4-triazol-1-yl |
| 3-SiMe₃-Ph | 1H-1,2,4-triazol-1-yl |
| 2,3-di-Me-Ph | 1H-1,2,4-triazol-1-yl |
| 2-F-Ph | 2-pyridinyl |
| 3-F-Ph | 2-pyridinyl |
| 4-F-Ph | 2-pyridinyl |
| 2,3-di-F-Ph | 2-pyridinyl |
| 2,4-di-F-Ph | 2-pyridinyl |
| 2,5-di-F-Ph | 2-pyridinyl |
| 2,6-di-F-Ph | 2-pyridinyl |
| 3,4-di-F-Ph | 2-pyridinyl |
| 3,5-di-F-Ph | 2-pyridinyl |
| 2,3-di-Cl-Ph | 2-pyridinyl |
| 2,4-di-Cl-Ph | 2-pyridinyl |
| 2,5-di-Cl-Ph | 2-pyridinyl |
| 2,6-di-Cl-Ph | 2-pyridinyl |
| 3,4-di-Cl-Ph | 2-pyridinyl |
| 3,5-di-Cl-Ph | 2-pyridinyl |
| 2-OMe-Ph | 2-pyridinyl |
| 3-OMe-Ph | 2-pyridinyl |
| 4-OMe-Ph | 2-pyridinyl |
| 2-Me-Ph | 2-pyridinyl |
| 3-Me-Ph | 2-pyridinyl |
| 4-Me-Ph | 2-pyridinyl |
| 2-CF₃-Ph | 2-pyridinyl |
| 3-CF₃-Ph | 2-pyridinyl |
| 4-CF₃-Ph | 2-pyridinyl |
| 2-CN-Ph | 2-pyridinyl |
| 3-CN-Ph | 2-pyridinyl |
| 4-CN-Ph | 2-pyridinyl |
| 2-NO₂-Ph | 2-pyridinyl |
| 3-NO₂-Ph | 2-pyridinyl |
| 4-NO₂-Ph | 2-pyridinyl |
| 3-(CH═CH₂)-Ph | 2-pyridinyl |
| 3-(CCH)-Ph | 2-pyridinyl |
| 4-c-Pr-Ph | 2-pyridinyl |
| 3-(CH═CCl₂)-Ph | 2-pyridinyl |
| 3-(CCCl)-Ph | 2-pyridinyl |
| 3-(2,2-di-Cl-c-Pr)-Ph | 2-pyridinyl |
| 2-OCF₃-Ph | 2-pyridinyl |
| 3-OCF₃-Ph | 2-pyridinyl |
| 4-OCF₃-Ph | 2-pyridinyl |
| 3-SMe-Ph | 2-pyridinyl |
| 3-S(O)Me-Ph | 2-pyridinyl |
| 3-SO2Me-Ph | 2-pyridinyl |
| 3-NHMe-Ph | 2-pyridinyl |
| 3-NMe₂-Ph | 2-pyridinyl |
| 3-NH-c-Pr-Ph | 2-pyridinyl |
| 3-COMe-Ph | 2-pyridinyl |
| 3-CO₂Me-Ph | 2-pyridinyl |
| 3-CONHMe-Ph | 2-pyridinyl |
| 3-CONMe₂-Ph | 2-pyridinyl |
| 3-SiMe₃-Ph | 2-pyridinyl |
| 2,3-di-Me-Ph | 2-pyridinyl |
| 2-F-Ph | CONH₂ |
| 3-F-Ph | CONH₂ |
| 4-F-Ph | CONH₂ |
| 2,3-di-F-Ph | CONH₂ |
| 2,4-di-F-Ph | CONH₂ |
| 2,5-di-F-Ph | CONH₂ |
| 2,6-di-F-Ph | CONH₂ |
| 3,4-di-F-Ph | CONH₂ |
| 3,5-di-F-Ph | CONH₂ |
| 2,3-di-Cl-Ph | CONH₂ |
| 2,4-di-Cl-Ph | CONH₂ |
| 2,5-di-Cl-Ph | CONH₂ |
| 2,6-di-Cl-Ph | CONH₂ |
| 3,4-di-Cl-Ph | CONH₂ |
| 3,5-di-Cl-Ph | CONH₂ |
| 2-OMe-Ph | CONH₂ |
| 3-OMe-Ph | CONH₂ |
| 4-OMe-Ph | CONH₂ |
| 2-Me-Ph | CONH₂ |
| 3-Me-Ph | CONH₂ |
| 4-Me-Ph | CONH₂ |
| 2-CF₃-Ph | CONH₂ |
| 3-CF₃-Ph | CONH₂ |
| 4-CF₃-Ph | CONH₂ |
| 2-CN-Ph | CONH₂ |
| 3-CN-Ph | CONH₂ |
| 4-CN-Ph | CONH₂ |
| 2-NO₂-Ph | CONH₂ |
| 3-NO₂-Ph | CONH₂ |
| 4-NO₂-Ph | CONH₂ |
| 3-(CH═CH₂)-Ph | CONH₂ |
| 3-(CCH)-Ph | CONH₂ |
| 4-c-Pr-Ph | CONH₂ |
| 3-(CH═CCl)-Ph | CONH₂ |
| 3-(CCCl)-Ph | CONH₂ |
| 3-(2,2-di-Cl-c-Pr)-Ph | CONH₂ |
| 2-OCF₃-Ph | CONH₂ |
| 3-OCF₃-Ph | CONH₂ |
| 4-OCF₃-Ph | CONH₂ |
| 3-SMe-Ph | CONH₂ |
| 3-S(O)Me-Ph | CONH₂ |
| 3-SO₂Me-Ph | CONH₂ |
| 3-NHMe-Ph | CONH₂ |
| 3-NMe₂-Ph | CONH₂ |
| 3-NH-c-Pr-Ph | CONH₂ |
| 3-COMe-Ph | CONH₂ |
| 3-CO₂Me-Ph | CONH₂ |
| 3-CONHMe-Ph | CONH₂ |
| 3-CONMe₂-Ph | CONH₂ |
| 3-SiMe₃-Ph | CONH₂ |
| 2,3-di-Me-Ph | CONH₂ |

TABLE 1g

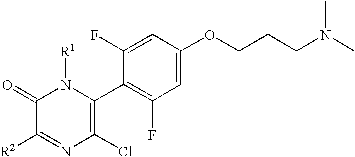

| R¹ | R² |
|---|---|
| Me | 1H-pyrazol-1-yl |
| Et | 1H-pyrazol-1-yl |
| i-Pr | 1H-pyrazol-1-yl |
| n-Pr | 1H-pyrazol-1-yl |
| i-Bu | 1H-pyrazol-1-yl |
| n-Bu | 1H-pyrazol-1-yl |
| s-Bu | 1H-pyrazol-1-yl |
| 3-Me-Bu | 1H-pyrazol-1-yl |
| n-pentyl | 1H-pyrazol-1-yl |
| n-Hex | 1H-pyrazol-1-yl |
| 2-propenyl | 1H-pyrazol-1-yl |
| 2-Me-2-propenyl | 1H-pyrazol-1-yl |
| 3-butenyl | 1H-pyrazol-1-yl |
| 3-pentenyl | 1H-pyrazol-1-yl |
| 2-propynyl | 1H-pyrazol-1-yl |
| 3-butynyl | 1H-pyrazol-1-yl |
| 4-butynyl | 1H-pyrazol-1-yl |
| c-Pr | 1H-pyrazol-1-yl |
| c-pentyl | 1H-pyrazol-1-yl |
| c-Hex | 1H-pyrazol-1-yl |
| 2-cyclohexenyl | 1H-pyrazol-1-yl |
| 3-cyclohexenyl | 1H-pyrazol-1-yl |
| CH₂-c-Pr | 1H-pyrazol-1-yl |
| CH₂-c-Hex | 1H-pyrazol-1-yl |
| CH₂-2-cyclohexenyl | 1H-pyrazol-1-yl |
| 4-tetrahydropyranyl | 1H-pyrazol-1-yl |
| 3-tetrahydropyranyl | 1H-pyrazol-1-yl |
| 3-tetrahydrofuranyl | 1H-pyrazol-1-yl |
| Ph | 1H-pyrazol-1-yl |
| 2-Cl-phenyl | 1H-pyrazol-1-yl |
| 3-Cl-phenyl | 1H-pyrazol-1-yl |
| 4-Cl-phenyl | 1H-pyrazol-1-yl |
| 2-pyridinyl | 1H-pyrazol-1-yl |
| 2-pyrimidyl | 1H-pyrazol-1-yl |
| 2-pyrazinyl | 1H-pyrazol-1-yl |
| 2-thiazolyl | 1H-pyrazol-1-yl |
| 2-oxazolyl | 1H-pyrazol-1-yl |
| CF₃ | 1H-pyrazol-1-yl |
| CF₂CF₃ | 1H-pyrazol-1-yl |
| CH₂CF₃ | 1H-pyrazol-1-yl |
| CH(Me)CF₃ | 1H-pyrazol-1-yl |
| CH₂CH₂F | 1H-pyrazol-1-yl |
| CH₂CH₂CH₂F | 1H-pyrazol-1-yl |
| CH₂CF₂CF₃ | 1H-pyrazol-1-yl |
| CH₂CH₂CF₃ | 1H-pyrazol-1-yl |
| CH₂CH(Me)CF₃ | 1H-pyrazol-1-yl |
| (S)-CH₂CH(Me)CF₃ | 1H-pyrazol-1-yl |
| CH₂CH₂CH₂CH₂F | 1H-pyrazol-1-yl |
| 2-chloro-2-propenyl | 1H-pyrazol-1-yl |
| 3,3 dichloro-2-propenyl | 1H-pyrazol-1-yl |
| CH₂-2-tetrahydrofuranyl | 1H-pyrazol-1-yl |
| CH₂-2-tetrahydropyranyl | 1H-pyrazol-1-yl |
| CH₂CN | 1H-pyrazol-1-yl |
| CH₂NO₂ | 1H-pyrazol-1-yl |
| CH₂CH₂OH | 1H-pyrazol-1-yl |
| CH₂CH₂OMe | 1H-pyrazol-1-yl |
| CH₂CH(Me)OMe | 1H-pyrazol-1-yl |
| CH(Me)CH₂OMe | 1H-pyrazol-1-yl |
| CH(Me)CH(OMe)₂ | 1H-pyrazol-1-yl |
| CH₂-2-dioxolanyl | 1H-pyrazol-1-yl |
| CH₂CH₂OCF₃ | 1H-pyrazol-1-yl |
| CH₂CH₂SMe | 1H-pyrazol-1-yl |
| CH₂CH(Me)SMe | 1H-pyrazol-1-yl |
| CH₂CH₂S(O)Me | 1H-pyrazol-1-yl |
| CH₂CH₂S(O)₂Me | 1H-pyrazol-1-yl |
| CH₂CO₂Me | 1H-pyrazol-1-yl |
| CH₂CO₂-i-Pr | 1H-pyrazol-1-yl |
| CH(Me)CO₂Me | 1H-pyrazol-1-yl |
| CH₂C(O)Me | 1H-pyrazol-1-yl |
| CH₂CH₂C(O)Me | 1H-pyrazol-1-yl |
| CH₂SiMe₃ | 1H-pyrazol-1-yl |
| CH₂CH₂SiMe₃ | 1H-pyrazol-1-yl |
| CH₂OPh | 1H-pyrazol-1-yl |
| CH₂Ph | 1H-pyrazol-1-yl |
| CH₂CH₂Ph | 1H-pyrazol-1-yl |
| CH(Me)Ph | 1H-pyrazol-1-yl |
| CH₂-2-Cl-Ph | 1H-pyrazol-1-yl |
| CH₂-3-Cl-Ph | 1H-pyrazol-1-yl |
| CH₂-4-Cl-Ph | 1H-pyrazol-1-yl |
| CH₂-2-thienyl | 1H-pyrazol-1-yl |
| CH₂-2-pyridinyl | 1H-pyrazol-1-yl |
| CH₂-3-pyridinyl | 1H-pyrazol-1-yl |
| CH(Et)₂ | 1H-pyrazol-1-yl |
| CH₂CH(Et)₂ | 1H-pyrazol-1-yl |
| CH₂CH(n-Pr)Me | 1H-pyrazol-1-yl |
| CH(Me)Et | 1H-pyrazol-1-yl |
| CH(Me)-n-Pr | 1H-pyrazol-1-yl |
| CH(CF₃)Et | 1H-pyrazol-1-yl |
| CH(Et)-n-Pr | 1H-pyrazol-1-yl |
| CH(Me)-n-Bu | 1H-pyrazol-1-yl |
| 2,2-dimethylpropyl | 1H-pyrazol-1-yl |
| CH₂CH₂CH(Me)₂ | 1H-pyrazol-1-yl |
| Me | 1H-1,2,4-triazol-1-yl |
| Et | 1H-1,2,4-triazol-1-yl |
| i-Pr | 1H-1,2,4-triazol-1-yl |
| n-Pr | 1H-1,2,4-triazol-1-yl |
| i-Bu | 1H-1,2,4-triazol-1-yl |
| n-Bu | 1H-1,2,4-triazol-1-yl |
| s-Bu | 1H-1,2,4-triazol-1-yl |
| 3-Me-Bu | 1H-1,2,4-triazol-1-yl |
| n-pentyl | 1H-1,2,4-triazol-1-yl |
| n-Hex | 1H-1,2,4-triazol-1-yl |
| 2-propenyl | 1H-1,2,4-triazol-1-yl |
| 2-Me-2-propenyl | 1H-1,2,4-triazol-1-yl |
| 3-butenyl | 1H-1,2,4-triazol-1-yl |
| 3-pentenyl | 1H-1,2,4-triazol-1-yl |
| 2-propynyl | 1H-1,2,4-triazol-1-yl |
| 3-butynyl | 1H-1,2,4-triazol-1-yl |
| 4-butynyl | 1H-1,2,4-triazol-1-yl |
| c-Pr | 1H-1,2,4-triazol-1-yl |
| c-pentyl | 1H-1,2,4-triazol-1-yl |
| c-Hex | 1H-1,2,4-triazol-1-yl |
| 2-cyclohexenyl | 1H-1,2,4-triazol-1-yl |
| 3-cyclohexenyl | 1H-1,2,4-triazol-1-yl |
| CH₂-c-Pr | 1H-1,2,4-triazol-1-yl |
| CH₂-c-Hex | 1H-1,2,4-triazol-1-yl |
| CH₂-2-cyclohexenyl | 1H-1,2,4-triazol-1-yl |
| 4-tetrahydropyranyl | 1H-1,2,4-triazol-1-yl |
| 3-tetrahydropyranyl | 1H-1,2,4-triazol-1-yl |
| 3-tetrahydrofuranyl | 1H-1,2,4-triazol-1-yl |
| Ph | 1H-1,2,4-triazol-1-yl |
| 2-Cl-phenyl | 1H-1,2,4-triazol-1-yl |
| 3-Cl-phenyl | 1H-1,2,4-triazol-1-yl |
| 4-Cl-phenyl | 1H-1,2,4-triazol-1-yl |
| 2-pyridinyl | 1H-1,2,4-triazol-1-yl |
| 2-pyrimidyl | 1H-1,2,4-triazol-1-yl |
| 2-pyrazinyl | 1H-1,2,4-triazol-1-yl |
| 2-thiazolyl | 1H-1,2,4-triazol-1-yl |
| 2-oxazolyl | 1H-1,2,4-triazol-1-yl |
| CF₃ | 1H-1,2,4-triazol-1-yl |
| CF₂CF₃ | 1H-1,2,4-triazol-1-yl |
| CH₂CF₃ | 1H-1,2,4-triazol-1-yl |
| CH(Me)CF₃ | 1H-1,2,4-triazol-1-yl |
| CH₂CH₂F | 1H-1,2,4-triazol-1-yl |
| 2-chloro-2-propenyl | 1H-1,2,4-triazol-1-yl |
| 3,3-dichloro-2-propenyl | 1H-1,2,4-triazol-1-yl |
| CH₂-2-tetrahydrofuranyl | 1H-1,2,4-triazol-1-yl |
| CH₂-2-tetrahydropyranyl | 1H-1,2,4-triazol-1-yl |
| CH₂CN | 1H-1,2,4-triazol-1-yl |
| CH₂NO₂ | 1H-1,2,4-triazol-1-yl |
| CH₂CH₂OH | 1H-1,2,4-triazol-1-yl |
| CH₂CH₂OMe | 1H-1,2,4-triazol-1-yl |
| CH₂CH(Me)OMe | 1H-1,2,4-triazol-1-yl |
| CH(Me)CH₂OMe | 1H-1,2,4-triazol-1-yl |
| CH(Me)CH(OMe)₂ | 1H-1,2,4-triazol-1-yl |
| CH_{2-2-dioxolanyl} | 1H-1,2,4-triazol-1-yl |
| CH₂CH₂OCF₃ | 1H-1,2,4-triazol-1-yl |
| CH₂CH₂SMe | 1H-1,2,4-triazol-1-yl |
| CH₂CH(Me)SMe | 1H-1,2,4-triazol-1-yl |
| CH₂CH₂S(O)Me | 1H-1,2,4-triazol-1-yl |
| CH₂CH₂S(O)₂Me | 1H-1,2,4-triazol-1-yl |
| CH₂CO₂Me | 1H-1,2,4-triazol-1-yl |
| CH₂CO₂-i-Pr | 1H-1,2,4-triazol-1-yl |
| CH(Me)CO₂Me | 1H-1,2,4-triazol-1-yl |
| CH₂C(O)Me | 1H-1,2,4-triazol-1-yl |
| CH₂CH₂C(O)Me | 1H-1,2,4-triazol-1-yl |
| CH₂SiMe₃ | 1H-1,2,4-triazol-1-yl |
| CH₂CH₂SiMe₃ | 1H-1,2,4-triazol-1-yl |
| CH₂OPh | 1H-1,2,4-triazol-1-yl |
| CH₂Ph | 1H-1,2,4-triazol-1-yl |
| CH₂CH₂Ph | 1H-1,2,4-triazol-1-yl |
| CH(Me)Ph | 1H-1,2,4-triazol-1-yl |
| CH₂-2-Cl-Ph | 1H-1,2,4-triazol-1-yl |
| CH₂-3-Cl-Ph | 1H-1,2,4-triazol-1-yl |
| CH₂-4-Cl-Ph | 1H-1,2,4-triazol-1-yl |
| CH₂-2-thienyl | 1H-1,2,4-triazol-1-yl |
| CH₂-2-pyridinyl | 1H-1,2,4-triazol-1-yl |
| CH₂-3-pyridinyl | 1H-1,2,4-triazol-1-yl |
| CH(Et)₂ | 1H-1,2,4-triazol-1-yl |
| CH₂CH(Et)₂ | 1H-1,2,4-triazol-1-yl |
| CH₂CH(n-Pr)Me | 1H-1,2,4-triazol-1-yl |
| CH(Me)Et | 1H-1,2,4-triazol-1-yl |
| CH(Me)-n-Pr | 1H-1,2,4-triazol-1-yl |
| CH(CF₃)Et | 1H-1,2,4-triazol-1-yl |
| CH(Et)-n-Pr | 1H-1,2,4-triazol-1-yl |
| CH(Me)-n-Bu | 1H-1,2,4-triazol-1-yl |
| 2,2-dimethyipropyl | 1H-1,2,4-triazol-1-yl |
| CH₂CH₂CH(Me)₂ | 1H-1,2,4-triazol-1-yl |
| 2-F-Ph | 1H-pyrazol-1-yl |
| 3-F-Ph | 1H-pyrazol-1-yl |
| 4-F-Ph | 1H-pyrazol-1-yl |
| 2,3-di-F-Ph | 1H-pyrazol-1-yl |
| 2,4-di-F-Ph | 1H-pyrazol-1-yl |
| 2,5-di-F-Ph | 1H-pyrazol-1-yl |
| 2,6-di-F-Ph | 1H-pyrazol-1-yl |
| 3,4-di-F-Ph | 1H-pyrazol-1-yl |
| 3,5-di-F-Ph | 1H-pyrazol-1-yl |
| 2,3-di-Cl-Ph | 1H-pyrazol-1-yl |
| 2,4-di-Cl-Ph | 1H-pyrazol-1-yl |
| 2,5-di-Cl-Ph | 1H-pyrazol-1-yl |
| 2,6-di-Cl-Ph | 1H-pyrazol-1-yl |
| 3,4-di-Cl-Ph | 1H-pyrazol-1-yl |
| 3,5-di-Cl-Ph | 1H-pyrazol-1-yl |
| 2-OMe-Ph | 1H-pyrazol-1-yl |
| 3-OMe-Ph | 1H-pyrazol-1-yl |
| 4-OMe-Ph | 1H-pyrazol-1-yl |
| 2-Me-Ph | 1H-pyrazol-1-yl |
| 3-Me-Ph | 1H-pyrazol-1-yl |
| 4-Me-Ph | 1H-pyrazol-1-yl |
| 2-CF₃-Ph | 1H-pyrazol-1-yl |
| 3-CF₃-Ph | 1H-pyrazol-1-yl |
| 4-CF₃-Ph | 1H-pyrazol-1-yl |
| 2-CN-Ph | 1H-pyrazol-1-yl |
| 3-CN-Ph | 1H-pyrazol-1-yl |
| 4-CN-Ph | 1H-pyrazol-1-yl |
| 2-NO₂-Ph | 1H-pyrazol-1-yl |
| 3-NO₂-Ph | 1H-pyrazol-1-yl |
| 4-NO₂-Ph | 1H-pyrazol-1-yl |
| 3-(CH═CH₂)-Ph | 1H-pyrazol-1-yl |
| 3-(CCH)-Ph | 1H-pyrazol-1-yl |
| 4-c-Pr-Ph | 1H-pyrazol-1-yl |
| 3-(CH═CCl₂)-Ph | 1H-pyrazol-1-yl |
| 3-(CCCl)-Ph | 1H-pyrazol-1-yl |
| 3-(2,2-di-Cl-c-Pr)-Ph | 1H-pyrazol-1-yl |
| 2-OCF₃-Ph | 1H-pyrazol-1-yl |
| 3-OCF₃-Ph | 1H-pyrazol-1-yl |
| 4-OCF₃-Ph | 1H-pyrazol-1-yl |
| 3-SMe-Ph | 1H-pyrazol-1-yl |
| 3-S(O)Me-Ph | 1H-pyrazol-1-yl |
| 3-SO₂Me-Ph | 1H-pyrazol-1-yl |
| 3-NHMe-Ph | 1H-pyrazol-1-yl |
| 3-NMe₂-Ph | 1H-pyrazol-1-yl |
| 3-NH-c-Pr-Ph | 1H-pyrazol-1-yl |
| 3-COMe-Ph | 1H-pyrazol-1-yl |
| 3-CO₂Me-Ph | 1H-pyrazol-1-yl |
| 3-CONHMe-Ph | 1H-pyrazol-1-yl |
| 3-CONMe₂-Ph | 1H-pyrazol-1-yl |
| 3-SiMe₃-Ph | 1H-pyrazol-1-yl |
| 2,3-di-Me-Ph | 1H-pyrazol-1-yl |
| 2-F-Ph | 1H-1,2,4-triazol-1-yl |
| 3-F-Ph | 1H-1,2,4-triazol-1-yl |
| 4-F-Ph | 1H-1,2,4-triazol-1-yl |
| 2,3-di-F-Ph | 1H-1,2,4-triazol-1-yl |
| 2,4-di-F-Ph | 1H-1,2,4-triazol-1-yl |
| 2,5-di-F-Ph | 1H-1,2,4-triazol-1-yl |
| 2,6-di-F-Ph | 1H-1,2,4-triazol-1-yl |
| 3,4-di-F-Ph | 1H-1,2,4-triazol-1-yl |
| 3,5-di-F-Ph | 1H-1,2,4-triazol-1-yl |
| 2,3-di-Cl-Ph | 1H-1,2,4-triazol-1-yl |
| 2,4-di-Cl-Ph | 1H-1,2,4-triazol-1-yl |
| 2,5-di-Cl-Ph | 1H-1,2,4-triazol-1-yl |
| 2,6-di-Cl-Ph | 1H-1,2,4-triazol-1-yl |
| 3,4-di-Cl-Ph | 1H-1,2,4-triazol-1-yl |
| 3,5-di-Cl-Ph | 1H-1,2,4-triazol-1-yl |
| 2-OMe-Ph | 1H-1,2,4-triazol-1-yl |
| 3-OMe-Ph | 1H-1,2,4-triazol-1-yl |
| 4-OMe-Ph | 1H-1,2,4-triazol-1-yl |
| 2-Me-Ph | 1H-1,2,4-triazol-1-yl |
| 3-Me-Ph | 1H-1,2,4-triazol-1-yl |
| 4-Me-Ph | 1H-1,2,4-triazol-1-yl |
| 2-CF₃-Ph | 1H-1,2,4-triazol-1-yl |
| 3-CF₃-Ph | 1H-1,2,4-triazol-1-yl |
| 4-CF₃-Ph | 1H-1,2,4-triazol-1-yl |
| 2-CN-Ph | 1H-1,2,4-triazol-1-yl |
| 3-CN-Ph | 1H-1,2,4-triazol-1-yl |
| 4-CN-Ph | 1H-1,2,4-triazol-1-yl |
| 2-NO₂-Ph | 1H-1,2,4-triazol-1-yl |
| 3-NO₂-Ph | 1H-1,2,4-triazol-1-yl |
| 4-NO₂-Ph | 1H-1,2,4-triazol-1-yl |
| 3-(CH═CH₂)-Ph | 1H-1,2,4-triazol-1-yl |
| 3-(CCH)-Ph | 1H-1,2,4-triazol-1-yl |
| 4-c-Pr-Ph | 1H-1,2,4-triazol-1-yl |
| 3-(CHCCl₂)-Ph | 1H-1,2,4-triazol-1-yl |
| 3-(CCCl)-Ph | 1H-1,2,4-triazol-1-yl |
| 3-(2,2-di-Cl-c-Pr)-Ph | 1H-1,2,4-triazol-1-yl |
| 2-OCF₃-Ph | 1H-1,2,4-triazol-1-yl |
| 3-OCF₃-Ph | 1H-1,2,4-triazol-1-yl |
| 4-OCF₃-Ph | 1H-1,2,4-triazol-1-yl |
| 3-SMe-Ph | 1H-1,2,4-triazol-1-yl |
| 3-S(O)Me-Ph | 1H-1,2,4-triazol-1-yl |
| 3-SO₂Me-Ph | 1H-1,2,4-triazol-1-yl |
| 3-NHMe-Ph | 1H-1,2,4-triazol-1-yl |
| 3-NMe₂-Ph | 1H-1,2,4-triazol-1-yl |
| 3-NH-c-Pr-Ph | 1H-1,2,4-triazol-1-yl |
| 3-COMe-Ph | 1H-1,2,4-triazol-1-yl |
| 3-CO₂Me-Ph | 1H-1,2,4-triazol-1-yl |
| 3-CONHMe-Ph | 1H-1,2,4-triazol-1-yl |
| 3-CONMe₂-Ph | 1H-1,2,4-triazol-1-yl |
| 3-SiMe₃-Ph | 1H-1,2,4-triazol-1-yl |
| 2,3-di-Me-Ph | 1H-1,2,4-triazol-1-yl |
| Me | 1-methyl-1H-pyrazol-3-yl |
| Et | 1-methyl-1H-pyrazol-3-yl |
| i-Pr | 1-methyl-1H-pyrazol-3-yl |
| n-Pr | 1-methyl-1H-pyrazol-3-yl |
| i-Bu | 1-methyl-1H-pyrazol-3-yl |
| n-Bu | 1-methyl-1H-pyrazol-3-yl |
| s-Bu | 1-methyl-1H-pyrazol-3-yl |
| 3-Me-Bu | 1-methyl-1H-pyrazol-3-yl |
| n-pentyl | 1-methyl-1H-pyrazol-3-yl |
| n-Hex | 1-methyl-1H-pyrazol-3-yl |
| 2-propenyl | 1-methyl-1H-pyrazol-3-yl |
| 2-Me-₂-propenyl | 1-methyl-1H-pyrazol-3-yl |
| 3-butenyl | 1-methyl-1H-pyrazol-3-yl |
| 3-pentenyl | 1-methyl-1H-pyrazol-3-yl |
| 2-propynyl | 1-methyl-1H-pyrazol-3-yl |
| 3-butynyl | 1-methyl-1H-pyrazol-3-yl |
| 4-butynyl | 1-methyl-1H-pyrazol-3-yl |
| c-Pr | 1-methyl-1H-pyrazol-3-yl |
| c-pentyl | 1-methyl-1H-pyrazol-3-yl |
| 2-cyclohexenyl | 1-methyl-1H-pyrazol-3-yl |
| 3-cyclohexenyl | 1-methyl-1H-pyrazol-3-yl |
| CH₂-c-Pr | 1-methyl-1H-pyrazol-3-yl |
| CH₂-c-Hex | 1-methyl-1H-pyrazol-3-yl |
| CH₂-2-cyclohexenyl | 1-methyl-1H-pyrazol-3-yl |
| 4-tetrahydropyranyl | 1-methyl-1H-pyrazol-3-yl |
| 3-tetrahydropyranyl | 1-methyl-1H-pyrazol-3-yl |
| 3-tetrahydrofuranyl | 1-methyl-1H-pyrazol-3-yl |
| 2-pyridinyl | 1-methyl-1H-pyrazol-3-yl |
| 2-pyrimidyl | 1-methyl-1H-pyrazol-3-yl |
| 2-pyrazinyl | 1-methyl-1H-pyrazol-3-yl |
| 2-thiazolyl | 1-methyl-1H-pyrazol-3-yl |
| 2-oxazolyl | 1-methyl-1H-pyrazol-3-yl |
| CF₃ | 1-methyl-1H-pyrazol-3-yl |
| CF₂CF₃ | 1-methyl-1H-pyrazol-3-yl |
| CH₂CF₃ | 1-methyl-1H-pyrazol-3-yl |
| CH(Me)CF₃ | 1-methyl-1H-pyrazol-3-yl |
| CH₂CH₂F | 1-methyl-1H-pyrazol-3-yl |
| CH₂CH₂CH₂F | 1-methyl-1H-pyrazol-3-yl |
| CH₂CF₂CF₃ | 1-methyl-1H-pyrazol-3-yl |
| CH₂CH₂CF₃ | 1-methyl-1H-pyrazol-3-yl |
| CH₂CH(Me)CF₃ | 1-methyl-1H-pyrazol-3-yl |
| (S)-CH₂CH(Me)CF₃ | 1-methyl-1H-pyrazol-3-yl |
| CH₂CH₂CH₂CH₂F | 1-methyl-1H-pyrazol-3-yl |
| 2-chloro-2-propenyl | 1-methyl-1H-pyrazol-3-yl |
| 3,3-dichloro-2-propenyl | 1-methyl-1H-pyrazol-3-yl |
| CH₂-2-tetrahydrofuranyl | 1-methyl-1H-pyrazol-3-yl |
| CH₂-2-tetrahydropyranyl | 1-methyl-1H-pyrazol-3-yl |
| CH₂CN | 1-methyl-1H-pyrazol-3-yl |
| CH₂NO₂ | 1-methyl-1H-pyrazol-3-yl |
| CH₂CH₂OH | 1-methyl-1H-pyrazol-3-yl |
| CH₂CH₂OMe | 1-methyl-1H-pyrazol-3-yl |
| Me | 2-pyridinyl |
| Et | 2-pyridinyl |
| i-Pr | 2-pyridinyl |
| n-Pr | 2-pyridinyl |
| i-Bu | 2-pyridinyl |
| n-Bu | 2-pyridinyl |
| s-Bu | 2-pyridinyl |
| 3-Me-Bu | 2-pyridinyl |
| n-pentyl | 2-pyridinyl |
| n-Hex | 2-pyridinyl |
| 2-propenyl | 2-pyridinyl |
| 2-Me-2-propenyl | 2-pyridinyl |
| 3-butenyl | 2-pyridinyl |
| 3-pentenyl | 2-pyridinyl |
| 2-propynyl | 2-pyridinyl |
| 3-butynyl | 2-pyridinyl |
| 4-butynyl | 2-pyridinyl |
| c-Pr | 2-pyridinyl |
| c-pentyl | 2-pyridinyl |
| c-Hex | 2-pyridinyl |
| 2-cyclohexenyl | 2-pyridinyl |
| 3-cyclohexenyl | 2-pyridinyl |
| CH₂-c-Pr | 2-pyridinyl |
| CH₂-c-Hex | 2-pyridinyl |
| CH₂-2-cyclohexenyl | 2-pyridinyl |
| 4-tetrahydropyranyl | 2-pyridinyl |
| 3-tetrahydropyranyl | 2-pyridinyl |
| 3-tetrahydrofuranyl | 2-pyridinyl |
| Ph | 2-pyridinyl |
| 2-Cl-phenyl | 2-pyridinyl |
| 3-Cl-phenyl | 2-pyridinyl |
| 4-Cl-phenyl | 2-pyridinyl |
| 2-pyridinyl | 2-pyridinyl |
| 2-pyrimidyl | 2-pyridinyl |
| 2-pyrazinyl | 2-pyridinyl |
| 2-thiazolyl | 2-pyridinyl |
| 2-oxazolyl | 2-pyridinyl |
| CF₃ | 2-pyridinyl |
| CF₂CF₃ | 2-pyridinyl |
| CH₂CF₃ | 2-pyridinyl |
| CH(Me)CF₃ | 2-pyridinyl |
| CH₂CH₂F | 2-pyridinyl |
| CH₂CH₂CH₂F | 2-pyridinyl |
| CH₂CF₂CF₃ | 2-pyridinyl |
| CH₂CH₂CF₃ | 2-pyridinyl |
| CH₂CH(Me)CF₃ | 2-pyridinyl |
| (S)-CH₂CH(Me)CF₃ | 2-pyridinyl |
| CH₂CH₂CH₂CH₂F | 2-pyridinyl |
| 2-chloro-2-propenyl | 2-pyridinyl |
| 3,3 dichloro-2-propenyl | 2-pyridinyl |
| CH₂-2-tetrahydrofuranyl | 2-pyridinyl |
| CH₂-2-tetrahydropyranyl | 2-pyridinyl |
| CH₂CN | 2-pyridinyl |
| CH₂NO₂ | 2-pyridinyl |
| CH₂CH₂OH | 2-pyridinyl |
| CH₂CH₂OMe | 2-pyridinyl |
| CH₂CH(Me)OMe | 2-pyridinyl |
| CH(Me)CH₂OMe | 2-pyridinyl |
| CH(Me)CH(OMe)₂ | 2-pyridinyl |
| CH₂-2-dioxolanyl | 2-pyridinyl |
| CH₂CH₂OCF₃ | 2-pyridinyl |
| CH₂CH₂SMe | 2-pyridinyl |
| CH₂CH(Me)SMe | 2-pyridinyl |
| CH₂CH₂S(O)Me | 2-pyridinyl |
| CH₂CH₂S(O)₂Me | 2-pyridinyl |
| CH₂CO₂Me | 2-pyridinyl |
| CH₂CO₂-i-Pr | 2-pyridinyl |
| CH(Me)CO₂Me | 2-pyridinyl |
| CH₂C(O)Me | 2-pyridinyl |
| CH₂CH₂C(O)Me | 2-pyridinyl |
| CH₂SiMe₃ | 2-pyridinyl |
| CH₂CH₂SiMe₃ | 2-pyridinyl |
| CH₂OPh | 2-pyridinyl |
| CH₂Ph | 2-pyridinyl |
| CH₂CH₂Ph | 2-pyridinyl |
| CH(Me)Ph | 2-pyridinyl |
| CH₂-2-Cl-Ph | 2-pyridinyl |
| CH₂-3-Cl-Ph | 2-pyridinyl |
| CH₂-4-Cl-Ph | 2-pyridinyl |
| CH₂-2-thienyl | 2-pyridinyl |
| CH₂-2-pyridinyl | 2-pyridinyl |
| CH₂-3-pyridinyl | 2-pyridinyl |
| CH(Et)₂ | 2-pyridinyl |
| CH₂CH(Et)₂ | 2-pyridinyl |
| CH₂CH(n-Pr)Me | 2-pyridinyl |
| CH(Me)Et | 2-pyridinyl |
| CH(Me)-n-Pr | 2-pyridinyl |
| CH(CF₃)Et | 2-pyridinyl |
| CH(Et)-n-Pr | 2-pyridinyl |
| CH(Me)-n-Bu | 2-pyridinyl |
| 2,2-dimethylpropyl | 2-pyridinyl |
| CH₂CH₂CH(Me)₂ | 2-pyridinyl |
| Me | CONH₂ |
| Et | CONH₂ |
| i-Pr | CONH₂ |
| n-Pr | CONH₂ |
| i-Bu | CONH₂ |
| n-Bu | CONH₂ |
| s-Bu | CONH₂ |
| 3-Me-Bu | CONH₂ |
| n-pentyl | CONH₂ |
| n-Hex | CONH₂ |
| 2-propenyl | CONH₂ |
| 2-Me-2-propenyl | CONH₂ |
| 3-butenyl | CONH₂ |
| 3-pentenyl | CONH₂ |
| 2-propynyl | CONH₂ |
| 3-butynyl | CONH₂ |
| 4-butynyl | CONH₂ |
| c-Pr | CONH₂ |
| c-pentyl | CONH₂ |
| c-Hex | CONH₂ |
| 2-cyclohexenyl | CONH₂ |
| 3-cyclohexenyl | CONH₂ |
| CH₂-c-Pr | CONH₂ |
| CH₂-c-Hex | CONH₂ |
| CH₂-2-cyclohexenyl | CONH₂ |
| 4-tetrahydropyranyl | CONH₂ |
| 3-tetrahydropyranyl | CONH₂ |
| 3-tetrahydrofuranyl | CONH₂ |
| Ph | CONH₂ |
| 2-Cl-phenyl | CONH₂ |
| 3-Cl-phenyl | CONH₂ |
| 4-Cl-phenyl | CONH₂ |
| 2-pyridinyl | CONH₂ |
| 2-pyrimidyl | CONH₂ |
| 2-pyrazinyl | CONH₂ |
| 2-thiazolyl | CONH₂ |
| 2-oxazolyl | CONH₂ |
| CF₃ | CONH₂ |
| CF₂CF₃ | CONH₂ |
| CH₂CF₃ | CONH₂ |
| CH(Me)CF₃ | CONH₂ |
| CH₂CH₂F | CONH₂ |
| 2-chloro-2-propenyl | CONH₂ |
| 3,3-dichloro-2-propenyl | CONH₂ |
| CH₂-2-tetrahydrofuranyl | CONH₂ |
| CH₂-2-tetrahydropyranyl | CONH₂ |
| CH₂CN | CONH₂ |
| CH₂NO₂ | CONH₂ |
| CH₂CH₂OH | CONH₂ |
| CH₂CH₂OMe | CONH₂ |
| CH₂CH(Me)OMe | CONH₂ |
| CH(Me)CH₂OMe | CONH₂ |
| CH(Me)CH(OMe)₂ | CONH₂ |
| CH_{2-2-dioxolanyl} | CONH₂ |
| CH₂CH₂OCF₃ | CONH₂ |
| CH₂CH₂SMe | CONH₂ |
| CH₂CH(Me)SMe | CONH₂ |
| CH₂CH₂S(O)Me | CONH₂ |
| CH₂CH₂S(O)₂Me | CONH₂ |
| CH₂CO₂Me | CONH₂ |
| CH₂CO₂-i-Pr | CONH₂ |
| CH(Me)CO₂Me | CONH₂ |
| CH₂C(O)Me | CONH₂ |
| CH₂CH₂C(O)Me | CONH₂ |
| CH₂SiMe₃ | CONH₂ |
| CH₂CH₂SiMe₃ | CONH₂ |
| CH₂OPh | CONH₂ |
| CH₂Ph | CONH₂ |
| CH₂CH₂Ph | CONH₂ |
| CH(Me)Ph | CONH₂ |
| CH₂-2-Cl-Ph | CONH₂ |
| CH₂-3-Cl-Ph | CONH₂ |
| CH₂-4-Cl-Ph | CONH₂ |
| CH₂-2-thienyl | CONH₂ |
| CH₂-2-pyridinyl | CONH₂ |
| CH₂-3-pyridinyl | CONH₂ |
| CH(Et)₂ | CONH₂ |
| CH₂CH(Et)₂ | CONH₂ |
| CH₂CH(n-Pr)Me | CONH₂ |
| CH(Me)Et | CONH₂ |
| CH(Me)-n-Pr | CONH₂ |
| CH(CF₃)Et | CONH₂ |
| CH(Et)-n-Pr | CONH₂ |
| CH(Me)-n-Bu | CONH₂ |
| 2,2-dimethyipropyl | CONH₂ |
| CH₂CH₂CH(Me)₂ | CONH₂ |
| 2-F-Ph | 2-pyridinyl |
| 3-F-Ph | 2-pyridinyl |
| 4-F-Ph | 2-pyridinyl |
| 2,3-di-F-Ph | 2-pyridinyl |
| 2,4-di-F-Ph | 2-pyridinyl |
| 2,5-di-F-Ph | 2-pyridinyl |
| 2,6-di-F-Ph | 2-pyridinyl |
| 3,4-di-F-Ph | 2-pyridinyl |
| 3,5-di-F-Ph | 2-pyridinyl |
| 2,3-di-Cl-Ph | 2-pyridinyl |
| 2,4-di-Cl-Ph | 2-pyridinyl |
| 2,5-di-Cl-Ph | 2-pyridinyl |
| 2,6-di-Cl-Ph | 2-pyridinyl |
| 3,4-di-Cl-Ph | 2-pyridinyl |
| 3,5-di-Cl-Ph | 2-pyridinyl |
| 2-OMe-Ph | 2-pyridinyl |
| 3-OMe-Ph | 2-pyridinyl |
| 4-OMe-Ph | 2-pyridinyl |
| 2-Me-Ph | 2-pyridinyl |
| 3-Me-Ph | 2-pyridinyl |
| 4-Me-Ph | 2-pyridinyl |
| 2-CF₃-Ph | 2-pyridinyl |
| 3-CF₃-Ph | 2-pyridinyl |
| 4-CF₃-Ph | 2-pyridinyl |
| 2-CN-Ph | 2-pyridinyl |
| 3-CN-Ph | 2-pyridinyl |
| 4-CN-Ph | 2-pyridinyl |
| 2-NO₂-Ph | 2-pyridinyl |
| 3-NO₂-Ph | 2-pyridinyl |
| 4-NO₂-Ph | 2-pyridinyl |
| 3-(CH═CH₂)-Ph | 2-pyridinyl |
| 3-(CCH)-Ph | 2-pyridinyl |
| 4-c-Pr-Ph | 2-pyridinyl |
| 3-(CH═CCl₂)-Ph | 2-pyridinyl |
| 3-(CCCl)-Ph | 2-pyridinyl |
| 3-(2,2-di-Cl-c-Pr)-Ph | 2-pyridinyl |
| 2-OCF₃-Ph | 2-pyridinyl |
| 3-OCF₃-Ph | 2-pyridinyl |
| 4-OCF₃-Ph | 2-pyridinyl |
| 3-SMe-Ph | 2-pyridinyl |
| 3-S(O)Me-Ph | 2-pyridinyl |
| 3-SO₂Me-Ph | 2-pyridinyl |
| 3-NHMe-Ph | 2-pyridinyl |
| 3-NMe₂-Ph | 2-pyridinyl |
| 3-NH-c-Pr-Ph | 2-pyridinyl |
| 3-COMe-Ph | 2-pyridinyl |
| 3-CO₂Me-Ph | 2-pyridinyl |
| 3-CONHMe-Ph | 2-pyridinyl |
| 3-CONMe₂-Ph | 2-pyridinyl |
| 3-SiMe₃-Ph | 2-pyridinyl |
| 2,3-di-Me-Ph | 2-pyridinyl |
| 2-F-Ph | CONH₂ |
| 3-F-Ph | CONH₂ |
| 4-F-Ph | CONH₂ |
| 2,3-di-F-Ph | CONH₂ |
| 2,4-di-F-Ph | CONH₂ |
| 2,5-di-F-Ph | CONH₂ |
| 2,6-di-F-Ph | CONH₂ |
| 3,4-di-F-Ph | CONH₂ |
| 3,5-di-F-Ph | CONH₂ |
| 2,3-di-Cl-Ph | CONH₂ |
| 2,4-di-Cl-Ph | CONH₂ |
| 2,5-di-Cl-Ph | CONH₂ |
| 2,6-di-Cl-Ph | CONH₂ |
| 3,4-di-Cl-Ph | CONH₂ |
| 3,5-di-Cl-Ph | CONH₂ |
| 2-OMe-Ph | CONH₂ |
| 3-OMe-Ph | CONH₂ |
| 4-OMe-Ph | CONH₂ |
| 2-Me-Ph | CONH₂ |
| 3-Me-Ph | CONH₂ |
| 4-Me-Ph | CONH₂ |
| 2-CF₃-Ph | CONH₂ |
| 3-CF₃-Ph | CONH₂ |
| 4-CF₃-Ph | CONH₂ |
| 2-CN-Ph | CONH₂ |
| 3-CN-Ph | CONH₂ |
| 4-CN-Ph | CONH₂ |
| 2-NO₂-Ph | CONH₂ |
| 3-NO₂-Ph | CONH₂ |
| 4-NO₂-Ph | CONH₂ |
| 3-(CH═CH₂)-Ph | CONH₂ |
| 3-(CCH)-Ph | CONH₂ |
| 4-c-Pr-Ph | CONH₂ |
| 3-(CHCCl₂)-Ph | CONH₂ |
| 3-(CCCl)-Ph | CONH₂ |
| 3-(2,2-di-Cl-c-Pr)-Ph | CONH₂ |
| 2-OCF₃-Ph | CONH₂ |
| 3-OCF₃-Ph | CONH₂ |
| 4-OCF₃-Ph | CONH₂ |
| 3-SMe-Ph | CONH₂ |
| 3-S(O)Me-Ph | CONH₂ |
| 3-SO₂Me-Ph | CONH₂ |
| 3-NHMe-Ph | CONH₂ |
| 3-NMe₂-Ph | CONH₂ |
| 3-NH-c-Pr-Ph | CONH₂ |
| 3-COMe-Ph | CONH₂ |
| 3-CO₂Me-Ph | CONH₂ |
| 3-CONHMe-Ph | CONH₂ |
| 3-CONMe₂-Ph | CONH₂ |
| 3-SiMe₃-Ph | CONH₂ |
| 2,3-di-Me-Ph | CONH₂ |
| CH(Me)CH₂OMe | 1-methyl-1H-pyrazol-3-yl |
| CH(Me)CH(OMe)₂ | 1-methyl-1H-pyrazol-3-yl |
| CH₂-2-dioxolanyl | 1-methyl-1H-pyrazol-3-yl |
| CH₂CH₂OCF₃ | 1-methyl-1H-pyrazol-3-yl |
| CH₂CH₂SMe | 1-methyl-1H-pyrazol-3-yl |
| CH₂CH(Me)SMe | 1-methyl-1H-pyrazol-3-yl |
| CH₂CH₂S(O)Me | 1-methyl-1H-pyrazol-3-yl |
| CH₂CH₂S(O)2Me | 1-methyl-1H-pyrazol-3-yl |
| CH₂CO₂Me | 1-methyl-1H-pyrazol-3-yl |
| CH₂CO₂-i-Pr | 1-methyl-1H-pyrazol-3-yl |
| CH(Me)CO₂Me | 1-methyl-1H-pyrazol-3-yl |
| CH₂C(O)Me | 1-methyl-1H-pyrazol-3-yl |
| CH₂CH₂C(O)Me | 1-methyl-1H-pyrazol-3-yl |
| CH₂SiMe₃ | 1-methyl-1H-pyrazol-3-yl |
| CH₂CH₂SiMe₃ | 1-methyl-1H-pyrazol-3-yl |
| CH₂OPh | 1-methyl-1H-pyrazol-3-yl |
| CH₂Ph | 1-methyl-1H-pyrazol-3-yl |
| CH₂CH₂Ph | 1-methyl-1H-pyrazol-3-yl |
| CH(Me)Ph | 1-methyl-1H-pyrazol-3-yl |
| CH₂-2-Cl-Ph | 1-methyl-1H-pyrazol-3-yl |
| CH₂-3-Cl-Ph | 1-methyl-1H-pyrazol-3-yl |
| CH₂-4-Cl-Ph | 1-methyl-1H-pyrazol-3-yl |
| CH₂-2-thienyl | 1-methyl-1H-pyrazol-3-yl |
| CH₂-2-pyridinyl | 1-methyl-1H-pyrazol-3-yl |
| CH₂-3-pyridinyl | 1-methyl-1H-pyrazol-3-yl |
| CH(Et)₂ | 1-methyl-1H-pyrazol-3-yl |
| CH₂CH(Et)₂ | 1-methyl-1H-pyrazol-3-yl |
| CH₂CH(n-Pr)Me | 1-methyl-1H-pyrazol-3-yl |
| CH(Me)Et | 1-methyl-1H-pyrazol-3-yl |
| CH(Me)-n-Pr | 1-methyl-1H-pyrazol-3-yl |
| CH(CF₃)Et | 1-methyl-1H-pyrazol-3-yl |
| CH(Et)-n-Pr | 1-methyl-1H-pyrazol-3-yl |
| CH(Me)-n-Bu | 1-methyl-1H-pyrazol-3-yl |
| 2,2-dimethylpropyl | 1-methyl-1H-pyrazol-3-yl |
| CH₂CH₂CH(Me)₂ | 1-methyl-1H-pyrazol-3-yl |
| CH₂-2-F-Ph | 1-methyl-1H-pyrazol-3-yl |
| CH₂-3-F-Ph | 1-methyl-1H-pyrazol-3-yl |
| CH₂-4-F-Ph | 1-methyl-1H-pyrazol-3-yl |
| CH₂-2-Me-Ph | 1-methyl-1H-pyrazol-3-yl |
| CH₂-3-Me-Ph | 1-methyl-1H-pyrazol-3-yl |
| CH₂-4-Me-Ph | 1-methyl-1H-pyrazol-3-yl |
| CH₂-2-OMe-Ph | 1-methyl-1H-pyrazol-3-yl |
| CH₂-3-OMe-Ph | 1-methyl-1H-pyrazol-3-yl |
| CH₂-4-OMe-Ph | 1-methyl-1H-pyrazol-3-yl |
| cis-2-Me-c-Hex | 1-methyl-1H-pyrazol-3-yl |
| trans-2-Me-c-Hex | 1-methyl-1H-pyrazol-3-yl |
| cis-3-Me-c-Hex | 1-methyl-1H-pyrazol-3-yl |
| trans-3-Me-c-Hex | 1-methyl-1H-pyrazol-3-yl |
| cis-4-Me-c-Hex | 1-methyl-1H-pyrazol-3-yl |
| trans-4-Me-c-Hex | 1-methyl-1H-pyrazol-3-yl |
| CH₂CH(Me)OMe | 1-methyl-1H-pyrazol-3-yl |

TABLE 2

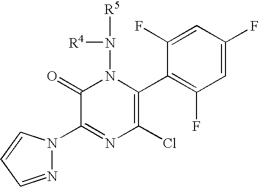

| R⁴ | R⁵ |
|---|---|
| Me | H |
| Et | H |
| i-Pr | H |
| n-Pr | H |
| i-Bu | H |
| n-Bu | H |
| s-Bu | H |
| 3-Me-Bu | H |
| n-pentyl | H |
| n-Hex | H |
| 2-propenyl | H |
| 2-Me-2-propenyl | H |
| 3-butenyl | H |
| 3-pentenyl | H |
| 2-propynyl | H |
| 3-butynyl | H |
| 4-butynyl | H |
| c-Pr | H |
| c-pentyl | H |
| c-Hex | H |
| 2-cyclohexenyl | H |
| 3-cyclohexenyl | H |
| CH₂-c-Pr | H |
| CH₂-c-Hex | H |
| CH₂-2-cyclohexenyl | H |
| 4-tetrahydropyranyl | H |
| 3-tetrahydropyranyl | H |
| 3-tetrahydrofuranyl | H |
| Ph | H |
| 2-Cl-phenyl | H |
| 3-Cl-phenyl | H |
| 4-Cl-phenyl | H |
| 2-pyridinyl | H |
| 2-pyrimidyl | H |
| 2-pyrazinyl | H |
| 2-thiazolyl | H |
| 2-oxazolyl | H |
| CF₃ | H |
| CF₂CF₃ | H |
| CH₂CF₃ | H |
| CH(Me)CF₃ | H |
| CH₂CH₂F | H |
| 2-chloro-2-propenyl | H |
| 3,3-dichloro-2-propenyl | H |
| CH₂-2-tetrahydrofuranyl | H |
| CH₂-2-tetrahydropyranyl | H |
| CH₂CN | H |
| CH₂NO₂ | H |
| CH₂CH₂OH | H |
| CH₂CH₂OMe | H |
| CH₂CH(Me)OMe | H |
| CH(Me)CH₂OMe | H |
| CH(Me)CH(OMe)₂ | H |
| CH₂-2-dioxolanyl | H |
| CH₂CH₂OCF₃ | H |
| CH₂CH₂SMe | H |
| CH₂CH(Me)SMe | H |
| CH₂CH₂S(O)Me | H |
| CH₂CH₂S(O)₂Me | H |
| CH₂CO₂Me | H |
| CH₂CO₂-i-Pr | H |
| CH(Me)CO₂Me | H |
| CH₂C(O)Me | H |
| CH₂CH₂C(O)Me | H |
| CH₂SiMe₃ | H |
| CH₂CH₂SiMe₃ | H |
| CH₂OPh | H |
| CH₂Ph | H |
| CH₂CH₂Ph | H |
| CH(Me)Ph | H |
| CH₂-2-Cl-Ph | H |
| CH₂-3-Cl-Ph | H |
| CH₂-4-Cl-Ph | H |
| CH₂-2-thienyl | H |
| CH₂-2-pyridinyl | H |
| CH₂-3-pyridinyl | H |
| CH(Et)₂ | H |
| CH₂CH(Et)₂ | H |
| CH₂CH₂(n-Pr)Me | H |
| CH(Me)Et | H |
| CH(Me)-n-Pr | H |
| CH(CF₃)Et | H |
| CH(Et)-n-Pr | H |
| CH(Me)-n-Bu | H |
| 2,2-dimethylpropyl | H |
| CH₂CH₂CH(Me)₂ | H |
| Me | Me |
| Et | Me |
| i-Pr | Me |
| n-Pr | Me |
| i-Bu | Me |
| n-Bu | Me |
| s-Bu | Me |
| 3-Me-Bu | Me |
| n-pentyl | Me |
| n-Hex | Me |
| 2-propenyl | Me |
| 2-Me-2-propenyl | Me |
| 3-butenyl | Me |
| 3-pentenyl | Me |
| 2-propynyl | Me |
| 3-butynyl | Me |
| 4-butynyl | Me |
| c-Pr | Me |
| c-pentyl | Me |
| c-Hex | Me |
| 2-cyclohexenyl | Me |
| 3-cyclohexenyl | Me |
| CH₂-c-Pr | Me |
| CH₂-c-Hex | Me |
| CH₂-2-cyclohexenyl | Me |
| 4-tetrahydropyranyl | Me |
| 3-tetrahydropyranyl | Me |
| 3-tetrahydrofuranyl | Me |
| Ph | Me |
| 2-Cl-phenyl | Me |
| 3-Cl-phenyl | Me |
| 4-Cl-phenyl | Me |
| 2-pyridinyl | Me |
| 2-pyrimidyl | Me |
| 2-pyrazinyl | Me |
| 2-thiazolyl | Me |
| 2-oxazolyl | Me |
| CF₃ | Me |
| CF₂CF₃ | Me |
| CH₂CF₃ | Me |
| CH(Me)CF₃ | Me |
| CH₂CH₂F | Me |
| 2-chloro-2-propenyl | Me |
| 3,3-dichloro-2-propenyl | Me |
| CH₂-2-tetrahydrofuranyl | Me |
| CH₂-2-tetrahydropyranyl | Me |
| CH₂CN | Me |
| CH₂NO₂ | Me |
| CH₂CH₂OH | Me |
| CH₂CH₂OMe | Me |
| CH₂CH(Me)OMe | Me |
| CH(Me)CH₂OMe | Me |
| CH(Me)CH(OMe)₂ | Me |
| CH₂-2-dioxolanyl | Me |
| CH₂CH₂OCF₃ | Me |
| CH₂CH₂SMe | Me |
| CH₂CH(Me)SMe | Me |
| CH₂CH₂S(O)Me | Me |
| CH₂CH₂S(O)₂Me | Me |
| CH₂CO₂Me | Me |
| CH₂CO₂-i-Pr | Me |
| CH(Me)CO₂Me | Me |
| CH₂C(O)Me | Me |
| CH₂CH₂C(O)Me | Me |
| CH₂SiMe₃ | Me |
| CH₂CH₂SiMe₃ | Me |
| CH₂OPh | Me |
| CH₂Ph | Me |
| CH₂CH₂Ph | Me |
| CH(Me)Ph | Me |
| CH₂-2-Cl-Ph | Me |
| CH₂-3-Cl-Ph | Me |
| CH₂-4-Cl-Ph | Me |
| CH₂-2-thienyl | Me |
| CH₂-2-pyridinyl | Me |
| CH₂-3-pyridinyl | Me |
| CH(Et)₂ | Me |
| CH₂CH(Et)₂ | Me |
| CH₂CH(n-Pr)Me | Me |
| CH(Me)Et | Me |
| CH(Me)-n-Pr | Me |
| CH(CF₃)Et | Me |
| CH(Et)-n-Pr | Me |
| CH(Me)-n-Bu | Me |
| 2,2-dimethylpropyl | Me |
| CH₂CH₂CH(Me)₂ | Me |

TABLE 3a

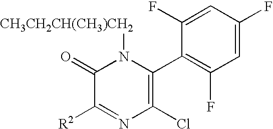

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CHHD 2S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NHN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N-OH
C(Me)═N-OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 3b

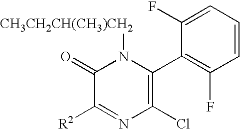

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NRN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N-OH
C(Me)═N-OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 3c

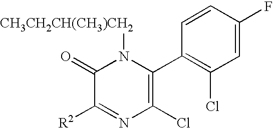

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NRN═C(CH₂)₄
NHN═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N-OH
C(Me)═N-OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 3d

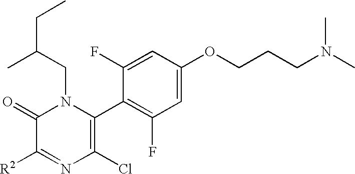

R²
3-Cl-2-pyridinyl
5-Cl-2-pyridinyl
6-Cl-2-pyridinyl
2-pyrimidinyl
5-Cl-2-pyrimidinyl
4-Cl-2-pyrimidinyl
2-thiazolyl
4-thiazolyl
2-oxazolyl
4-oxazolyl
3-Me-2-pyridinyl
5-Me-2-pyridinyl
6-Me-2-pyridinyl
4-Me-2-pyrimidinyl
4-pyrimidinyl
2-Me-4-pyrimidinyl
2,6-di-Me-4-pyrimidinyl
2-pyrazinyl
6-Cl-2-pyrazinyl
3-Cl-2-pyrazinyl
3-pyridazinyl
6-Cl-3-pyridazinyl
6-Me-3-pyridazinyl
4-OMe-2-pyrimidyl
2-Cl-4-pyrimidinyl
3-Me-2-pyrazinyl
1,2,4-triazin-3-yl
1,2,4-triazin-5-yl
4,6-di-Cl-1,3,5-triazin-2-yl
2-benzothiazolyl
2-benzoxazolyl
2-quinolinyl
4-Me-2-quinolinyl
2-quinoxalinyl
1,2,4-benzotriazin-3-yl
N-Me-2-benzimidazolyl
1-isoquinolinyl
3-isoxazolyl
3-isothiazolyl
CONHMe
CONHEt
CONH-n-Pr
NHCHO
NHCO₂Me
2-cinnolinyl
1,8-naphthyridin-2-yl
4-Me-2-quinazolinyl
2-Me-4-quinazolinyl
2-Cl-4-quinazolinyl
6-Cl-2-quinoxalinyl
7-Cl-2-quinoxalinyl
CONH-i-Pr
CONH-c-Pr
CONMe₂
CONEt₂
6,7-di-Cl-2-quinoxalinyl
6-Cl-2-benzothiazolyl
6-NO₂-2-benzothiazolyl
1-Me-1H-imidazol-2-yl
2-Me-1H-1,2,3-triazol-4-yl
1,2,3-oxadiazol-4-yl
1,2,3-thiadiazol-4-yl
1,3,4-thiadiazol-2-yl
3-Cl-1,2,4-thiadiazol-5-yl
3-Me-1,2,4-thiadiazol-5-yl
3-Me-1,2,4-oxadiazol-5-yl
1,3,4-oxadiazol-2-yl
3-Me-1H-pyrazol-1-yl
3-CF₃-1H-pyrazol-1-yl
3-t-Bu-1H-pyrazol-1-yl
3-Br-1H-pyrazol-1-yl
3-Ph-1H-pyrazol-1-yl
3-CN-1H-pyrazol-1-yl
4-CN-1H-pyrazol-1-yl
4-Me-1H-pyrazol-1-yl
4-Ph-1H-pyrazol-1-yl
4-Cl-1H-pyrazol-1-yl
4-Br-1H-pyrazol-1-yl
4-Ph-1H-pyrazol-1-yl
5-Me-1H-pyrazol-1-yl
3,5-di-Me-1H-pyrazol-1-yl
3-CF₃-5-Me-1H-pyrazol-1-yl
3,4,5-tri-Me-1H-pyrazol-1-yl
1H-pyrazolin-2-yl
3-Me-1H-pyrazolin-2-yl
3-Ph-1H-pyrazolin-2-yl
NHCOMe
NHCO₂Et
1H-1,2,4-triazol-1-yl
3-Me-1H-1,2,4-triazol-1-yl
3,5-di-Me-1H-1,2,4-triazol-1-yl
3-SMe-1H-1,2,4-triazol-1-yl
3-Br-1H-1,2,4-triazol-1-yl
3-Cl-1H-1,2,4-triazol-1-yl
1H-1,2,3-triazol-1-yl
4-Me-1H-pyrazolin-2-yl
CONHCH₂CH₂OMe
CONHCH₂CH₂SMe
CONHCH₂CH₂NMe₂
CONHCH₂CCH
CONHCH₂C═CH₂
CONHCH₂CH₂S(O)Me
CONHCH₂CH₂SO₂Me
1H-1,2,3-triazol-2-yl
4,5-di-Br-1H-1,2,3-triazol-1-yl
4,5-di-Br-1H-1,2,3-triazol-1-yl
4,5-di-Me-1H-1,2,3-triazol-1-yl
4,5-di-Me-1H-1,2,3-triazol-1-yl
3-CF₃-1H-1,2,4-triazol-1-yl
NHN═C(Me)₂
NRN═C(CH₂)₄
NH7N═C(CH₂)₅
ON═C(Me)₂
ON═C(CH₂)₅
ON═C(CH₂)₄
NHNMe₂
ONMe₂
NHN(CH₂)₅
NHN(CH₂CH₂OCH₂CH₂)
C(S)NH₂
C(Me)═NNHMe
C(Me)═N-1-piperidino
C(Me)═N-OH
C(Me)═N-OMe
C(Me)═NO-i-Pr
CONHCH₂CF₃
CONHCH₂CN
CONHCH₂CO₂Me
CONHCH₂SiMe₃
CON(CH₂)₅
NHCOEt
NHCONHMe

TABLE 4a

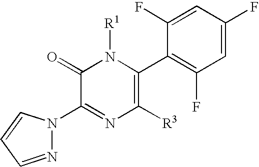

| R¹ | R³ |
|---|---|
| 2-Me-Bu | Cl |
| 2-Me-Bu | F |
| 2-Me-Bu | Br |
| 2-Me-Bu | Me |
| 2-Me-Bu | Et |
| 2-Me-Bu | c-Pr |
| 2-Me-Bu | CF₃ |
| 2-Me-Bu | OMe |
| 2-Me-Bu | SMe |
| 2-Me-Bu | SCF₃ |
| 2-Me-Bu | OCF₂H |
| 2-Me-Bu | CO₂Me |
| 2-Me-Bu | ethenyl |
| 2-Me-Bu | ethynyl |
| 2-Me-Bu | 2,2-di-Cl-c-Pr |
| NH-i-Pr | Cl |
| NH-i-Pr | F |
| NH-i-Pr | Br |
| NH-i-Pr | Me |
| NH-i-Pr | Et |
| NH-i-Pr | c-Pr |
| NH-i-Pr | CF₃ |
| NH-i-Pr | OMe |
| NH-i-Pr | SMe |
| NH-i-Pr | SCF₃ |
| NH-i-Pr | OCF₂H |
| NH-i-Pr | CO₂Me |
| NH-i-Pr | ethenyl |
| NH-i-Pr | ethynyl |
| NH-i-Pr | 2,2-di-Cl-c-Pr |
| i-Bu | Cl |
| i-Bu | F |
| i-Bu | Br |
| i-Bu | Me |
| i-Bu | Et |
| i-Bu | c-Pr |
| i-Bu | CF₃ |
| i-Bu | OMe |
| i-Bu | SMe |
| i-Bu | SCF₃ |
| i-Bu | OCF₂H |
| i-Bu | CO₂Me |
| i-Bu | ethenyl |
| i-Bu | ethynyl |
| i-Bu | 2,2-di-Cl-c-Pr |
| 3-F-Ph | Cl |
| 3-F-Ph | F |
| 3-F-Ph | Br |
| 3-F-Ph | Me |
| 3-F-Ph | Et |
| 3-F-Ph | c-Pr |
| 3-F-Ph | CF₃ |
| 3-F-Ph | OMe |
| 3-F-Ph | SMe |
| 3-F-Ph | SCF₃ |
| 3-F-Ph | OCF₂H |
| 3-F-Ph | CO₂Me |
| 3-F-Ph | ethenyl |
| 3-F-Ph | ethynyl |
| 3-F-Ph | 2,2-di-Cl-c-Pr |

TABLE 4b

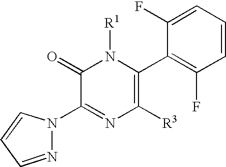

| R¹ | R³ |
|---|---|
| 2-Me-Bu | Cl |
| 2-Me-Bu | F |
| 2-Me-Bu | Br |
| 2-Me-Bu | Me |
| 2-Me-Bu | Et |
| 2-Me-Bu | c-Pr |
| 2-Me-Bu | CF₃ |
| 2-Me-Bu | OMe |
| 2-Me-Bu | SMe |
| 2-Me-Bu | SCF₃ |
| 2-Me-Bu | OCF₂H |
| 2-Me-Bu | CO₂Me |
| 2-Me-Bu | ethenyl |
| 2-Me-Bu | ethynyl |
| 2-Me-Bu | 2,2-di-Cl-c-Pr |
| NH-i-Pr | Cl |
| NH-i-Pr | F |
| NH-i-Pr | Br |
| NH-i-Pr | Me |
| NH-i-Pr | Et |
| NH-i-Pr | c-Pr |
| NH-i-Pr | CF₃ |
| NH-i-Pr | OMe |
| NH-i-Pr | SMe |
| NH-i-Pr | SCF₃ |
| NH-i-Pr | OCF₂H |
| NH-i-Pr | CO₂Me |
| NH-i-Pr | ethenyl |
| NH-i-Pr | ethynyl |
| NH-i-Pr | 2,2-di-Cl-c-Pr |
| i-Bu | Cl |
| i-Bu | F |
| i-Bu | Br |
| i-Bu | Me |
| i-Bu | Et |
| i-Bu | c-Pr |
| i-Bu | CF₃ |
| i-Bu | OMe |
| i-Bu | SMe |
| i-Bu | SCF₃ |
| i-Bu | OCF₂H |
| i-Bu | CO₂Me |
| i-Bu | ethenyl |
| i-Bu | ethynyl |
| i-Bu | 2,2-di-Cl-c-Pr |
| 3-F-Ph | Cl |
| 3-F-Ph | F |
| 3-F-Ph | Br |
| 3-F-Ph | Me |
| 3-F-Ph | Et |
| 3-F-Ph | c-Pr |
| 3-F-Ph | CF₃ |
| 3-F-Ph | OMe |
| 3-F-Ph | SMe |
| 3-F-Ph | SCF₃ |
| 3-F-Ph | OCF₂H |
| 3-F-Ph | CO₂Me |
| 3-F-Ph | ethenyl |
| 3-F-Ph | ethynyl |
| 3-F-Ph | 2,2-di-Cl-c-Pr |

TABLE 4c

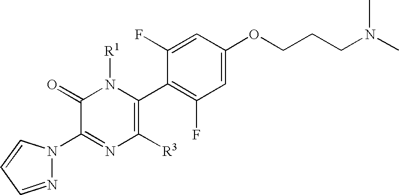

| R¹ | R³ |
|---|---|
| 2-Me-Bu | Cl |
| 2-Me-Bu | F |
| 2-Me-Bu | Br |
| 2-Me-Bu | Me |
| 2-Me-Bu | Et |
| 2-Me-Bu | c-Pr |
| 2-Me-Bu | CF₃ |
| 2-Me-Bu | OMe |
| 2-Me-Bu | SMe |
| 2-Me-Bu | SCF₃ |
| 2-Me-Bu | OCF₂H |
| 2-Me-Bu | CO₂Me |
| 2-Me-Bu | ethenyl |
| 2-Me-Bu | ethynyl |
| 2-Me-Bu | 2,2-di-Cl-c-Pr |
| NH-i-Pr | Cl |
| NH-i-Pr | F |
| NH-i-Pr | Br |
| NH-i-Pr | Me |
| NH-i-Pr | Et |
| NH-i-Pr | c-Pr |
| NH-i-Pr | CF₃ |
| NH-i-Pr | OMe |
| NH-i-Pr | SMe |
| NH-i-Pr | SCF₃ |
| NH-i-Pr | OCF₂H |
| NH-i-Pr | CO₂Me |
| NH-i-Pr | ethenyl |
| NH-i-Pr | ethynyl |
| NH-i-Pr | 2,2-di-Cl-c-Pr |
| i-Bu | Cl |
| i-Bu | F |
| i-Bu | Br |
| i-Bu | Me |
| i-Bu | Et |
| i-Bu | c-Pr |
| i-Bu | CF₃ |
| i-Bu | OMe |
| i-Bu | SMe |
| i-Bu | SCF₃ |
| i-Bu | OCF₂H |
| i-Bu | CO₂Me |
| i-Bu | ethenyl |
| i-Bu | ethynyl |
| i-Bu | 2,2-di-Cl-c-Pr |
| 3-F-Ph | Cl |
| 3-F-Ph | F |
| 3-F-Ph | Br |
| 3-F-Ph | Me |
| 3-F-Ph | Et |
| 3-F-Ph | c-Pr |
| 3-F-Ph | CF₃ |
| 3-F-Ph | OMe |
| 3-F-Ph | SMe |
| 3-F-Ph | SCF₃ |
| 3-F-Ph | OCF₂H |
| 3-F-Ph | CO₂Me |
| 3-F-Ph | ethenyl |
| 3-F-Ph | ethynyl |
| 3-F-Ph | 2,2-di-Cl-c-Pr |

TABLE 5a

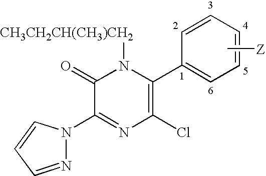

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 5b

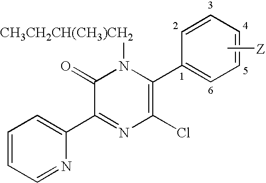

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 5c

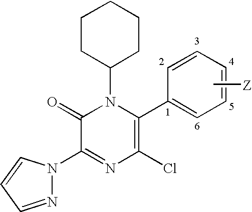

Z
2,3,4,5,6-penta-F
2-F
3-F
4-F
2-Cl
3-Cl
4-Cl
2-OMe
3-OMe
4-OMe
2-Me
3-Me
4-Me
2-CF₃
3-CF₃
4-CF₃
2-Et
2-i-Pr
2-c-Pr
2-Br
2-CN
2-SMe
2-OCF₃
2-SCF₃
2-ethenyl
2-ethynyl
2-OEt
2,4-di-F
2,3-di-F
2,6-di-F
2,5-di-F
2,3,4-tri-F
2,3,5-tri-F
2,3,6-tri-F
2,4,5-tri-F
3,4,5-tri-F
2-F-6-Cl
2-F-4-Cl
2-F-3-Cl
2-F-5-Cl
2-F-6-Me
2-F-4-Me
2-F-4-OMe
2-F-6-OMe
2-F-4-Br
2-F-6-Br
2-F-6-CN
2-F-6-CF₃
2-F-4-CF₃
2,6-di-F-4-Cl
2,6-di-F-4-OMe
2,6-di-F-4-Me
2,6-di-F-4-CF₃
2,6-di-F-4-CN
2,6-di-F-4-SMe
2-Cl-4-F
2-Me-4-F
2-CF₃-4-F
2-CF₃-6-F
2,6-di-Me-4-F
2-OMe-4-F
2-Et-4-F
2,6-di-Me-4-Cl
2,6-di-Me-4-OMe
2,6-di-Me-4-CF₃
2,6-di-Me-4-Br
2,6-di-Me-4-SMe
2-Cl-4-Me
2-CF₃-4-Me
2-OMe-4-Me
2-Br-4-Me
2-Et-4-Me
2-CN-4-Me
2,6-di-Cl-4-F
2,6-di-Cl-4-Me
2,6-di-Cl-4-Br
2,6-di-Cl-4-OMe
2,6-di-Cl-4-SMe
2,4,6-tri-Cl
2,4,6-tri-Me
2,4,5-tri-Me
2,3,6-tri-Me
2,3,4-tri-Me
2,4,5-tri-Cl
2,3,6-tri-Cl
2,3,4-tri-Cl
2,6-di-Et
2,6-di-Et-4-F
2,6-di-Et-4-Cl
2,6-di-F-4-Cl

TABLE 6a

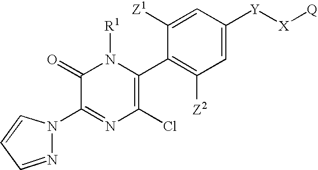

| R¹ | Z¹ | Z² | Y | X | Q |
|---|---|---|---|---|---|
| 2-Me-Bu | F | F | O | —(CH₂)₃— | NMe(Et) |
| 2-Me-Bu | F | F | O | —(CH₂)₃— | NHMe |
| 2-Me-Bu | F | F | O | —(CH₂)₃— | NHEt |
| 2-Me-Bu | F | F | O | —(CH₂)₃— | NH-n-Pr |
| 2-Me-Bu | F | F | O | —(CH₂)₃— | 1-pyrrolidinyl |
| 2-Me-Bu | F | F | O | —(CH₂)₃— | 1-azetidinyl |
| 2-Me-Bu | F | F | O | —(CH₂)₃— | 1-aziridinyl |
| 2-Me-Bu | F | F | O | —(CH₂)₃— | 1-morpholino |
| 2-Me-Bu | F | F | O | —(CH₂)₃— | 1-piperidinyl |
| 2-Me-Bu | F | F | O | —(CH₂)₃— | N(Et)₂ |
| 2-Me-Bu | F | F | O | —(CH₂)₃— | NH-i-Pr |
| 2-Me-Bu | F | F | O | —(CH₂)₃— | NMe(n-Pr) |
| 2-Me-Bu | F | F | O | —(CH₂)₃— | NMe(i-Pr) |
| 2-Me-Bu | F | F | O | —(CH₂)₃— | NMe(CHO) |
| 2-Me-Bu | F | F | O | —(CH₂)₃— | NMe(COCH₃) |
| 2-Me-Bu | F | F | O | —(CH₂)₃— | NMe(CO₂Me) |
| 2-Me-Bu | F | F | O | —(CH₂)₃— | NMe(COEt) |
| 2-Me-Bu | F | F | O | —(CH₂)₃— | NMe(CO-c-Pr) |
| 2-Me-Bu | F | F | O | —(CH₂)₃— | NMe(CO₂Et) |
| 2-Me-Bu | F | F | O | —(CH₂)₃— | NMe(COCF₃) |
| 2-Me-Bu | F | F | O | —(CH₂)₃— | NMe(Boc) |
| 2-Me-Bu | F | F | O | —(CH₂)₃— | NMe(CH₂CF₃) |
| 2-Me-Bu | F | F | O | —(CH₂)₃— | OH |
| 2-Me-Bu | F | H | O | —(CH₂)₃— | NMe(Et) |
| 2-Me-Bu | F | H | O | —(CH₂)₃— | NHMe |
| 2-Me-Bu | F | H | O | —(CH₂)₃— | NHEt |
| 2-Me-Bu | F | H | O | —(CH₂)₃— | NH-n-Pr |
| 2-Me-Bu | F | H | O | —(CH₂)₃— | 1-pyrrolidinyl |
| 2-Me-Bu | F | H | O | —(CH₂)₃— | 1-azetidinyl |
| 2-Me-Bu | F | H | O | —(CH₂)₃— | 1-aziridinyl |
| 2-Me-Bu | F | H | O | —(CH₂)₃— | 1-morpholino |
| 2-Me-Bu | F | H | O | —(CH₂)₃— | 1-piperidinyl |
| 2-Me-Bu | F | H | O | —(CH₂)₃— | N(Et)₂ |
| 2-Me-Bu | F | H | O | —(CH₂)₃— | NH-i-Pr |
| 2-Me-Bu | F | H | O | —(CH₂)₃— | NMe(n-Pr) |
| 2-Me-Bu | F | H | O | —(CH₂)₃— | NMe(i-Pr) |
| 2-Me-Bu | F | H | O | —(CH₂)₃— | NMe(CHO) |
| 2-Me-Bu | F | H | O | —(CH₂)₃— | NMe(COCH₃) |
| 2-Me-Bu | F | H | O | —(CH₂)₃— | NMe(CO₂Me) |
| 2-Me-Bu | F | H | O | —(CH₂)₃— | NMe(COEt) |
| 2-Me-Bu | F | H | O | —(CH₂)₃— | NMe(CO-c-Pr) |
| 2-Me-Bu | F | H | O | —(CH₂)₃— | NMe(CO₂Et) |
| 2-Me-Bu | F | H | O | —(CH₂)₃— | NMe(COCF₃) |
| 2-Me-Bu | F | H | O | —(CH₂)₃— | NMe(Boc) |
| 2-Me-Bu | F | H | O | —(CH₂)₃— | NMe(CH₂CF₃) |
| 2-Me-Bu | F | H | O | —(CH₂)₃— | NMe₂ |
| 2-Me-Bu | Cl | H | O | —(CH₂)₃— | NMe(Et) |
| 2-Me-Bu | Cl | H | O | —(CH₂)₃— | NHMe |
| 2-Me-Bu | Cl | H | O | —(CH₂)₃— | NHEt |
| 2-Me-Bu | Cl | H | O | —(CH₂)₃— | NH-n-Pr |
| 2-Me-Bu | Cl | H | O | —(CH₂)₃— | 1-pyrrolidinyl |
| 2-Me-Bu | Cl | H | O | —(CH₂)₃— | 1-azetidinyl |
| 2-Me-Bu | Cl | H | O | —(CH₂)₃— | 1-aziridinyl |
| 2-Me-Bu | Cl | H | O | —(CH₂)₃— | 1-morpholino |
| 2-Me-Bu | Cl | H | O | —(CH₂)₃— | 1-piperidinyl |
| 2-Me-Bu | Cl | H | O | —(CH₂)₃— | N(Et)₂ |
| 2-Me-Bu | Cl | H | O | —(CH₂)₃— | NH-i-Pr |
| 2-Me-Bu | Cl | H | O | —(CH₂)₃— | NMe(n-Pr) |
| 2-Me-Bu | Cl | H | O | —(CH₂)₃— | NMe(i-Pr) |
| 2-Me-Bu | Cl | H | O | —(CH₂)₃— | NMe(CHO) |
| 2-Me-Bu | Cl | H | O | —(CH₂)₃— | NMe(COCH₃) |
| 2-Me-Bu | Cl | H | O | —(CH₂)₃— | NMe(CO₂Me) |
| 2-Me-Bu | Cl | H | O | —(CH₂)₃— | NMe(COEt) |
| 2-Me-Bu | Cl | H | O | —(CH₂)₃— | NMe(CO-c-Pr) |
| 2-Me-Bu | Cl | H | O | —(CH₂)₃— | NMe(CO₂Et) |
| 2-Me-Bu | Cl | H | O | —(CH₂)₃— | NMe(COCF₃) |
| 2-Me-Bu | Cl | H | O | —(CH₂)₃— | NMe(Boc) |
| 2-Me-Bu | Cl | H | O | —(CH₂)₃— | NMe(CH₂CF₃) |
| 2-Me-Bu | Cl | H | O | —(CH₂)₃— | NMe₂ |
| 2-Me-Bu | F | Cl | O | —(CH₂)₃— | NMe(Et) |
| 2-Me-Bu | F | Cl | O | —(CH₂)₃— | NHMe |
| 2-Me-Bu | F | Cl | O | —(CH₂)₃— | NHEt |
| 2-Me-Bu | F | Cl | O | —(CH₂)₃— | NH-n-Pr |
| 2-Me-Bu | F | Cl | O | —(CH₂)₃— | 1-pyrrolidinyl |
| 2-Me-Bu | F | Cl | O | —(CH₂)₃— | 1-azetidinyl |
| 2-Me-Bu | F | Cl | O | —(CH₂)₃— | 1-aziridinyl |
| 2-Me-Bu | F | Cl | O | —(CH₂)₃— | 1-morpholino |
| 2-Me-Bu | F | Cl | O | —(CH₂)₃— | 1-piperidinyl |
| 2-Me-Bu | F | Cl | O | —(CH₂)₃— | N(Et)₂ |
| 2-Me-Bu | F | Cl | O | —(CH₂)₃— | NH-i-Pr |
| 2-Me-Bu | F | Cl | O | —(CH₂)₃— | NMe(n-Pr) |
| 2-Me-Bu | F | Cl | O | —(CH₂)₃— | NMe(i-Pr) |
| 2-Me-Bu | F | Cl | O | —(CH₂)₃— | NMe(CHO) |
| 2-Me-Bu | F | Cl | O | —(CH₂)₃— | NMe(COCH₃) |
| 2-Me-Bu | F | Cl | O | —(CH₂)₃— | NMe(CO₂Me) |
| 2-Me-Bu | F | Cl | O | —(CH₂)₃— | NMe(COEt) |
| 2-Me-Bu | F | Cl | O | —(CH₂)₃— | NMe(CO-c-Pr) |
| 2-Me-Bu | F | Cl | O | —(CH₂)₃— | NMe(CO₂Et) |
| 2-Me-Bu | F | Cl | O | —(CH₂)₃— | NMe(COCF₃) |
| 2-Me-Bu | F | Cl | O | —(CH₂)₃— | NMe(Boc) |
| 2-Me-Bu | F | Cl | O | —(CH₂)₃— | NMe(CH₂CF₃) |
| 2-Me-Bu | F | Cl | O | —(CH₂)₃— | NMe₂ |
| 2-Me-Bu | F | F | O | —(CH₂)₂— | NMe(Et) |
| 2-Me-Bu | F | F | O | —(CH₂)₂— | NHMe |
| 2-Me-Bu | F | F | O | —(CH₂)₂— | NHEt |
| 2-Me-Bu | F | F | O | —(CH₂)₂— | NH-n-Pr |
| 2-Me-Bu | F | F | O | —(CH₂)₂— | 1-pyrrolidinyl |
| 2-Me-Bu | F | F | O | —(CH₂)₂— | 1-azetidinyl |
| 2-Me-Bu | F | F | O | —(CH₂)₂— | 1-aziridinyl |
| 2-Me-Bu | F | F | O | —(CH₂)₂— | 1-morpholino |
| 2-Me-Bu | F | F | O | —(CH₂)₂— | 1-piperidinyl |
| 2-Me-Bu | F | F | O | —(CH₂)₂— | N(Et)₂ |
| 2-Me-Bu | F | F | O | —(CH₂)₂— | NH-i-Pr |
| 2-Me-Bu | F | F | O | —(CH₂)₂— | NMe(n-Pr) |
| 2-Me-Bu | F | F | O | —(CH₂)₂— | NMe(i-Pr) |
| 2-Me-Bu | F | F | O | —(CH₂)₂— | NMe(CHO) |
| 2-Me-Bu | F | F | O | —(CH₂)₂— | NMe(COCH₃) |
| 2-Me-Bu | F | F | O | —(CH₂)₂— | NMe(CO₂Me) |
| 2-Me-Bu | F | F | O | —(CH₂)₂— | NMe(COEt) |
| 2-Me-Bu | F | F | O | —(CH₂)₂— | NMe(CO-c-Pr) |
| 2-Me-Bu | F | F | O | —(CH₂)₂— | NMe(CO₂Et) |
| 2-Me-Bu | F | F | O | —(CH₂)₂— | NMe(COCF₃) |
| 2-Me-Bu | F | F | O | —(CH₂)₂— | NMe(Boc) |
| 2-Me-Bu | F | F | O | —(CH₂)₂— | NMe(CH₂CF₃) |
| 2-Me-Bu | F | F | O | —(CH₂)₂— | NMe₂ |
| 2-Me-Bu | F | F | O | —(CH₂)₄— | NMe(Et) |
| 2-Me-Bu | F | F | O | —(CH₂)₄— | NHMe |
| 2-Me-Bu | F | F | O | —(CH₂)₄— | NHEt |
| 2-Me-Bu | F | F | O | —(CH₂)₄— | NH-n-Pr |
| 2-Me-Bu | F | F | O | —(CH₂)₄— | 1-pyrrolidinyl |
| 2-Me-Bu | F | F | O | —(CH₂)₄— | 1-azetidinyl |
| 2-Me-Bu | F | F | O | —(CH₂)₄— | 1-aziridinyl |
| 2-Me-Bu | F | F | O | —(CH₂)₄— | 1-morpholino |
| 2-Me-Bu | F | F | O | —(CH₂)₄— | 1-piperidinyl |
| 2-Me-Bu | F | F | O | —(CH₂)₄— | N(Et)₂ |
| 2-Me-Bu | F | F | O | —(CH₂)₄— | NH-i-Pr |
| 2-Me-Bu | F | F | O | —(CH₂)₄— | NMe(n-Pr) |
| 2-Me-Bu | F | F | O | —(CH₂)₄— | NMe(i-Pr) |
| 2-Me-Bu | F | F | O | —(CH₂)₄— | NMe(CH0) |
| 2-Me-Bu | F | F | O | —(CH₂)₄— | NMe(COCH₃) |
| 2-Me-Bu | F | F | O | —(CH₂)₄— | NMe(CO₂Me) |
| 2-Me-Bu | F | F | O | —(CH₂)₄— | NMe(COEt) |
| 2-Me-Bu | F | F | O | —(CH₂)₄— | NMe(CO-c-Pr) |
| 2-Me-Bu | F | F | O | —(CH₂)₄— | NMe(CO₂Et) |
| 2-Me-Bu | F | F | O | —(CH₂)₄— | NMe(COCF₃) |
| 2-Me-Bu | F | F | O | —(CH₂)₄— | NMe(Boc) |
| 2-Me-Bu | F | F | O | —(CH₂)₄— | NMe(CH₂CF₃) |
| 2-Me-Bu | F | F | O | —(CH₂)₄— | NMe₂ |
| 2-Me-Bu | F | F | S | —(CH₂)₃— | NMe(Et) |
| 2-Me-Bu | F | F | S | —(CH₂)₃— | NHMe |
| 2-Me-Bu | F | F | S | —(CH₂)₃— | NHEt |
| 2-Me-Bu | F | F | S | —(CH₂)₃— | NH-n-Pr |
| 2-Me-Bu | F | F | S | —(CH₂)₃— | 1-pyrrolidinyl |
| 2-Me-Bu | F | F | S | —(CH₂)₃— | 1-azetidinyl |
| 2-Me-Bu | F | F | S | —(CH₂)₃— | 1-aziridinyl |
| 2-Me-Bu | F | F | S | —(CH₂)₃— | 1-morpholino |
| 2-Me-Bu | F | F | S | —(CH₂)₃— | 1-piperidinyl |
| 2-Me-Bu | F | F | S | —(CH₂)₃— | N(Et)₂ |
| 2-Me-Bu | F | F | S | —(CH₂)₃— | NH-i-Pr |
| 2-Me-Bu | F | F | S | —(CH₂)₃— | NMe(n-Pr) |
| 2-Me-Bu | F | F | S | —(CH₂)₃— | NMe(i-Pr) |
| 2-Me-Bu | F | F | S | —(CH₂)₃— | NMe(CHO) |
| 2-Me-Bu | F | F | S | —(CH₂)₃— | NMe(COCH₃) |
| 2-Me-Bu | F | F | S | —(CH₂)₃— | NMe(CO₂Me) |
| 2-Me-Bu | F | F | S | —(CH₂)₃— | NMe(COEt) |
| 2-Me-Bu | F | F | S | —(CH₂)₃— | NMe(CO-c-Pr) |
| 2-Me-Bu | F | F | S | —(CH₂)₃— | NMe(CO₂Et) |
| 2-Me-Bu | F | F | S | —(CH₂)₃— | NMe(COCF₃) |
| 2-Me-Bu | F | F | S | —(CH₂)₃— | NMe(Boc) |
| 2-Me-Bu | F | F | S | —(CH₂)₃— | NMe(CH₂CF₃) |
| 2-Me-Bu | F | F | S | —(CH₂)₃— | NMe₂ |
| 2-Me-Bu | F | F | NMe | —(CH₂)₃— | NMe(Et) |
| 2-Me-Bu | F | F | NMe | —(CH₂)₃— | NHMe |
| 2-Me-Bu | F | F | NMe | —(CH₂)₃— | NHEt |
| 2-Me-Bu | F | F | NMe | —(CH₂)₃— | NH-n-Pr |
| 2-Me-Bu | F | F | NMe | —(CH₂)₃— | 1-pyrrolidinyl |
| 2-Me-Bu | F | F | NMe | —(CH₂)₃— | 1-azetidinyl |
| 2-Me-Bu | F | F | NMe | —(CH₂)₃— | 1-aziridinyl |
| 2-Me-Bu | F | F | NMe | —(CH₂)₃— | 1-morpholino |
| 2-Me-Bu | F | F | NMe | —(CH₂)₃— | 1-piperidinyl |
| 2-Me-Bu | F | F | NMe | —(CH₂)₃— | N(Et)₂ |
| 2-Me-Bu | F | F | NMe | —(CH₂)₃— | NH-i-Pr |
| 2-Me-Bu | F | F | NMe | —(CH₂)₃— | NMe(n-Pr) |
| 2-Me-Bu | F | F | NMe | —(CH₂)₃— | NMe(i-Pr) |
| 2-Me-Bu | F | F | NMe | —(CH₂)₃— | NMe(CHO) |
| 2-Me-Bu | F | F | NMe | —(CH₂)₃— | NMe(COCH₃) |
| 2-Me-Bu | F | F | NMe | —(CH₂)₃— | NMe(CO₂Me) |
| 2-Me-Bu | F | F | NMe | —(CH₂)₃— | NMe(COEt) |
| 2-Me-Bu | F | F | NMe | —(CH₂)₃— | NMe(CO-c-Pr) |
| 2-Me-Bu | F | F | NMe | —(CH₂)₃— | NMe(CO₂Et) |
| 2-Me-Bu | F | F | NMe | —(CH₂)₃— | NMe(COCF₃) |
| 2-Me-Bu | F | F | NMe | —(CH₂)₃— | NMe(Boc) |
| 2-Me-Bu | F | F | NMe | —(CH₂)₃— | NMe(CH₂CF₃) |
| 2-Me-Bu | F | F | NMe | —(CH₂)₃— | NMe₂ |
| 2-Me-Bu | F | F | O | —(CH₂)₃— | NMe(Et) |
| i-Bu | F | F | O | —(CH₂)₃— | NMe(Et) |
| i-Bu | F | F | O | —(CH₂)₃— | NHMe |
| i-Bu | F | F | O | —(CH₂)₃— | NHEt |
| i-Bu | F | F | O | —(CH₂)₃— | NH-n-Pr |
| i-Bu | F | F | O | —(CH₂)₃— | 1-pyrrolidinyl |
| i-Bu | F | F | O | —(CH₂)₃— | 1-azetidinyl |
| i-Bu | F | F | O | —(CH₂)₃— | 1-aziridinyl |
| i-Bu | F | F | O | —(CH₂)₃— | 1-morpholino |
| i-Bu | F | F | O | —(CH₂)₃— | 1-piperidinyl |
| i-Bu | F | F | O | —(CH₂)₃— | N(Et)₂ |
| i-Bu | F | F | O | —(CH₂)₃— | NH-i-Pr |
| i-Bu | F | F | O | —(CH₂)₃— | NMe(n-Pr) |
| i-Bu | F | F | O | —(CH₂)₃— | NMe(i-Pr) |
| i-Bu | F | F | O | —(CH₂)₃— | NMe(CHO) |
| i-Bu | F | F | O | —(CH₂)₃— | NMe(COCH₃) |
| i-Bu | F | F | O | —(CH₂)₃— | NMe(CO₂Me) |
| i-Bu | F | F | O | —(CH₂)₃— | NMe(COEt) |
| i-Bu | F | F | O | —(CH₂)₃— | NMe(CO-c-Pr) |
| i-Bu | F | F | O | —(CH₂)₃— | NMe(CO₂Et) |
| i-Bu | F | F | O | —(CH₂)₃— | NMe(COCF₃) |
| i-Bu | F | F | O | —(CH₂)₃— | NMe(Boc) |
| i-Bu | F | F | O | —(CH₂)₃— | NMe(CH₂CF₃) |
| i-Bu | F | F | O | —(CH₂)₃— | NMe₂ |
| 4-Cl-Ph | F | F | O | —(CH₂)₃— | NHMe |
| 4-Cl-Ph | F | F | O | —(CH₂)₃— | NHEt |
| 4-Cl-Ph | F | F | O | —(CH₂)₃— | NH-n-Pr |
| 4-Cl-Ph | F | F | O | —(CH₂)₃— | 1-pyrrolidinyl |
| 4-Cl-Ph | F | F | O | —(CH₂)₃— | 1-azetidinyl |
| 4-Cl-Ph | F | F | O | —(CH₂)₃— | 1-aziridinyl |
| 4-Cl-Ph | F | F | O | —(CH₂)₃— | 1-morpholino |
| 4-Cl-Ph | F | F | O | —(CH₂)₃— | 1-piperidinyl |
| 4-Cl-Ph | F | F | O | —(CH₂)₃— | N(Et)₂ |
| 4-Cl-Ph | F | F | O | —(CH₂)₃— | NH-i-Pr |
| 4-Cl-Ph | F | F | O | —(CH₂)₃— | NMe(n-Pr) |
| 4-Cl-Ph | F | F | O | —(CH₂)₃— | NMe(i-Pr) |
| 4-Cl-Ph | F | F | O | —(CH₂)₃— | NMe(CHO) |
| 4-Cl-Ph | F | F | O | —(CH₂)₃— | NMe(COCH₃) |
| 4-Cl-Ph | F | F | O | —(CH₂)₃— | NMe(CO₂Me) |
| 4-Cl-Ph | F | F | O | —(CH₂)₃— | NMe(COEt) |
| 4-Cl-Ph | F | F | O | —(CH₂)₃— | NMe(CO-c-Pr) |
| 4-Cl-Ph | F | F | O | —(CH₂)₃— | NMe(CO₂Et) |
| 4-Cl-Ph | F | F | O | —(CH₂)₃— | NMe(COCF₃) |
| 4-Cl-Ph | F | F | O | —(CH₂)₃— | NMe(Boc) |
| 4-Cl-Ph | F | F | O | —(CH₂)₃— | NMe(CH₂CF₃) |
| 4-Cl-Ph | F | F | O | —(CH₂)₃— | NMe₂ |
| 3-F-Ph | F | F | O | —(CH₂)₃— | NHMe |
| 3-F-Ph | F | F | O | —(CH₂)₃— | NHEt |
| 3-F-Ph | F | F | O | —(CH₂)₃— | NH-n-Pr |
| 3-F-Ph | F | F | O | —(CH₂)₃— | 1-pyrrolidinyl |
| 3-F-Ph | F | F | O | —(CH₂)₃— | 1-azetidinyl |
| 3-F-Ph | F | F | O | —(CH₂)₃— | 1-aziridinyl |
| 3-F-Ph | F | F | O | —(CH₂)₃— | 1-morpholino |
| 3-F-Ph | F | F | O | —(CH₂)₃— | 1-piperidinyl |
| 3-F-Ph | F | F | O | —(CH₂)₃— | N(Et)₂ |
| 3-F-Ph | F | F | O | —(CH₂)₃— | NH-i-Pr |
| 3-F-Ph | F | F | O | —(CH₂)₃— | NMe(n-Pr) |
| 3-F-Ph | F | F | O | —(CH₂)₃— | NMe(i-Pr) |
| 3-F-Ph | F | F | O | —(CH₂)₃— | NMe(CHO) |
| 3-F-Ph | F | F | O | —(CH₂)₃— | NMe(COCH₃) |
| 3-F-Ph | F | F | O | —(CH₂)₃— | NMe(CO₂Me) |
| 3-F-Ph | F | F | O | —(CH₂)₃— | NMe(COEt) |
| 3-F-Ph | F | F | O | —(CH₂)₃— | NMe(CO-c-Pr) |
| 3-F-Ph | F | F | O | —(CH₂)₃— | NMe(CO₂Et) |
| 3-F-Ph | F | F | O | —(CH₂)₃— | NMe(COCF₃) |
| 3-F-Ph | F | F | O | —(CH₂)₃— | NMe(Boc) |
| 3-F-Ph | F | F | O | —(CH₂)₃— | NMe(CH₂CF₃) |
| 3-F-Ph | F | F | O | —(CH₂)₃— | NMe₂ |
| 2-Me-Bu | F | F | —(CH₂)— | —(CH₂)₃— | NMe(Et) |
| 2-Me-Bu | F | F | —(CH₂)— | —(CH₂)₃— | NHMe |
| 2-Me-Bu | F | F | —(CH₂)— | —(CH₂)₃— | NHEt |
| 2-Me-Bu | F | F | —(CH₂)— | —(CH₂)₃— | NH-n-Pr |
| 2-Me-Bu | F | F | —(CH₂)— | —(CH₂)₃— | 1-pyrrolidinyl |
| 2-Me-Bu | F | F | —(CH₂)— | —(CH₂)₃— | 1-azetidinyl |
| 2-Me-Bu | F | F | —(CH₂)— | —(CH₂)₃— | 1-aziridinyl |
| 2-Me-Bu | F | F | —(CH₂)— | —(CH₂)₃— | 1-morpholino |
| 2-Me-Bu | F | F | —(CH₂)— | —(CH₂)₃— | 1-piperidinyl |
| 2-Me-Bu | F | F | —(CH₂)— | —(CH₂)₃— | N(Et)₂ |
| 2-Me-Bu | F | F | —(CH₂)— | —(CH₂)₃— | NH-i-Pr |
| 2-Me-Bu | F | F | —(CH₂)— | —(CH₂)₃— | NMe(n-Pr) |
| 2-Me-Bu | F | F | —(CH₂)— | —(CH₂)₃— | NMe(I-Pr) |
| 2-Me-Bu | F | F | —(CH₂)— | —(CH₂)₃— | NMe(CHO) |
| 2-Me-Bu | F | F | —(CH₂)— | —(CH₂)₃— | NMe(COCH₃) |
| 2-Me-Bu | F | F | —(CH₂)— | —(CH₂)₃— | NMe(CO₂Me) |
| 2-Me-Bu | F | F | —(CH₂)— | —(CH₂)₃— | NMe(COEt) |
| 2-Me-Bu | F | F | —(CH₂)— | —(CH₂)₃— | NMe(CO-c-Pr) |
| 2-Me-Bu | F | F | —(CH₂)— | —(CH₂)₃— | NMe(CO₂Et) |
| 2-Me-Bu | F | F | —(CH₂)— | —(CH₂)₃— | NMe(COCF₃) |
| 2-Me-Bu | F | F | —(CH₂)— | —(CH₂)₃— | NMe(Boc) |
| 2-Me-Bu | F | F | —(CH₂)— | —(CH₂)₃— | NMe(CH₂CF₃) |
| 2-Me-Bu | F | F | —(CH₂)— | —(CH₂)₃— | OH |

TABLE 6b

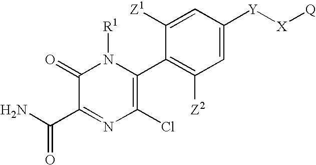

| R¹ | Z¹ | Z² | Y | X | Q |
|---|---|---|---|---|---|
| 2-Me-Bu | F | F | O | —(CH₂)₃— | NMe(Et) |
| 2-Me-Bu | F | F | O | —(CH₂)₃— | NHMe |
| 2-Me-Bu | F | F | O | —(CH₂)₃— | NHEt |
| 2-Me-Bu | F | F | O | —(CH₂)₃— | NH-n-Pr |
| 2-Me-Bu | F | F | O | —(CH₂)₃— | 1-pyrrolidinyl |
| 2-Me-Bu | F | F | O | —(CH₂)₃— | 1-azetidinyl |
| 2-Me-Bu | F | F | O | —(CH₂)₃— | 1-aziridinyl |
| 2-Me-Bu | F | F | O | —(CH₂)₃— | 1-morpholino |
| 2-Me-Bu | F | F | O | —(CH₂)₃— | 1-piperidinyl |
| 2-Me-Bu | F | F | O | —(CH₂)₃— | N(Et)₂ |
| 2-Me-Bu | F | F | O | —(CH₂)₃— | NH-i-Pr |
| 2-Me-Bu | F | F | O | —(CH₂)₃— | NMe(n-Pr) |
| 2-Me-Bu | F | F | O | —(CH₂)₃— | NMe(i-Pr) |
| 2-Me-Bu | F | F | O | —(CH₂)₃— | NMe(CHO) |
| 2-Me-Bu | F | F | O | —(CH₂)₃— | NMe(COCH₃) |
| 2-Me-Bu | F | F | O | —(CH₂)₃— | NMe(CO₂Me) |
| 2-Me-Bu | F | F | O | —(CH₂)₃— | NMe(COEt) |
| 2-Me-Bu | F | F | O | —(CH₂)₃— | NMe(CO-c-Pr) |
| 2-Me-Bu | F | F | O | —(CH₂)₃— | NMe(CO₂Et) |
| 2-Me-Bu | F | F | O | —(CH₂)₃— | NMe(COCF₃) |
| 2-Me-Bu | F | F | O | —(CH₂)₃— | NMe(Boc) |
| 2-Me-Bu | F | F | O | —(CH₂)₃— | NMe(CH₂CF₃) |
| 2-Me-Bu | F | F | O | —(CH₂)₃— | OH |
| 2-Me-Bu | F | H | O | —(CH₂)₃— | NMe(Et) |
| 2-Me-Bu | F | H | O | —(CH₂)₃— | NHMe |
| 2-Me-Bu | F | H | O | —(CH₂)₃— | NHEt |
| 2-Me-Bu | F | H | O | —(CH₂)₃— | NH-n-Pr |
| 2-Me-Bu | F | H | O | —(CH₂)₃— | 1-pyrrolidinyl |
| 2-Me-Bu | F | H | O | —(CH₂)₃— | 1-azetidinyl |
| 2-Me-Bu | F | H | O | —(CH₂)₃— | 1-aziridinyl |
| 2-Me-Bu | F | H | O | —(CH₂)₃— | 1-morpholino |
| 2-Me-Bu | F | H | O | —(CH₂)₃— | 1-piperidinyl |
| 2-Me-Bu | F | H | O | —(CH₂)₃— | N(Et)₂ |
| 2-Me-Bu | F | H | O | —(CH₂)₃— | NH-i-Pr |
| 2-Me-Bu | F | H | O | —(CH₂)₃— | NMe(n-Pr) |
| 2-Me-Bu | F | H | O | —(CH₂)₃— | NMe(i-Pr) |
| 2-Me-Bu | F | H | O | —(CH₂)₃— | NMe(CHO) |
| 2-Me-Bu | F | H | O | —(CH₂)₃— | NMe(COCH₃) |
| 2-Me-Bu | F | H | O | —(CH₂)₃— | NMe(CO₂Me) |
| 2-Me-Bu | F | H | O | —(CH₂)₃— | NMe(COEt) |
| 2-Me-Bu | F | H | O | —(CH₂)₃— | NMe(CO-c-Pr) |
| 2-Me-Bu | F | H | O | —(CH₂)₃— | NMe(CO₂Et) |
| 2-Me-Bu | F | H | O | —(CH₂)₃— | NMe(COCF₃) |
| 2-Me-Bu | F | H | O | —(CH₂)₃— | NMe(Boc) |
| 2-Me-Bu | F | H | O | —(CH₂)₃— | NMe(CH₂CF₃) |
| 2-Me-Bu | F | H | O | —(CH₂)₃— | NMe₂ |
| 2-Me-Bu | Cl | H | O | —(CH₂)₃— | NMe(Et) |
| 2-Me-Bu | Cl | H | O | —(CH₂)₃— | NHMe |
| 2-Me-Bu | Cl | H | O | —(CH₂)₃— | NHEt |
| 2-Me-Bu | Cl | H | O | —(CH₂)₃— | NH-n-Pr |
| 2-Me-Bu | Cl | H | O | —(CH₂)₃— | 1-pyrrolidinyl |
| 2-Me-Bu | Cl | H | O | —(CH₂)₃— | 1-azetidinyl |
| 2-Me-Bu | Cl | H | O | —(CH₂)₃— | 1-aziridinyl |
| 2-Me-Bu | Cl | H | O | —(CH₂)₃— | 1-morpholino |
| 2-Me-Bu | Cl | H | O | —(CH₂)₃— | 1-piperidinyl |
| 2-Me-Bu | Cl | H | O | —(CH₂)₃— | N(Et)₂ |
| 2-Me-Bu | Cl | H | O | —(CH₂)₃— | NH-i-Pr |
| 2-Me-Bu | Cl | H | O | —(CH₂)₃— | NMe(n-Pr) |
| 2-Me-Bu | Cl | H | O | —(CH₂)₃— | NMe(i-Pr) |
| 2-Me-Bu | Cl | H | O | —(CH₂)₃— | NMe(CHO) |
| 2-Me-Bu | Cl | H | O | —(CH₂)₃— | NMe(COCH₃) |
| 2-Me-Bu | Cl | H | O | —(CH₂)₃— | NMe(CO₂Me) |
| 2-Me-Bu | Cl | H | O | —(CH₂)₃— | NMe(COEt) |
| 2-Me-Bu | Cl | H | O | —(CH₂)₃— | NMe(CO-c-Pr) |
| 2-Me-Bu | Cl | H | O | —(CH₂)₃— | NMe(CO₂Et) |
| 2-Me-Bu | Cl | H | O | —(CH₂)₃— | NMe(COCF₃) |
| 2-Me-Bu | Cl | H | O | —(CH₂)₃— | NMe(Boc) |
| 2-Me-Bu | Cl | H | O | —(CH₂)₃— | NMe(CH₂CF₃) |
| 2-Me-Bu | Cl | H | O | —(CH₂)₃— | NMe₂ |
| 2-Me-Bu | F | Cl | O | —(CH₂)₃— | NMe(Et) |
| 2-Me-Bu | F | Cl | O | —(CH₂)₃— | NHMe |
| 2-Me-Bu | F | Cl | O | —(CH₂)₃— | NHEt |
| 2-Me-Bu | F | Cl | O | —(CH₂)₃— | NH-n-Pr |
| 2-Me-Bu | F | Cl | O | —(CH₂)₃— | 1-pyrrolidinyl |
| 2-Me-Bu | F | Cl | O | —(CH₂)₃— | 1-azetidinyl |
| 2-Me-Bu | F | Cl | O | —(CH₂)₃— | 1-aziridinyl |
| 2-Me-Bu | F | Cl | O | —(CH₂)₃— | 1-morpholino |
| 2-Me-Bu | F | Cl | O | —(CH₂)₃— | 1-piperidinyl |
| 2-Me-Bu | F | Cl | O | —(CH₂)₃— | N(Et)₂ |
| 2-Me-Bu | F | Cl | O | —(CH₂)₃— | NH-i-Pr |
| 2-Me-Bu | F | Cl | O | —(CH₂)₃— | NMe(n-Pr) |
| 2-Me-Bu | F | Cl | O | —(CH₂)₃— | NMe(i-Pr) |
| 2-Me-Bu | F | Cl | O | —(CH₂)₃— | NMe(CHO) |
| 2-Me-Bu | F | Cl | O | —(CH₂)₃— | NMe(COCH₃) |
| 2-Me-Bu | F | Cl | O | —(CH₂)₃— | NMe(CO₂Me) |
| 2-Me-Bu | F | Cl | O | —(CH₂)₃— | NMe(COEt) |
| 2-Me-Bu | F | Cl | O | —(CH₂)₃— | NMe(CO-c-Pr) |
| 2-Me-Bu | F | Cl | O | —(CH₂)₃— | NMe(CO₂Et) |
| 2-Me-Bu | F | Cl | O | —(CH₂)₃— | NMe(COCF₃) |
| 2-Me-Bu | F | Cl | O | —(CH₂)₃— | NMe(Boc) |
| 2-Me-Bu | F | Cl | O | —(CH₂)₃— | NMe(CH₂CF₃) |
| 2-Me-Bu | F | Cl | O | —(CH₂)₃— | NMe₂ |
| 2-Me-Bu | F | F | O | —(CH₂)₂— | NMe(Et) |
| 2-Me-Bu | F | F | O | —(CH₂)₂— | NHMe |
| 2-Me-Bu | F | F | O | —(CH₂)₂— | NHEt |
| 2-Me-Bu | F | F | O | —(CH₂)₂— | NH-n-Pr |
| 2-Me-Bu | F | F | O | —(CH₂)₂— | 1-pyrrolidinyl |
| 2-Me-Bu | F | F | O | —(CH₂)₂— | 1-azetidinyl |
| 2-Me-Bu | F | F | O | —(CH₂)₂— | 1-aziridinyl |
| 2-Me-Bu | F | F | O | —(CH₂)₂— | 1-morpholino |
| 2-Me-Bu | F | F | O | —(CH₂)₂— | 1-piperidinyl |
| 2-Me-Bu | F | F | O | —(CH₂)₂— | N(Et)₂ |
| 2-Me-Bu | F | F | O | —(CH₂)₂— | NH-i-Pr |
| 2-Me-Bu | F | F | O | —(CH₂)₂— | NMe(n-Pr) |
| 2-Me-Bu | F | F | O | —(CH₂)₂— | NMe(i-Pr) |
| 2-Me-Bu | F | F | O | —(CH₂)₂— | NMe(CHO) |
| 2-Me-Bu | F | F | O | —(CH₂)₂— | NMe(COCH₃) |
| 2-Me-Bu | F | F | O | —(CH₂)₂— | NMe(CO₂Me) |
| 2-Me-Bu | F | F | O | —(CH₂)₂— | NMe(COEt) |
| 2-Me-Bu | F | F | O | —(CH₂)₂— | NMe(CO-c-Pr) |
| 2-Me-Bu | F | F | O | —(CH₂)₂— | NMe(CO₂Et) |
| 2-Me-Bu | F | F | O | —(CH₂)₂— | NMe(COCF₃) |
| 2-Me-Bu | F | F | O | —(CH₂)₂— | NMe(Boc) |
| 2-Me-Bu | F | F | O | —(CH₂)₂— | NMe(CH₂CF₃) |
| 2-Me-Bu | F | F | O | —(CH₂)₂— | NMe₂ |
| 2-Me-Bu | F | F | O | —(CH₂)₄— | NMe(Et) |
| 2-Me-Bu | F | F | O | —(CH₂)₄— | NHMe |
| 2-Me-Bu | F | F | O | —(CH₂)₄— | NHEt |
| 2-Me-Bu | F | F | O | —(CH₂)₄— | NH-n-Pr |
| 2-Me-Bu | F | F | O | —(CH₂)₄— | 1-pyrrolidinyl |
| 2-Me-Bu | F | F | O | —(CH₂)₄— | 1-azetidinyl |
| 2-Me-Bu | F | F | O | —(CH₂)₄— | 1-aziridinyl |
| 2-Me-Bu | F | F | O | —(CH₂)₄— | 1-morpholino |
| 2-Me-Bu | F | F | O | —(CH₂)₄— | 1-piperidinyl |
| 2-Me-Bu | F | F | O | —(CH₂)₄— | N(Et)₂ |
| 2-Me-Bu | F | F | O | —(CH₂)₄— | NH-i-Pr |
| 2-Me-Bu | F | F | O | —(CH₂)₄— | NMe(n-Pr) |
| 2-Me-Bu | F | F | O | —(CH₂)₄— | NMe(i-Pr) |
| 2-Me-Bu | F | F | O | —(CH₂)₄— | NMe(CHO) |
| 2-Me-Bu | F | F | O | —(CH₂)₄— | NMe(COCH₃) |
| 2-Me-Bu | F | F | O | —(CH₂)₄— | NMe(CO₂Me) |
| 2-Me-Bu | F | F | O | —(CH₂)₄— | NMe(COEt) |
| 2-Me-Bu | F | F | O | —(CH₂)₄— | NMe(CO-c-Pr) |
| 2-Me-Bu | F | F | O | —(CH₂)₄— | NMe(CO₂Et) |
| 2-Me-Bu | F | F | O | —(CH₂)₄— | NMe(COCF₃) |
| 2-Me-Bu | F | F | O | —(CH₂)₄— | NMe(Boc) |
| 2-Me-Bu | F | F | O | —(CH₂)₄— | NMe(CH₂CF₃) |
| 2-Me-Bu | F | F | O | —(CH₂)₄— | NMe₂ |
| 2-Me-Bu | F | F | S | —(CH₂)₃— | NMe(Et) |
| 2-Me-Bu | F | F | S | —(CH₂)₃— | NHMe |
| 2-Me-Bu | F | F | S | —(CH₂)₃— | NHEt |
| 2-Me-Bu | F | F | S | —(CH₂)₃— | NH-n-Pr |
| 2-Me-Bu | F | F | S | —(CH₂)₃— | 1-pyrrolidinyl |
| 2-Me-Bu | F | F | S | —(CH₂)₃— | 1-azetidinyl |
| 2-Me-Bu | F | F | S | —(CH₂)₃— | 1-aziridinyl |
| 2-Me-Bu | F | F | S | —(CH₂)₃— | 1-morpholino |
| 2-Me-Bu | F | F | S | —(CH₂)₃— | 1-piperidinyl |
| 2-Me-Bu | F | F | S | —(CH₂)₃— | N(Et)₂ |
| 2-Me-Bu | F | F | S | —(CH₂)₃— | NH-i-Pr |
| 2-Me-Bu | F | F | S | —(CH₂)₃— | NMe(n-Pr) |
| 2-Me-Bu | F | F | S | —(CH₂)₃— | NMe(i-Pr) |
| 2-Me-Bu | F | F | S | —(CH₂)₃— | NMe(CHO) |
| 2-Me-Bu | F | F | S | —(CH₂)₃— | NMe(COCH₃) |
| 2-Me-Bu | F | F | S | —(CH₂)₃— | NMe(CO₂Me) |
| 2-Me-Bu | F | F | S | —(CH₂)₃— | NMe(COEt) |
| 2-Me-Bu | F | F | S | —(CH₂)₃— | NMe(CO-c-Pr) |
| 2-Me-Bu | F | F | S | —(CH₂)₃— | NMe(CO₂Et) |
| 2-Me-Bu | F | F | S | —(CH₂)₃— | NMe(COCF₃) |
| 2-Me-Bu | F | F | S | —(CH₂)₃— | NMe(Boc) |
| 2-Me-Bu | F | F | S | —(CH₂)₃— | NMe(CH₂CF₃) |
| 2-Me-Bu | F | F | S | —(CH₂)₃— | NMe₂ |
| 2-Me-Bu | F | F | NMe | —(CH₂)₃— | NMe(Et) |
| 2-Me-Bu | F | F | NMe | —(CH₂)₃— | NHMe |
| 2-Me-Bu | F | F | NMe | —(CH₂)₃— | NHEt |
| 2-Me-Bu | F | F | NMe | —(CH₂)₃— | NH-n-Pr |
| 2-Me-Bu | F | F | NMe | —(CH₂)₃— | 1-pyrrolidinyl |
| 2-Me-Bu | F | F | NMe | —(CH₂)₃— | 1-azetidinyl |
| 2-Me-Bu | F | F | NMe | —(CH₂)₃— | 1-aziridinyl |
| 2-Me-Bu | F | F | NMe | —(CH₂)₃— | 1-morpholino |
| 2-Me-Bu | F | F | NMe | —(CH₂)₃— | 1-piperidinyl |
| 2-Me-Bu | F | F | NMe | —(CH₂)₃— | N(Et)₂ |
| 2-Me-Bu | F | F | NMe | —(CH₂)₃— | NH-i-Pr |
| 2-Me-Bu | F | F | NMe | —(CH₂)₃— | NMe(n-Pr) |
| 2-Me-Bu | F | F | NMe | —(CH₂)₃— | NMe(i-Pr) |
| 2-Me-Bu | F | F | NMe | —(CH₂)₃— | NMe(CHO) |
| 2-Me-Bu | F | F | NMe | —(CH₂)₃— | NMe(COCH₃) |
| 2-Me-Bu | F | F | NMe | —(CH₂)₃— | NMe(CO₂Me) |
| 2-Me-Bu | F | F | NMe | —(CH₂)₃— | NMe(COEt) |
| 2-Me-Bu | F | F | NMe | —(CH₂)₃— | NMe(CO-c-Pr) |
| 2-Me-Bu | F | F | NMe | —(CH₂)₃— | NMe(CO₂Et) |
| 2-Me-Bu | F | F | NMe | —(CH₂)₃— | NMe(COCF₃) |
| 2-Me-Bu | F | F | NMe | —(CH₂)₃— | NMe(Boc) |
| 2-Me-Bu | F | F | NMe | —(CH₂)₃— | NMe(CH₂CF₃) |
| 2-Me-Bu | F | F | NMe | —(CH₂)₃— | NMe₂ |
| 2-Me-Bu | F | F | O | —(CH₂)₃— | NMe(Et) |
| i-Bu | F | F | O | —(CH₂)₃— | NMe(Et) |
| i-Bu | F | F | O | —(CH₂)₃— | NHMe |
| i-Bu | F | F | O | —(CH₂)₃— | NHEt |
| i-Bu | F | F | O | —(CH₂)₃— | NH-n-Pr |
| i-Bu | F | F | O | —(CH₂)₃— | 1-pyrrolidinyl |
| i-Bu | F | F | O | —(CH₂)₃— | 1-azetidinyl |
| i-Bu | F | F | O | —(CH₂)₃— | 1-aziridinyl |
| i-Bu | F | F | O | —(CH₂)₃— | 1-morpholino |
| i-Bu | F | F | O | —(CH₂)₃— | 1-piperidinyl |
| i-Bu | F | F | O | —(CH₂)₃— | N(Et)₂ |
| i-Bu | F | F | O | —(CH₂)₃— | NH-i-Pr |
| i-Bu | F | F | O | —(CH₂)₃— | NMe(n-Pr) |
| i-Bu | F | F | O | —(CH₂)₃— | NMe(i-Pr) |
| i-Bu | F | F | O | —(CH₂)₃— | NMe(CHO) |
| i-Bu | F | F | O | —(CH₂)₃— | NMe(COCH₃) |
| i-Bu | F | F | O | —(CH₂)₃— | NMe(CO₂Me) |
| i-Bu | F | F | O | —(CH₂)₃— | NMe(COEt) |
| i-Bu | F | F | O | —(CH₂)₃— | NMe(CO-c-Pr) |
| i-Bu | F | F | O | —(CH₂)₃— | NMe(CO₂Et) |
| i-Bu | F | F | O | —(CH₂)₃— | NMe(COCF₃) |
| i-Bu | F | F | O | —(CH₂)₃— | NMe(Boc) |
| i-Bu | F | F | O | —(CH₂)₃— | NMe(CH₂CF₃) |
| i-Bu | F | F | O | —(CH₂)₃— | NMe₂ |
| 4-Cl-Ph | F | F | O | —(CH₂)₃— | NHMe |
| 4-Cl-Ph | F | F | O | —(CH₂)₃— | NHEt |
| 4-Cl-Ph | F | F | O | —(CH₂)₃— | NH-n-Pr |
| 4-Cl-Ph | F | F | O | —(CH₂)₃— | 1-pyrrolidinyl |
| 4-Cl-Ph | F | F | O | —(CH₂)₃— | 1-azetidinyl |
| 4-Cl-Ph | F | F | O | —(CH₂)₃— | 1-aziridinyl |
| 4-Cl-Ph | F | F | O | —(CH₂)₃— | 1-morpholino |
| 4-Cl-Ph | F | F | O | —(CH₂)₃— | 1-piperidinyl |
| 4-Cl-Ph | F | F | O | —(CH₂)₃— | N(Et)₂ |
| 4-Cl-Ph | F | F | O | —(CH₂)₃— | NH-i-Pr |
| 4-Cl-Ph | F | F | O | —(CH₂)₃— | NMe(n-Pr) |
| 4-Cl-Ph | F | F | O | —(CH₂)₃— | NMe(i-Pr) |
| 4-Cl-Ph | F | F | O | —(CH₂)₃— | NMe(CHO) |
| 4-Cl-Ph | F | F | O | —(CH₂)₃— | NMe(COCH₃) |
| 4-Cl-Ph | F | F | O | —(CH₂)₃— | NMe(CO₂Me) |
| 4-Cl-Ph | F | F | O | —(CH₂)₃— | NMe(COEt) |
| 4-Cl-Ph | F | F | O | —(CH₂)₃— | NMe(CO-c-Pr) |
| 4-Cl-Ph | F | F | O | —(CH₂)₃— | NMe(CO₂Et) |
| 4-Cl-Ph | F | F | O | —(CH₂)₃— | NMe(COCF₃) |
| 4-Cl-Ph | F | F | O | —(CH₂)₃— | NMe(Boc) |
| 4-Cl-Ph | F | F | O | —(CH₂)₃— | NMe(CH₂CF₃) |
| 4-Cl-Ph | F | F | O | —(CH₂)₃— | NMe₂ |
| 3-F-Ph | F | F | O | —(CH₂)₃— | NHMe |
| 3-F-Ph | F | F | O | —(CH₂)₃— | NHEt |
| 3-F-Ph | F | F | O | —(CH₂)₃— | NH-n-Pr |
| 3-F-Ph | F | F | O | —(CH₂)₃— | 1-pyrrolidinyl |
| 3-F-Ph | F | F | O | —(CH₂)₃— | 1-azetidinyl |
| 3-F-Ph | F | F | O | —(CH₂)₃— | 1-aziridinyl |
| 3-F-Ph | F | F | O | —(CH₂)₃— | 1-morpholino |
| 3-F-Ph | F | F | O | —(CH₂)₃— | 1-piperidinyl |
| 3-F-Ph | F | F | O | —(CH₂)₃— | N(Et)₂ |
| 3-F-Ph | F | F | O | —(CH₂)₃— | NH-i-Pr |
| 3-F-Ph | F | F | O | —(CH₂)₃— | NMe(n-Pr) |
| 3-F-Ph | F | F | O | —(CH₂)₃— | NMe(i-Pr) |
| 3-F-Ph | F | F | O | —(CH₂)₃— | NMe(CHO) |
| 3-F-Ph | F | F | O | —(CH₂)₃— | NMe(COCH₃) |
| 3-F-Ph | F | F | O | —(CH₂)₃— | NMe(CO₂Me) |
| 3-F-Ph | F | F | O | —(CH₂)₃— | NMe(COEt) |
| 3-F-Ph | F | F | O | —(CH₂)₃— | NMe(CO-c-Pr) |
| 3-F-Ph | F | F | O | —(CH₂)₃— | NMe(CO₂Et) |
| 3-F-Ph | F | F | O | —(CH₂)₃— | NMe(COCF₃) |
| 3-F-Ph | F | F | O | —(CH₂)₃— | NMe(Boc) |
| 3-F-Ph | F | F | O | —(CH₂)₃— | NMe(CH₂CF₃) |
| 3-F-Ph | F | F | O | —(CH₂)₃— | NMe₂ |
| 2-Me-Bu | F | F | —(CH₂)— | —(CH₂)₃— | NMe(Et) |
| 2-Me-Bu | F | F | —(CH₂)— | —(CH₂)₃— | NHMe |
| 2-Me-Bu | F | F | —(CH₂)— | —(CH₂)₃— | NHEt |
| 2-Me-Bu | F | F | —(CH₂)— | —(CH₂)₃— | NH-n-Pr |
| 2-Me-Bu | F | F | —(CH₂)— | —(CH₂)₃— | 1-pyrrolidinyl |
| 2-Me-Bu | F | F | —(CH₂)— | —(CH₂)₃— | 1-azetidinyl |
| 2-Me-Bu | F | F | —(CH₂)— | —(CH₂)₃— | 1-aziridinyl |
| 2-Me-Bu | F | F | —(CH₂)— | —(CH₂)₃— | 1-morpholino |
| 2-Me-Bu | F | F | —(CH₂)— | —(CH₂)₃— | 1-piperidinyl |
| 2-Me-Bu | F | F | —(CH₂)— | —(CH₂)₃— | N(Et)₂ |
| 2-Me-Bu | F | F | —(CH₂)— | —(CH₂)₃— | NH-i-Pr |
| 2-Me-Bu | F | F | —(CH₂)— | —(CH₂)₃— | NMe(n-Pr) |
| 2-Me-Bu | F | F | —(CH₂)— | —(CH₂)₃— | NMe(I-Pr) |
| 2-Me-Bu | F | F | —(CH₂)— | —(CH₂)₃— | NMe(CHO) |
| 2-Me-Bu | F | F | —(CH₂)— | —(CH₂)₃— | NMe(COCH₃) |
| 2-Me-Bu | F | F | —(CH₂)— | —(CH₂)₃— | NMe(CO₂Me) |
| 2-Me-Bu | F | F | —(CH₂)— | —(CH₂)₃— | NMe(COEt) |
| 2-Me-Bu | F | F | —(CH₂)— | —(CH₂)₃— | NMe(CO-c-Pr) |
| 2-Me-Bu | F | F | —(CH₂)— | —(CH₂)₃— | NMe(CO₂Et) |
| 2-Me-Bu | F | F | —(CH₂)— | —(CH₂)₃— | NMe(COCF₃) |
| 2-Me-Bu | F | F | —(CH₂)— | —(CH₂)₃— | NMe(Boc) |
| 2-Me-Bu | F | F | —(CH₂)— | —(CH₂)₃— | NMe(CH₂CF₃) |
| 2-Me-Bu | F | F | —(CH₂)— | —(CH₂)₃— | OH |

TABLE 6c

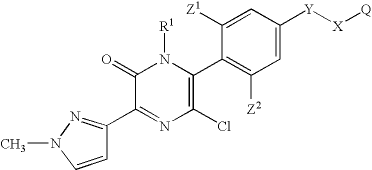

| R¹ | Z¹ | Z² | Y | X | Q |
|---|---|---|---|---|---|
| 2-Me-Bu | F | F | O | —(CH₂)₃— | NMe(Et) |
| 2-Me-Bu | F | F | O | —(CH₂)₃— | NHMe |
| 2-Me-Bu | F | F | O | —(CH₂)₃— | NHEt |
| 2-Me-Bu | F | F | O | —(CH₂)₃— | NH-n-Pr |
| 2-Me-Bu | F | F | O | —(CH₂)₃— | 1-pyrrolidinyl |
| 2-Me-Bu | F | F | O | —(CH₂)₃— | 1-azetidinyl |
| 2-Me-Bu | F | F | O | —(CH₂)₃— | 1-aziridinyl |
| 2-Me-Bu | F | F | O | —(CH₂)₃— | 1-morpholino |
| 2-Me-Bu | F | F | O | —(CH₂)₃— | 1-piperidinyl |
| 2-Me-Bu | F | F | O | —(CH₂)₃— | N(Et)₂ |
| 2-Me-Bu | F | F | O | —(CH₂)₃— | NH-i-Pr |
| 2-Me-Bu | F | F | O | —(CH₂)₃— | NMe(n-Pr) |
| 2-Me-Bu | F | F | O | —(CH₂)₃— | NMe(i-Pr) |
| 2-Me-Bu | F | F | O | —(CH₂)₃— | NMe(CHO) |
| 2-Me-Bu | F | F | O | —(CH₂)₃— | NMe(COCH₃) |
| 2-Me-Bu | F | F | O | —(CH₂)₃— | NMe(CO₂Me) |
| 2-Me-Bu | F | F | O | —(CH₂)₃— | NMe(COEt) |
| 2-Me-Bu | F | F | O | —(CH₂)₃— | NMe(CO-c-Pr) |
| 2-Me-Bu | F | F | O | —(CH₂)₃— | NMe(CO₂Et) |
| 2-Me-Bu | F | F | O | —(CH₂)₃— | NMe(COCF₃) |
| 2-Me-Bu | F | F | O | —(CH₂)₃— | NMe(Boc) |
| 2-Me-Bu | F | F | O | —(CH₂)₃— | NMe(CH₂CF₃) |
| 2-Me-Bu | F | F | O | —(CH₂)₃— | OH |
| 2-Me-Bu | F | H | O | —(CH₂)₃— | NMe(Et) |
| 2-Me-Bu | F | H | O | —(CH₂)₃— | NHMe |
| 2-Me-Bu | F | H | O | —(CH₂)₃— | NHEt |
| 2-Me-Bu | F | H | O | —(CH₂)₃— | NH-n-Pr |
| 2-Me-Bu | F | H | O | —(CH₂)₃— | 1-pyrrolidinyl |
| 2-Me-Bu | F | H | O | —(CH₂)₃— | 1-azetidinyl |
| 2-Me-Bu | F | H | O | —(CH₂)₃— | 1-aziridinyl |
| 2-Me-Bu | F | H | O | —(CH₂)₃— | 1-morpholino |
| 2-Me-Bu | F | H | O | —(CH₂)₃— | 1-piperidinyl |
| 2-Me-Bu | F | H | O | —(CH₂)₃— | N(Et)₂ |
| 2-Me-Bu | F | H | O | —(CH₂)₃— | NH-i-Pr |
| 2-Me-Bu | F | H | O | —(CH₂)₃— | NMe(n-Pr) |
| 2-Me-Bu | F | H | O | —(CH₂)₃— | NMe(i-Pr) |
| 2-Me-Bu | F | H | O | —(CH₂)₃— | NMe(CHO) |
| 2-Me-Bu | F | H | O | —(CH₂)₃— | NMe(COCH₃) |
| 2-Me-Bu | F | H | O | —(CH₂)₃— | NMe(CO₂Me) |
| 2-Me-Bu | F | H | O | —(CH₂)₃— | NMe(COEt) |
| 2-Me-Bu | F | H | O | —(CH₂)₃— | NMe(CO-c-Pr) |
| 2-Me-Bu | F | H | O | —(CH₂)₃— | NMe(CO₂Et) |
| 2-Me-Bu | F | H | O | —(CH₂)₃— | NMe(COCF₃) |
| 2-Me-Bu | F | H | O | —(CH₂)₃— | NMe(Boc) |
| 2-Me-Bu | F | H | O | —(CH₂)₃— | NMe(CH₂CF₃) |
| 2-Me-Bu | F | H | O | —(CH₂)₃— | NMe₂ |
| 2-Me-Bu | Cl | H | O | —(CH₂)₃— | NMe(Et) |
| 2-Me-Bu | Cl | H | O | —(CH₂)₃— | NHMe |
| 2-Me-Bu | Cl | H | O | —(CH₂)₃— | NHEt |
| 2-Me-Bu | Cl | H | O | —(CH₂)₃— | NH-n-Pr |
| 2-Me-Bu | Cl | H | O | —(CH₂)₃— | 1-pyrrolidinyl |
| 2-Me-Bu | Cl | H | O | —(CH₂)₃— | 1-azetidinyl |
| 2-Me-Bu | Cl | H | O | —(CH₂)₃— | 1-aziridinyl |
| 2-Me-Bu | Cl | H | O | —(CH₂)₃— | 1-morpholino |
| 2-Me-Bu | Cl | H | O | —(CH₂)₃— | 1-piperidinyl |
| 2-Me-Bu | Cl | H | O | —(CH₂)₃— | N(Et)₂ |
| 2-Me-Bu | Cl | H | O | —(CH₂)₃— | NH-i-Pr |
| 2-Me-Bu | Cl | H | O | —(CH₂)₃— | NMe(n-Pr) |
| 2-Me-Bu | Cl | H | O | —(CH₂)₃— | NMe(i-Pr) |
| 2-Me-Bu | Cl | H | O | —(CH₂)₃— | NMe(CHO) |
| 2-Me-Bu | Cl | H | O | —(CH₂)₃— | NMe(COCH₃) |
| 2-Me-Bu | Cl | H | O | —(CH₂)₃— | NMe(CO₂Me) |
| 2-Me-Bu | Cl | H | O | —(CH₂)₃— | NMe(COEt) |
| 2-Me-Bu | Cl | H | O | —(CH₂)₃— | NMe(CO-c-Pr) |
| 2-Me-Bu | Cl | H | O | —(CH₂)₃— | NMe(CO₂Et) |
| 2-Me-Bu | Cl | H | O | —(CH₂)₃— | NMe(COCF₃) |
| 2-Me-Bu | Cl | H | O | —(CH₂)₃— | NMe(Boc) |
| 2-Me-Bu | Cl | H | O | —(CH₂)₃— | NMe(CH₂CF₃) |
| 2-Me-Bu | Cl | H | O | —(CH₂)₃— | NMe₂ |
| 2-Me-Bu | F | Cl | O | —(CH₂)₃— | NMe(Et) |
| 2-Me-Bu | F | Cl | O | —(CH₂)₃— | NHMe |
| 2-Me-Bu | F | Cl | O | —(CH₂)₃— | NHEt |
| 2-Me-Bu | F | Cl | O | —(CH₂)₃— | NH-n-Pr |
| 2-Me-Bu | F | Cl | O | —(CH₂)₃— | 1-pyrrolidinyl |
| 2-Me-Bu | F | Cl | O | —(CH₂)₃— | 1-azetidinyl |
| 2-Me-Bu | F | Cl | O | —(CH₂)₃— | 1-aziridinyl |
| 2-Me-Bu | F | Cl | O | —(CH₂)₃— | 1-morpholino |
| 2-Me-Bu | F | Cl | O | —(CH₂)₃— | 1-piperidinyl |
| 2-Me-Bu | F | Cl | O | —(CH₂)₃— | N(Et)₂ |
| 2-Me-Bu | F | Cl | O | —(CH₂)₃— | NH-i-Pr |
| 2-Me-Bu | F | Cl | O | —(CH₂)₃— | NMe(n-Pr) |
| 2-Me-Bu | F | Cl | O | —(CH₂)₃— | NMe(i-Pr) |
| 2-Me-Bu | F | Cl | O | —(CH₂)₃— | NMe(CHO) |
| 2-Me-Bu | F | Cl | O | —(CH₂)₃— | NMe(COCH₃) |
| 2-Me-Bu | F | Cl | O | —(CH₂)₃— | NMe(CO₂Me) |
| 2-Me-Bu | F | Cl | O | —(CH₂)₃— | NMe(COEt) |
| 2-Me-Bu | F | Cl | O | —(CH₂)₃— | NMe(CO-c-Pr) |
| 2-Me-Bu | F | Cl | O | —(CH₂)₃— | NMe(CO₂Et) |
| 2-Me-Bu | F | Cl | O | —(CH₂)₃— | NMe(COCF₃) |
| 2-Me-Bu | F | Cl | O | —(CH₂)₃— | NMe(Boc) |
| 2-Me-Bu | F | Cl | O | —(CH₂)₃— | NMe(CH₂CF₃) |
| 2-Me-Bu | F | Cl | O | —(CH₂)₃— | NMe₂ |
| 2-Me-Bu | F | F | O | —(CH₂)₂— | NMe(Et) |
| 2-Me-Bu | F | F | O | —(CH₂)₂— | NHMe |
| 2-Me-Bu | F | F | O | —(CH₂)₂— | NHEt |
| 2-Me-Bu | F | F | O | —(CH₂)₂— | NH-n-Pr |
| 2-Me-Bu | F | F | O | —(CH₂)₂— | 1-pyrrolidinyl |
| 2-Me-Bu | F | F | O | —(CH₂)₂— | 1-azetidinyl |
| 2-Me-Bu | F | F | O | —(CH₂)₂— | 1-aziridinyl |
| 2-Me-Bu | F | F | O | —(CH₂)₂— | 1-morpholino |
| 2-Me-Bu | F | F | O | —(CH₂)₂— | 1-piperidinyl |
| 2-Me-Bu | F | F | O | —(CH₂)₂— | N(Et)₂ |
| 2-Me-Bu | F | F | O | —(CH₂)₂— | NH-i-Pr |
| 2-Me-Bu | F | F | O | —(CH₂)₂— | NMe(n-Pr) |
| 2-Me-Bu | F | F | O | —(CH₂)₂— | NMe(i-Pr) |
| 2-Me-Bu | F | F | O | —(CH₂)₂— | NMe(CHO) |
| 2-Me-Bu | F | F | O | —(CH₂)₂— | NMe(COCH₃) |
| 2-Me-Bu | F | F | O | —(CH₂)₂— | NMe(CO₂Me) |
| 2-Me-Bu | F | F | O | —(CH₂)₂— | NMe(COEt) |
| 2-Me-Bu | F | F | O | —(CH₂)₂— | NMe(CO-c-Pr) |
| 2-Me-Bu | F | F | O | —(CH₂)₂— | NMe(CO₂Et) |
| 2-Me-Bu | F | F | O | —(CH₂)₂— | NMe(COCF₃) |
| 2-Me-Bu | F | F | O | —(CH₂)₂— | NMe(Boc) |
| 2-Me-Bu | F | F | O | —(CH₂)₂— | NMe(CH₂CF₃) |
| 2-Me-Bu | F | F | O | —(CH₂)₂— | NMe₂ |
| 2-Me-Bu | F | F | O | —(CH₂)₄— | NMe(Et) |
| 2-Me-Bu | F | F | O | —(CH₂)₄— | NHMe |
| 2-Me-Bu | F | F | O | —(CH₂)₄— | NHEt |
| 2-Me-Bu | F | F | O | —(CH₂)₄— | NH-n-Pr |
| 2-Me-Bu | F | F | O | —(CH₂)₄— | 1-pyrrolidinyl |
| 2-Me-Bu | F | F | O | —(CH₂)₄— | 1-azetidinyl |
| 2-Me-Bu | F | F | O | —(CH₂)₄— | 1-aziridinyl |
| 2-Me-Bu | F | F | O | —(CH₂)₄— | 1-morpholino |
| 2-Me-Bu | F | F | O | —(CH₂)₄— | 1-piperidinyl |
| 2-Me-Bu | F | F | O | —(CH₂)₄— | N(Et)₂ |
| 2-Me-Bu | F | F | O | —(CH₂)₄— | NH-i-Pr |
| 2-Me-Bu | F | F | O | —(CH₂)₄— | NMe(n-Pr) |
| 2-Me-Bu | F | F | O | —(CH₂)₄— | NMe(i-Pr) |
| 2-Me-Bu | F | F | O | —(CH₂)₄— | NMe(CHO) |
| 2-Me-Bu | F | F | O | —(CH₂)₄— | NMe(COCH₃) |
| 2-Me-Bu | F | F | O | —(CH₂)₄— | NMe(CO₂Me) |
| 2-Me-Bu | F | F | O | —(CH₂)₄— | NMe(COEt) |
| 2-Me-Bu | F | F | O | —(CH₂)₄— | NMe(CO-c-Pr) |
| 2-Me-Bu | F | F | O | —(CH₂)₄— | NMe(CO₂Et) |
| 2-Me-Bu | F | F | O | —(CH₂)₄— | NMe(COCF₃) |
| 2-Me-Bu | F | F | O | —(CH₂)₄— | NMe(Boc) |
| 2-Me-Bu | F | F | O | —(CH₂)₄— | NMe(CH₂CF₃) |
| 2-Me-Bu | F | F | O | —(CH₂)₄— | NMe₂ |
| 2-Me-Bu | F | F | S | —(CH₂)₃— | NMe(Et) |
| 2-Me-Bu | F | F | S | —(CH₂)₃— | NHMe |
| 2-Me-Bu | F | F | S | —(CH₂)₃— | NHEt |
| 2-Me-Bu | F | F | S | —(CH₂)₃— | NH-n-Pr |
| 2-Me-Bu | F | F | S | —(CH₂)₃— | 1-pyrrolidinyl |
| 2-Me-Bu | F | F | S | —(CH₂)₃— | 1-azetidinyl |
| 2-Me-Bu | F | F | S | —(CH₂)₃— | 1-aziridinyl |
| 2-Me-Bu | F | F | S | —(CH₂)₃— | 1-morpholino |
| 2-Me-Bu | F | F | S | —(CH₂)₃— | 1-piperidinyl |
| 2-Me-Bu | F | F | S | —(CH₂)₃— | N(Et)₂ |
| 2-Me-Bu | F | F | S | —(CH₂)₃— | NH-i-Pr |
| 2-Me-Bu | F | F | S | —(CH₂)₃— | NMe(n-Pr) |
| 2-Me-Bu | F | F | S | —(CH₂)₃— | NMe(i-Pr) |
| 2-Me-Bu | F | F | S | —(CH₂)₃— | NMe(CHO) |
| 2-Me-Bu | F | F | S | —(CH₂)₃— | NMe(COCH₃) |
| 2-Me-Bu | F | F | S | —(CH₂)₃— | NMe(CO₂Me) |
| 2-Me-Bu | F | F | S | —(CH₂)₃— | NMe(COEt) |
| 2-Me-Bu | F | F | S | —(CH₂)₃— | NMe(CO-c-Pr) |
| 2-Me-Bu | F | F | S | —(CH₂)₃— | NMe(CO₂Et) |
| 2-Me-Bu | F | F | S | —(CH₂)₃— | NMe(COCF₃) |
| 2-Me-Bu | F | F | S | —(CH₂)₃— | NMe(Boc) |
| 2-Me-Bu | F | F | S | —(CH₂)₃— | NMe(CH₂CF₃) |
| 2-Me-Bu | F | F | S | —(CH₂)₃— | NMe₂ |
| 2-Me-Bu | F | F | NMe | —(CH₂)₃— | NMe(Et) |
| 2-Me-Bu | F | F | NMe | —(CH₂)₃— | NHMe |
| 2-Me-Bu | F | F | NMe | —(CH₂)₃— | NHEt |
| 2-Me-Bu | F | F | NMe | —(CH₂)₃— | NH-n-Pr |
| 2-Me-Bu | F | F | NMe | —(CH₂)₃— | 1-pyrrolidinyl |
| 2-Me-Bu | F | F | NMe | —(CH₂)₃— | 1-azetidinyl |
| 2-Me-Bu | F | F | NMe | —(CH₂)₃— | 1-aziridinyl |
| 2-Me-Bu | F | F | NMe | —(CH₂)₃— | 1-morpholino |
| 2-Me-Bu | F | F | NMe | —(CH₂)₃— | 1-piperidinyl |
| 2-Me-Bu | F | F | NMe | —(CH₂)₃— | N(Et)₂ |
| 2-Me-Bu | F | F | NMe | —(CH₂)₃— | NH-i-Pr |
| 2-Me-Bu | F | F | NMe | —(CH₂)₃— | NMe(n-Pr) |
| 2-Me-Bu | F | F | NMe | —(CH₂)₃— | NMe(i-Pr) |
| 2-Me-Bu | F | F | NMe | —(CH₂)₃— | NMe(CHO) |
| 2-Me-Bu | F | F | NMe | —(CH₂)₃— | NMe(COCH₃) |
| 2-Me-Bu | F | F | NMe | —(CH₂)₃— | NMe(CO₂Me) |
| 2-Me-Bu | F | F | NMe | —(CH₂)₃— | NMe(COEt) |
| 2-Me-Bu | F | F | NMe | —(CH₂)₃— | NMe(CO-c-Pr) |
| 2-Me-Bu | F | F | NMe | —(CH₂)₃— | NMe(CO₂Et) |
| 2-Me-Bu | F | F | NMe | —(CH₂)₃— | NMe(COCF₃) |
| 2-Me-Bu | F | F | NMe | —(CH₂)₃— | NMe(Boc) |
| 2-Me-Bu | F | F | NMe | —(CH₂)₃— | NMe(CH₂CF₃) |
| 2-Me-Bu | F | F | NMe | —(CH₂)₃— | NMe₂ |
| 2-Me-Bu | F | F | O | —(CH₂)₃— | NMe(Et) |
| i-Bu | F | F | O | —(CH₂)₃— | NMe(Et) |
| i-Bu | F | F | O | —(CH₂)₃— | NHMe |
| i-Bu | F | F | O | —(CH₂)₃— | NHEt |
| i-Bu | F | F | O | —(CH₂)₃— | NH-n-Pr |
| i-Bu | F | F | O | —(CH₂)₃— | 1-pyrrolidinyl |
| i-Bu | F | F | O | —(CH₂)₃— | 1-azetidinyl |
| i-Bu | F | F | O | —(CH₂)₃— | 1-aziridinyl |
| i-Bu | F | F | O | —(CH₂)₃— | 1-morpholino |
| i-Bu | F | F | O | —(CH₂)₃— | 1-piperidinyl |
| i-Bu | F | F | O | —(CH₂)₃— | N(Et)₂ |
| i-Bu | F | F | O | —(CH₂)₃— | NH-i-Pr |
| i-Bu | F | F | O | —(CH₂)₃— | NMe(n-Pr) |
| i-Bu | F | F | O | —(CH₂)₃— | NMe(i-Pr) |
| i-Bu | F | F | O | —(CH₂)₃— | NMe(CHO) |
| i-Bu | F | F | O | —(CH₂)₃— | NMe(COCH₃) |
| i-Bu | F | F | O | —(CH₂)₃— | NMe(CO₂Me) |
| i-Bu | F | F | O | —(CH₂)₃— | NMe(COEt) |
| i-Bu | F | F | O | —(CH₂)₃— | NMe(CO-c-Pr) |
| i-Bu | F | F | O | —(CH₂)₃— | NMe(CO₂Et) |
| i-Bu | F | F | O | —(CH₂)₃— | NMe(COCF₃) |
| i-Bu | F | F | O | —(CH₂)₃— | NMe(Boc) |
| i-Bu | F | F | O | —(CH₂)₃— | NMe(CH₂CF₃) |
| i-Bu | F | F | O | —(CH₂)₃— | NMe₂ |
| 4-Cl-Ph | F | F | O | —(CH₂)₃— | NHMe |
| 4-Cl-Ph | F | F | O | —(CH₂)₃— | NHEt |
| 4-Cl-Ph | F | F | O | —(CH₂)₃— | NH-n-Pr |
| 4-Cl-Ph | F | F | O | —(CH₂)₃— | 1-pyrrolidinyl |
| 4-Cl-Ph | F | F | O | —(CH₂)₃— | 1-azetidinyl |
| 4-Cl-Ph | F | F | O | —(CH₂)₃— | 1-aziridinyl |
| 4-Cl-Ph | F | F | O | —(CH₂)₃— | 1-morpholino |
| 4-Cl-Ph | F | F | O | —(CH₂)₃— | 1-piperidinyl |
| 4-Cl-Ph | F | F | O | —(CH₂)₃— | N(Et)₂ |
| 4-Cl-Ph | F | F | O | —(CH₂)₃— | NH-i-Pr |
| 4-Cl-Ph | F | F | O | —(CH₂)₃— | NMe(n-Pr) |
| 4-Cl-Ph | F | F | O | —(CH₂)₃— | NMe(i-Pr) |
| 4-Cl-Ph | F | F | O | —(CH₂)₃— | NMe(CHO) |
| 4-Cl-Ph | F | F | O | —(CH₂)₃— | NMe(COCH₃) |
| 4-Cl-Ph | F | F | O | —(CH₂)₃— | NMe(CO₂Me) |
| 4-Cl-Ph | F | F | O | —(CH₂)₃— | NMe(COEt) |
| 4-Cl-Ph | F | F | O | —(CH₂)₃— | NMe(CO-c-Pr) |
| 4-Cl-Ph | F | F | O | —(CH₂)₃— | NMe(CO₂Et) |
| 4-Cl-Ph | F | F | O | —(CH₂)₃— | NMe(COCF₃) |
| 4-Cl-Ph | F | F | O | —(CH₂)₃— | NMe(Boc) |
| 4-Cl-Ph | F | F | O | —(CH₂)₃— | NMe(CH₂CF₃) |
| 4-Cl-Ph | F | F | O | —(CH₂)₃— | NMe₂ |
| 3-F-Ph | F | F | O | —(CH₂)₃— | NHMe |
| 3-F-Ph | F | F | O | —(CH₂)₃— | NHEt |
| 3-F-Ph | F | F | O | —(CH₂)₃— | NH-n-Pr |
| 3-F-Ph | F | F | O | —(CH₂)₃— | 1-pyrrolidinyl |
| 3-F-Ph | F | F | O | —(CH₂)₃— | 1-azetidinyl |
| 3-F-Ph | F | F | O | —(CH₂)₃— | 1-aziridinyl |
| 3-F-Ph | F | F | O | —(CH₂)₃— | 1-morpholino |
| 3-F-Ph | F | F | O | —(CH₂)₃— | 1-piperidinyl |
| 3-F-Ph | F | F | O | —(CH₂)₃— | N(Et)₂ |
| 3-F-Ph | F | F | O | —(CH₂)₃— | NH-i-Pr |
| 3-F-Ph | F | F | O | —(CH₂)₃— | NMe(n-Pr) |
| 3-F-Ph | F | F | O | —(CH₂)₃— | NMe(i-Pr) |
| 3-F-Ph | F | F | O | —(CH₂)₃— | NMe(CHO) |
| 3-F-Ph | F | F | O | —(CH₂)₃— | NMe(COCH₃) |
| 3-F-Ph | F | F | O | —(CH₂)₃— | NMe(CO₂Me) |
| 3-F-Ph | F | F | O | —(CH₂)₃— | NMe(COEt) |
| 3-F-Ph | F | F | O | —(CH₂)₃— | NMe(CO-c-Pr) |
| 3-F-Ph | F | F | O | —(CH₂)₃— | NMe(CO₂Et) |
| 3-F-Ph | F | F | O | —(CH₂)₃— | NMe(COCF₃) |
| 3-F-Ph | F | F | O | —(CH₂)₃— | NMe(Boc) |
| 3-F-Ph | F | F | O | —(CH₂)₃— | NMe(CH₂CF₃) |
| 3-F-Ph | F | F | O | —(CH₂)₃— | NMe₂ |
| 2-Me-Bu | F | F | —(CH₂)— | —(CH₂)₃— | NMe(Et) |
| 2-Me-Bu | F | F | —(CH₂)— | —(CH₂)₃— | NHMe |
| 2-Me-Bu | F | F | —(CH₂)— | —(CH₂)₃— | NHEt |
| 2-Me-Bu | F | F | —(CH₂)— | —(CH₂)₃— | NH-n-Pr |
| 2-Me-Bu | F | F | —(CH₂)— | —(CH₂)₃— | 1-pyrrolidinyl |
| 2-Me-Bu | F | F | —(CH₂)— | —(CH₂)₃— | 1-azetidinyl |
| 2-Me-Bu | F | F | —(CH₂)— | —(CH₂)₃— | 1-aziridinyl |
| 2-Me-Bu | F | F | —(CH₂)— | —(CH₂)₃— | 1-morpholino |
| 2-Me-Bu | F | F | —(CH₂)— | —(CH₂)₃— | 1-piperidinyl |
| 2-Me-Bu | F | F | —(CH₂)— | —(CH₂)₃— | N(Et)₂ |
| 2-Me-Bu | F | F | —(CH₂)— | —(CH₂)₃— | NH-i-Pr |
| 2-Me-Bu | F | F | —(CH₂)— | —(CH₂)₃— | NMe(n-Pr) |
| 2-Me-Bu | F | F | —(CH₂)— | —(CH₂)₃— | NMe(I-Pr) |
| 2-Me-Bu | F | F | —(CH₂)— | —(CH₂)₃— | NMe(CHO) |
| 2-Me-Bu | F | F | —(CH₂)— | —(CH₂)₃— | NMe(COCH₃) |
| 2-Me-Bu | F | F | —(CH₂)— | —(CH₂)₃— | NMe(CO₂Me) |
| 2-Me-Bu | F | F | —(CH₂)— | —(CH₂)₃— | NMe(COEt) |
| 2-Me-Bu | F | F | —(CH₂)— | —(CH₂)₃— | NMe(CO-c-Pr) |
| 2-Me-Bu | F | F | —(CH₂)— | —(CH₂)₃— | NMe(CO₂Et) |
| 2-Me-Bu | F | F | —(CH₂)— | —(CH₂)₃— | NMe(COCF₃) |
| 2-Me-Bu | F | F | —(CH₂)— | —(CH₂)₃— | NMe(Boc) |
| 2-Me-Bu | F | F | —(CH₂)— | —(CH₂)₃— | NMe(CH₂CF₃) |
| 2-Me-Bu | F | F | —(CH₂)— | —(CH₂)₃— | OH |

TABLE 7a

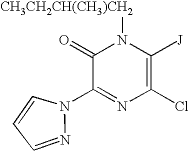

| J | J | J |
|---|---|---|
| 3-Cl-2-pyridinyl | 3-F-2-thienyl | 6-Cl-3-pyridazinyl |
| 3-CF₃-2-pyridinyl | 3,5-di-Cl--2-thienyl | 2-thiazolyl |
| 3-Me-2-pyridinyl | 3,5-di-Me-2-thienyl | 2-oxazolyl |
| 3-F-2-pyridinyl | 2,4-di-Me-2-thienyl | 2,4-di-Me-5-thiazolyl |
| 3-Br-2-pyridinyl | 1-naphthalenyl | 2,4-di-Cl-5-thiazolyl |
| 3-CN-2-pyridinyl | 2-Me-1-naphthalenyl | 2,5-di-Cl-4-thiazolyl |
| 3-MeO-2-pyridinyl | 2-Cl-1-naphthalenyl | 3,5-di-Me-4-iso- |
| | | xazolyl |
| 3,5-di-Me-2-pyridinyl | 3-Cl-2-quinolinyl | 3,5-di-Cl-4-isothia- |
| | | zolyl |
| 3,6-di-Me-2-pyridinyl | 3-Cl-2-quinoxalinyl | 1,2,3-oxadiazol-4-yl |
| 3,5-di-Cl-2-pyridinyl | 2-naphthalenyl | 5-Me-1,2,3-thia- |
| | | diazol-4-yl |
| 2-Cl-3-pyridinyl | 1-Me-2-naphtthalenyl | 1,3,4-thiadiazol-2-yl |
| 2-Me-3-pyridinyl | 1-Cl-2-naphthalenyl | 1,3,4-oxadiazol-2-yl |
| 2-F-3-pyridinyl | 3,6-di-Cl-2-quinolinyl | 5-Cl-1,2,3-thia- |
| | | diazol-4-yl |
| 2-MeO-3-pyridinyl | 3,6-di-Cl-2-quinoxalinyl | 2,5-di-Me-1,2,3-tri- |
| | | azol-4-yl |
| 2-MeS-3-pyridinyl | 3-Me-2-quinolinyl | 2,5-di-Me-1H- |
| | | pyrrol-1-yl |
| 4-Cl-3-pyridinyl | 2-Cl-3-quinolinyl | 2,5-di-Cl-1H-pyrrol- |
| | | 1-yl |
| 4-Me-3-pyridinyl | 2-D-3-quinolinyl | 2,5-di-Br-1H-pyrrol- |
| | | 1-yl |
| 4-F-3-pyridinyl | 2-benzoxazolyl | 2-Me-1H-pyrrol- |
| | | 1-yl |
| 4-MeO-3-pyridinyl | 2-benzothiazolyl | 2,4-di-Me-1H- |
| | | pyrrol-1-yl |
| 4-MeS-3-pyridinyl | 4-quinazolyl | 3,5-di-Me-1H-pyra- |
| | | zol-1-yl |
| 2,4-di-Cl-3-pyridinyl | 1-issoquinolinyl | 3,5-di-Me-1H-1,2,4- |
| | | triazol-1-yl |
| 2,4-di-Me-3-pyridinyl | 4-quinolinyl | 3-CF₃-5-Me-1H- |
| | | pyrazol-1-yl |
| 2,4-di-F-3-pyridinyl | 3-Cl-4-quinolinyl | 1,3,5-tri-Me-1H- |
| | | pyrazol-4-yl |
| 2,4,6-tri-Me-3-pyridinyl | 3-Cl-2-pyrazinyl | 1,3-di-Me-5-Cl-1H- |
| | | pyrazol-4-yl |
| 2,4,6-tri-F-3- | 3-CF₃-2-pyrazinyl | 2,5-di-Me-1H- |
| pyridinyl | | imidazol-1-yl |
| 3,5-di-Me-F-4-pyridinyl | 3-Me-2-pyrazinyl | 2-Me-1H-imidazol- |
| | | 1-yl |
| 3-Cl-4-pyridinyl | 3-F-2-pyrazinyl | 5-Me-1H-imidazol- |
| | | 1-yl |
| 3-Me-4-pyridinyl | 3-Br-2-pyrazinyl | 4-Me-5-thiazolyl |
| 3,5-di-Cl-4-pyridinyl | 3-CN-2-pyrazinyl | 5-Me-1H-imidazol- |
| | | 1-yl |
| 3,5-di-Me-4-pyridinyl | 3-MeO-2-pyrazinyl | 5-Cl-4-thiazolyl |
| 2-Cl-3-thienyl | 3,5-di-Me-2-pyrazinyl | 5-Me-4-thiazolyl |
| 2-Me-3-thienyl | 3,6-di-Me-2-pyrazinyl | 3,4,5-tri-Me-1H- |
| | | pyrazol-1-yl |
| 2-F-3-thienyl | 3,5-di-Cl-22-pyrazinyl | 3,5-di-Me-2-furanyl |
| 2,4-di-Cl-3-thienyl | 5-Cl-4-pyrimidinyl | 2,4-di-Me-3-furanyl |
| 2,5-di-Me-3-thienyl | 5-Me-4-pyrimidinyl | 3-CF₃-1,5-di-Me- |
| | | 1H-pyrazol-4-yl |
| 3-Cl-2-thienyl | 5-F-4-pyrimidinyl | 5-Me-1,2,3-oxadi- |
| | | azol-4-yl |
| 3-Me-2-thienyl | 5-CF₃-4-pyrimidinyl | 5-Cl-1,2,3-oxadi- |
| | | azol-4-yl |

TABLE 7b

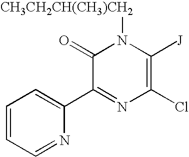

| J | J | J |
|---|---|---|
| 3-Cl-2-pyridinyl | 3-F-2-thienyl | 6-Cl-3-pyridazinyl |
| 3-CF₃-2-pyridinyl | 3,5-di-Cl-2-thienyl | 2-thiazolyl |
| 3-Me-2-pyrridinyl | 3,5-di-Me-2-thienyl | 2-oxazolyl |
| 3-F-2-pyridinyl | 2,4-di-Me-2-thienyl | 2,4-di-Me-5-thia- |
| | | zolyl |
| 3-Br-2-pyrridinyl | 1-naphthalenyl | 2,4-di-Cl-5-thia- |
| | | zolyl |
| 3-CN-2-pyridinyl | 2-Me-1-naphthalenyl | 2,5-di-Cl-4-thiazolyl |
| 3-MeO-2-pyrridinyl | 2-Cl-1-naphthalenyl | 3,5-di-Me-4-iso- |
| | | xazolyl |
| 3,5-di-Me-2-pyridinyl | 3-Cl-2-quinolinyl | 3,5-di-Cl-4-iso- |
| | | thiazolyl |
| 3,6-di-Me-2-pyridinyl | 3-Cl-2-quinoxalinyl | 1,2,3-oxadiazol-4-yl |
| 3,5-di-Cl-2-pyridinyl | 2-naphthalenyl | 5-Me-1,2,3-thiadi- |
| | | azol-4-yl |
| 2-Cl-3-pyridinyl | 1-Me-2-naphthalenyl | 1,3,4-thiadiazol-2-yl |
| 2-Me-3-pyridinyl | 1-Cl-2-naphtthalenyl | 1,3,4-oxadiazol-2-yl |
| 2-F-3-pyridinyl | 3,6-di-Cl-2-quinolinyl | 5-Cl-1,2,3-thia- |
| | | diazol-4-yl |
| 2-MeO-3-pyridinyl | 3,6-di-Cl-2-quinoxalinyl | 2,5-di-Me-1,2,3-tri- |
| | | azol-4-yl |
| 2-MeS-3-pyridinyl | 3-Me-2-quinolinyl | 2,5-di-Me-1H- |
| | | pyrrol-1-yl |
| 4-Cl-3-pyridinyl | 2-Cl-3-quinolinyl | 2,5-di-Cl-1H-pyrrol- |
| | | 1-yl |
| 4-Me-3-pyridinyl | 2-F-3-quinolinyl | 2,5-di-Br-1H-pyrrol- |
| | | 1-yl |
| 4-F-3-pyridinyl | 2-benzoxazolyl | 2-Me-1H-pyrrol- |
| | | 1-yl |
| 4-MeO-3-pyridinyl | 2-benzothiazolyl | 2,4-di-Me-1H- |
| | | pyrrol-1-yl |
| 4-MeS-3-pyridinyl | 4-quinazolinyl | 3,5-di-Me-1H-pyra- |
| | | zol-1-yl |
| 2,4-di-Cl-3-pyridinyl | 1-isoquinolinyl | 3,5-di-Me-1H-1,2,4- |
| | | triazol-1-yl |
| 2,4-di-Me-3-pyridinyl | 4-quinolinyl | 3-CF₃-5-Me-1H- |
| | | pyrazol-1-yl |
| 2,4-di-F-3-pyridinyl | 3-Cl-4-quinolinyl | 1,3,5-tri-Me-1H- |
| | | pyrazol-4-yl |
| 2,4,6-tri-Me-3-pyridinyl | 3-Cl-2-pyrazinyl | 1,3-di-Me-5-Cl-1H- |
| | | pyrazol-4-yl |
| 2,4,6-tri-F-3-pyridinyl | 3-CF₃-2-pyrazinyl | 2,5-di-Me-1H- |
| | | imidazol-1-yl |
| 3,5-di-F-4-pyridinyl | 3-Me-2-pyrazinyl | 2-Me-1H-imidazol- |
| | | 1-yl |
| 3-Cl-4-pyridinyl | 3-F-2-pyrazinyl | 5-Me-1H-imidazol- |
| | | 1-yl |
| 3-Me-4-pyridinyl | 3-Br-2-pyrazinyl | 4-Me-5-thiazolyl |
| 3,5-di-Cl-4-pyridinyl | 3-CN-2-pyrazinyl | 4-Cl-5-thiazolyl |
| 3,5-di-Me-4-pyridinyl | 3-MeO-2-pyrazinyl | 5-Cl-4-thiazolyl |
| 2-Cl-3-thienyl | 3,5-di-Me-2-pyrazinyl | 5-Me-4-thiazolyl |
| 2-Me-3-thienyl | 3,6-di-Me-2-pyrazinyl | 3,4,5-tri-Me-1H- |
| | | pyrazol-1-yl |
| 2-F-3-thienyl | 3,5-di-Cl-2-pyrazinyl | 3,5-di-Me-2-furanyl |
| 2,4-di-Cl-3-thienyl | 5-Cl-4-pyrimidinyl | 2,4-di-Me-3-furanyl |
| 2,5-di-Me-3-thienyl | 5-Me-4-pyrimidinyl | 3-CF₃-1,5-di-Me- |
| | | 1H-pyrazol-4-yl |
| 3-Cl-2-thienyl | 5-F-4-pyrimidinyl | 5-Me-1,2,3-oxa- |
| | | diazol-4-yl |
| 3-Me-2-thienyl | 5-CF₃-4-pyrimidinyl | 5-Cl-1,2,3-oxa- |
| | | diazol-4-yl |

TABLE 7c

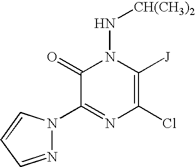

J
3-Cl-2-pyridinyl
3-CF₃-2-pyridinyl
3-Me-2-pyridinyl
3-F-2-pyridinyl
3-Br-2-pyridinyl
3-CN-2-pyridinyl
3-MeO-2-pyridinyl
3,5-di-Me-2-pyridinyl
3,6-di-Me-2-pyridinyl
3,5-di-Cl-2-pyridinyl
2-Cl-3-pyridinyl
2-Me-3-pyridinyl
2-F-3-pyridinyl
2-MeO-3-pyridinyl
2-MeS-3-pyridinyl
4-Cl-3-pyridinyl
4-Me-3-pyridinyl
4-F-3-pyridinyl
4-MeO-3-pyridinyl
4-MeS-3-pyridinyl
2,4-di-Cl-3-pyridinyl
2,4-di-Me-3-pyridinyl
2,4-di-F-3-pyridinyl
2,4,6-tri-Me-3-pyridinyl
2,4,6-tri-F-3-pyridinyl
3,5-di-F-4-pyridinyl
3-Cl-4-pyridinyl
3-F-2-thienyl
3,5-di-Cl-2-thienyl
3,5-di-Me-2-thienyl
2,4-di-Me-2-thienyl
1-naphthalenyl
2-Me-1-naphthalenyl
2-Cl-1-naphthalenyl
3-Cl-2-quinolinyl
3-Cl-2-quinoxalinyl
2-naphthalenyl
1-Me-2-naphthalenyl
1-Cl-2-naphthalenyl
3,6-di-Cl-2-quinolinyl
3,6-di-Cl-2-quinoxalinyl
3-Me-2-quinolinyl
2-Cl-3-quinolinyl
2-F-3-quinolinyl
2-benzoxazolyl
2-benzothiazolyl
4-quinazolinyl
1-isoquinolinyl
4-quinolinyl
3-Cl-4-quinolinyl
3-Cl-2-pyrazinyl
3-CF₃-2-pyrazinyl
3-Me-2-pyrazinyl
3-F-2-pyrazinyl
6-Cl-3-pyridazinyl
2-thiazolyl
2-oxazolyl
2,4-di-Me-5-thiazolyl
2,4-di-Cl-5-thiazolyl
2,5-di-Cl-4-thiazolyl
3,5-di-Me-4-isoxazolyl
3,5-di-Cl-4-isothiazolyl
1,2,3-oxadiazol-4-yl
5-Me-1,2,3-thiadiazol-4-yl
1,3,4-thiadiazol-2-yl
1,3,4-oxadiazol-2-yl
5-Cl-1,2,3-thiadiazol-4-yl
2,5-di-Me-1,2,3-triazol-4-yl
2,5-di-Me-1H-pyrrol-1-yl
2,5-di-Ci-1H-pyrrol-1-yl
2,5-di-Br-1H-pyrrol-1-yl
2-Me-1H-pyrrol-1-yl
2,4-di-Me-1H-pyrrol-1-yl
3,5-di-Me-1H-pyrazol-1-yl
3,5-di-Me-1H-1,2,4-triazol-1-yl
3-CF₃-5-Me-1H-pyrazol-1-yl
1,3,5-tri-Me-1H-pyrazol-4-yl
1,3-di-Me-5-Cl-1H-pyrazol-4-yl
2,5-di-Me-1H-imidazol-1-yl
2-Me-1H-imidazol-1-yl
5-Me-1H-imidazol-1-yl
3-Me-4-pyridinyl
3,5-di-Cl-4-pyridinyl
3,5-di-Me-4-pyridinyl
2-Cl-3-thienyl
2-Me-3-thienyl
2-F-3-thienyl
2,4-di-Cl-3-thienyl
2,5-di-Me-3-thienyl
3-Cl-2-thienyl
3-Me-2-thienyl
3-Br-2-pyrazinyl
3-CN-2-pyrazinyl
3-MeO-2-pyrazinyl
3,5-di-Me-2-pyrazinyl
3,6-di-Me-2-pyrazinyl
3,5-di-Cl-2-pyrazinyl
5-Cl-4-pyrimidinyl
5-Me-4-pyrimidinyl
5-F-4-pyrimidinyl
5-CF₃-4-pyrimidinyl
4-Me-5-thiazolyl
4-Cl-5-thiazolyl
5-Cl-4-thiazolyl
5-Me-4-thiazolyl
3,4,5-tri-Me-1H-pyrazol-1-yl
3,5-di-Me-2-furanyl
2,4-di-Me-3-furanyl
3-CF₃-1,5-di-Me-1H-pyrazol-4-yl
5-Me-1,2,3-oxadiazol-4-yl
5-Cl-1,2,3-oxadiazol-4-yl

TABLE 7d

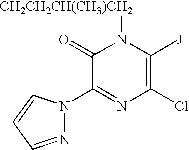

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
CH₂CH₂SMe
CH₂CH(Me)SMe
CH₂CH₂S(O)Me
CH₂CH₂S(O)2Me
CH₂CO₂Me
CH₂CO₂-i-Pr
CH(Me)CO₂Me
CH₂C(O)Me
CH₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
c-Pr
c-pentyl
c-Hex
2-cyclohexenyl
3-cyclohexenyl
CH₂-4-Cl-Ph
CH(Et)₂
CH₂CH(Et)₂
CH₂-c-Hex
CH(CF₃)Et
CH(Et)-n-Pr
CH(Me)-n-Bu
t-Bu

TABLE 7e

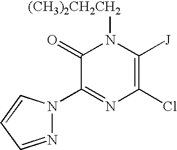

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
c-Pr
c-pentyl
c-Hex
2-cyclohexenyl
3-cyclohexenyl
CH₂CH₂SMe
CH₂CH(Me)SMe
CH₂CH₂S(O)Me
CH₂CH₂S(O)2Me
CH₂CO₂Me
CH₂CO₂-i-Pr
CH(Me)CO₂Me
CH₂C(O)Me
CH₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂-4-Cl-Ph
CH(Et)₂
CH₂CH(Et)₂
CH₂-c-Hex
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
CH(CF₃)Et
CH(Et)-n-Pr
CH(Me)-n-Bu
t-Bu

TABLE 7f

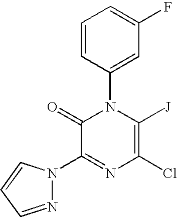

J
Me
Et
i-Pr
n-Pr
i-Bu
n-Bu
s-Bu
3-Me-Bu
n-pentyl
n-Hex
2-propenyl
2-Me-2-propenyl
3-butenyl
3-pentenyl
2-propynyl
3-butynyl
4-butynyl
c-Pr
c-pentyl
c-Hex
2-cyclohexenyl
3-cyclohexenyl
CH₂CH₂SMe
CH₂CH(Me)SMe
CH₂CH₂S(O)Me
CH₂CH₂S(O)2Me
CH₂CO₂Me
CH₂CO₂-i-Pr
CH(Me)CO₂Me
CH₂C(O)Me
H₂CH₂C(O)Me
CH₂SiMe₃
CH₂CH₂SiMe₃
2,2-dimethylpropyl
CH₂Ph
CH₂-c-Pr
CH₂CH(n-Pr)Me
CH₂-2-Cl-Ph
CH₂-3-Cl-Ph
CH₂-4-Cl-Ph
CH(Et)₂
CH₂CH(Et)₂
CH₂-c-Hex
CH₂CN
CH₂NO₂
CH₂CH₂OH
CH₂CH₂OMe
CH₂CH(Me)OMe
CH(Me)CH₂OMe
CH(Me)CH(OMe)₂
CH₂-2-dioxolanyl
CH₂CH₂OCF₃
CH₂-2-cyclohexenyl
4-tetrahydropyranyl
3-tetrahydropyranyl
3-tetrahydrofuranyl
CH₂CH₂CH(Me)₂
t-Amyl
CH(Me)Et
CH(Me)-n-Pr
CH(CF₃)Et
CH(Et)-n-Pr
CH(Me)-n-Bu
t-Bu

### Utility

This invention pertains to a method of inhibiting undesired cellular proliferation said method comprising contacting said cells or a tissue or organ in which proliferation of said cell is not desired with a compound of Formula 1 prodrugs thereof, and all pharmaceutically acceptable salts, N-oxides, hydrates, solvates, crystal forms or geometric and stereoisomers thereof.

Inhibition of undesired cellular proliferation can be brought about by several mechanisms, including inter alia: alkylating agents, topoisomerase inhibitors, nucleotide analogues, antibiotics, hormone antagonists, and nucleic acid damaging agents. One pharmacologically important mechanism of inhibiting cellular proliferation is by means of impairing the function of microtubules. Microtubules facilitate and make possible, among other things, chromosome and organelle movement and segregation during cell mitosis (Stryer, L., Biochemistry (1988)). Preventing or interfering with microtubule function leads to mitotic arrest and frequently to apoptosis. In addition to neoplasia and cancer, many diseases are characterized by undesirable cell proliferation, and the value of compounds and methods that prevent such undesirable cell proliferation is of great importance to the treatment of such diseases. Microtubule function is also critical to cell maintenance, locomotion and the movement of specialized cell structures such as cilia and flagella (Stryer, L., Biochemistry (1988)).

To function properly, cilia and flagella require proper microtubule function (U.S. Pat. No. 6,162,930). Certain compounds are known to inhibit tubulin polymerization or to cause the formation of tubulin polymer with altered morphology and stability. By interfering with normal microtubule function such compositions may be used to treat those diseases characterized by abnormal proliferation.

As in mammalian cells, microtubule function plays a critical roll in eukaryotic cells. Thus the disruption of microtubule function can be an effective way of preventing the proliferation of pathogenic fungi in a host organism.

Tubulin is an asymmetric dimer composed of alpha and beta subunits that polymerizes to form microtubules. Microtubules must be highly dynamic in order to carry out many of their functions. At certain stages of the cell cycle, or in particular cell types or organelles, stable microtubules are required, such as for transport within axons or for ciliary and flagellar movement. Microtubules assemble during the G2 phase of the cell cycle and participate in the formation of the mitotic spindle which facilitates the segregation of sister chromatids during the process of cell division. The essential role of microtubules in cell division and the ability of drugs that interact with tubulin to interfere with the cell cycle have made tubulin a successful target for applications that include anti-cancer drugs, fungicides, and herbicides. Typical tubulin ligands such as colchicine, paclitaxel, the Vinca alkaloids such as vinblastine, the epothilones, the halicondrins, benomyl and mebendazole directly inhibit cell division by affecting microtubule function which leads to the arrest of the cell cycle at the G2/M boundary of mitosis. This mechanism is the basis of the therapeutic value of compounds of this type, such as treating gout with colchicine, restenosis with paclitaxel, cancer with paclitaxel, vinblastine, epothilones and halichondrins, and fungal infections with benomyl and malaria and helminthes with mebendazole.

Interfering with microtubule function can inhibit cell division in several ways. Both stabilizing microtubules and inhibiting their polymerization will prevent the cytoskeleton restructuring that is required at several points in the cell cycle and lead to an arrest of the cell's progression from one stage in the cell cycle to the next. Three main classes of tubulin-binding drugs (namely, colchicine analogues, Vinca alkaloids, and the taxanes) have been identified, each of which occupies different sites on the 13-tubulin molecule. Paclitaxel (Taxol™) and related taxanes represent a class of drugs that stabilize microtubules, a process that ultimately leads to “freezing” of the microtubule structures so that they cannot be restructured (Jordan M. A. and Wilson, L., 1998). Subsequent arrest at mitosis induces the apoptotic mechanism to cause cell death. A number of colchicine analogs (as well as several other compounds that bind to the same site on 13-tubulin as colchicine) disrupt tubulin polymerization and disrupt microtubular formation. Vinblastine and several other vinca-related drugs bind to a site that is distinct from the colchicine site. Compounds that bind at the Vinca-site prevent microtubule formation and destabilize microtubules (Jordan et al, 1986; Rai and Wolff (1996).

This invention is directed to compounds and methods designed to inhibit undesired cell proliferation generally in vivo or in-vitro. Although not wishing to be bound by theory it appears that the compounds of the invention accomplish this result by inhibition of microtubule function. Examples are disclosed that show a concentration dependent effect on microtubule stability. At low concentration, the compounds act like paclitaxel by stabilizing microtubule formation throughout the course of the assay. At higher concentrations, tubulin polymerization is apparently inhibited over the initial phases of the assay, but eventually the degree of polymerization turbidometry exceeds that of paclitaxel.

Accordingly, the present invention aims to provide compounds which are directly or indirectly toxic to actively dividing cells. The present invention is also directed to therapeutic compositions for treating said conditions caused by cellular hyperproliferation. The invention is therefore directed to compounds and methods of treatment of cellular hyperproliferation disorders. This broad class of disorders includes neoplasms. The neoplasms may be mammary, small-cell lung, non-small-cell lung, colorectal, leukemia, lymphoma, melanoma, pancreatic, renal, liver, myeloma, multiple mycloma, mesothelioma, central nervous system, ovarian, prostate, sarcoma of soft tissue or bone, head and neck, esophageal, stomach, bladder, retinoblastoma, squamous cell, testicular, vaginal, and neuroendocrine-related neoplasms. The neoplasms may be cancerous or non-cancerous. More broadly the invention is intended to provide compounds and methods for killing actively proliferating cells, besides neoplastic cells such as, bacterial, or epithelial cells, and treating infections (viral and bacterial), inflammatory, and generally proliferative conditions. A further aspect relates to provide methods for treating other cellular hyperproliferation disorders characterized by the presence of rapidly proliferating cells, such as psoriasis, vascular restenosis, atherersclerotic lesions, inflammatory diseases, autoimmune diseases, or psoriasis. Inflammatory disease include those where endothelial cells, inflammatory cells and glomerular cells are involved; myocardial infarction, where heart muscle cells are involved; glomerular nephritis, where kidney cells are involved; transplant rejection, where endothelial cells are involved; and infectious diseases such as HIV infection and malaria, where certain immune cells and/or other infected cells are involved. One further aspect provides a method for treating disease caused by the presence of pathogenic fungi.

In one embodiment, the method of the invention is used in the treatment of sarcomas, carcinomas and/or leukemias. Exemplary disorders for which the subject method can be used alone or as part of a treatment regimen include: fibrosarcoma, myxosarcoma, liposarcoma, chondrosarcoma, osteogenic sarcoma, choroma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In certain embodiments, the method of the invention is used to treat disorders such as carcinomas forming from tissue of the breast, prostate, kidney, bladder or colon.

In other embodiments, the method of the invention is used to treat hyperplastic or neoplastic disorders arising in adipose tissue, such as adipose cell tumors (e.g., lipomas, fibrolipomas, lipoblastomas, lipomatosis, hibermomas, hemangiomas and/or liposarcomas).

In still other embodiments, infectious and parasitic agents (e.g. bacteria, trypanosomes, fungi, etc.) can also be controlled using the subject compositions and compounds. For example the compositions and methods of the present invention can also be used to treat diseases, in which normal tubulin polymerization and function plays a role. Chagas' disease, for example, is caused by Trypanosoma cruzi, a flagellate protozoa which has a substantial protein composition containing tubulin both as a component of the subpellicular microtubule system and the flagellum. Chagas' disease is characterized by lesions in the heart, alimentary tract and nervous system. The disease is the leading cause of myocarditis in the Americas. Inhibition of tubulin polymerization, crucial to the parasite's mobility, would provide an effective treatment. Indeed, the use of agents that selectively affect tubulin polymerization has precedence in the therapy of other parasitic diseases. The benzimidazoles are very effective anti-helmenthic drugs, and the dinitroanilines have shown promise against Leishmania, a parasite closely related to Trypanosoma (U.S. Pat. No. 6,162,930). The compositions of the present invention may be used to contact such parasites or sites of parasitic infection and thereby treat the associated disease.

As will be appreciated by one skilled in the art, the dosage of the composition comprising the compounds of Formula 1 will depend on the condition being treated, the particular compound used, the type and severity of the disease or malady, and other clinical factors such as weight, sex, age and condition of the patient, the patient's tolerance to drugs and/or treatment, and the route of administration. Those skilled in the art will be able to determine the appropriate dosages depending on these and other factors.

In the treatment or prevention of hyperproliferation-related disorders an appropriate dosage level will generally be about 0.01 to 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

This specification makes reference to treating “individuals”. In addition to individuals such as humans, a variety of other mammals including other primates can be treated according to the method of the present invention. For instance, mammals including, but not limited to, cows, sheep, goats, horses, dogs, cats, guinea pigs, rats or other bovine, ovine, equine, canine, feline, rodent or murine species can be treated. Furthermore, the method can also be practiced in other species, such as avian species (e.g., chickens).

The present invention provides pharmaceutical compositions comprising at least one of the compounds of the Formula 1 capable of treating a hyperproliferation-related disorder in an effective amount in a pharmaceutically acceptable vehicle or diluent. The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques well known in the art of pharmaceutical formulation.

The compounds of the Formula 1 may be administered by any suitable means, for example, orally, such as in the form of tablets, capsules, granules or powders; sublingually; buccally; parenterally, such as by subcutaneous, intravenous, intramuscular or intracisternal injection or infusion techniques (e.g., as sterile injectable aqueous or non-aqueous solutions or suspensions); nasally, such as by inhalation spray; topically, such as in the form of a cream or ointment; or rectally, such as in the form of suppositories; or in dosage unit formulations containing non-toxic, pharmaceutically acceptable vehicles or diluents. The compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents such as calcium carbonate, sodium carbonate, lactose, calcium phosphate or sodium phosphate; granulating and disintegrating agents such as corn starch, or alginic acid; binding agents such as starch, gelatin or acacia, and lubricating agents such as magnesium stearate, stearic acid or talc. The tablets may be uncoated, or they may be coated by known techniques to delay disintegration and absorption in the gastrointestinal tract, and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent such as calcium carbonate, calcium phosphate or kaolin, or as soft gelatin capsules wherein the active ingredient is mixed with water or an oil medium such as peanut oil, liquid paraffin, or olive oil.

Aqueous suspensions contain the active materials in admixture with excipients suitable for the manufacture of aqueous suspensions. Such excipients are suspending agents such as sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide such as lecithin, or condensation products of an alkylene oxide with fatty acids such as polyoxyethylene stearate, or condensation products of ethylene oxide with long chain aliphatic alcohols such as heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate. The aqueous suspensions may also contain one or more preservatives such as ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil such as peanut oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil such as olive oil or peanut oil, or a mineral oil such as liquid paraffin or mixtures of these. Suitable emulsifying agents may be naturally-occurring gums such as gum acacia or gum tragacanth; naturally-occurring phosphatides such as soybean, lecithin, and esters or partial esters derived from fatty acids and hexitol anhydrides such as sorbitan monooleate, and condensation products of the said partial esters with ethylene oxide such as polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolisable lipid capable of forming liposomes can be used. The present compositions in liposome form can contain, in addition to a compound of the present invention, stabilizers, preservatives, excipients and the like. The preferred lipids are the phospholipids and phosphatidylcholines, both natural and synthetic. Methods to form liposomes are known in the art. The liposomes may or may not be form part of a targeted drug delivery system for example in a liposome coated with a tumor specific antibody. Such liposomes will be targeted to and taken up selectively by the site of interest (e.g. a tumor cell). Further long-circulating or “stealth” liposomes may be employed (U.S. Pat. No. 5,013,556).

Generally, such liposomes or other drug delivery systems typically have a targeting moiety, i.e., ligand, conjugated thereto that is specific for the target site of interest (e.g., tumor cell). For instance, some property (biochemical, architectural or genetic) of the tumor that is different from normal tissue can be exploited to concentrate the compounds of the present invention in, or at least near, the target tumor. Tumor vasculature, which is composed primarily of endothelial cells, is inherently different than normal differentiated vasculature. For example, the architecture of tumor vasculature is known to be leaky, and blood flow through them is mostly intermittent, with periods of perfusion and periods of occlusion and subsequent hypoxia. This aberrant microenvironment may be caused by and, in turn, leads to, additional differential gene expression in tumor vasculature relative to normal vasculature. This abnormal architecture and function, at the molecular level is characterized by differences in surface markers in tumor microvessels relative to normal vessels and such differences can be exploited to target the liposome or other drug delivery system to the site of interest. Liposomes offer the added advantage of shielding the drug from most normal tissues. When coated with polyethylene glycol (PEG) (i.e., stealth liposomes) to minimize uptake by phagocytes and with a tumor vasculature-specific targeting moiety, liposomes offer longer plasma half-lives, lower non-target tissue toxicity and delivery, and increased efficacy over non-targeted drug.

Other targeting strategies include, but are not limited to, ADEPT (antibody-directed enzyme prodrug therapy), GDEPT (gene-directed EPT) and VDEPT (virus-directed EPT). In ADEPT, the targeting of an inactive prodrug to a tumor mass is effected by an antibody against a tumor-associated marker. The enzyme milieu in or about the tumor transforms the prodrug into an active toxic agent that then acts on the tumor tissue. Similarly, differential gene expression or viral targeting at the tumor site is used to activate a prodrug into its active, toxic form in GDEPT and VDEPT, respectively. Other strategies include targeting differentially expressed genes, enzymes or surface markers that appear on, for example, tumor-associated vasculature to effect control of tumor progression or to other sites of interest (e.g., endothelial cells, TNF-α, TNF-α receptor, etc.).

Additionally, standard pharmaceutical formulation techniques may be employed such as those described in Remington's Pharmaceutical Sciences, Mack Publishing Company, Easton, Pa. Additional methods of encapsulating compounds or compositions comprising the compound are known to those skilled in the art (Baker et al., “Controlled Release of Biological Active Agents”, John Wiley and Sons, 1986).

Preferred unit dosage formulations are those containing a daily dose or unit, daily sub-dose, as herein above recited, or an appropriate fraction thereof, of the administered ingredient.

It should be understood that in addition to the ingredients specifically set forth above, the formulations of this invention may include other agents conventional in the art having regard to the type of formulation in question, for example, those suitable for oral administration may include flavoring and other agents.

In addition, the compounds may be incorporated into biodegradable polymers allowing for sustained release, the polymers being implanted in the vicinity of where delivery is desired, for example, at the site of a tumor. Biodegradable polymers and their use are described in detail in Brem et al., J. Neurosurg. 74, 441-446 (1991), and are familiar to those skilled in the art.

The pharmaceutical composition and method of the present invention may further comprise other therapeutically active compounds as noted herein which are usually applied in the treatment of the above mentioned pathological conditions. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Examples of other therapeutic agents include the following: Tyrosine kinase inhibitors, such as imatinib (Glivec™), and gefitinib (Iressa™) inter alia, cyclosporins (e.g., cyclosporine A), CTLA4-Tg, antibodies such as ICAM-3, anti-IL-2 receptor (Anti-Tac), anti-CD45R^{B}, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD401g and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal anti-inflammatory drugs (NSAIDs) such as ibuprofen, aspirin, acetaminophen and cyclooxygenase inhibitors such as refecoxib, steroids such as prednisolone or dexamethasone, gold compounds, antiproliferative agents such as methotrexate, FK506 (tacrolimus, Prograf), mycophenolate mofetil, antineoplastic agents such as azathioprine, VP-16, etoposide, fludarabine, cisplatin, bortezomib, doxorubicin, adriamycin, amsacrine, camptothecin, cytarabine, gemcitabine, fluorodeoxyuridine, melphalan and cyclophosphamide, TNF-α inhibitors such as tenidap, anti-TNF antibodies or soluble TNF receptor, and rapamycin (sirolimus or Rapamune) or derivatives thereof.

When other therapeutic agents are employed in combination with the compounds of the present invention they may be used for example in amounts as noted in the Physician Desk Reference (PDR) or as otherwise determined by one of ordinary skill in the art.

The pharmaceutical composition and method of the present invention may further comprise other therapeutically active compounds as noted herein which are known inhibitors or substrates of drug efflux systems or drug detoxification and excretory systems. Such systems include P-glycoprotein, multidrug resistance-associated protein, lung resistance protein and glutathione S-transferase isoenzymes alpha, mu, pi, sigma, theta, zeta and kappa. Co-administration of drugs known to inhibit or reduce the activity of these systems may increase the efficacy of the compounds described in the present invention through increasing the amount of therapeutic agent in the cell. Using this approach, one may be able to achieve therapeutic efficacy with lower dosages, thus reducing the potential for adverse side effects. Examples of inhibitors or substrates for these systems include; verapamil, probenecid, dipyridamole, ethacrynic acid, indomethacin, sulfasalazine, buthionine sulfoximine, cyclosporine A and tamoxifen.

In order that the nature of the present invention may be more clearly understood preferred forms thereof will now be described by reference to the following non-limiting Examples.

The following TESTS demonstrate the microtubule inhibition and antiproliferative efficacy of compounds of this invention. The activity afforded by the compounds is not limited, however, to these species. See Index TablesA through D for compound descriptions. The following abbreviations are used in the Index Tables which follow: t means tertiary, s means secondary, n means normal, i means iso, c means cyclo, Me means methyl, Et means ethyl, Pr means propyl, i-Pr means isopropyl, Bu means butyl, i-Bumeansisobutyl, Hex means hexyl, Ac means acetyl, c-Hex means cyclohexyl, Ph means phenyl, OMe meansmethoxy, SMe means methylthio, CN means cyano, NO₂ means nitro, 2-Cl-4-F means 2-chloro-4-fluoro, TMS meanstrimethylsilyl, and other substituent abbreviations are defined analogously. The abbreviation “Ex.” stands for “Example” and is followed by a number indicating in which example the compound is prepared.

INDEX TABLE A

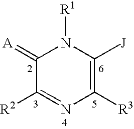

| Cmpd | | | | | |
|---|---|---|---|---|---|
| No. | R¹ | R² | R³ | J | M.P. (° C.) |
| 1 | i-Bu | 1H-pyrazol-1-yl | Cl | 2,6-di-F-Ph | * |
| (Ex. 1) | | | | | |
| 2 | i-Bu | 2-pyridinyl | Cl | 2,6-di-F-Ph | * |
| (Ex. 2) | | | | | |
| 3 | i-Bu | 1H-1,2,4-triazol-1-yl | Cl | 2,6-di-F-Ph | * |
| 4 | i-Bu | 4,5-dihydro-1H-pyrazol-1-yl | Cl | 2,6-di-F-Ph | * |
| 5 | i-Bu | CN | Cl | 2,6-di-F-Ph | 100-102 |
| (Ex. 10) | | | | | |
| 6 | i-Bu | 1H-pyrazol-1-yl | Cl | 2,4-di-F-Ph | 147-149 |
| 7 | i-Bu | 1H-pyrazol-1-yl | Cl | 2,4,6-tn-F-Ph | 111-113 |
| 8 | i-Bu | 1H-1,2,4-triazol-1-yl | Cl | 2,4,6-tn-F-Ph | 128-129 |
| 9 | i-Bu | 1H-pyrazol-1-yl | Cl | 2-Cl-6-F-Ph | * |
| 10 | i-Bu | 1H-1,2,4-triazol-1-yl | Cl | 2-Cl-6-F-Ph | * |
| 11 | 2-Me-Bu | 1H-pyrazol-1-yl | Cl | 2,6-di-F-Ph | 108-109 |
| 12 | 2-Me-Bu | 1H-1,2,4-triazol-1-yl | Cl | 2,6-di-F-Ph | 68-70 |
| 13 | i-Bu | 1H-pyrazol-1-yl | OMe | 2-Cl-6-F-Ph | * |
| 14 | Me | 1H-pyrazol-1-yl | Cl | 2-F-Ph | * |
| 15 | Me | 1H-1,2,4-triazol-1-yl | Cl | 2-F-Ph | * |
| 16 | CH₂-c-Pr | 4,5-dihydro-1H-pyrazol-1-yl | Cl | 2,6-di-F-Ph | * |
| 17 | CH₂-c-Pr | 1H-1,2,4-triazol-1-yl | Cl | 2,6-di-F-Ph | * |
| 18 | CH₂-c-Pr | 1H-pyrazol-1-yl | Cl | 2,6-di-F-Ph | * |
| 19 | 2-Me-Bu | 1H-pyrazol-1-yl | Cl | 2,4,6-tn-F-Ph | * |
| 20 | 2-Me-Bu | 1H-1,2,4-triazol-1-yl | Cl | 2,4,6-tri-F-Ph | * |
| 21 | 2-Me-Bu | CN | Cl | 2,6-di-F-Ph | * |
| 22 | i-Bu | 1H-pyrazol-1-yl | Cl | 4-Cl-Ph | * |
| 23 | i-Bu | 1H-1,2,4-triazol-1-yl | Cl | 4-Cl-Ph | * |
| 24 | i-Bu | 3,-5-diMe-1H-1,2,4-triazol-1- | Cl | 4-Cl-Ph | * |
| | | yl | | | |
| 25 | 1-Me-Bu | 1H-pyrazol-1-yl | Cl | 2,6-di-F-Ph | * |
| 26 | i-Pr | 1H-pyrazol-1-yl | Cl | 2,6-di-F-Ph | * |
| 27 | n-Pr | 1H-pyrazol-1-yl | Cl | 2,6-di-F-Ph | * |
| 28 | n-Pentyl | 1H-pyrazol-1-yl | Cl | 2,6-di-F-Ph | * |
| 29 | 1,2-di-Me-Pr | 1H-pyrazol-1-yl | Cl | 2,6-di-F-Ph | * |
| 30 | 2-Me-2-Propenyl | 1H-pyrazol-1-yl | Cl | 2,6-di-F-Ph | * |
| 31 | CH₂-c-Hex | 1H-pyrazol-1-yl | Cl | 2,6-di-F-Ph | * |
| 32 | s-Bu | 1H-1,2,4-triazol-1-yl | Cl | 2,6-di-F-Ph | * |
| 33 | i-Pr | 1H-1,2,4-triazol-1-yl | Cl | 2,6-di-F-Ph | * |
| 34 | n-Pr | 1H-1,2,4-triazol-1-yl | Cl | 2,6-di-F-Ph | * |
| 35 | 3-Me-Bu | 1H-pyrazol-1-yl | Cl | 2,6-di-F-Ph | * |
| 36 | i-Bu | 1H-pyrazol-1-yl | Cl | 4-CF₃-Ph | * |
| 37 | i-Bu | 1H-pyrazol-1-yl | Cl | 4-OMe-Ph | * |
| 38 | i-Bu | 1H-pyrazol-1-yl | Cl | 4-OCF₃-Ph | * |
| 39 | i-Bu | 1H-1,2,4-triazol-1-yl | Cl | 2,3-di-Cl-Ph | * |
| 40 | i-Bu | 1H-1,2,4-triazol-1-yl | Cl | 2,6-di-Cl-Ph | 176-181 |
| 41 | i-Bu | 1H-pyrazol-1-yl | Cl | 2,3-di-Cl-Ph | 104-109 |
| 42 | i-Bu | 1H-pyrazol-1-yl | Cl | 3,5-di-Cl-Ph | 182-187 |
| 43 | i-Bu | 1H-pyrazol-1-yl | Cl | 2,6-di-Cl-Ph | 145-148 |
| 44 | i-Bu | 1H-1,2,4-triazol-1-yl | Cl | 2-F-4-Cl-Ph | * |
| 45 | i-Bu | 1H-pyrazol-1-yl | Cl | 2-F-4-Cl-Ph | * |
| 46 | i-Bu | 1H-pyrazol-1-yl | Cl | 2-F-4-CF₃-Ph | * |
| 47 | c-Hex | 1H-pyrazol-1-yl | Cl | 2,6-di-F-Ph | * |
| 48 | i-Bu | NHN═C(Me)₂ | Cl | 2,6-di-F-Ph | * |
| 49 | c-Hex | 1H-pyrazol-1-yl | OMe | 2,6-di-F-Ph | 174-176 |
| 50 | i-Bu | 1H-pyrazol-1-yl | Cl | 2,3-di-F-Ph | * |
| 51 | i-Bu | 1H-1,2,4-triazol-1-yl | Cl | 2-Cl-4-F-Ph | 147-148 |
| 52 | i-Bu | 1H-1,2,4-triazol-1-yl | Cl | 2,3-di-F-Ph | 144-145 |
| 53 | i-Bu | 1H-pyrazol-1-yl | Cl | 2-Cl-4-F-Ph | 137-139 |
| (Ex. 6) | | | | | |
| 54 | i-Bu | 1H-1,2,4-triazol-1-yl | Cl | 2,5-di-F-Ph | 152-154 |
| 55 | i-Bu | 1H-1,2,4-triazol-1-yl | Cl | 2,3,6-tri-F-Ph | * |
| 56 | i-Bu | 1H-pyrazol-1-yl | Cl | 2,5-di-F-Ph | * |
| 57 | i-Bu | 1H-pyrazol-1-yl | Cl | 2,3,6-tri-F-Ph | * |
| 58 | CH₂CH═CH₂ | 1H-pyrazol-1-yl | Cl | 2,6-di-F-Ph | * |
| 59 | 2,2-di-Me-Pr | 1H-pyrazol-1-yl | Cl | 2,6-di-F-Ph | * |
| 60 | 2-Me-Bu | 1H-1,2,3-triazol-1-yl | Cl | 2,6-di-F-Ph | * |
| 61 | 2-Me-Bu | 1H-imidazol-1-yl | Cl | 2,6-di-F-Ph | * |
| 62 | 2-Me-Bu | 3,5-di-Me-1H-pyrazol-1-yl | Cl | 2,6-di-F-Ph | * |
| 63 | 2-Me-Bu | 3-CF₃-1H-pyrazol-1-yl | Cl | 2,6-di-F-Ph | * |
| 64 | 2-Me-Bu | 3-Me-5-CF₃-1H-pyrazol-1-yl | Cl | 2,6-di-F-Ph | * |
| 65 | 2-Me-Bu | 3-Br-1H-pyrazol-1-yl | Cl | 2,6-di-F-Ph | * |
| 66 | 2-Me-Bu | 3-Me-1H-pyrazol-1-yl | Cl | 2,6-di-F-Ph | * |
| 67 | 2-Me-Bu | 4,5-di-CN-1H-imidazol-1-yl | Cl | 2,6-di-F-Ph | * |
| 68 | 2-Me-Bu | 4,5-di-Cl-1H-imidazol-1-yl | Cl | 2,6-di-F-Ph | * |
| 69 | 2-Me-Bu | 2-Me-1H-imidazol-1-yl | Cl | 2,6-di-F-Ph | * |
| 70 | 2-Me-Bu | 2-Et-1H-imidazol-1-yl | Cl | 2,6-di-F-Ph | * |
| 71 | 2-Me-Bu | 2-n-Pr-1H-imidazol-1-yl | Cl | 2,6-di-F-Ph | * |
| 72 | 2-Me-Bu | 2-i-Pr-1H-imidazol-1-yl | Cl | 2,6-di-F-Ph | * |
| 73 | i-Bu | NHN═C(CH₂)₄ | Cl | 2,6-di-F-Ph | * |
| 74 | i-Bu | 1H-pyrazol-1-yl | Cl | 4-t-Bu-Ph | * |
| 75 | i-Bu | 1H-1,2,4-triazol-1-yl | Cl | 4-t-Bu-Ph | * |
| 76 | i-Bu | 1H-1,2,4-triazol-1-yl | Cl | 4-CF₃-Ph | * |
| 77 | i-Bu | 1H-1,2,4-triazol-1-yl | Cl | 4-OCF₃-Ph | * |
| 78 | i-Bu | NHN═C(Me)(CO₂Me) | Cl | 2,6-di-F-Ph | * |
| 79 | i-Bu | 1H-1,2,4-triazol-1-yl | Cl | 2,4-di-Me-Ph | * |
| 80 | i-Bu | 1H-pyrazol-1-yl | Cl | 2,4-di-Me-Ph | * |
| 81 | 2-Me-Bu | 3-CN-1H-pyrazol-1-yl | Cl | 2,6-di-F-Ph | * |
| 82 | 2-Me-Bu | 1H-pyrrol-1-yl | Cl | 2,6-di-F-Ph | * |
| 83 | i-Bu | 1-Me-1H-imidazol-5-yl | Cl | 2,6-di-F-Ph | * |
| 84 | i-Bu | 4-pyridinyl | Cl | 2,6-di-F-Ph | * |
| 85 | i-Bu | 1-Me-1H-imidazol-4-yl | Cl | 2,6-di-F-Ph | 191-193 |
| (Ex. 11) | | | | | |
| 86 | i-Bu | 1H-pyrazol-1-yl | Cl | 2,4,6-tri-OMe-Ph | 166-171 |
| 87 | i-Bu | 1H-pyrazol-1-yl | Cl | 2-F-6-CF₃-Ph | * |
| 88 | i-Bu | 1H-pyrazol-1-yl | Cl | 3,4- | 178-182 |
| | | | | methylenedioxy-Ph | |
| 89 | i-Bu | 1H-pyrazol-1-yl | Cl | 5-Br-3,4- | 221-223 |
| | | | | methylenedioxy-Ph | |
| 90 | i-Bu | 1H-pyrazol-1-yl | Cl | 2-Naphthalenyl | * |
| 91 | CH₂-tetrahydrofuran- | 1H-pyrazol-1-yl | Cl | 2,6-di-F-Ph | * |
| | 2-yl | | | | |
| 92 | CH₂-tetrahydrofuran- | 1H-1,2,4-triazol-1-yl | Cl | 2,6-di-F-Ph | * |
| | 2-yl | | | | |
| 93 | 1-Me-2-OMe-Et | 1H-pyrazol-1-yl | Cl | 2,6-di-F-Ph | 165-168 |
| 94 | 1-Me-2-OMe-Et | 1H-1,2,4-triazol-1-yl | Cl | 2,6-di-F-Ph | 153-156 |
| 95 | 2-OMe-Et | 1H-pyrazol-1-yl | Cl | 2,6-di-F-Ph | 166-168 |
| 96 | 2-OMe-Et | 1H-1,2,4-triazol-1-yl | Cl | 2,6-di-F-Ph | 131-134 |
| 97 | 2-SMe-Et | 1H-pyrazol-1-yl | Cl | 2,6-di-F-Ph | 99-100 |
| 98 | 2-SMe-Et | 1H-1,2,4-triazol-1-yl | Cl | 2,6-di-F-Ph | 143-145 |
| 99 | i-Bu | 1H-pyrazol-1-yl | Br | 2,6-di-F-Ph | * |
| (Ex. 9) | | | | | |
| 100 | i-Bu | 1-Me-1H-pyrazol-4-yl | Cl | 2,6-di-F-Ph | * |
| 101 | i-Bu | 2-thiazolyl | Cl | 2,6-di-F-Ph | 120-124 |
| 102 | i-Bu | 1-Me-1H-imidazol-2-yl | Cl | 2,6-di-F-Ph | 220-224 |
| 103 | i-Bu | 1-Me-1H-pyrazol-5-yl | Cl | 2,6-di-F-Ph | * |
| 104 | benzyl | 1H-pyrazol-1-yl | Cl | 2,6-di-F-Ph | 155-156 |
| 105 | benzyl | 1H-1,2,4-triazol-1-yl | Cl | 2,6-di-F-Ph | 73-76 |
| 106 | benzyl | CN | Cl | 2,6-di-F-Ph | 125-130 |
| 107 | CH(Me)Ph | 1H-pyrazol-1-yl | Cl | 2,6-di-F-Ph | 170-172 |
| 108 | CH(Me)Ph | 1H-1,2,4-triazol-1-yl | Cl | 2,6-di-F-Ph | 197-200 |
| 109 | CH(Me)Ph | CN | Cl | 2,6-di-F-Ph | 193-197 |
| 110 | 2-Me-Bu | SCN | Cl | 2,6-di-F-Ph | * |
| 111 | i-Bu | 1H-pyrazol-1-yl | Cl | 2,4-di-Cl-Ph | * |
| 112 | i-Bu | 1H-1,2,4-triazol-1-yl | Cl | 2,4,6-tri-OMe-Ph | * |
| 113 | i-Bu | 1H-1,2,4-triazol-1-yl | Cl | 2-F-6-CF₃-Ph | * |
| 114 | i-Bu | 1H-1,2,4-triazol-1-yl | Cl | 3,4- | 188-194 |
| | | | | methylenedioxy-Ph | |
| 115 | i-Bu | 1H-1,2,4-triazol-1-yl | Cl | 5-Br-3,4- | 198-202 |
| | | | | methylenedioxy-Ph | |
| 116 | i-Bu | 1H-1,2,4-triazol-1-yl | Cl | 3,5-di-Cl-Ph | * |
| 117 | i-Bu | 1H-1,2,4-triazol-1-yl | Cl | 2-F-4-CF₃-Ph | 159-163 |
| 118 | i-Bu | 1H-1,2,4-triazol-1-yl | Cl | 2,4-di-Cl-Ph | 147-149 |
| 119 | i-Bu | 1H-1,2,4-triazol-1-yl | Cl | 2-Naphthanenyl | * |
| 120 | i-Bu | 1H-1,2,4-triazol-1-yl | Cl | 2,3,6-tri-Cl-Ph | 81-84 |
| 121 | i-Bu | 1H-pyrazol-1-yl | Cl | 2,3,6-tri-Cl-Ph | 73-78 |
| 122 | CH₂CO₂Me | 1H-pyrazol-1-yl | Cl | 2,6-di-F-Ph | 158-160 |
| 123 | CH₂CO₂Me | 1H-1,2,4-triazol-1-yl | Cl | 2,6-di-F-Ph | 72-76 |
| 124 | CH₂CH₂Cl | 1H-pyrazol-1-yl | Cl | 2,6-di-F-Ph | 196-197 |
| 125 | CH₂CH₂Cl | 1H-1,2,4-triazol-1-yl | Cl | 2,6-di-F-Ph | 212-215 |
| 126 | CH₂CH₂SO₂Me | 1H-pyrazol-1-yl | Cl | 2,6-di-F-Ph | 207-212 |
| 127 | CH₂CH₂SO₂Me | 1H-1,2,4-triazol-1-yl | Cl | 2,6-di-F-Ph | 186-190 |
| 128 | i-Bu | 1H-1,2,4-triazol-1-yl | Cl | CH₂Ph | * |
| 129 | i-Bu | 1H-1,2,4-triazol-1-yl | Cl | 1-Naphthanenyl | * |
| 130 | i-Bu | 1H-pyrazol-1-yl | Cl | CH₂Ph | * |
| 131 | i-Bu | 1H-pyrazol-1-yl | Cl | 1-Naphthanenyl | * |
| 132 | i-Bu | 1H-pyrazol-1-yl | Cl | CH(Me)Ph | * |
| 133 | i-Bu | 1H-1,2,4-triazol-1-yl | Cl | CH(Me)Ph | * |
| 134 | 2-Me-Bu | NHNMe₂ | Cl | 2,6-di-F-Ph | * |
| 135 | 2-Me-Bu | NH-N-Morpholinyl | Cl | 2,6-di-F-Ph | * |
| 136 | i-Bu | 1H-1,2,4-triazol-1-yl | Cl | 3,5-di-Me-Ph | * |
| 137 | i-Bu | 1H-pyrazol-1-yl | Cl | 3,5-di-Me-Ph | 129-13 1 |
| 138 | i-Bu | 1H-1,2,4-triazol-1-yl | Cl | 2,3,5-tri-F-Ph | * |
| 139 | i-Bu | 1H-pyrazol-1-yl | Cl | 2,3,5-tri-F-Ph | * |
| 140 | i-Bu | 1H-1,2,4-triazol-1-yl | Cl | 2-CF₃-Ph | * |
| 141 | i-Bu | 1H-pyrazol-1-yl | Cl | 2-CF₃-Ph | * |
| 142 | i-Bu | 1H-1,2,4-triazol-1-yl | Cl | 4-F-Ph | * |
| 143 | i-Bu | 1H-pyrazol-1-yl | Cl | 4-F-Ph | * |
| 144 | i-Bu | 1H-1,2,4-triazol-1-yl | Cl | 4-CN-Ph | * |
| 145 | i-Bu | 1H-pyrazol-1-yl | Cl | 4-CN-Ph | * |
| 146 | i-Bu | 1H-1,2,4-triazol-1-yl | Cl | 4-CO₂Me-Ph | * |
| 147 | i-Bu | 1H-pyrazol-1-yl | Cl | 4-CO₂Me-Ph | * |
| 148 | n-Bu | 1H-pyrazol-1-yl | Cl | 2,6-di-F-Ph | 127-128 |
| 149 | i-Bu | 1H-pyrazol-1-yl | Me | 2,6-di-F-Ph | 105-106 |
| (Ex. 9) | | | | | |
| 150 | i-Bu | 1H-pyrazol-1-yl | Cl | 3,5-bis-CF₃-Ph | * |
| 151 | i-Bu | 1H-pyrazol-1-yl | Cl | 5-Cl-2-furyl | 148-152 |
| 152 | i-Bu | 1H-pyrazol-1-yl | Cl | 5-Cl-2-thienyl | * |
| 153 | i-Bu | 1H-pyrazol-1-yl | Cl | 3-furyl | 123-126 |
| 154 | i-Bu | 1H-pyrazol-1-yl | Cl | 3-thienyl | 117-119 |
| 155 | (2S)-Me-Bu | 1H-pyrazol-1-yl | Cl | 2,4,6-tri-F-Ph | 112-113 |
| 156 | i-Bu | 1H-1,2,4-triazol-1-yl | Cl | 5-Cl-2-furyl | 145-146 |
| 157 | i-Bu | 1H-1,2,4-triazol-1-yl | Cl | 5-Cl-2-thienyl | 133-135 |
| 158 | i-Bu | 1H-1,2,4-triazol-1-yl | Cl | 3-furyl | 134-138 |
| 159 | i-Bu | 1H-1,2,4-triazol-1-yl | Cl | 3-thienyl | 144-148 |
| 160 | i-Bu | 1H-1,2,4-triazol-1-yl | Cl | 3-thienyl | 156-158 |
| 161 | Ph | 1H-pyrazol-1-yl | Cl | 2,6-di-F-Ph | * |
| 162 | Ph | 1H-1,2,4-triazol-1-yl | Cl | 2,6-di-F-Ph | 220-225 |
| 163 | Ph | CN | Cl | 2,6-di-F-Ph | 193-198 |
| 164 | 2-Me-Bu | 1H-pyrazol-1-yl | Cl | 2-Cl-4-F-Ph | * |
| 165 | 2-Me-Bu | 1H-1,2,4-triazol-1-yl | Cl | 2-Cl-4-F-Ph | * |
| 166 | 2-Me-Bu | 4-Cl-1H-pyrazol-1-yl | Cl | 2-Cl-4-F-Ph | * |
| 167 | 2-Me-Bu | 3-CF₃-1H-pyrazol-1-yl | Cl | 2-Cl-4-F-Ph | 116-119 |
| 168 | 2-Me-Bu | 3,5-di-Me-1H-pyrazol-1-yl | Cl | 2-Cl-4-F-Ph | 139-142 |
| 169 | 2-Me-Bu | 4-Me-1H-pyrazol-1-yl | Cl | 2-Cl-4-F-Ph | * |
| 170 | 2-Me-Bu | 3-Me-1H-pyrazol-1-yl | Cl | 2-Cl-4-F-Ph | * |
| 171 | i-Bu | 1H-pyrazol-1-yl | Cl | 2,3,4,5,6-penta-F- | 136-138 |
| | | | | Ph | |
| 172 | i-Bu | 1H-pyrazol-1-yl | Cl | Ph | * |
| 173 | i-Bu | 1H-1,2,4-triazol-1-yl | Cl | Ph | * |
| 174 | i-Bu | 1H-pyrazol-1-yl | Cl | 2-F-Ph | * |
| 175 | i-Bu | 1H-1,2,4-triazol-1-yl | Cl | 2-F-Ph | * |
| 176 | i-Bu | 1H-pyrazol-1-yl | Cl | 2-OMe-Ph | * |
| 177 | i-Bu | 1H-1,2,4-triazol-1-yl | Cl | 2-OMe-Ph | * |
| 178 | i-Bu | 3-pyridinyl | Cl | 2,6-di-F-Ph | 175-178 |
| 179 | 2-OMe-Et | 3-CF₃-1H-imidazol-1-yl | Cl | 2,6-di-F-Ph | * |
| 180 | 1-Me-Bu | 1H-pyrazol-1-yl | Cl | 2,6-di-F-Ph | * |
| 181 | 1-Et-Pr | 1H-pyrazol-1-yl | Cl | 2,6-di-F-Ph | * |
| 182 | 3,3-di-Me-Bu | 1H-pyrazol-1-yl | Cl | 2,6-di-F-Ph | * |
| 183 | 2-Et-Bu | 1H-pyrazol-1-yl | Cl | 2,6-di-F-Ph | * |
| 184 | Et | 1H-pyrazol-1-yl | Cl | 2,6-di-F-Ph | * |
| 185 | Me | 1H-pyrazol-1-yl | Cl | 2,6-di-F-Ph | * |
| 186 | 2-propynyl | 1H-pyrazol-1-yl | Cl | 2,6-di-F-Ph | * |
| 187 | CH₂-2-Furyl | 1H-pyrazol-1-yl | Cl | 2,6-di-F-Ph | 200-204 |
| 188 | CH₂-2-Furyl | 1H-1,2,4-triazol-1-yl | Cl | 2,6-di-F-Ph | 188-190 |
| 189 | CH₂-2-Furyl | CN | Cl | 2,6-di-F-Ph | 138-140 |
| 190 | i-Bu | imidazo[1,2-a]pyridin-3-yl | Cl | 2,6-di-F-Ph | 194-197 |
| 191 | 2-Me-Bu | 1H-1,2,3-triazol-2-yl | Cl | 2,6-di-F-Ph | * |
| 192 | i-Bu | 1H-1,2,4-triazol-1-yl | Cl | 2-Cl-Ph | * |
| 193 | i-Bu | 1H-pyrazol-1-yl | Cl | 2-Cl-Ph | * |
| 194 | i-Bu | 1H-1,2,4-triazol-1-yl | Cl | 2-Br-Ph | * |
| 195 | i-Bu | 1H-pyrazol-1-yl | Cl | 2-Br-Ph | * |
| 196 | i-Bu | 1H-1,2,4-triazol-1-yl | Cl | 2-Et-Ph | * |
| 197 | i-Bu | 1H-pyrazol-1-yl | Cl | 2-Et-Ph | * |
| 198 | 1-Me-2-SMe-Et | 1H-pyrazol-1-yl | Cl | 2,6-di-F-Ph | 126-127 |
| 199 | 1-Me-2-SMe-Et | 1H-1,2,4-triazol-1-yl | Cl | 2,6-di-F-Ph | 192-194 |
| 200 | 1-Me-2-SO₂Me-Et | 1H-pyrazol-1-yl | Cl | 2,6-di-F-Ph | 232-236 |
| 201 | 1-Me-2-SO₂Me-Et | 1H-1,2,4-triazol-1-yl | Cl | 2,6-di-F-Ph | 209-211 |
| 202 | i-Bu | 1H-pyrazol-1-yl | Cl | 2,6-di-Me-Ph | * |
| 203 | i-Bu | 1H-pyrazol-1-yl | Cl | t-Bu | 119-122 |
| 204 | i-Bu | 1H-pyrazol-1-yl | Cl | i-Pr | 87-89 |
| 205 | i-Bu | 1H-1,2,4-triazol-1-yl | Cl | 2,6-di-Me-Ph | 148-151 |
| 206 | i-Bu | 1H-1,2,4-triazol-1-yl | Cl | t-Bu | * |
| 207 | i-Bu | 1H-1,2,4-triazol-1-yl | Cl | i-Pr | 102-105 |
| 208 | i-Bu | 4-Me-2-pyridinyl | Cl | 2,6-di-F-Ph | * |
| 209 | i-Bu | 5-Me-2-pyridinyl | Cl | 2,6-di-F-Ph | * |
| (Ex. 12) | | | | | |
| 210 | i-Bu | 3-Me-2-pyridinyl | Cl | 2,6-di-F-Ph | * |
| 211 | 1-Me-2-OMe-ethenyl | 1H-pyrazol-1-yl | Cl | 2,6-di-F-Ph | 210-215 |
| 212 | 1-Me-2-OMe-ethenyl | 1H-1,2,4-triazol-1-yl | Cl | 2,6-di-F-Ph | 152-156 |
| 213 | 1-Me-2-OH-Et | 1H-pyrazol-1-yl | Cl | 2,6-di-F-Ph | 229-234 |
| 214 | 1-Me-2-Cl-Et | 1H-1,2,4-triazol-1-yl | Cl | 2,6-di-F-Ph | 202-205 |
| 215 | i-Bu | 1H-1,2,3-triazol-1-yl | Cl | 2,6-di-F-Ph | * |
| 216 | 2-Me-Bu | 4,5-dihydro-1H-pyrazol-1-yl | Cl | 2-Cl-4-F-Ph | * |
| 217 | 2-Me-Bu | 4,5-dihydro-1H-pyrazol-3- | Cl | 2-Cl-4-F-Ph | 154-156 |
| | | one-1-yl | | | |
| 218 | (2S)-Me-Bu | 4,5-dihydro-1H-pyrazol-1-yl | Cl | 2,4,6-tri-F-Ph | * |
| 219 | (2S)-Me-Bu | 3-Me-1H-pyrazol-1-yl | Cl | 2,4,6-tri-F-Ph | * |
| 220 | 4-Cl-benzyl | CN | Cl | 2,6-di-F-Ph | 140-145 |
| 221 | 4-Cl-benzyl | 1H-pyrazol-1-yl | Cl | 2,6-di-F-Ph | 205 |
| 222 | 4-Cl-benzyl | 1H-1,2,4-triazol-1-yl | Cl | 2,6-di-F-Ph | 160-165 |
| 223 | 2-Me-c-Hex | CN | Cl | 2,6-di-F-Ph | 135-140 |
| 224 | 2-Me-c-Hex | 1H-pyrazol-1-yl | Cl | 2,6-di-F-Ph | 204-208 |
| 225 | 2-Me-c-Hex | 1H-1,2,4-triazol-1-yl | Cl | 2,6-di-F-Ph | 155-160 |
| 226 | n-Bu | 4,5-dihydro-1H-pyrazol-1-yl | Cl | 2,4,6-tri-F-Ph | * |
| 227 | n-Bu | 1H-pyrazol-1-yl | Cl | 2,4,6-tri-F-Ph | 128-129 |
| 228 | n-Bu | 3-Me-1H-pyrazol-1-yl | Cl | 2,4,6-tri-F-Ph | 123-124 |
| 229 | n-Bu | 1H-1,2,4-triazol-1-yl | Cl | 2,4,6-tri-F-Ph | 134-135 |
| 230 | i-Bu | 1H-1,2,4-triazol-1-yl | Cl | 3-F-Ph | * |
| 231 | i-Bu | 1H-pyrazol-1-yl | Cl | 3-F-Ph | * |
| 232 | i-Bu | 1H-1,2,4-triazol-1-yl | Cl | 3-CF₃-Ph | * |
| 233 | i-Bu | 1H-pyrazol-1-yl | Cl | 3-CF₃-Ph | * |
| 234 | i-Bu | 1H-1,2,4-triazol-1-yl | Cl | 3-Br-Ph | * |
| 235 | i-Bu | 1H-1,2,4-triazol-1-yl | Cl | 3-pyridinyl | * |
| 236 | i-Bu | 1H-1,2,4-triazol-1-yl | Cl | 2-Cl-3-pyridinyl | * |
| 237 | i-Bu | 1H-pyrazol-1-yl | Cl | 3-pyridinyl | * |
| 238 | 1-Me-2-F-Et | 1H-pyrazol-1-yl | Cl | 2,6-di-F-Ph 214-216 | |
| 239 | n-Bu | 5-Me-1H-pyrazol-1-yl | Cl | 2,4,6-tri-F-Ph | * |
| 240 | n-Bu | 2-pyridinyl | Cl | 2,4,6-tri-F-Ph | 134-135 |
| 241 | i-Bu | 3-Me-1H-pyrazol-1-yl | Cl | 2,6-di-F-Ph | 135-136 |
| 242 | i-Bu | 3-Me-1H-pyrazol-1-yl | Br | 2,6-di-F-Ph | 131-132 |
| 243 | i-Bu | 3-Me-1H-pyrazol-1-yl | Me | 2,6-di-F-Ph | 113-114 |
| 244 | i-Bu | 1H-pyrazol-1-yl | Cl | 2-Cl-3-pyridinyl | * |
| 245 | i-Bu | 1H-1,2,4-triazol-1-yl | Cl | 3-OMe-Ph | * |
| 246 | i-Bu | 1H-pyrazol-1-yl | Cl | 3-OMe-Ph | * |
| 247 | i-Bu | 1H-1,2,4-triazol-1-yl | Cl | 3-Me-Ph | * |
| 248 | i-Bu | 1H-pyrazol-1-yl | Cl | 3-Me-Ph | * |
| 249 | i-Bu | 1H-1,2,4-triazol-1-yl | Cl | 3-CN-Ph | * |
| 250 | i-Bu | 1H-pyrazol-1-yl | Cl | 3-CN-Ph | * |
| 251 | i-Bu | 1H-pyrazol-1-yl | Cl | 2,3- | * |
| | | | | methylenedioxy-Ph | |
| 252 | i-Bu | 1H-pyrazol-1-yl | Cl | 2,2-difluoro- | 151-154 |
| | | | | benzodioxol-6-yl | |
| 253 | i-Bu | 1H-pyrazol-1-yl | Cl | 2-Cl-4,5- | 207-210 |
| | | | | methylenedioxy-Ph | |
| 254 | i-Bu | 1H-pyrazol-1-yl | Cl | 2,4,6-tri-Me-Ph | 144-146 |
| 255 | i-Bu | 3-Me-1H-pyrazol-1-yl | Cl | 2,3- | 145-146 |
| | | | | methylenedioxy-Ph | |
| 256 | i-Bu | 3-Me-1H-pyrazol-1-yl | Cl | 2,2-difluoro- | 164-165 |
| | | | | benzodioxol-6-yl | |
| 257 | i-Bu | 3-Me-1H-pyrazol-1-yl | Cl | 2-Cl-4,5- | 201-204 |
| | | | | methylenedioxy-Ph | |
| 258 | i-Bu | 3-Me-1H-pyrazol-1-yl | Cl | 2,4,6-tri-Me-Ph | * |
| 259 | 2-Me-Bu | 3-Br-1H-pyrazol-1-yl | Cl | 2-Cl-4-F-Ph | * |
| 260 | 2-Me-Bu | 3-Me-4-Br-1H-pyrazol-1-yl | Cl | 2-Cl-4-F-Ph | * |
| 261 | 2-Me-Bu | 3-(MeO)₂CH-1H-pyrazol-1- | Cl | 2-Cl-4-F-Ph | 128-132 |
| | | yl | | | |
| 262 | (2S)-Me-Bu | 3-Me-1H-pyrazol-1-yl | Cl | 2-Cl-4-F-Ph | * |
| 263 | (2S)-Me-Bu | 1H-pyrazol-1-yl | Cl | 2-Cl-4-F-Ph | * |
| 264 | (2S)-Me-Bu | 4,5-dihydro-1H-pyrazol-1-yl | Cl | 2-Cl-4-F-Ph | * |
| 265 | n-Bu | 3-Me-1H-pyrazol-1-yl | Cl | 2-Cl-4-F-Ph | * |
| 266 | n-Bu | 1H-pyrazol-1-yl | Cl | 2-Cl-4-F-Ph | 134-137 |
| 267 | n-Bu | 4,5-dihydro-1H-pyrazol-1-yl | Cl | 2-Cl-4-F-Ph | * |
| 268 | 2-OH-Et | 1H-pyrazol-1-yl | Cl | 2,6-di-F-Ph | 229-232 |
| 269 | 2-F-Et | 1H-pyrazol-1-yl | Cl | 2,6-di-F-Ph | 194-196 |
| 270 | 2-Br-Et | 1H-pyrazol-1-yl | Cl | 2,6-di-F-Ph | 211-212 |
| 271 | 4-OMe-benzyl | 1H-pyrazol-1-yl | Cl | 2,6-di-F-Ph | 235-236 |
| (Ex. 4) | | | | | |
| 272 | 4-F-benzyl | 1H-pyrazol-1-yl | Cl | 2,6-di-F-Ph | 151-154 |
| 273 | 4-F-benzyl | 1H-1,2,4-triazol-1-yl | Cl | 2,6-di-F-Ph | 92-97 |
| 274 | 4-F-benzyl | CN | Cl | 2,6-di-F-Ph | * |
| 275 | 3-Cl-benzyl | 1H-pyrazol-1-yl | Cl | 2,6-di-F-Ph | 174-175 |
| 276 | 3-Cl-benzyl | 1H-1,2,4-triazol-1-yl | Cl | 2,6-di-F-Ph | 143-145 |
| 277 | 3-Cl-benzyl | CN | Cl | 2,6-di-F-Ph | 134-136 |
| 278 | Ph | 3-Me-1H-pyrazol-1-yl | Cl | 2,6-di-F-Ph | 184-185 |
| 279 | benzyl | 3-Me-1H-pyrazol-1-yl | Cl | 2,6-di-F-Ph | * |
| 280 | 2-F-benzyl | 3-Me-1H-pyrazol-1-yl | Cl | 2,6-di-F-Ph | * |
| 281 | (2S)-Me-Bu | 1H-pyrazol-1-yl | Br | 2-Cl-4-F-Ph | * |
| 282 | (2S)-Me-Bu | 3-Me-1H-pyrazol-1-yl | Br | 2-Cl-4-F-Ph | * |
| 283 | (2S)-Me-Bu | 4,5-dihydro-1H-pyrazol-1-yl | Br | 2-Cl-4-F-Ph | * |
| 284 | n-Bu | 1H-pyrazol-1-yl | Br | 2-Cl-4-F-Ph | 130-132 |
| 285 | n-Bu | 3-Me-1H-pyrazol-1-yl | Br | 2-Cl-4-F-Ph | * |
| 286 | n-Bu | 4,5-dihydro-1H-pyrazol-1-yl | Br | 2-Cl-4-F-Ph | * |
| 287 | Ph | 1H-pyrazol-1-yl | Cl | 2,4,6-tri-F-Ph | 180-182 |
| 288 | benzyl | 1H-pyrazol-1-yl | Cl | 2-Cl-Ph | 123-126 |
| 289 | i-Bu | 3-Me-1H-pyrazol-1-yl | Cl | i-Pr | 147-149 |
| 290 | i-Bu | 1H-pyrazol-1-yl | Cl | c-Pentyl | * |
| 291 | i-Bu | 1H-pyrazol-1-yl | Cl | c-Hex | 112-114 |
| 292 | i-Bu | 1H-pyrazol-1-yl | Cl | i-Bu | * |
| 293 | i-Bu | 3-Me-1H-pyrazol-1-yl | Cl | c-Pentyl | 154-159 |
| 294 | i-Bu | 3-Me-1H-pyrazol-1-yl | Cl | c-Hex | 155-159 |
| 295 | i-Bu | 3-Me-1H-pyrazol-1-yl | Cl | i-Bu | 113-114 |
| 296 | 2-Me-Bu | CONH-i-Pr | Cl | 2,6-di-F-Ph | * |
| 297 | 2-Me-Bu | CONHMe | Cl | 2,6-di-F-Ph | * |
| 298 | 2-Me-Bu | CONH2 | Cl | 2,6-di-F-Ph | * |
| 299 | 2-Me-Bu | CONH-n-Pr | Cl | 2,6-di-F-Ph | * |
| 300 | 2-Me-Bu | CONHCH₂-c-Pr | Cl | 2,6-di-F-Ph | * |
| 301 | 2-Me-Bu | CONH-2-Me-Bu | Cl | 2,6-di-F-Ph | * |
| 302 | i-Bu | 1H-pyrazol-1-yl | Cl | 2˜Cl˜4˜F˜Pha,c | * |
| (Ex. 7) | | | | | |
| 303 | i-Bu | 1H-pyrazol-1-yl | Cl | 2-Cl-4-F-Ph^{b,c} | * |
| (Ex. 7) | | | | | |
| 304 | benzyl | 1H-pyrazol-1-yl | Cl | i-Pr | 117-119 |
| 305 | benzyl | 3-Me-1H-pyrazol-1-yl | Cl | i-Pr | 95-99 |
| 306 | Ph | 1H-pyrazol-1-yl | Cl | i-Pr | 190-193 |
| 307 | Ph | 3-Me-1H-pyrazol-1-yl | Cl | i-Pr | 148-151 |
| 308 | CH₂CO₂Me | 3-Me-1H-pyrazol-1-yl | Cl | 2,6-di-F-Ph | 147-149 |
| 309 | 1-Me-2-SMe-Et | 3-Me-1H-pyrazol-1-yl | Cl | 2,6-di-F-Ph | 127-129 |
| 310 | 2-OMe-Et | 3-Me-1H-pyrazol-1-yl | Cl | 2,6-di-F-Ph | 135-138 |
| 311 | 2-SMe-Et | 3-Me-1H-pyrazol-1-yl | Cl | 2,6-di-F-Ph | 145-146 |
| 312 | 1-Me-2-OMe-Et | 2-CN-1H-pyrrol-1-yl | Cl | 2,6-di-F-Ph | 139-142 |
| 313 | CH₂CO₂Me | 3-Me-4-1H-pyrazol-1-yl | Cl | 2,6-di-F-Ph | 69-72 |
| 314 | i-Bu | 1-pyrrolidinyl | Cl | 2,6-di-F-Ph | * |
| 315 | i-Bu | 1H-pyrazol-1-yl | Cl | c-Pr | * |
| 316 | i-Bu | 1H-pyrazol-1-yl | Cl | Et | * |
| 317 | i-Bu | 1H-pyrazol-1-yl | Cl | n-Pentyl | * |
| 318 | i-Bu | 1H-pyrazol-1-yl | Cl | 2,4,6-tri-Cl-Ph | * |
| 319 | i-Bu | 4,5-dihydro-1H-pyrazol-3- | Cl | 2,4,6-tri-Cl-Ph | * |
| | | one-1-yl | | | |
| 320 | 2-F-Ph | 1H-pyrazol-1-yl | Cl | 2,4,6-tri-F-Ph | * |
| 321 | Ph | 3-Me-1H-pyrazol-1-yl | Cl | 2,4,6-tri-F-Ph | 200-202 |
| 322 | Ph | 1H-pyrazol-1-yl | Cl | 2-Cl-4-F-Ph | 123-125 |
| 323 | Ph | 3-Me-1H-pyrazol-1-yl | Cl | 2-Cl-4-F-Ph | 128-129 |
| 324 | 3-F-Ph | 1H-pyrazol-1-yl | Cl | 2,4,6-tri-F-Ph | * |
| 325 | i-Bu | 2-CN-1H-pyrrol-1-yl | Cl | 2,6-di-F-Ph | 156-157 |
| 326 | 3-Me-Ph | 1H-pyrazol-1-yl | Cl | 2,6-di-F-Ph | 127-129 |
| 327 | 3-Me-Ph | 3-Me-1H-pyrazol-1-yl | Cl | 2,6-di-F-Ph | 75-79 |
| 328 | 2-Me-Ph | 1H-pyrazol-1-yl | Cl | 2,6-di-F-Ph | 193-197 |
| 329 | 2-Me-Ph | 3-Me-1H-pyrazol-1-yl | Cl | 2,6-di-F-Ph | 175-176 |
| 330 | 2-F-benzyl | 1H-pyrazol-1-yl | Cl | 2,6-di-F-Ph | 143-145 |
| 331 | 4-F-benzyl | 3-Me-1H-pyrazol-1-yl | Cl | 2,6-di-F-Ph | 153-155 |
| 332 | 4-Me-Ph | 3-Me-1H-pyrazol-1-yl | Cl | 2,6-di-F-Ph | 234-236 |
| 333 | 4-Me-Ph | 1H-pyrazol-1-yl | Cl | 2,6-di-F-Ph | 219-222 |
| 334 | 2,4-di-Me-Ph | 1H-pyrazol-1-yl | Cl | 2,6-di-F-Ph | 203-206 |
| 335 | 2,4-di-Me-Ph | 3-Me-1H-pyrazol-1-yl | Cl | 2,6-di-F-Ph | 218-221 |
| 336 | 2-F-benzyl | 1H-pyrazol-1-yl | Cl | 2,6-di-F-Ph | 163-165 |
| 337 | 3-F-Ph | 1H-pyrazol-1-yl | Cl | 2,6-di-F-Ph | 235-240 |
| 338 | 3-F-Ph | 3-Me-1H-pyrazol-1-yl | Cl | 2,6-di-F-Ph | 194-196 |
| 339 | 2-F-Ph | 1H-pyrazol-1-yl | Cl | 2,6-di-F-Ph | 200-220 |
| 340 | 2-F-Ph | 3-Me-1H-pyrazol-1-yl | Cl | 2,6-di-F-Ph | 207-208 |
| 341 | 4-F-benzyl | 3-Me-1H-pyrazol-1-yl | Cl | 2,6-di-F-Ph | * |
| 342 | i-Bu | 1H-pyrazol-1-yl | H | 2,6-di-F-Ph | 91-92 |
| (Ex. 3) | | | | | |
| 343 | i-Bu | 2-pyridinyl | Cl | 2,6-di-F-Ph | HCl Salt |
| 344 | 3-Br-benzyl | 1H-pyrazol-1-yl | Cl | 2,4,6-tri-F-Ph | * |
| 345 | 2-Br-benzyl | 1H-pyrazol-1-yl | Cl | 2,4,6-tri-F-Ph | * |
| 346 | 4-CF₃-benzyl | 1H-pyrazol-1-yl | Cl | 2,4,6-tri-F-Ph | * |
| 347 | 3-CF₃-benzyl | 1H-pyrazol-1-yl | Cl | 2,4,6-tri-F-Ph | * |
| 348 | 2-CF₃-benzyl | 1H-pyrazol-1-yl | Cl | 2,4,6-tri-F-Ph | * |
| 349 | 2-Me-Bu | CONHCH₂CH₂OH | Cl | 2,6-di-F-Ph | * |
| 350 | i-Bu | 2-pyrazinyl | Cl | 2,6-di-F-Ph | 102-102 |
| 351 | 3-Br-Ph | 1H-pyrazol-1-yl | Cl | 2,4,6-tri-F-Ph | * |
| 352 | 4-Br-Ph | 1H-pyrazol-1-yl | Cl | 2,4,6-tri-F-Ph | * |
| 353 | 3-CF₃-Ph | 1H-pyrazol-1-yl | Cl | 2,4,6-tri-F-Ph | * |
| 354 | 4-CF₃-Ph | 1H-pyrazol-1-yl | Cl | 2,4,6-tri-F-Ph | * |
| 355 | 2-F-Ph | 3-Me-1H-pyrazol-1-yl | Cl | 2,4,6-tri-F-Ph | 192-194 |
| 356 | 3-F-Ph | 3-Me-1H-pyrazol-1-yl | Cl | 2,4,6-tri-F-Ph | 183-185 |
| 357 | Ph | 1H-pyrazol-1-yl | Cl | 2,3,6-tri-F-Ph | 250-260 |
| 358 | Ph | 3-Me-1H-pyrazol-1-yl | Cl | 2,3,6-tri-F-Ph | 193-194 |
| 359 | Ph | 2-pyridinyl | Me | 2,6-di-F-Ph | * |
| 360 | 2-Br-Ph | 1H-pyrazol-1-yl | Cl | 2,6-di-F-Ph | * |
| 361 | c-Pr | 1H-pyrazol-1-yl | Cl | 2,6-di-F-Ph | * |
| 362 | 2-Ph-c-Pr | 1H-pyrazol-1-yl | Cl | 2,6-di-F-Ph | * |
| 363 | c-Bu | 1H-pyrazol-1-yl | Cl | 2,6-di-F-Ph | * |
| 364 | i-Bu | 2-pyrazinyl | Cl | 2,6-di-F-Ph | HCl Salt |
| 365 | 3,4-di-F-Ph | 1H-pyrazol-1-yl | Cl | 2,4,6-tri-F-Ph | 188-190 |
| 366 | 3,4-di-F-Ph | 3-Me-1H-pyrazol-1-yl | Cl | 2,4,6-tri-F-Ph | 198-200 |
| 367 | 3,5-di-F-Ph | 1H-pyrazol-1-yl | Cl | 2,4,6-tri-F-Ph | 220-221 |
| 368 | 3,5-di-F-Ph | 3-Me-1H-pyrazol-1-yl | Cl | 2,4,6-tri-F-Ph | 221-223 |
| 369 | 2,3-di-F-Ph | 1H-pyrazol-1-yl | Cl | 2,4,6-tri-F-Ph | 174-176 |
| 370 | 2,3-di-F-Ph | 3-Me-1H-pyrazol-1-yl | Cl | 2,4,6-tri-F-Ph | 210-211 |
| 371 | 4-Cl-Ph | 1H-pyrazol-1-yl | Cl | 2,4,6-tri-F-Ph | 205-206 |
| 372 | 4-Cl-Ph | 3-Me-1H-pyrazol-1-yl | Cl | 2,4,6-tri-F-Ph | 188-191 |
| 373 | 3-Cl-Ph | 1H-pyrazol-1-yl | Cl | 2,4,6-tri-F-Ph | 170-173 |
| 374 | 3-Cl-Ph | 3-Me-1H-pyrazol-1-yl | Cl | 2,4,6-tri-F-Ph | 178-179 |
| 375 | 2-Cl-Ph | 1H-pyrazol-1-yl | Cl | 2,4,6-tri-F-Ph | 188-190 |
| 376 | 2-Cl-Ph | 3-Me-1H-pyrazol-1-yl | Cl | 2,4,6-tri-F-Ph | 215-217 |
| 377 | 4-Br-benzyl | 1H-pyrazol-1-yl | Cl | 2,4,6-tri-F-Ph | * |
| 378 | 2,3-di-Me-Ph | 1H-pyrazol-1-yl | Cl | 2,6-di-F-Ph | 177-179 |
| 379 | 2,3-di-Me-Ph | 3-Me-1H-pyrazol-1-yl | Cl | 2,6-di-F-Ph | 214-215 |
| 380 | 2,3-di-F-Ph | 1H-pyrazol-1-yl | Cl | 2,6-di-F-Ph | 218-219 |
| 381 | 2,3-di-F-Ph | 3-Me-1H-pyrazol-1-yl | Cl | 2,6-di-F-Ph | 193-194 |
| 382 | 4-F-Ph | 1H-pyrazol-1-yl | Cl | 2,6-di-F-Ph | 195-196 |
| 383 | 4-F-Ph | 3-Me-1H-pyrazol-1-yl | Cl | 2,6-di-F-Ph | 198-199 |
| 384 | 3,5-di-F-Ph | 1H-pyrazol-1-yl | Cl | 2,6-di-F-Ph | 233-235 |
| 385 | 3,5-di-F-Ph | 3-Me-1H-pyrazol-1-yl | Cl | 2,6-di-F-Ph | 210-211 |
| 386 | 2,4-di-F-Ph | 1H-pyrazol-1-yl | Cl | 2,6-di-F-Ph | 111-113 |
| 387 | 2,4-di-F-Ph | 3-Me-1H-pyrazol-1-yl | Cl | 2,6-di-F-Ph | 178-179 |
| 388 | 3,4-di-F-Ph | 1H-pyrazol-1-yl | Cl | 2,6-di-F-Ph | 233-235 |
| 389 | 3,4-di-F-Ph | 3-Me-1H-pyrazol-1-yl | Cl | 2,6-di-F-Ph | 205-206 |
| 390 | i-Bu | 3-Me-1H-pyrazol-1-yl | Cl | s-Bu | * |
| 391 | i-Bu | 1H-pyrazol-1-yl | Cl | 2,6-di-Cl-4-CF₃-Ph | * |
| 392 | N═C(Me)₂ | 1H-pyrazol-1-yl | Cl | 2,6-di-F-Ph | * |
| (Ex. 4) | | | | | |
| 393 | 2-OMe-Ph | 1H-pyrazol-1-yl | Cl | 2,6-di-F-Ph | * |
| 394 | 3-OMe-Ph | 1H-pyrazol-1-yl | Cl | 2,6-di-F-Ph | * |
| 395 | 4-OMe-Ph | 1H-pyrazol-1-yl | Cl | 2,6-di-F-Ph | * |
| 396 | 2-OCF₃-Ph | 1H-pyrazol-1-yl | Cl | 2,6-di-F-Ph | * |
| 397 | 2-OCF₃-Ph | 1H-pyrazol-1-yl | Cl | 2,6-di-F-Ph | * |
| 398 | 4-OCF₃-Ph | 1H-pyrazol-1-yl | Cl | 2,6-di-F-Ph | * |
| 399 | 2-CF₃-Ph | 1H-pyrazol-1-yl | Cl | 2,6-di-F-Ph | * |
| 400 | NH2 | 1H-pyrazol-1-yl | Cl | 2,6-di-F-Ph | * |
| (Ex. 4) | | | | | |
| 401 | i-Bu | 2-pyridinyl | I | 2,6-di-F-Ph | * |
| 402 | 3,4-di-Cl-Ph | 1H-pyrazol-1-yl | Cl | 2,4,6-tri-F-Ph | 219-220 |
| 403 | 3,4-di-Cl-Ph | 3-Me-1H-pyrazol-1-yl | Cl | 2,4,6-tri-F-Ph | 218-220 |
| 404 | 3,5-di-Cl-Ph | 1H-pyrazol-1-yl | Cl | 2,4,6-tri-F-Ph | 210-211 |
| 405 | 3,5-di-Cl-Ph | 3-Me-1H-pyrazol-1-yl | Cl | 2,4,6-tri-F-Ph | 208-210 |
| 406 | 3-Cl-4-F-Ph | 1H-pyrazol-1-yl | Cl | 2,4,6-tri-F-Ph | 198-200 |
| 407 | 3-Cl-4-F-Ph | 3-Me-1H-pyrazol-1-yl | Cl | 2,4,6-tri-F-Ph | 118-121 |
| 408 | 4-F-Ph | 1H-pyrazol-1-yl | Cl | 2,4,6-tri-F-Ph | 150-152 |
| 409 | 4-F-Ph | 3-Me-1H-pyrazol-1-yl | Cl | 2,4,6-tri-F-Ph | 188-192 |
| 410 | 3,4,5-tri-F-Ph | 1H-pyrazol-1-yl | Cl | 2,4,6-tri-F-Ph | 238-240 |
| 411 | 3,4,5-tri-F-Ph | 3-Me-1H-pyrazol-1-yl | Cl | 2,4,6-tri-F-Ph | 190-193 |
| 412 | Et | CONH2 | Cl | 2-Cl-4-F-Ph | * |
| 413 | n-Pr | CONH2 | Cl | 2,4,6-tri-F-Ph | * |
| 414 | 3-F-Ph | CONH2 | Cl | 2,4,6-tri-F-Ph | * |
| (Ex. 8) | | | | | |
| 415 | 2-Me-Bu | CONH2 | Cl | 2,4,6-tri-F-Ph | * |
| 416 | 2-Me-Bu | CONH-Propargyl | Cl | 2,4,6-tri-F-Ph | * |
| 417 | 2-Me-Bu | CONHOMe | Cl | 2,4,6-tri-F-Ph | * |
| 418 | c-Hex | 1H-pyrazol-1-yl | Cl | 2,4,6-tri-F-Ph | * |
| 419 | Ph | CONH2 | Cl | 2,3,6-tri-F-Ph | * |
| 420 | Ph | CONH2 | Cl | 2,4,6-tri-F-Ph | * |
| 421 | 2-Me-Bu | 1H-pyrazol-1-yl | Cl | 2-F-4-OMe-Ph | * |
| 422 | i-Bu | NHC(═O)H | Cl | 2,6-di-F-Ph | * |
| (Ex. 13) | | | | | |
| 423 | i-Bu | NH(C═O)CH3 | Cl | 2,6-di-F-Ph | * |
| 424 | i-Bu | 1H-pyrazol-1-yl | Cl | s-Bu | * |
| (Ex. 5) | | | | | |
| 425 | 2-Me-Bu | 3-Me-1H-pyrazol-1-yl | Cl | 2-F-4-OMe-Ph | * |
| 426 | 2-Me-Bu | 1H-pyrazol-1-yl | Cl | 2-F-4-OH-Ph | * |
| 427 | 3-F-Ph | 3-CF₃-1H-pyrazol-1-yl | Cl | 2,4,6-tri-F-Ph | * |
| 428 | 3-F-Ph | 3-t-Bu-1H-pyrazol-1-yl | Cl | 2,4,6-tri-F-Ph | * |
| 429 | 3-F-Ph | 3-Br-1H-pyrazol-1-yl | Cl | 2,4,6-tri-F-Ph | * |
| 430 | 3-F-Ph | 3-CN-1H-pyrazol-1-yl | Cl | 2,4,6-tri-F-Ph | * |
| 431 | 3-F-Ph | pyrrolo-1-yl | Cl | 2,4,6-tri-F-Ph | * |
| 432 | 3-F-Ph | imidazol-1-yl | Cl | 2,4,6-tri-F-Ph | * |
| 433 | 3-F-Ph | 1H-1,2,4-triazol-1-yl | Cl | 2,4,6-tri-F-Ph | * |
| 434 | 3-F-Ph | 4,5-dihydro-1H-pyrazol-1-yl | Cl | 2,4,6-tri-F-Ph | * |
| 435 | c-hexyl | 3-Me-1H-pyrazol-1-yl | Cl | 2,6-di-F-Ph | * |
| 436 | c-hexyl | 3-Me-1H-pyrazol-1-yl | Cl | 2,4,6-tri-F-Ph | * |
| 437 | 3-MeO-propyl | 1H-pyrazol-1-yl | Cl | 2,4,6-tri-F-Ph | 128-129 |
| 438 | 3-OH-propyl | 1H-pyrazol-1-yl | Cl | 2,4,6-tri-F-Ph | 232-233 |
| 439 | 3-Br-propyl | 1H-pyrazol-1-yl | Cl | 2,4,6-tri-F-Ph | 132-133 |
| 440 | 2-Me-Bu | CONH2 | Cl | 2,3,5-tri-F-Ph | * |
| 441 | 2-Me-Bu | CONH2 | Cl | 2,3,6-tri-F-Ph | * |
| 442 | i-Bu | 1H-pyrazol-1-yl | Cl | 2,4-di-Cl-Ph | * |
| 443 | 3-F-propyl | 1H-pyrazol-1-yl | Cl | 2,4,6-tri-F-Ph | 181-182 |
| 444 | 3-Cl-Ph | 1H-pyrazol-1-yl | Cl | 2,3,6-tri-F-Ph | 211-214 |
| 445 | 3-Cl-Ph | 3-Me-1H-pyrazol-1-yl | Cl | 2,3,6-tri-F-Ph | 189-191 |
| 446 | 2-Cl-Ph | 1H-pyrazol-1-yl | Cl | 2,3,6-tri-F-Ph | 208-210 |
| 447 | 2-Cl-Ph | 3-Me-1H-pyrazol-1-yl | Cl | 2,3,6-tri-F-Ph | 200-201 |
| 448 | 3-F-Ph | 3-Cl-1H-pyrazol-1-yl | Cl | 2,4,6-tri-F-Ph | * |
| 449 | 3-F-Ph | 3-Me-1H-pyrazol-1-yl | Cl | 2,3,6-tri-F-Ph | 145-147 |
| 450 | 3, 3-di-F-propyl | 1H-pyrazol-1-yl | Cl | 2,4,6-tri-F-Ph | 153-154 |
| 451 | 2-Me-Bu | 1H-pyrazol-1-yl | Cl | 2,6-di-F, 4-OMe-Ph | * |
| (Ex. 15) | | | | | |
| 452 | 2-Me-Bu | 3-Me-1H-pyrazol-1-yl | Cl | 2,6-di-F, 4-OMe-Ph | * |
| 453 | 2-Me-Bu | 1H-pyrazol-1-yl | Cl | 2,6-di-F, 4-OH-Ph | * |
| (Ex. 15) | | | | | |
| 454 | 2,3-di-F-Ph | 1H-pyrazol-1-yl | Cl | 2,3,6-tri-F-Ph | 204-206 |
| 455 | 2,3-di-F-Ph | 3-Me-1H-pyrazol-1-yl | Cl | 2,3,6-tri-F-Ph | 181-184 |
| 456 | 3-F-Ph | 1H-pyrazol-1-yl | Cl | 2,3,6-tri-F-Ph | 210-212 |
| 457 | ($-2-Me-Bu | 1H-pyrazol-1-yl | Cl | 2,6-di-F, 4-OMe-Ph | * |
| 458 | 3,5-di-F-Ph | 1H-pyrazol-1-yl | Cl | 2,3,6-tri-F-Ph | 267-268 |
| 459 | 3,5-di-F-Ph | 3-Me-1H-pyrazol-1-yl | Cl | 2,3,6-tri-F-Ph | 218-220 |
| 460 | CH₂-c-Pr | 1H-pyrazol-1-yl | Cl | 2,4,6-tri-F-Ph | * |
| 461 | 3-F-Ph | 1H-pyrazol-1-yl | Cl | 2,3,4,5,6-penta-F- | 251-255 |
| | | | | Ph | |
| 462 | 2,6-di-F-Ph | 1H-pyrazol-1-yl | Cl | 2,6-di-F-Ph | 260-265 |
| 463 | 2,6-di-F-Ph | 3-Me-1H-pyrazol-1-yl | Cl | 2,6-di-F-Ph | 227-228 |
| 464 | 3-F-Ph | 3-Me-1H-pyrazol-1-yl | Cl | 2,3,4,5,6-penta-F- | 180-182 |
| | | | | Ph | |
| 465 | CH₂CH═CH₂ | 1H-pyrazol-1-yl | Cl | 2,4,6-tri-F-Ph | * |
| 466 | CF₃CH₂ | 1H-pyrazol-1-yl | Cl | 2,6-di-F-Ph | 223-224 |
| 467 | CF₃CF₂CF₂CH₂ | 1H-pyrazol-1-yl | Cl | 2,4,6-tri-F-Ph | 155-156 |
| 468 | CF₃CF₂CH₂ | 1H-pyrazol-1-yl | Cl | 2,4,6-tri-F-Ph | 147-149 |
| (Ex. 16) | | | | | |
| 469 | CF₃CF₂CH₂ | CONH₂ | Cl | 2,4,6-tri-F-Ph | 242-243 |
| 487 | i-Bu | 1-Me-1H-pyrazol-3-yl | Cl | 2,4,6-tri-F-Ph | * |
| 488 | CH₂-c-Pr | 3-Me-1H-pyrazol-1-yl | Cl | 2,6-di-F, 4-OMe-Ph | 149-152 |
| 489 | CH₂-c-Pr | 1H-pyrazol-1-yl | Cl | 2,6-di-F, 4-OMe-Ph | 144-147 |
| 490 | (S)-2-Me-Bu | 1-Me-1H-pyrazol-3-yl | Cl | 2,6-di-F, 4-OMe-Ph | * |
| (Ex. 18) | | | | | |
| 501 | (S)-2-Me-Bu | 1H-pyrazol-1-yl | Cl | 2,6-di-F, 4-OAc-Ph | * |
| QEV46 | | | | | |
| 503 | (S)-2-Me-Bu | 1H-pyrazol-1-yl | Cl | 2,6-di-F, 4-OH-Ph | * |
| 504 | (S)-2-Me-Bu | 3-Me-1H-pyrazol-1-yl | Cl | 2,6-di-F, 4-OMe-Ph | * |

* See Index Table d for ¹H NMR data.
^{a}Compound 302 has a retention time of 22.6 minutes; see Example 7.
^{b}Compound 303 has a retention time of 18.9 minutes; see Example 7.
^{c}Compounds 302 and 303 are atropisomers of each other.

INDEX TABLE B

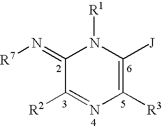

| Compd. No. | R¹ | R² | R7 | R³ | J | M.P. (° C.) |
|---|---|---|---|---|---|---|
| 470 | i-Bu | CN | H | Me | 2,4-di-F-Ph | 95-97 |
| 471 | 2-Me-Bu | CN | H | Me | 2,4-di-F-Ph | 73-77 |
| (Ex. 14) | | | | | | |
| 472 | i-Bu | CONH₂ | H | Me | 2,4-di-F-Ph | 172-177 |
| 473 | 2-Me-Bu | CONH₂ | H | Me | 2,4-di-F-Ph | 148-156 |
| 474 | i-Bu | CONH₂ | H | F | 2,4-di-F-Ph | 152-158 |
| 475 | 2-Me-Bu | CN | Ac | Me | 2,4-di-F-Ph | * |
| (Ex. 14) | | | | | | |
| 476 | i-Bu | CN | Ac | Me | Ph | * |

* See Index Table D for ¹H NMR data.

INDEX TABLE C

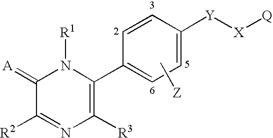

| Compd. | | | | | | | | M.P. |
|---|---|---|---|---|---|---|---|---|
| No. | R¹ | R² | R³ | Z | Y | X | Q | (° C.) |
| 477 | 2-Me-Bu | 1H-pyrazol- | Cl | 2-F-Ph | O | (CH₂)₂ | N(CH₃)₂ | * |
| | | 1-yl | | | | | | |
| 478 | 2-Me-Bu | 1H-pyrazol- | Cl | 2-F-Ph | O | (CH₂)₂ | 1- | * |
| | | 1-yl | | | | | pyrrolidine | |
| 479 | 2-Me-Bu | 1H-pyrazol- | Cl | 2,6-di- | O | (CH₂)₂ | N(CH₃)₂ | * |
| (Ex. 15) | | 1-yl | | F-Ph | | | | |
| 480 | 2-Me-Bu | 1H-pyrazol- | Cl | 2,6-di- | O | (CH₂)₂ | 1- | * |
| | | 1-yl | | F-Ph | | | pyrrolidine | |
| 481 | CH₂-c-Pr | 1H-pyrazol- | Cl | 2,6-di- | O | (CH₂)₃ | N(CH₃)₂ | * |
| | | 1-yl | | F-Ph | | | | |
| 482 | (S)-2-Me-Bu | 1H-pyrazol- | Cl | 2,6-di- | O | (CH₂)₃ | N(CH₃)₂ | * |
| | | 1-yl | | F-Ph | | | | |
| 483 | (S)-2-Me-Bu | 1H-pyrazol- | Cl | 2,6-di- | O | (CH₂)₂ | N(CH₃)₂ | * |
| | | 1-yl | | F-Ph | | | | |
| 484 | (S)-2-Me-Bu | CONH₂ | Cl | 2,6-di- | O | (CH₂)₂ | N(CH₃)₂ | * |
| | | | | F-Ph | | | | |
| 485 | 3,3,3-trifluoro-2- | 1H-pyrazol- | Cl | 2,6-di- | O | (CH₂)₃ | N(CH₃)₂ | * |
| | methylpropyl | 1-yl | | F-Ph | | | | |
| 486 | (S)-2-Me-Bu | CONH₂ | Cl | 2,6-di- | O | (CH₂)₃ | N(CH₃)₂ | * |
| | | | | F-Ph | | | | |
| 491 | 3-F-Ph | 1H-pyrazol- | Cl | 2,6-di- | O | (CH₂)₃ | N(CH₃)₂ | * |
| | | 1-yl | | F-Ph | | | | |
| 492 | 3-F-Ph | 1H-pyrazol- | Cl | 2,6-di- | O | (CH₂)₃ | NH(CH₃)—HCl | # |
| | | 1-yl | | F-Ph | | | | |
| 493 | (S)-2-Me-Bu | 1H-pyrazol- | Cl | 2,6-di- | O | (CH₂)₃ | NH(CH₃)—HCl | * |
| (Ex. 17) | | 1-yl | | F-Ph | | | | |
| 494 | (S)-2-Me-Bu | 3-Me-1H- | Cl | 2,6-di- | O | (CH₂)₃ | NH(CH₃)—HCl | * |
| | | pyrazol-1-yl | | F-Ph | | | | |
| 495 | i-Bu | 1H-pyrazol- | Cl | 2,6-di- | O | (CH₂)₃ | N(CH₃)₂ | * |
| | | 1-yl | | F-Ph | | | | |
| 496 | i-Bu | 3-Me-1H- | Cl | 2,6-di- | O | (CH₂)₃ | NH(CH₃) | * |
| | | pyrazol-1-yl | | F-Ph | | | | |
| 497 | i-Bu | 3-Me-1H- | Cl | 2,6-di- | O | (CH₂)₃ | N(CH₃)₂ | * |
| | | pyrazol-1-yl | | F-Ph | | | | |
| 498 | (S)-2-Me-Bu | 3-Me-1H- | Cl | 2,6-di- | O | (CH₂)₃ | N(CH₃)₂ | * |
| | | pyrazol-1-yl | | F-Ph | | | | |
| 499 | (S)-2-Me-Bu | 1H-pyrazol- | Cl | 2,6-di- | O | (CH₂)₃ | N(CH₃) | * |
| | | 1-yl | | F-Ph | | | (COCH₃) | |
| 500 | i-Bu | 1H-pyrazol- | Cl | 2,6-di- | O | (CH₂)₃ | NH(CH₃)—HCl | * |
| | | 1-yl | | F-Ph | | | | |
| 502 | (S)-2-Me-Bu | 1-Me-1H- | Cl | 2,6-di- | O | (CH₂)₃ | NH(CH₃)—HCl | ## |
| | | pyrazol-3-yl | | F-Ph | | | | |

* See Index Table D for ¹H NMR data.
# MS (AP+) 490.1, molecular weight of the highest isotopic abundance parent ion (M + 1) formed by addition of H⁺ (molecular weight of 1) to the molecule of monochloro compound, observed by mass spectrometry using atmospheric pressure chemical ionization (AP+).
## MS (AP+) 480.1, molecular weight of the highest isotopic abundance parent ion (M + 1) formed by addition of H⁺ (molecular weight of 1) to the molecule of monochloro compound, observed by mass spectrometry using atmospheric pressure chemical ionization (AP+).

INDEX TABLE D

| Compd. No. | ¹H NMR Data (CDCl₃ solution unless indicated otherwise)^{a} |
|---|---|
| 1 | δ 9.1 (m, 1H), 7.9 (m, 1H), 7.5 (m, 1H), 7.1 (m, 2H), 6.5 (m, 1H), 3.8 (d, 2H), |
| | 2.0 (m, 1H), 0.8 (d, 6H). |
| 2 | δ 8.86 (m, 1H), 8.43 (m, 1H), 7.83 (m, 1H), 7.59 (m, 1H), 7.38 (m, 1H), 7.12 (m, |
| | 2H), 3.79 (d, 2H), 2.00 (m, 1H), 0.79 (d, 6H). |
| 3 | δ 9.81 (s, 1H), 8.21 (s, 1H), 7.62 (m, 1H), 7.14 (m, 2H), 3.84 (d, 2H), 2.00 (m, 1H), |
| | 0.80 (d, 6H). |
| 4 | δ 7.46 (m, 1H), 7.21 (d, 1H), 7.04 (m, 2H), 4.33 (m, 2H), 3.61 (d, 2H), 2.97 (m, 2H), |
| | 1.94 (m, 1H), 0.75 (d, 6H). |
| 9 | δ 9.12 (m, 1H), 7.89 (m, 1H), 7.53 (m, 1H), 7.42 (m, 1H), 7.21 (m, 1H), 6.51 (m, |
| | 1H), 3.77 (m, 2H), 1.95 (m, 1H), 0.81 (m, 6H). |
| 10 | δ 9.81 (s, 1H), 8.21 (s, 1H), 7.57 (m, 1H), 7.45 (m, 1H), 7.24 (m, 1H), 3.84 (m, 2H), |
| | 1.96 (m, 1H), 0.82 (m, 6H). |
| 13 | δ 9.2 (m, 1H), 7.91 (m, 1H), 7.45 (m, 1H), 7.35 (m, 1H), 6.52 (m, 1H), 3.94 (m, 1H), |
| | 3.92 (s, 3H), 3.65 (m, 1H), 2.02 (m, 1H), 0.81 (d, 6H). |
| 14 | δ 9.11 (m, 1H), 7.88 (m, 1H), 7.55 (m, 1H), 7.36 (m, 2H), 7.29 (m, 1H), 6.44 (m, |
| | 1H), 3.45 (s, 3H). |
| 15 | δ 9.81 (s, 1H), 8.21 (m, 1H), 7.62 (m, 1H), 7.41 (m, 2H), 7.31 (m, 1H), 3.45 (s, 3H). |
| 16 | δ 7.5 (m, 1H), 7.20 (s, 1H), 7.0 (t, 2H), 4.3 (t, 2H), 3.6 (d, 2H), 2.9 (t, 2H), 0.9 (m, |
| | 1H), 0.4 (m, 2H), 0.9 (m, 2H). |
| 17 | δ 9.8 (s, 1H), 8.2 (s, 1H), 7.6 (m, 1H), 7.1 (t, 2H) 3.9 (d, 2H), 1.0 (m, 1H), 0.5 (m, |
| | 2H), 0.2 (m, 2H). |
| 18 | δ 9.1 (d, 1H), 7.8 (d, 1H), 7.6 (m, 1H), 7.1 (t, 2H), 6.5 (t, 1H), 3.8 (d, 2H), 1.0 (m, |
| | 1H), 1.4 (m, 2H), 0.2 (m, 2H). |
| 19 | δ 9.1 (d, 1H), 7.9 (d, 1H), 6.9 (t, 2H), 6.5 (m, 1H), 3.8 (d, 2H), 1.2 (m, 1H), 1.0 (m, |
| | 1H), 0.77-0.75 (m, 6H). |
| 20 | δ 9.7 (s, 1H), 8.2 (s, 1H), 6.9 (t, 2H), 3.8 (d, 2H), 1.7 (m, 1H), 1.2 (m, 1H), 1.0 (m, |
| | 1H), 0.79-0.73 (m, 6H). |
| 21 | δ 7.65 (m, 1H), 7.15 (t, 2H), 3.8 (d, 2H), 1.7 (m, 1H), 1.2 (m, 1H), 1.0 (m, 1H), |
| | 0.75-0.68 (m, 6H). |
| 22 | δ 9.07 (d, 1H), 7.88 (s, 1H), 7.59-7.50 (m, 2H), 7.29 (d, 2H), 6.55-6.49 (m, 1H), |
| | 3.82 (d, 2H), 2.07-1.96 (m, 1H), 0.76 (d, 6H). |
| 23 | δ 9.77 (s, 1H), 8.20 (s, 1H), 7.57 (d, 2H), 7.30 (d, 2H), 3.86 (d, 2H), 2.03 (d, 1H), |
| | 0.78 (d, 6H). |
| 24 | δ 7.57 (d, 2H), 7.30 (d, 2H), 3.81 (d, 2H), 2.57 (s, 3H), 2.44 (s, 3H), 2.07-1.96 (m, |
| | 1H), 0.77 (d, 6H). |
| 25 | δ 9.05 (d, 1H), 7.90 (d, 1H), 7.55 (m, 1H), 7.11 (t, 2H), 6.50 (t, 1H), 2.35 (m, 1H), |
| | 1.85 (m, 1H), 1.57 (m, 4H), 0.75 (t, 3H). |
| 26 | δ 9.04 (d, 1H), 7.89 (d, 1H), 7.58 (m, 1H), 7.12 (t, 2H), 6.50 (t, 1H), 4.15 (m, 1H), |
| | 1.58 (m, 6H). |
| 27 | δ 9.15 (d, 1H), 7.90 (d, 1H), 7.60 (m, 1H), 7.13 (t, 2H), 6.50 (t, 1H), 3.85 (t, 2H), |
| | 1.62 (m, 2H), 0.80 (t, 3H). |
| 28 | δ 9.11 (d, 1H), 7.88 (d, 1H), 7.60 (m, 1H), 7.13 (t, 2H), 6.51 (t, 1H), 3.85 (t, 2H), |
| | 1.58 (m, 2H), 1.16 (m, 4H), 0.79 (t, 3H). |
| 29 | δ 9.05 (d, 1H), 7.90 (d, 1H), 7.58 (m, 1H), 7.12 (t, 2H), 6.52 (t, 1H), 3.40 (m, 1H), |
| | 2.90 (m, 1H), 1.60 (d, 3H), 0.82 (d, 3H), 0.73 (d, 3H). |
| 30 | δ 9.13 (d, 1H), 7.91 (d, 1H), 7.58 (m, 1H), 7.06 (t, 2H), 6.52 (t, 1H), 4.78 (s, 1H), |
| | 4.56 (s, 2H), 4.37 (s, 1H), 1.58 (s, 3H). |
| 31 | δ 9.10 (d, 1H), 7.90 (d, 1H), 7.58 (m, 1H), 7.12 (t, 2H), 6.50 (t, 1H), 3.80 (d, 2H), |
| | 1.60 (m, 3H), 1.50 (m, 2H), 1.10 (m, 2H), 0.90 (m, 2H), 0.78 (m, 2H). |
| 32 | δ 9.76 (s, 1H), 8.20 (s, 1H), 7.60 (m, 1H), 7.15 (t, 2H), 3.85 (m, 1H), 2.35 (m, 1H), |
| | 1.88 (m, 1H), 1.60 (d, 3H), 0.78 (t, 3H). |
| 33 | δ 9.77 (s, 1H), 8.20 (s, 2H), 7.61 (m, 1H), 7.13 (t, 2H), 4.20 (m, 1H), 1.60 (m, 6H). |
| 34 | δ 9.81 (s, 1H), 8.21 (s, 1H), 7.62 (m, 1H), 7.16 (t, 2H), 3.88 (t, 2H), 1.62 (m, 2H), |
| | 0.82 (t, 3H). |
| 35 | δ 9.10 (d, 1H), 7.88 (d, 1H), 7.57 (m, 1H), 7.13 (t, 2H), 6.50 (t, 1H), 3.89 (t, 2H), |
| | 1.45 (m, 3H), 0.73 (m, 6H). |
| 36 | δ 9.08 (d, 1H), 7.89 (d, 1H), 7.83 (d, 2H), 7.51 (d, 2H), 6.52 (d, 1H), 3.80 (d, 2H), |
| | 2.04 (s, 1H), 0.76 (d, 6H). |
| 37 | δ 9.06 (d, 1H), 7.87 (s, 1H), 7.32-7.16 (m, 2H), 7.05 (d, 2H), 6.57-6.42 (m, 1H), |
| | 3.97-3.76 (m, 5H), 2.11-1.97 (m, 1H), 0.75 (d, 6H). |
| 38 | δ 9.07 (d, 1H), 7.88 (s, 1H), 7.41 (s, 4H), 6.52-6.49 (m, 1H), 3.82 (d, 2H), 1.99 (s, |
| | 1H), 0.76 (d, 6H). |
| 39 | δ 9.10 (d, 1H), 7.92-7.87 (m, 1H), 7.72-7.64 (m, 1H), 7.42 (t, 1H), 7.35-7.30 (m, 1H), |
| | 6.52 (m, 1H), 4.15 (m, 1H), 3.41-3.31 (m, 1H), 0.85 (d, 3H), 0.74 (d, 3H). |
| 44 | δ 9.79 (s, 1H), 8.21 (s, 1H), 8.13-8.00 (m, 1H), 7.48-7.28 (m, 2H), 3.39-3.24 (m, 2H), |
| | 2.01-1.82 (m, 1H), 0.85-0.66 (m, 6H). |
| 45 | δ 9.09 (d, 1H), 7.93-7.88 (m, 1H), 7.37-7.28 (m, 3H), 6.53-6.49 (m, 1H), |
| | 4.01-3.93 (m, 1H), 3.67-3.58 (m, 1H), 2.04-1.95 (m, 1H), 0.80 (d, 3H), 0.76 (d, 3H). |
| 46 | δ 9.10 (d, 1H), 7.92-7.88 (m, 1H), 7.67-7.62 (m, 1H), 7.58-7.52 (m, 2H), 6.52 (m, |
| | 1H), 4.00-3.91 (m, 1H), 3.65-3.55 (m, 1H), 2.03-1.96 (m, 1H), 0.80 (d, 3H), 0.76 (d, |
| | 3H). |
| 47 | δ 9.0 (d, 1H), 7.8 (s, 1H), 7.5 (m, 1H), 7.1 (m, 2H), 6.5 (d, 1H), 3.6 (br s, 1H), 2.6 (br |
| | s, 2H), 1.8 (d, 2H), 1.7 (d, 2H), 1.6 (d, 1H), 1.2 (m, 1H), 1.0 (m, 2H). |
| 48 | δ 9.01 (m, 1H), 7.44 (m, 1H), 7.08 (m, 2H), 3.62 (d, 2H), 2.19 (s, 3H), 2.03 (s, 3H), |
| | 1.85 (m, 1H), 0.75 (d, 6H). |
| 50 | δ 9.10 (d, 1H), 7.92-7.87 (m, 1H), 7.45-7.37 (m, 1H), 7.35-7.28 (m, 1H), |
| | 7.16-7.11 (m, 1H), 6.51 (m, 1H), 3.99-3.89 (m, 1H), 3.73-3.63 (m, 1H), 2.01 (m, 1H), |
| | 0.81-0.75 (m, 6H). |
| 55 | δ 9.81 (s, 1H), 8.22 (s, 1H), 7.51-7.43 (m, 1H), 7.14-7.09 (m, 1H), 3.90-3.80 (m, 2H), |
| | 2.04-1.97 (m, 1H), 0.84-0.80 (m, 6H). |
| 56 | δ 9.09 (d, 1H), 7.89 (d, 1H), 7.29-7.22 (m, 2H), 7.12-7.06 (m, 1H), 6.51 (m, 1H), |
| | 4.04-3.94 (m, 1H), 3.70-3.61 (m, 1H), 2.04-1.98 (m, 1H), 0.82-0.76 (m, 6H). |
| 57 | δ 9.11 (d, 1H), 7.91 (d, 1H), 7.48-7.39 (m, 1H), 7.12-7.05 (m, 1H), 6.51 (m, 1H), |
| | 3.84-3.74 (m, 2H), 2.03-1.94 (m, 1H), 0.83-0.78 (m, 6H). |
| 58 | δ 9.11 (d, 1H), 7.89 (d, 1H), 7.58 (m, 1H), 7.09 (t, 2H), 6.51 (t, 1H), 5.77 (m, 1H), |
| | 5.16 (d, 1H), 4.89 (d, 1H), 4.55 (d, 2H). |
| 59 | δ 9.08 (d, 1H), 7.89 (d, 1H), 7.57 (m, 1H), 7.09 (t, 2H), 6.50 (t, 1H), 3.97 (s, 2H), |
| | 0.82 (s, 9H). |
| 60 | δ 8.98 (d, 1H), 7.85 (d, 1H), 7.63 (m, 1H), 7.15 (t, 2H), 3.87 (d, 2H), 1.72 (m, 1H), |
| | 1.26 (m, 1H), 1.06 (m, 1H), 0.42 (m, 6H). |
| 61 | δ 9.08 (d, 1H), 8.08 (d, 1H), 7.60 (m, 1H), 7.18 (s, 1H), 7.12 (t, 2H), 3.83 (d, 2H), |
| | 1.68 (m, 1H), 1.22 (m, 1H), 1.03 (m, 1H), 0.75 (d, 3H), 0.71 (t, 3H). |
| 62 | δ 7.60 (m, 1H), 7.15 (t, 2H), 6.04 (s, 1H), 3.80 (d, 2H), 2.42 (s, 3H), 2.32 (s, 3H), |
| | 1.75 (m, 1H), 1.23 (m, 1H), 1.03 (m, 1H), 0.74 (d, 3H), 0.69 (t, 3H). |
| 63 | δ 9.08 (d, 1H), 7.60 (m, 1H), 7.13 (t, 2H), 6.74 (d, 1H), 3.86 (d, 2H), 1.70 (m, 1H), |
| | 1.22 (m, 1H), 1.04 (m, 1H), 0.74 (d, 3H), 0.70 (t, 3H). |
| 64 | δ 7.62 (m, 1H), 7.15 (t, 2H), 6.47 (s, 1H), 3.83 (d, 2H), 2.46 (s, 3H), 1.70 (m, 1H), |
| | 1.23 (m, 1H), 1.02 (m, 1H), 0.75 (d, 3H), 0.70 (t, 3H). |
| 65 | δ 9.01 (d, 1H), 7.59 (m, 1H), 7.12 (t, 2H), 6.51 (d, 1H), 3.83 (d, 2H), 1.69 (m, 1H), |
| | 1.22 (m, 1H), 1.01 (m, 1H), 0.74 (d, 3H), 0.69 (t, 3H). |
| 66 | δ 9.03 (d, 1H), 7.57 (m, 1H), 7.10 (t, 2H), 6.31 (d, 1H), 3.82 (d, 2H), 2.44 (s, 3H), |
| | 1.70 (m, 1H), 1.23 (m, 1H), 1.01 (m, 1H), 0.75 (d, 3H), 0.70 (t, 3H). |
| 67 | δ 9.21 (s, 1H), 7.66 (m, 1H), 7.17 (t, 2H), 3.90 (d, 2H), 1.72 (m, 1H), 1.23 (m, 1H), |
| | 1.03 (m, 1H), 0.77 (d, 3H), 0.72 (t, 3H). |
| 68 | δ 8.31 (s, 1H), 7.63 (m, 1H), 7.15 (t, 2H), 3.84 (d, 2H), 1.70 (m, 1H), 1.22 (m, 1H), |
| | 1.05 (m, 1H), 0.76 (d, 3H), 0.71 (t, 3H). |
| 69 | δ 7.89 (d, 1H), 7.60 (m, 1H), 7.13 (t, 2H), 6.98 (d, 1H), 3.81 (d, 2H), 2.69 (s, 3H), |
| | 1.70 (m, 1H), 1.22 (m, 1H), 1.02 (m, 1H), 0.75 (d, 3H), 0.70 (t, 3H). |
| 70 | δ 8.85 (d, 1H), 7.60 (m, 1H), 7.15 (t, 2H), 7.02 (d, 1H), 3.82 (d, 2H), 3.03 (q, 2H), |
| | 1.71 (m, 1H), 1.39 (t, 3H), 1.24 (m, 1H), 1.03 (m, 1H), 0.75 (d, 3H), 0.73 (t, 3H). |
| 71 | δ 8.04 (d, 1H), 7.60 (m, 1H), 7.15 (t, 2H), 7.01 (d, 1H), 3.81 (d, 2H), 3.0 (t, 2H), |
| | 1.82 (q, 2H), 1.70 (m, 1H), 1.23 (m, 1H), 1.02 (t, 4H), 0.75 (d, 3H), 0.72 (t, 3H). |
| 72 | δ 7.71 (d, 1H), 7.60 (m, 1H), 7.14 (t, 2H), 7.04 (d, 1H), 3.81 (d, 2H), 3.50 (m, 1H), |
| | 1.7 (m, 1H), 1.39 (d, 6H), 1.21 (m, 1H), 1.04 (m, 1H), 0.75 (d, 3H), 0.71 (t, 3H). |
| 73 | δ 8.85 (m, 1H), 7.50 (m, 1H), 7.04 (m, 2H), 3.65 (d, 2H), 2.63 (m, 2H), 2.43 (m, 2H), |
| | 1.95 (m, 1H), 1.81 (m, 3H), 0.75 (d, 6H). |
| 74 | δ 9.06 (d, 1H), 7.87 (s, 1H), 7.54 (d, 2H), 7.31-7.20 (m, 2H), 6.49 (d, 1H), 3.85 (d, |
| | 2H), 2.06-1.96 (m, 1H), 1.38 (s, 9H), 0.74 (d, 6H). |
| 75 | δ 9.77 (s, 1H), 8.19 (s, 1H), 7.57 (d, 2H), 7.26 (d, 2H), 3.89 (d, 2H), 2.05-1.97 (m, |
| | 1H), 1.39 (s, 9H), 0.75 (d, 6H). |
| 76 | δ 9.78 (s, 1H), 8.21 (s, 1H), 7.86 (d, 2H), 7.52 (d, 2H), 3.84 (d, 2H), 2.02 (dt, 1H), |
| | 0.78 (d, 6H). |
| 77 | δ 9.77 (s, 1H), 8.20 (s, 1H), 7.42 (s, 4H), 3.86 (d, 2H), 2.01 (s, 1H), 0.78 (d, 6H). |
| 78 | δ 9.38 (m, 1H), 7.52 (m, 1H), 7.10 (m, 2H), 3.90 (s, 3H), 3.63 (m, 2H), 2.33 (d, 3H), |
| | 1.85 (m, 1H), 0.75 (d, 6H). |
| 79 | δ 9.77 (s, 1H), 8.20 (s, 1H), 7.21-7.17 (m, 2H), 7.12 (m, 1H), 4.10-4.03 (m, 1H), |
| | 3.57-3.49 (m, 1H), 2.43 (s, 3H), 2.14 (s, 3H), 2.03-1.96 (m, 1H), 0.82 (d, 3H), 0.75 (d, |
| | 3H). |
| 80 | δ 9.07 (d, 1H), 7.87 (m, 1H), 7.21-7.09 (m, 3H), 6.50 (m, 1H), 4.04 (m, 1H), |
| | 3.52-3.42 (m, 1H), 2.41 (s, 3H), 2.13 (s, 3H), 2.04-1.96 (m, 1H), 0.80 (d, 3H), 0.74 (d, |
| | 3H). |
| 81 | δ 9.12 (d, 1H), 7.61 (m, 1H), 7.14 (t, 2H), 6.86 (d, 1H), 3.87 (d, 2H), 1.72 (m, 1H), |
| | 1.22 (m, 1H), 1.03 (m, 1H), 0.75 (d, 3H), 0.72 (t, 3H). |
| 82 | δ 8.10 (t, 2H), 7.58 (m, 1H), 7.10 (t, 2H), 6.34 (t, 2H), 3.79 (d, 2H), 1.68 (m, 1H), |
| | 1.22 (m, 1H), 1.02 (m, 1H), 0.74 (d, 3H), 0.70 (t, 3H). |
| 83 | δ 8.57 (s, 1H), 7.67-7.60 (m, 2H), 7.10 (t, 2H), 4.06 (s, 3H), 3.75 (d, 2H), |
| | 2.05-1.93 (m, 1H), 0.77 (d, 6H). |
| 84 | δ 8.75 (d, 2H), 8.34 (d, 2H), 7.65-7.57 (m, 1H), 7.14 (t, 2H), 3.79 (d, 2H), |
| | 2.04-1.95 (m, 1H), 0.79 (d, 6H). |
| 87 | δ 9.12 (d, 1H), 7.90 (d, 1H), 7.79-7.70 (m, 2H), 7.54 (m, 1H), 6.51 (m, 1H), |
| | 4.24-4.17 (m, 1H), 3.15 (m, 1H), 2.03-1.95 (m, 1H), 0.87 (d, 3H), 0.74 (d, 3H). |
| 90 | δ 9.10 (d, 1H), 8.02 (d, 1H), 7.97-7.89 (m, 3H), 7.85 (s, 1H), 7.63 (m, 2H), 7.38 (m, |
| | 1H), 6.51 (m, 1H), 3.93-3.83 (m, 2H), 2.12-2.01 (m, 1H), 0.72 (m, 6H). |
| 91 | δ 9.07 (s, 1H), 7.97-7.85 (m, 1H), 7.63-7.49 (m, 1H), 7.15-7.02 (m, 2H), |
| | 6.59-6.45 (m, 1H), 4.33-4.16 (m, 2H), 3.72-3.56 (m, 2H), 3.41-3.31 (m, 1H), 2.03-1.94 (m, |
| | 1H), 1.81-1.72 (m, 1H), 1.69-1.59 (m, 1H), 1.49-1.38 (m, 1H). |
| 92 | δ 9.77 (s, 1H), 8.29-8.19 (m, 1H), 7.65-7.55 (m, 1H), 7.19-7.02 (m, 2H), |
| | 4.40-4.27 (m, 1H), 4.28-4.19 (m, 1H), 3.77-3.65 (m, 1H), 3.67-3.51 (m, 2H), 2.02 (m, 1H), |
| | 1.83-1.73 (m, 1H), 1.72-1.57 (m, 1H), 1.51-1.36 (m, 1H). |
| 99 | δ 9.0 (d, 1H), 7.8 (s, 1H), 7.6 (m, 1H), 7.1 (m, 2H), 6.5 (d, 1H), 3.8 (d, 2H), 1.9 (m, |
| | 1H), 0.7 (d, 6H). |
| 100 | δ 8.57 (s, 1H), 8.35 (s, 1H), 7.55 (m, 1H), 7.09 (t, 2H), 3.97 (s, 3H), 3.73 (d, 2H), |
| | 1.95 (m, 1H), 0.77 (d, 6H). |
| 103 | δ 7.62-7.55 (m, 3H), 7.12 (t, 2H), 4.29 (s, 3H), 3.76 (d, 2H), 2.03-1.94 (m, 1H), |
| | 0.78 (d, 6H). |
| 110 | δ 7.6 (m, 1H), 7.1 (t, 2H), 3.7 (d, 2H), 1.6 (m, 1H), 1.2 (m, 1H), 1.0 (m, 1H), 0.70 (m, |
| | 6H). |
| 111 | δ 9.04 (d, 1H), 7.83 (d, 1H), 7.55 (d, 1H), 7.41 (m, 1H), 7.30 (d, 1H), 6.45 (m, 1H), |
| | 4.02 (m, 1H), 3.40-3.31 (m, 1H), 1.95 (m, 1H), 0.79 (d, 3H), 0.69 (d, 3H). |
| 112 | δ 9.73 (s, 1H), 8.13 (s, 1H), 6.18 (s, 2H), 3.87 (s, 3H), 3.76 (m, 8H), 1.99-1.88 (m, |
| | 1H), 0.74 (d, 6H). |
| 113 | δ 9.81 (s, 1H), 8.22 (s, 1H), 7.83-7.72 (m, 2H), 7.55 (t, 1H), 4.24 (m, 1H), 3.22 (m, |
| | 1H), 2.00 (m, 1H), 0.88 (d, 3H), 0.76 (d, 3H). |
| 116 | δ 9.77 (s, 1H), 8.20 (s, 1H), 7.60 (m, 1H), 7.27 (m, 2H), 3.85 (d, 2H), 2.05 (m, 1H), |
| | 0.82 (d, 6H). |
| 119 | δ 9.80 (s, 1H), 8.21 (s, 1H), 8.04 (d, 1H), 7.95 (m, 2H), 7.87 (s, 1H), 7.65 (m, 2H), |
| | 7.39 (m, 1H), 4.00-3.88 (m, 2H), 2.07 (m, 1H), 0.74 (m, 6H). |
| 128 | δ 9.71 (s, 1H), 8.18 (s, 1H), 7.39-7.28 (m, 3H), 7.12 (d, 2H), 4.35 (s, 2H), 3.95 (d, |
| | 2H), 2.24 (m, 1H), 0.98 (d, 6H). |
| 129 | δ 9.83 (s, 1H), 8.23 (s, 1H), 8.08 (d, 1H), 8.00 (d, 1H), 7.68-7.52 (m, 4H), 7.41 (d, |
| | 1H), 4.06 (dd, 1H), 3.38 (m, 1H), 1.92 (m, 1H), 0.69 (m, 6H). |
| 130 | δ 9.01 (d, 1H), 7.86 (d, 1H), 7.37-7.26 (m, 3H), 7.12 (d, 2H), 6.47 (m, 1H), 4.31 (s, |
| | 2H), 3.89 (d, 2H), 2.22 (m, 1H), 0.96 (d, 6H). |
| 131 | δ 9.13 (d, 1H), 8.05 (d, 1H), 7.98 (m, 1H), 7.91 (d, 1H), 7.67-7.50 (m, 4H), 7.44 (d, |
| | 1H), 6.53 (m, 1H), 4.02 (m, 1H), 3.31 (m, 1H), 1.92 (m, 1H), 0.66 (m, 6H). |
| 132 | δ 8.99 (d, 1H), 7.84 (d, 1H), 7.35 (t, 2H), 7.27 (m, 1H), 7.21 (d, 2H), 6.46 (m, 1H), |
| | 4.76 (s, 1H), 4.09 (br s, 2H), 2.13 (m, 1H), 1.86 (d, 3H), 0.93 (d, 3H), 0.83 (br s, 3H). |
| 133 | δ 9.69 (s, 1H), 8.16 (s, 1H), 7.36 (m, 2H), 7.29 (m, 1H), 7.20 (d, 2H), 4.79 (br s, 1H), |
| | 4.14 (br s, 2H), 2.16 (m, 1H), 1.88 (d, 3H), 0.96 (d, 3H), 0.88 (br s, 3H). |
| 134 | δ 7.50 (m, 1H), 7.14 (m, 1H), 7.03 (m, 2H), 3.65 (m, 2H), 2.76 (m, 6H), 1.61 (m, |
| | 1H), 0.72 (d, 6H). |
| 135 | δ 7.48 (m, 1H), 7.24 (s, 1H), 7.02 (m, 2H), 3.89 (d, 3H), 3.62 (m, 2H), 3.03 (m, 3H), |
| | 1.25 (m, 2H), 0.99 (m, 2H), 0.71 (m, 3H), 0.66 (m, 3H). |
| 136 | δ 9.76 (s, 1H), 8.19 (s, 1H), 7.19 (s, 1H), 6.93 (s, 2H), 3.87 (d, 2H), 2.41 (s, 6H), |
| | 2.09 (m, 1H), 0.78 (d, 6H). |
| 138 | δ 9.79 (s, 1H), 8.21 (s, 1H), 7.22 (m, 1H), 6.90 (m, 1H), 3.98 (m, 1H), 3.72 (m, 1H), |
| | 2.02 (m, 1H), 0.82 (m, 6H). |
| 139 | δ 9.09 (d, 1H), 7.89 (d, 1H), 7.20 (m, 1H), 6.92 (m, 1H), 6.51 (m, 1H), 3.92 (m, 1H), |
| | 3.68 (m, 1H), 2.02 (m, 1H), 0.80 (m, 6H). |
| 140 | δ 9.80 (s, 1H), 8.21 (s, 1H), 7.92 (m, 1H), 7.83-7.73 (m, 2H), 7.49 (d, 1H), 4.24 (dd, |
| | 1H), 3.11 (dd, 1H), 2.14 (dd, 1H), 0.87 (d, 3H), 0.73 (d, 3H). |
| 141 | δ 9.10 (d, 1H), 7.88 (m, 2H), 7.75 (m, 2H), 7.47 (d, 1H), 6.51 (dd, 1H), 4.22 (dd, |
| | 1H), 3.05 (dd, 1H), 2.12 (m, 1H), 0.85 (d, 3H), 0.71 (d, 3H). |
| 142 | δ 9.77 (s, 1H), 8.20 (s, 1H), 7.36 (m, 2H), 7.29 (d, 2H), 3.87 (d, 2H), 2.02 (s, 1H), |
| | 0.77 (d, 6H). |
| 143 | δ 9.07 (d, 1H), 7.88 (d, 1H), 7.34 (m, 2H), 7.26 (m, 2H), 6.50 (dd, 1H), 3.83 (d, 2H), |
| | 2.00 (m, 1H), 0.76 (d, 6H). |
| 144 | δ 9.78 (s, 1H), 8.21 (s, 1H), 7.90 (m, 2H), 7.52 (d, 2H), 3.82 (d, 2H), 2.00 (td, 1H), |
| | 0.78 (d, 6H). |
| 145 | δ 9.08 (d, 1H), 7.87 (m, 3H), 7.51 (d, 2H), 6.52 (dd, 1H), 3.78 (d, 2H), 1.98 (dt, 1H), |
| | 0.76 (d, 6H). |
| 146 | δ 9.78 (s, 1H), 8.27-8.19 (m, 3H), 7.47-7.43 (m, 2H), 4.00 (s, 3H), 3.85 (d, 2H), |
| | 2.05 (s, 1H), 0.76 (d, 6H). |
| 147 | δ 9.08 (d, 1H), 8.23 (d, 2H), 7.89 (d, 1H), 7.45 (d, 2H), 6.51 (m, 1H), 3.99 (s, 3H), |
| | 3.81 (d, 2H), 1.98 (m, 1H), 0.74 (d, 6H). |
| 150 | δ 9.09 (d, 1H), 8.09 (s, 1H), 7.90 (m, 1H), 7.86 (s, 2H), 6.54 (m, 1H), 3.75 (d, 2H), |
| | 2.00 (m, 1H), 0.78 (d, 6H). |
| 152 | δ 9.09 (d, 1H), 7.88 (d, 1H), 7.02 (d, 1H), 6.98 (d, 1H), 6.50 (m, 1H), 3.93 (d, 2H), |
| | 2.16 (m, 1H), 0.85 (d, 6H). |
| 161 | δ 9.10 (s, 1H), 7.90 (s, 1H), 7.30 (m, 6H), 6.85-6.80 (t, 2H), 6.50 (s, 1H). |
| 164 | δ 9.08 (m, 1H), 7.89 (d, 1H), 7.40 (m, 1H), 7.34 (m, 1H), 7.20 (m, 1H), 6.50 (m, 1H), |
| | 4.10 (m, 1H), 3.48 (m, 1H), 1.75 (m, 1H), 1.22-0.95 (m, 2H), 0.72 (m, 6H). |
| 165 | δ 9.80 (d, 1H), 8.20 (s, 1H), 7.40 (m, 2H), 7.24 (m, 1H), 4.12 (m, 1H), 3.54 (m, 1H), |
| | 1.89-1.62 (m, 1H), 1.29-0.92 (m, 2H), 0.72 (m, 6H). |
| 166 | δ 9.11 (s, 1H), 7.81 (s, 1H), 7.38 (m, 2H), 7.20 (m, 1H), 4.12 (m, 1H), 3.48 (m, 1H), |
| | 1.70 (m, 1H), 1.32-0.89 (m, 2H), 0.74 (m, 6H). |
| 169 | δ 8.85 (s, 1H), 7.71 (s, 1H), 7.40 (m, 1H), 7.32 (m, 1H), 7.18 (m, 1H), 4.10 (m, 1H), |
| | 3.46 (m, 1H), 2.16 (s, 3H), 1.72 (m, 1H), 1.10 (m, 2H), 0.72 (m, 6H). |
| 170 | δ 9.02 (d, 1H), 7.40 (m, 1H), 7.32 (m, 1H), 7.18 (m, 1H), 6.31 (d, 1H), 4.09 (m, 1H), |
| | 3.45 (m, 1H), 2.44 (s, 3H), 1.72 (m, 1H), 1.02 (m, 2H), 0.72 (m, 6H). |
| 172 | δ 9.07 (d, 1H), 7.88 (d, 1H), 7.54 (m, 3H), 7.32 (m, 2H), 6.50 (dd, 1H), 3.84 (d, 2H), |
| | 2.02 (m, 1H), 0.75 (d, 6H). |
| 173 | δ 9.78 (s, 1H), 8.20 (s, 1H), 7.58 (m, 3H), 7.35 (td, 2H), 3.88 (d, 2H), 2.02 (m, 1H), |
| | 0.76 (d, 6H). |
| 174 | δ 9.09 (d, 1H), 7.88 (d, 1H), 7.58 (m, 1H), 7.36 (m, 2H), 7.26 (m, 1H), 6.50 (dd, 1H), |
| | 3.98 (dd, 1H), 3.66 (dd, 1H), 1.98 (m, 1H), 0.78 (d, 3H), 0.74 (d, 3H). |
| 175 | δ 9.79 (s, 1H), 8.20 (m, 1H), 7.62 (m, 1H), 7.38 (m, 2H), 7.29 (t, 1H), 4.02 (dd, 1H), |
| | 3.71 (dd, 1H), 1.99 (m, 1H), 0.79 (d, 3H), 0.75 (d, 3H). |
| 176 | δ 9.07 (d, 1H), 7.87 (d, 1H), 7.52 (d, 1H), 7.26 (dd, 1H), 7.10 (m, 1H), 7.04 (d, 1H), |
| | 6.49 (dd, 1H), 4.07 (dd, 1H), 3.82 (s, 3H), 3.46 (dd, 1H), 2.00 (m, 1H), 0.79 (d, 3H), |
| | 0.70 (d, 3H). |
| 177 | δ 9.79 (s, 1H), 8.20 (s, 1H), 7.56 (s, 1H), 7.26 (dd, 1H), 7.16 (m, 1H), 7.06 (d, 1H), |
| | 4.10 (m, 1H), 3.83 (s, 3H), 3.52 (dd, 1H), 2.00 (q, 1H), 0.80 (d, 3H), 0.71 (d, 3H). |
| 179 | δ 9.05 (s, 1H), 8.48 (m, 1H), 7.58 (m, 1H), 7.10 (m, 2H), 4.18 (m, 2H), 3.52 (m, 2H), |
| | 3.17 (s, 3H). |
| 180 | δ 9.03 (d, 1H), 7.88 (d, 1H), 7.57 (m, 1H), 7.11 (t, 2H), 6.50 (t, 1H), 3.90 (m, 1H), |
| | 2.23 (m, 1H), 1.86 (m, 1H), 1.58 (d, 3H), 1.17 (m, 2H), 0.80 (t, 3H). |
| 181 | δ 9.02 (d, 1H), 7.90 (d, 1H), 7.59 (m, 1H), 7.13 (t, 2H), 6.50 (t, 1H), 3.51 (m, 1H), |
| | 2.22 (m, 2H), 2.01 (m, 2H), 0.79 (m, 6H). |
| 182 | δ 9.11 (d, 1H), 8.90 (d, 1H), 7.58 (m, 1H), 7.13 (t, 2H), 6.54 (t, 1H), 3.93 (m, 2H), |
| | 1.50 (m, 2H), 0.74 (s, 9H). |
| 183 | δ 9.11 (d, 1H), 7.90 (d, 1H), 7.59 (m, 1H), 7.12 (t, 2H), 6.51 (t, 1H), 3.92 (d, 2H), |
| | 1.46 (m, 1H), 1.18 (m, 4H), 0.65 (t, 6H). |
| 184 | δ 9.12 (d, 1H), 8.89 (d, 1H), 7.60 (m, 1H), 7.13 (t, 2H), 6.51 (t, 1H), 3.97 (q, 2H), |
| | 1.21 (t, 3H). |
| 185 | δ 9.13 (d, 1H), 7.90 (d, 1H), 7.60 (m, 1H), 7.13 (t, 2H), 6.52 (t, 1H), 3.48 (s, 3H). |
| 186 | δ 9.15 (d, 1H), 7.90 (d, 1H), 7.60 (m, 1H), 7.13 (t, 2H), 6.50 (t, 1H), 4.71 (s, 2H), |
| | 2.23 (s, 1H). |
| 191 | δ 8.00 (d, 2H), 7.60 (m, 1H), 7.15 (t, 2H), 3.85 (m, 2H), 1.80 (m, 1H), 1.25 (m, 1H), |
| | 1.1-1.0 (m, 1H), 0.74 (m, 6H). |
| 192 | δ 9.79 (s, 1H), 8.21 (s, 1H), 7.60 (m, 1H), 7.52 (m, 2H), 7.40 (dd, 1H), 4.13 (dq, 1H), |
| | 3.48 (dd, 1H), 2.02 (m, 1H), 0.84 (d, 3H), 0.75 (d, 3H). |
| 193 | δ 9.10 (d, 1H), 7.89 (d, 1H), 7.58 (m, 1H), 7.48 (m, 2H), 7.40 (m, 1H), 6.50 (m, 1H), |
| | 4.10 (m, 1H), 3.43 (dd, 1H), 2.02 (dt, 1H), 0.83 (d, 3H), 0.73 (d, 3H). |
| 194 | δ 9.80 (s, 1H), 8.21 (s, 1H), 7.78 (dd, 1H), 7.55 (td, 1H), 7.47 (td, 1H), 7.40 (dd, 1H), |
| | 4.15 (td, 1H), 3.44 (dd, 1H), 2.02 (m, 1H), 0.85 (d, 3H), 0.75 (d, 3H). |
| 195 | δ 9.11 (s, 1H), 7.89 (s, 1H), 7.75 (m, 1H), 7.52 (td, 1H), 7.42 (m, 2H), 6.51 (s, 1H), |
| | 4.12 (m, 1H), 3.39 (dd, 1H), 2.03 (dd, 1H), 0.84 (d, 3H), 0.73 (d, 3H). |
| 196 | δ 9.78 (s, 1H), 8.21 (s, 1H), 7.53 (dd, 1H), 7.45 (m, 1H), 7.39 (s, 1H), 7.24 (dd, 1H), |
| | 4.10 (m, 1H), 3.39 (s, 1H), 2.40 (m, 2H), 2.02 (m, 1H), 1.26 (t, 3H), 0.82 (d, 3H), |
| | 0.71 (d, 3H). |
| 197 | δ 9.08 (d, 1H), 7.88 (d, 1H), 7.50 (m, 1H), 7.44 (m, 1H), 7.36 (m, 1H), 7.24 (dd, 1H), |
| | 6.50 (dd, 1H), 4.11 (dd, 1H), 3.34 (dd, 1H), 2.42 (m, 2H), 2.00 (m, 1H), 1.20 (t, 3H), |
| | 0.81 (d, 3H), 0.70 (d, 3H). |
| 202 | δ 9.08 (d, 1H), 7.89 (d, 1H), 7.34 (m, 1H), 7.19 (d, 2H), 6.50 (m, 1H), 3.72 (d, 2H), |
| | 2.17 (s, 6H), 1.76 (m, 1H), 0.78 (d, 6H). |
| 206 | δ 9.68 (s, 1H), 8.17 (s, 1H), 4.33 (br s, 2H), 2.30 (m, 1H), 1.66 (s, 9H), 0.84 (d, 6H). |
| 208 | δ 8.70 (s, 1H), 8.26 (s, 1H), 7.58 (m, 1H), 7.21 (d, 1H), 7.12 (t, 2H), 3.78 (d, 2H), |
| | 2.45 (s, 3H), 2.02 (m, 1H), 0.78 (d, 6H). |
| 209 | δ 8.68 (s, 1H), 8.40 (s, 1H), 7.60 (m, 2H), 7.12 (t, 2H), 3.79 (d, 2H), 2.44 (s, 3H), |
| | 2.02 (m, 1H), 0.78 (d, 6H). |
| 210 | δ 8.69 (s, 1H), 7.64 (m, 1H), 7.50 (m, 1H), 7.45 (m, 1H), 7.16 (t, 2H), 3.87 (d, 2H), |
| | 2.42 (s, 3H), 1.22 (m, 1H), 0.80 (d, 6H). |
| 215 | δ 9.81 (s, 1H), 8.21 (m, 1H), 7.52 (m, 1H), 7.45 (m, 1H), 7.21 (m, 1H), 4.01 (m, 1H) |
| | 3.65 (m, 1H), 2.00 (m, 1H), 0.81 (d, 6H). |
| 216 | δ 7.38 (m, 1H), 7.27 (m, 1H), 7.20 (s, 1H), 7.12 (m, 1H), 4.30 (m, 2H), 3.98 (m, 1H), |
| | 3.26 (m, 1H), 2.97 (t, 2H), 1.82-1.56 (m, 1H), 1.23-0.86 (m, 2H), 0.71 (m, 6H). |
| 218 | δ 7.2 (s, 1H), 6.8 (m, 2H), 4.3 (d, 2H), 3.6 (d, 2H), 2.9 (d, 2H) 1.6 (m, 1H), 1.2 (m, |
| | 1H), 0.9 (m, 1H), 0.7 (m, 6H). |
| 219 | δ 9.0 (d, 1H), 6.8 (m, 2H), 6.3 (d, 1H), 3.8 (d, 2H), 2.4 (s, 3H), 1.6 (m, 1H), 1.2 (m, |
| | 1H), 1.0 (m, 1H), 0.7 (m, 6H). |
| 226 | δ 7.21 (s, 1H), 6.82 (m, 2H), 4.33 (m, 2H), 3.67 (m, 2H), 2.99 (m, 2H), 1.45 (m, 2H), |
| | 1.19 (m, 2H), 0.75 (m, 3H). |
| 230 | δ 9.77 (s, 1H), 8.20 (s, 1H), 7.58 (d, 1H), 7.33-7.26 (m, 1H), 7.17-7.07 (m, 2H), |
| | 3.87 (d, 2H), 2.04 (s, 1H), 0.79 (d, 6H). |
| 231 | δ 9.07 (s, 1H), 7.89 (s, 1H), 7.54 (s, 1H), 7.30-7.20 (m, 1H), 7.16-7.06 (m, 2H), |
| | 6.51 (s, 1H), 3.83 (d, 2H), 2.08-1.98 (m, 1H), 0.77 (d, 6H). |
| 232 | δ 9.79 (s, 1H), 8.21 (s, 1H), 7.87 (d, 1H), 7.75 (t, 1H), 7.65 (s, 1H), 7.58 (d, 1H), |
| | 3.83 (d, 2H), 2.02 (m, 1H), 0.78 (dd, 6H). |
| 233 | δ 9.08 (d, 1H), 7.89 (d, 1H), 7.83 (d, 1H), 7.72 (t, 1H), 7.63 (s, 1H), 7.56 (d, 1H), |
| | 6.51 (m, 1H), 3.79 (d, 2H), 2.00 (dt, 1H), 0.76 (d, 6H). |
| 234 | δ 9.78 (s, 1H), 8.20 (s, 1H), 7.73 (d, 1H), 7.52 (t, 1H), 7.47 (t, 1H), 7.30 (d, 1H), |
| | 3.86 (dd, 2H), 2.04 (m, 1H), 0.79 (s, 6H). |
| 235 | δ 9.78 (s, 1H), 8.84 (dd, 1H), 8.65 (d, 1H), 8.21 (s, 1H), 7.73 (dt, 1H), 7.55 (dd, 1H), |
| | 3.87 (d, 2H), 1.98 (m, 1H), 0.78 (s, 6H). |
| 236 | δ 9.81 (s, 1H), 8.66 (dd, 1H) 8.22 (s, 1H), 7.80 (dd, 1H), 7.51 (dd, 1H), 4.19 (dd, |
| | 1H), 3.42 (dd, 1H), 2.01 (dd, 1H), 0.87 (d, 3H), 0.76 (d, 3H). |
| 237 | δ 9.10 (s, 1H), 8.82 (d, 1H), 8.63 (s, 1H), 7.91 (s, 1H), 7.72 (dd, 1H), 7.55 (dd, 1H), |
| | 6.50 (s, 1H), 3.82 (d, 2H), 1.98 (m, 1H), 0.75 (dd, 6H). |
| 239 | δ 7.67 (s, 1H), 6.92 (m, 2H), 6.23 (m, 1H), 3.83 (m, 2H), 2.49 (s, 3H), 1.55 (m, 2H), |
| | 1.19 (m, 2H), 0.79 (m, 3H). |
| 244 | δ 9.11 (d, 1H), 8.63 (dd, 1H), 7.91 (d, 1H), 7.79 (dd, 1H), 7.48 (dd, 1H), 6.52 (m, |
| | 1H), 4.15 (d, 1H), 3.36 (dd, 1H), 1.98 (m, 1H), 0.85 (d, 3H), 0.74 (d, 3H). |
| 245 | δ 9.79 (s, 1H), 8.20 (s, 1H), 7.45 (t, 1H), 7.13 (d, 1H), 6.90 (d, 1H), 6.85 (s, 1H), |
| | 3.88 (s, 3H), 3.90 (d, 2H), 2.08 (m, 1H), 0.77 (d, 6H). |
| 246 | δ 9.05 (s, 1H), 7.88 (s, 1H), 7.44 (t, 1H), 7.10 (d, 1H), 6.90 (d, 1H), 6.85 (s, 1H), |
| | 3.87 (s, 3H), 3.86 (d, 2H), 2.10 (m, 1H), 0.78 (d, 6H). |
| 247 | δ 9.73 (s, 1H), 8.19 (s, 1H), 7.50-7.35 (m, 2H), 7.16 (s, 1H), 3.88 (m, 1H), 2.46 (s, |
| | 3H), 2.10 (m, 1H), 0.76 (m, 6H). |
| 248 | δ 9.07 (s, 1H), 7.85 (s, 1H), 7.50-7.35 (m, 2H), 7.16 (s, 1H), 6.50 (s, 1H), 3.88 (m, |
| | 1H), 2.46 (s, 3H), 2.10 (m, 1H), 0.76 (m, 6H). |
| 249 | δ 9.78 (s, 1H), 8.21 (s, 1H), 7.90 (d, 1H), 7.75 (t, 1H), 7.70 (s, 1H), 7.61 (d, 1H), |
| | 3.82 (d, 2H), 1.98 (m, 1H), 0.79 (d, 6H). |
| 250 | δ 9.05 (s, 1H), 7.92 (s, 1H), 7.90 (d, 1H), 7.75 (t, 1H), 7.70 (s, 1H), 7.61 (d, 1H), |
| | 6.52 (s, 1H), 3.82 (d, 2H), 1.98 (m, 1H), 0.79 (d, 6H). |
| 251 | δ 9.08 (d, 1H), 7.87 (d, 1H), 7.00 (m, 2H), 6.78 (m, 1H), 6.49 (m, 1H), 6.02 (m, 2H), |
| | 4.00 (m, 1H), 3.82-3.72 (m, 1H), 2.02 (m, 1H), 0.76 (m, 6H). |
| 258 | δ 9.00 (d, 1H), 6.99 (s, 2H), 6.30 (d, 1H), 3.70 (d, 2H), 2.44 (s. 3H), 2.35 (s, 3H), |
| | 2.11 (s, 6H), 1.80 (m, 1H), 0.78 (d, 6H). |
| 259 | δ 9.00 (d, 1H), 7.45-7.30 (m, 2H), 7.26-7.15 (m, 1H), 6.52 (d, 1H), 4.10 (m, 1H), |
| | 3.52 (m, 1H), 1.87-1.58 (m, 1H), 1.24-0.96 (m, 2H), 0.72 (m, 6H). |
| 260 | δ 9.12 (s, 1H), 7.40 (m, 2H), 7.18 (m, 1H), 4.06 (m, 1H), 3.46 (m, 1H), 2.41 (s, 3H), |
| | 1.64 (m, 1H), 1.23-0.98 (m, 2H), 0.72 (m, 6H). |
| 262 | δ 9.02 (d, 1H), 7.40 (m, 1H), 7.33 (m, 1H), 7.18 (m, 1H), 6.31 (d, 1H), 4.10 (m, 1H), |
| | 3.44 (m, 1H), 2.44 (s, 3H), 1.70 (m, 1H), 1.23-0.89 (m, 2H), 0.72 (m, 6H). |
| 263 | δ 9.15-9.02 (m, 1H), 7.89 (d, 1H), 7.46-7.30 (m, 2H), 7.20 (m, 1H), 6.50 (m, 1H), |
| | 4.10 (m, 1H), 3.57-3.40 (m, 1H), 1.89-1.60 (m, 1H), 1.32-0.91 (m, 2H), 0.72 (m, 6H). |
| 264 | δ 7.34 (m, 1H), 7.28 (m, 1H), 7.20 (t, 1H), 7.12 (m, 1H), 4.28 (m, 2H), 3.96 (m, 1H), |
| | 3.26 (m, 1H), 2.97 (t, 2H), 1.83-1.54 (m, 1H), 1.25-0.85 (m, 2H), 0.72 (m, 6H). |
| 265 | δ 9.03 (d, 1H), 7.38 (m, 2H), 7.18 (m, 1H), 6.31 (d, 1H), 4.10 (m, 1H), 3.51 (m, 1H), |
| | 2.44 (s, 3H), 1.76-1.59 (m, 1H), 1.55-1.38 (m, 1H), 1.25-1.13 (m, 2H), 0.77 (t, 3H). |
| 267 | δ 7.38 (m, 1H), 7.28 (m, 1H), 7.18 (m, 1H), 7.12 (m, 1H), 4.28 (m, 2H), 3.90 (m, |
| | 1H), 3.30 (m, 1H), 2.98 (m, 2H), 1.69-1.53 (m, 1H), 1.47-1.32 (m, 1H), 1.22-1.09 (m, |
| | 2H), 0.75 (t, 3H). |
| 274 | δ 7.7-7.6 (m, 1H), 7.1-7.05 (t, 2H), 6.9-6.8 (m, 4H), 5.12 (s, 2H). |
| 279 | δ 9.05 (s, 1H), 7.5-7.45 (m, 1H), 7.2-7.15 (m, 3H), 7.0-6.95 (t, 2H), 6.85 (s, 2H), |
| | 6.25 (s, 1H), 5.18 (s, 2H), 2.45 (s, 3H). |
| 280 | δ 9.05 (s, 1H), 7.5-7.4 (m, 1H), 7.2 (m, 1H), 7.0-6.8 (m, 5H), 6.3 (s, 1H), 5.27 (s, |
| | 2H), 2.45 (s, 3H). |
| 281 | δ 9.07 (m, 1H), 7.89 (d, 1H), 7.40 (m, 1H), 7.34 (m, 1H), 7.18 (m, 1H), 6.50 (m, 1H), |
| | 4.18 (m, 1H), 3.46 (m, 1H), 1.90-1.59 (m, 1H), 1.33-0.90 (m, 2H), 0.72 (m, 6H). |
| 282 | δ 8.99 (d, 1H), 7.38 (m, 1H), 7.30 (m, 1H), 7.18 (m, 1H), 6.30 (d, 1H), 4.10 (m, 1H), |
| | 3.44 (m, 1H), 2.43 (s, 3H), 1.66 (m, 1H), 1.31-1.03 (m, 2H), 0.72 (m, 6H). |
| 283 | δ 7.36 (m, 1H), 7.26 (m, 1H), 7.20 (m, 1H), 7.12 (m, 1H), 4.36-4.22 (m, 2H), |
| | 4.00 (m, 1H), 3.26 (m, 1H), 2.96 (m, 2H), 1.62 (m, 1H), 0.85-1.22 (m, 2H), 0.72 (m, 6H). |
| 285 | δ 8.99 (d, 1H), 7.36 (m, 2H), 7.20 (m, 1H), 6.30 (d, 1H), 4.06 (m, 1H), 3.50 (m, 1H), |
| | 2.43 (s, 3H), 1.73-1.39 (m, 2H), 1.25-1.14 (m, 2H), 0.77 (t, 3H). |
| 286 | δ 7.35 (m, 1H), 7.28 (m, 1H), 7.20 (m, 1H), 7.14 (m, 1H), 4.28 (m, 2H), 3.96 (m, |
| | 1H), 3.32 (m, 1H), 2.96 (m, 2H), 1.58 (m, 1H), 1.36 (m, 1H), 1.14 (m, 2H), 0.74 (m, |
| | 3H). |
| 290 | δ 8.95 (s, 1H), 7.82 (s, 1H), 6.45 (s, 1H), 4.16 (m, 1H), 3.26 (m, 1H), 2.30 (m, 2H), |
| | 2.12 (m, 1H), 1.96 (m, 5H), 1.70 (m, 2H), 1.01 (d, 6H). |
| 292 | δ 8.98 (s, 1H), 7.84 (s, 1H), 6.47 (s, 1H), 4.03 (d, 2H), 2.78 (d, 2H), 2.20 (m, 1H) |
| | 2.05 (m, 1H), 1.06-0.96 (m, 12H). |
| 296 | δ 9.38 (d, 1H), 7.60 (m, 1H), 7.12 (t, 2H), 4.37 (m, 1H), 3.80 (d, 2H), 1.60 (s, 1H), |
| | 1.29 (d, 6H), 1.21 (m, 1H), 1.02 (m, 1H), 0.72 (m, 6H). |
| 297 | δ 9.48 (s, 1H), 7.60 (m, 1H), 7.13 (t, 2H), 3.83 (d, 2H), 3.07 (d, 3H), 1.68 (m, 1H), |
| | 1.21 (m, 1H), 1.03 (m, 1H), 0.72 (m, 6H). |
| 298 | δ 9.20 (s, 1H), 7.60 (m, 1H), 7.14 (t, 2H), 6.25 (s, 1H), 3.85 (d, 2H), 1.72 (m, 1H), |
| | 1.21 (t, 1H), 1.02 (m, 1H), 0.73 (m, 6H). |
| 299 | δ 9.50 (s, 1H), 7.60 (m, 1H), 7.13 (t, 2H), 3.85 (d, 2H), 3.48 (q, 2H), 1.65 (m, 2H), |
| | 1.64 (m, 1H), 1.22 (m, 1H), 1.01 (t, 3H), 0.72 (m, 6H). |
| 300 | δ 9.53 (s, 1H), 7.60 (m, 1H), 7.13 (t, 2H), 3.83 (d, 2H), 3.39 (m, 2H), 1.71 (m, 1H), |
| | 1.22 (m, 2H), 1.01 (m, 1H), 0.73 (m, 6H), 0.58 (d, 2H), 0.31 (d, 2H). |
| 301 | δ 9.46 (s, 1H), 7.62 (m, 1H), 7.17 (t, 2H), 3.82 (d, 2H), 3.50 (m, 2H), 1.67 (m, 2H), |
| | 1.57 (s, 3H), 1.38 (s, 2H), 1.22 (m, 1H), 1.03 (m, 1H), 0.92 (m, 3H), 0.73 (m, 6H). |
| 302 | δ 9.10 (br s, 1H), 7.89 (br s, 1H), 7.40 (m, 1H), 7.34 (m, 1H), 7.20 (m, 1H), 6.51 (br |
| | s, 1H), 4.10 (m, 1H), 3.40 (m, 1H), 2.00 (m, 1H), 0.85 (d, 3H), 0.75 (d, 3H). |
| 303 | δ 9.09 (br s, 1H), 7.89 (br s, 1H), 7.39 (m, 1H), 7.34 (m, 1H), 7.20 (m, 1H), 6.52 (br |
| | s, 1H), 4.10 (m, 1H), 3.38 (m, 1H), 2.00 (m, 1H), 0.84 (d, 3H), 0.75 (d, 3H). |
| 314 | δ 7.56 (m, 1H), 7.09 (t, 2H), 4.02 (t, 2H), 3.70 (d, 2H), 2.62 (t, 2H), 2.22 (m, 2H), |
| | 1.91 (s, 1H), 0.76 (d, 6H). |
| 315 | δ 8.99 (d, 1H), 7.84 (d, 1H), 6.46 (m, 1H), 4.34 (d, 2H), 2.28 (m, 1H), 1.78 (m, 1H), |
| | 1.35 (m, 2H), 0.96 (m, 8H). |
| 316 | δ 8.97 (s, 1H), 7.83 (s, 1H) 6.46 (s, 1H) 4.02 (d, 2H), 2.90 (m, 2H), 2.22 (m, 1H), |
| | 1.26 (m, 3H), 1.00 (d, 6H). |
| 317 | δ 8.96 (d, 1H), 7.83 (s, 1H), 6.46 (s, 1H), 4.01 (d, 2H), 2.83 (m, 2H), 2.22 (m, 1H), |
| | 1.60 (m, 2H), 1.42 (m, 4H), 1.00 (d, 6H), 0.94 (t, 3H). |
| 318 | δ 9.12 (d, 1H), 7.90 (s, 1H), 7.55 (s, 2H), 6.52 (m, 1H), 3.76 (d, 2H), 1.88 (m, 1H), |
| | 0.85 (d, 6H). |
| 319 | δ 7.49 (s, 2H), 4.38 (t, 2H), 3.55 (d, 2H), 2.80 (m, 2H), 1.76 (m, 1H), 0.80 (d, 6H). |
| 320 | δ 9.1 (s, 1H), 7.93 (s, 1H), 7.4-7.3 (m, 1H), 7.25-7.15 (m, 3H), 6.7-6.6 (m, 2H), |
| | 6.5 (s, 1H). |
| 341 | δ 9.05 (s, 1H), 7.35-7.25 (m, 1H), 7.2-7.15 (m, 1H), 7.0-6.85 (m, 3H), 6.6 (d, 1H), |
| | 6.55 (d, 1H), 6.3 (s, 1H), 5.16 (m, 2H), 2.45 (s, 3H). |
| 343 | δ 9.53 (s, 1H), 9.25 (s, 1H), 8.50 (s, 1H), 8.01 (s, 1H), 7.66 (s, 1H), 7.18 (m, 2H), |
| | 3.90 (s, 2H), 2.06 (m, 1H), 0.81 (d, 6H). |
| 344 | δ 9.12 (d, 1H), 7.93 (s, 1H), 7.40 (d, 1H), 7.12 (t, 1H), 6.90 (t, 2H), 6.80 (t, 2H), |
| | 6.57 (d, 1H), 5.15 (s, 2H). |
| 345 | δ 9.17 (d, 1H), 7.93 (d, 1H), 7.42 (d, 1H), 7.22 (t, 1H), 7.12 (t, 1H), 6.88 (d, 1H), |
| | 6.70 (t, 2H), 6.58 (d, 1H), 5.35 (s, 2H). |
| 346 | δ 9.10 (d, 1H), 7.92 (s, 1H), 7.50 (d, 2H), 7.06 (d, 2H), 6.77 (t, 2H), 6.57 (d, 1H), |
| | 5.23 (s, 2H). |
| 347 | δ 9.12 (d, 1H), 7.92 (d, 1H), 7.56 (d, 1H), 7.40 (t, 1H), 7.26 (d, 1H), 6.99 (s, 1H), |
| | 6.76 (t, 2H), 6.58 (t, 1H), 5.24 (s, 2H). |
| 348 | δ 9.16 (d, 1H), 7.97 (s, 1H), 7.59 (d, 1H), 7.49 (t, 1H), 7.38 (t, 1H), 6.97 (d, 1H), |
| | 6.67 (t, 2H), 6.56 (t, 1H), 5.41 (s, 2H). |
| 349 | δ 9.56 (s, 1H), 7.60 (m, 1H), 7.13 (t, 2H), 3.84 (d, 2H), 3.39 (t, 2H), 1.72 (m, 1H), |
| | 1.60 (s, 1H), 1.2 (m, 1H), 1.1 (m, 1H), 1.0 (m, 1H), 0.73 (m, 6H), 0.58 (q, 2H), |
| | 0.32 (q, 2H). |
| 351 | δ 9.08 (t, 1H), 7.92 (d, 1H), 7.54 (d, 1H), 7.39 (s, 1H), 7.25 (m, 1H), 7.18 (t, 1H), |
| | 6.64 (m, 2H), 6.52 (d, 1H). |
| 352 | δ 9.04 (t, 1H), 7.92 (d, 1H), 7.52 (d, 2H), 7.05 (d, 2H), 6.62 (t, 2H), 6.50 (d, 1H). |
| 353 | δ 9.05 (t, 1H), 7.93 (d, 1H), 7.64 (d, 1H), 7.55 (t, 1H), 7.48 (t, 1H), 7.42 (s, 1H), |
| | 6.63 (m, 2H), 6.51 (d, 1H). |
| 354 | δ 9.05 (t, 1H), 7.96 (d, 1H), 7.66 (d, 2H), 7.37 (d, 2H), 6.63 (t, 2H), 6.56 (d, 1H). |
| 359 | δ 8.84 (d, 1H), 8.42 (d, 1H), 7.80 (t, 1H), 7.55 (m, 1H), 7.35 (m, 1H), 7.11 (t, 2H), |
| | 3.75 (d, 2H), 2.21 (s, 3H), 1.98 (m, 1H), 0.75 (d, 6H). |
| 360 | δ 9.12 (t, 1H), 7.93 (d, 1H), 7.62 (d, 1H), 7.34 (m, 2H), 7.26 (t, 1H), 6.63 (t, 1H), |
| | 6.60 (t, 1H), 6.58 (d, 1H). |
| 361 | δ 9.08 (t, 1H), 7.88 (d, 1H), 7.58 (t, 1H), 7.10 (t, 2H), 6.50 (d, 1H), 3.02 (m, 1H), |
| | 0.91 (q, 2H), 0.77 (q, 2H). |
| 362 | δ 9.12 (t, 1H), 7.90 (d, 1H), 7.30 (t, 1H), 7.28 (d, 1H), 7.18 (m, 2H), 7.00 (t, 1H), |
| | 6.89 (d, 2H), 6.81 (t, 1H), 6.52 (d, 1H), 3.22 (m, 1H), 2.20 (m, 1H), 1.41 (t, 2H). |
| 363 | δ 9.03 (t, 1H), 7.88 (d, 1H), 7.57 (m, 1H), 7.09 (t, 2H), 6.50 (d, 1H), 4.44 (m, 1H), |
| | 2.78 (m, 2H), 2.08 (m, 2H), 1.30 (m, 1H), 0.90 (m, 1H). |
| 364 | δ 9.91 (s, 1H), 9.13 (s, 1H), 8.74 (m, 1H), 7.64 (s, 1H), 7.16 (t, 2H), 3.85 (d, 2H), |
| | 1.21 (t, 1H), 0.81 (d, 6H). |
| 377 | δ 9.01 (t, 1H), 7.92 (d, 1H), 7.39 (d, 2H), 6.80 (m, 4H), 6.55 (d, 1H), 5.13 (s, 2H). |
| 390 | δ 8.88 (d, 1H), 6.25 (d, 1H), 4.22 (m, 1H), 4.06 (m, 1H), 3.00 (m, 1H), 2.41 (s, 3H), |
| | 2.08 (m, 2H), 1.92 (m, 1H), 1.47 (d, 3H), 1.02 (m, 6H), 0.92 (m, 3H). |
| 391 | δ 9.13 (s, 1H), 7.92 (s, 1H), 7.78 (s, 2H), 6.53 (s, 1H), 3.76 (d, 2H), 1.88 (m, 1H), |
| | 0.85 (d, 6H). |
| 392 | δ 9.10 (s, 1H), 7.90 (s, 1H), 7.48 (m, 1H), 7.08 (m, 1H), 6.98 (m, 1H), 6.51 (s, 1H), |
| | 2.10 (s, 3H), 1.94 (s, 3H). |
| 393 | δ 9.11 (t, 1H), 7.91 (d, 1H), 7.26 (m, 3H), 6.96 (t, 1H), 6.85 (m, 2H), 6.75 (t, 1H), |
| | 6.48 (d, 1H), 3.69 (s, 3H). |
| 394 | δ 9.10 (t, 1H), 7.92 (d, 1H), 7.35 (t, 1H), 7.25 (t, 1H), 6.82 (m, 4H), 6.77 (s, 1H), |
| | 6.50 (d, 1H), 3.74 (s, 3H). |
| 395 | δ 9.10 (t, 1H), 7.91 (d, 1H), 7.33 (t, 1H), 7.11 (d, 2H), 6.83 (m, 4H), 6.50 (d, 1H), |
| | 3.76 (s, 3H). |
| 396 | δ 9.06 (t, 1H), 7.93 (d, 1H), 7.38 (d, 2H), 7.26 (m, 3H), 7.88 (t, 1H), 7.78 (t, 1H), |
| | 6.50 (d, 1H). |
| 397 | δ 9.06 (t, 1H), 7.92 (d, 1H), 7.41 (t, 1H), 7.32 (t, 1H), 7.20 (d, 2H), 7.13 (s, 1H), |
| | 6.83 (m, 2H), 6.52 (d, 1H). |
| 398 | δ 9.06 (t, 1H), 7.92 (d, 1H), 7.35 (m, 1H), 7.25 (d, 2H), 7.20 (d, 2H), 6.84 (t, 2H), |
| | 6.50 (d, 1H). |
| 399 | δ 9.06 (t, 1H), 7.92 (d, 1H), 7.68 (d, 1H), 7.55 (m, 2H), 7.35 (m, 2H), 6.84 (t, 2H), |
| | 6.50 (d, 1H). |
| 400 | δ 9.08 (m, 1H), 7.91 (s, 1H), 7.56 (m, 1H), 7.10 (m, 2H), 6.54 (s, 1H), 5.43 (s, 2H). |
| 401 | δ 8.94 (d, 1H), 8.69 (d, 1H), 8.15 (s, 1H), 7.69 (s, 1H), 7.63 (s, 1H), 7.15 (t, 2H), |
| | 3.85 (d, 2H); 2.07 (s, 1H) 0.81 (d, 6H). |
| 412 | δ 9.20 (s, 1H), 7.38 (m, 2H), 7.26 (m, 1H), 6.27 (s, 1H), 3.67 (q, 2H), 1.20 (t, 3H). |
| 413 | δ 9.18 (s, 1H), 6.94 (t, 2H), 6.23 (s, 1H), 3.86 (t, 2H), 1.61 (m, 2H), 0.84 (t, 3H). |
| 414 | δ 8.98 (s, 2H), 7.40 (m, 1H), 7.12 (m, 2H), 7.63 (m, 2H), 6.24 (s, 1H). |
| 415 | δ 9.18 (s, 1H), 6.92 (t, 2H), 6.13 (s, 1H), 3.82 (d, 2H), 1.75 (m, 1H), 1.25 (m, 1H), |
| | 1.08 (m, 1H), 0.75 (m, 6H). |
| 416 | δ 9.68 (s, 1H), 6.92 (t, 2H), 4.33 (t, 2H), 3.82 (t, 2H), 2.25 (t, 1H), 1.75 (m, 1H), |
| | 1.25 (m, 2H), 1.05 (m, 1H), 0.75 (m, 6H). |
| 417 | δ 7.91 (s, 1H), 6.92 (t, 2H), 3.93 (s, 3H), 3.81 (m, 2H), 1.75 (m, 1H), 1.25 (m, 1H), |
| | 1.05 (m, 1H), 0.75 (m, 6H). |
| 418 | δ 9.0 (d, 1H), 7.8 (s, 1H), 6.9 (m, 2H), 6.4 (d, 1H), 3.6 (br s, 1H), 2.6 (br s, 2H), |
| | 1.8 (d, 2H), 1.7 (d, 2H), 1.6 (d, 1H), 1.2 (m, 1H), 1.0 (m, 2H). |
| 419 | δ 9.02 (m, 1H), 7.42 (m, 3H), 7.18 (m, 3H), 6.80 (t, 1H), 6.18 (m, 1H). |
| 420 | δ 9.04 (m, 1H), 7.40 (m, 3H), 7.16 (m, 2H), 6.62 (t, 2H), 6.15 (m, 1H). |
| 421 | δ 9.08 (d, 1H) 7.88 (d, 1H), 7.24 (m, 1H), 6.88 (m, 1H), 6.76 (m, 1H), 6.48 (m, 1H), |
| | 4.04 (m, 1H), 3.89 (s, 3H), 3.70 (m, 1H), 1.74 (m, 1H), 1.22 (m, 1H), 0.95 (m, 1H), |
| | 0.72 (m, 6H). |
| 422 | δ 9.41 (d, 1H), 9.15-9.08 (m, 1H), 7.62-7.53 (m, 1H), 7.14-7.07 (m, 2H), 3.71 (d, |
| | 2H), 1.94-1.84 (m, 1H), 0.76 (d, 6H). |
| 423 | δ 8.97-8.89 (br s, 1H), 7.61-7.51 (m, 1H), 7.13-7.06 (m, 2H), 3.69 (d, 2H), 2.57 (s, |
| | 3H), 1.92-1.80 (m, 1H), 0.75 (d, 6H). |
| 424 | δ 8.96 (d, 1H), 7.83 (d, 1H), 6.48-6.43 (m, 1H), 4.31-4.19 (m, 1H), 4.13-4.01 (m, |
| | 1H), 3.07-2.96 (m, 1H), 2.13-2.02 (m, 2H), 2.01-1.89 (m, 1H), 1.49 (d, 3H), |
| | 1.04-0.92 (m, 9H). |
| 425 | δ 9.00 (m, 1H), 7.22 (m, 1H), 6.88 (m, 1H), 6.78 (m, 1H), 6.31 (m, 1H), 4.02 (m, |
| | 1H), 3.89 (s, 3H), 3.74 (m, 1H), 2.44 (s, 3H), 1.77 (m, 1H), 1.22 (m, 1H), 1.01 (m, |
| | 1H), 0.72 (m, 6H). |
| 426 | δ 9.79 (br s, 1H), 9.19 (m, 1H), 7.93 (m, 1H), 7.06 (m, 1H), 6.90 (m, 2H), 6.56 (m, |
| | 1H), 4.06 (m, 1H), 3.74 (m, 1H), 1.76 (m, 1H), 1.19 (m, 1H), 0.99 (m, 1H), 0.74 (m, |
| | 6H). |
| 427 | δ 9.05 (d, 1H), 7.40 (q, 1H), 7.10 (t, 1H), 7.00-6.6 1 (q, 2H), 6.98 (t, 2H), 6.75 (d, |
| | 1H). |
| 428 | δ 8.90 (d, 1H), 7.37 (q, 1H), 7.08 (t, 1H), 6.97 (t, 2H), 6.60 (q, 2H), 6.40 (d, 1H), |
| | 1.41 (s, 9H). |
| 429 | δ 8.96 (d, 1H), 7.40-7.38 (q, 1H), 7.12 (t, 1H), 6.97 (t, 2H), 6.63 (q, 2H), 6.52 (d, |
| | 1H). |
| 430 | δ 9.10 (d, 1H), 7.39 (q, 1H), 7.18 (t, 1H), 6.98 (t, 2H), 6.87 (d, 1H), 6.70-6.60 (q, |
| | 2H). |
| 431 | δ 8.09 (t, 2H), 7.35 (t, 1H), 7.08 (t, 1H), 6.98 (t, 2H), 6.60 (q, 2H), 6.36 (t, 2H). |
| 432 | δ 9.05 (d, 1H), 8.14 (d, 1H), 7.38 (q, 1H), 7.20 (s, 1H), 7.12 (t, 1H), 6.98 (t, 2H), |
| | 6.64 (q, 2H). |
| 433 | δ 9.74 (s, 1H), 8.24 (s, 1H), 7.40 (q, 1H), 7.17 (t, 1H), 6.99 (t, 2H), 6.66 (q, 2H). |
| 434 | δ 7.25 (m, 1H), 7.00 (t, 1H), 6.93 (t, 2H), 6.60-6.58 (q, 2H), 4.39 (t, 2H), 3.02 (t, 2H), |
| | 0.90 (t, 1H). |
| 435 | δ 8.95 (m, 1H), 7.56 (m, 1H), 7.11 (m, 2H), 6.29 (m, 1H), 3.62 (m, 1H), 2.65 (m, |
| | 2H), 2.43 (s, 3H), 1.80 (m, 2H), 1.69 (m, 2H), 1.54 (m, 1H), 1.19 (m, 1H), 0.98 (m, |
| | 2H). |
| 436 | δ 8.95 (d, 1H), 6.89 (m, 2H), 6.29 (d, 1H), 3.63 (m, 1H), 2.65 (m, 2H), 2.43 (s, 3H), |
| | 1.82 (m, 2H), 1.69 (m, 2H), 1.55 (m, 1H), 1.20 (m, 1H), 1.04 (m, 2H). |
| 440 | δ 9.15 (s, 1H), 7.22 (d, 1H), 6.94 (s, 1H), 6.32 (s, 1H), 3.78 (m, 2H), 1.77 (m, 1H), |
| | 1.04 (m, 1H), 0.91 (m, 1H), 0.75 (m, 6H). |
| 441 | δ 9.18 (s, 1H), 7.48 (m, 1H), 7.10 (t, 1H), 6.32 (s, 1H), 3.82 (d, 2H), 1.71 (m, 1H), |
| | 1.22 (m, 1H), 1.02 (m, 1H), 0.75 (m, 6H). |
| 442 | δ 9.02 (m, 1H), 7.60 (m, 1H), 7.46 (m, 1H), 7.35 (d, 1H), 6.31 (m, 1H), 4.07 (m, 1H), |
| | 3.39 (m, 1H), 2.43 (s, 3H), 1.98 (m, 1H), 0.83 (d, 3H), 0.74 (d, 3H). |
| 448 | δ 9.02 (d, 1H), 7.40 (q, 1H), 7.12 (t, 1H), 6.97 (t, 2H), 6.63 (q, 2H), 6.44 (d, 1H). |
| 451 | δ 9.10 (m, 1H), 7.88 (m, 1H), 6.65 (m, 2H), 6.50 (m, 1H), 3.87 (m, 5H), 1.75 (m, |
| | 1H), 1.25 (m, 1H), 1.05 (m, 1H), 0.75 (m, 6H). |
| 452 | δ 9.00 (m, 1H), 6.62 (m, 2H), 6.30 (m, 1H), 3.89 (s, 3H), 3.82 (m, 2H), 2.43 (s, 3H), |
| | 1.73 (m, 1H), 1.24 (m, 1H), 1.04 (m, 1H), 0.75 (m, 6H). |
| 453 | δ 10.53 (br s, 1H), 9.24 (d, 1H), 7.94 (d, 1H), 6.74 (d, 2H), 6.57 (m, 1H), 3.90 (d, 2H), |
| | 1.76 (m, 1H), 1.26 (m, 1H), 1.05 (m, 1H), 0.77 (m, 6H). |
| 457 | δ 9.09 (m, 1H), 7.88 (m, 1H), 6.63 (m, 2H), 6.50 (m, 1H), 3.90 (s, 3H), 3.85 (m, 2H), |
| | 1.74 (m, 1H), 1.25 (m, 1H), 1.04 (m, 1H), 0.75 (m, 6H). |
| 460 | δ 9.10 (m, 1H), 7.90 (m, 1H), 6.90 (m, 2H), 6.51 (m, 1H), 3.85 (d, 2H), 0.96 (m, 1H), |
| | 0.50 (m, 2H), 0.24 (m, 2H). |
| 465 | δ 9.11 (d, 1H), 7.90 (d, 1H), 6.87 (m, 2H), 6.52 (m, 1H), 5.72 (m, 1H), 5.17 (d, 1H), |
| | 4.92 (d, 1H), 4.55 (d, 2H). |
| 475 | δ 7.25 (m, 1H), 7.14 (dd, 1H), 7.07 (dd, 1H), 3.96 (br s, 1H), 3.84 (br s, 1H), 2.31 (s, |
| | 3H), 2.09 (s, 3H), 1.82 (m, 1H), 1.17 (m, 1H), 1.01 (m, 1H), 0.72 (m, 6H). |
| 476 | δ 7.59 (m, 3H), 7.24 (m, 2H), 3.82 (br s, 2H), 2.30 (s, 3H), 2.12 (m, 1H), 2.06 (s, |
| | 3H), 1.22 (m, 1H), 0.90 (m, 1H), 0.72 (d, 6H). |
| 477 | δ 9.07 (m, 1H), 7.87 (m, 1H), 7.22 (m, 1H), 6.88 (m, 1H), 6.80 (m, 1H), 6.49 (m, |
| | 1H), 4.14 (m, 2H), 4.03 (m, 1H), 3.72 (m, 1H), 2.80 (m, 2H), 2.39 (s, 6H), 1.76 (m, |
| | 1H), 1.20 (m, 1H), 0.99 (m, 1H), 0.72 (m, 6H). |
| 478 | δ 9.00 (m, 1H), 7.81 (m, 1H), 7.15 (m, 1H), 6.82 (m, 1H), 6.72 (m, 1H), 6.43 (m, |
| | 1H), 4.12 (m, 2H), 3.96 (m, 1H), 3.65 (m, 1H), 2.90 (m, 2H), 2.60 (m, 4H), 1.78 (m, |
| | 4H), 1.67 (m, 1H), 1.14 (m, 1H), 0.93 (m, 1H), 0.65 (m, 6H). |
| 479 | δ 9.09 (m, 1H), 7.88 (m, 1H), 6.65 (m, 2H), 6.50 (m, 1H), 4.12 (m, 2H), 3.85 (m, |
| | 2H), 2.77 (m, 2H), 2.36 (s, 6H), 1.73 (m, 1H), 1.24 (m, 1H), 1.04 (m, 1H), 0.75 (m, |
| | 6H). |
| 480 | δ (acetone-d₆) 9.07 (m, 1H), 7.83 (m, 1H), 7.08 (m, 2H), 6.55 (m, 1H), 4.71 (m, 2H), |
| | 3.90 (m, 2H), 3.63 (m, 2H), 3.39 (m, 4H), 2.06 (m, 4H), 1.78 (m, 1H), 1.30 (m, 1H), |
| | 1.06 (m, 1H), 0.76 (m, 6H). |
| 481 | δ 9.09 (d, 1H), 7.88 (m, 1H), 6.64 (m, 2H), 6.50 (m, 1H), 4.09 (m, 2H), 3.87 (d, 2H), |
| | 2.47 (t, 2H), 2.27 (s, 6H), 2.00 (m, 2H), 1.00 (m, 1H), 0.48 (m, 2H), 0.24 (m, 2H). |
| 482 | δ 9.09 (m, 1H), 7.88 (m, 1H), 6.63 (m, 2H), 6.50 (m, 1H), 4.08 (m, 2H), 3.85 (m, |
| | 2H), 2.48 (m, 2H), 2.28 (s, 6H), 2.00 (m, 2H), 1.74 (m, 1H), 1.25 (m, 1H), 1.04 (m, |
| | 1H), 0.75 (m, 6H). |
| 483 | δ 9.09 (m, 1H), 7.88 (m, 1H), 6.65 (m, 2H), 6.50 (m, 1H), 4.12 (m, 2H), 3.85 (m, |
| | 2H), 2.78 (m, 2H), 2.36 (s, 6H), 1.74 (m, 1H), 1.25 (m, 1H), 1.03 (m, 1H), 0.75 (m, |
| | 6H). |
| 484 | δ 9.22 (br s, 1H), 6.69-6.65 (m, 2H), 6.13 (br s, 1H), 4.15 (t, 2H), 4.00-3.81 (m, 1H), |
| | 2.78 (t, 2H), 2.41-2.28 (m, 7H), 2.78-2.65 (m, 1H), 1.30-1.19 (m, 1H), 1.09-1.00 (m, |
| | 1H), 0.79-0.68 (m, 6H). |
| 485 | δ 9.04 (d, 1H), 7.90 (d, 1H), 6.67-6.64 (m, 2H), 6.52 (m, 1H), 4.18-4.0 (m, 4H), |
| | 2.92-2.80 (m. 1H), 2.58-2.48 (m, 2H), 2.31 (s, 6H), 2.10-2.00 (m, 2H), 1.03 (d, 3H). |
| 486 | δ 9.20 (s, 1H), 6.70-6.60 (m, 2H), 6.13 (s, 1H), 4.17 (t, 2H), 3.98-3.78 (m, 2H), |
| | 2.78 (t, 2H), 2.45 (s, 6H), 2.22-2.10 (m, 2H), 1.82-1.70 (m, 1H), 1.31-1.18 (m, 1H), |
| | 1.10-0.98 (m, 1H), 0.80-0.71 (m, 6H). |
| 487 | δ 7.45 (d, 1H), 7.41 (d, 1H), 6.87 (m, 2H), 4.06 (s, 3H), 3.74 (d, 2H), 2.01 (m, 1H), |
| | 0.78 (d, 6H). |
| 490 | δ 7.44 (d, 1H), 7.40 (d, 1H), 6.61 (m, 2H), 4.05 (s, 3H), 3.88 (s, 3H), 3.81 (m, 2H), |
| | 1.77 (m, 1H), 1.25 (m, 1H), 1.01 (m, 1H), 0.74 (m, 6H). |
| 491 | δ 9.06 (d, 1H), 7.91 (d, 1H), 7.36 (m, 1H), 7.09 (m, 1H), 6.98 (m, 2H), 6.50 (m, 1H), |
| | 6.38 (m, 2H), 3.94 (m, 2H), 2.46 (m, 2H), 2.27 (s, 6H), 1.94 (m, 2H). |
| 493 | δ (methanol-d₆) 9.08 (d, 1H), 8.23 (m, 1H), 7.89 (d, 1H), 6.91 (m, 2H), 6.59 (s, 1H), |
| | 4.22 (t, 2H), 3.87 (m, 2H), 3.23 (m, 2H), 2.75 (s, 3H), 2.21 (m, 2H), 1.73 (m, 1H), |
| | 1.24 (m, 1H), 1.06 (m, 1H), 0.73 (m, 6H). |
| 494 | δ (methanol-d₆) 9.14 (m, 1H), 6.92 (m, 2H), 6.61 (m, 1H), 6.54 (s, 1H), 4.22 (m, |
| | 2H), 3.87 (d, 2H), 3.23 (m, 2H), 2.74 (s, 3H), 2.44 (s, 3H), 2.22 (m, 2H), 1.71 (m, |
| | 1H), 1.25 (m, 1H), 1.06 (m, 1H), 0.75 (m, 6H). |
| 495 | δ 9.09 (m, 1H), 7.49 (m, 1H), 6.63 (d, 2H), 6.51 (m, 1H), 4.09 (t, 2H), 3.83 (d, 2H), |
| | 2.47 (t, 2H), 2.28 (s, 6H), 1.99 (m, 3H). 0.80 (d, 6H). |
| 496 | δ 9.09 (d, 1H), 6.62 (d, 2H), 6.30 (d, 1H), 4.13 (t, 2H), 3.80 (d, 2H), 2.98 (t, 2H), |
| | 2.58 (s, 3H), 2.43 (s, 3H), 2.18 (m, 2H), 1.98 (m, 1H), 0.78 (d, 6H). |
| 497 | δ 9.01 (d, 1H), 6.62 (d, 2H), 6.30 (d, 1H), 4.08 (t, 2H), 3.80 (d, 2H), 2.59 (t, 2H), |
| | 2.43 (s, 3H), 2.34 (s, 6H), 2.05 (m, 2H), 1.98 (m, 1H), 0.78 (d, 6H). |
| 498 | δ (methanol-d₆) 9.03 (d, 1H), 6.88 (d, 2H), 6.42 (d, 1H), 4.16 (m, 2H), 3.88 (m, 2H), |
| | 2.56 (m, 2H), 2.40 (s, 3H), 2.32 (s, 6H), 2.03 (m, 2H), 1.71 (m, 1H), 1.25 (m, 1H), |
| | 1.07 (m, 1H), 0.77 (m, 6H). |
| 499 | δ (methanol-d₆) 9.11 (d, 1H), 7.91 (d, 1H), 6.90 (m, 2H), 6.61 (m, 1H), 4.14 (m, 2H), |
| | 3.90 (m, 2H), 3.60 (m, 2H), 3.11 (m, 2H), 2.96 (m, 1H), 2.11 (m, 5H), 1.73 (m, 1H), |
| | 1.27 (m, 1H), 1.09 (m, 1H), 0.75 (m, 6H). |
| 500 | δ (methanol-d₆) 9.12 (br s, 1H), 7.92 (br s, 1H), 6.90 (d, 2H), 6.63 (br s, 1H), 4.21 (t, |
| | 2H), 3.81 (d, 2H), 3.22 (t, 2H), 2.74 (s, 3H), 2.21 (m, 2H), 1.98 (br s, 1H), 0.78 (d, |
| | 6H). |
| 501 | δ 9.11 (d, 1H), 7.89 (d, 1H), 6.99 (m, 2H), 6.51 (m, 1H), 3.85 (d, 2H), 2.35 (s, 3H), |
| | 1.71 (m, 1H), 1.25 (m, 1H), 1.06 (m, 1H), 0.75 (m, 6H). |
| 503 | δ ppm 10.20 (s, 1H), 9.22 (d, 1H), 7.94 (d, 1H), 6.72 (d, 2H), 6.56 (m, 1H), |
| | 3.90 (d, 2H), 1.75 (m, 1H), 1.26 (m, 1H), 1.05 (m, 1H), 0.77 (m, 6H) |
| 504 | δ 9.01 (d, 1H), 6.62 (m, 2H), 6.30 (d, 1H), 3.89 (s, 3H), 3.84 (d, 2H), 2.43 (s, 3H), |
| | 1.73 (m, 1H), 1.24 (m, 1H), 1.02 (m, 1H), 0.74 (m, 6H). |

^{a}¹H NMR data are in ppm downfield from tetramethylsilane. Couplings are designated by (s)—singlet, (d)—doublet, (t)—triplet, (q)—quartet, (m)—multiplet, (dd)—doublet of doublets, (dt)—doublet of triplets, (dq)—doublet of quartets, (br s)—broad singlet and (td)—triplet of doublets.

### Biological Examples of the Invention

### Assay for Inhibition of Tubulin Polymerization

Bovine brain derived tubulin and reagents for tubulin polymerization were purchased from Cytoskeleton, Denver, Colo. (Catalog No. HTS02) and assays were carried out as recommended by Cytoskeleton. Briefly, >97% pure tubulin was dissolved in GPEM buffer solution composed of 80 mM piperazine-N,N-bis(2-ethanesulfonic acid) sequisodium salt, 2.0 mM magnesium chloride and 0.5 mM ethylene glycol-bis(β-aminoethylether)-N,N,N,N-tetraacetic acid at pH 6.9, containing 5% glycerol and 1 mM GTP (Guanosine 5′-triphosphate) to a concentration of 2 mg/mL. This tubulin solution was freshly made and stored on ice until needed.

The polymerization of the tubulin involved dispensing 100 μL of the protein solution to the wells of a half area 96-well microtiter plate already containing 10 μL of the compounds to be tested that had been pre-equilibrated to 37° C. for 30 minutes. The concentration of DMSO (dimethylsulfoxide) in all wells did not exceed 0.5%. Controlled reactions without compound were performed in wells containing only 10 μL of DMSO.

Compounds to be tested were initially dissolved in DMSO, and then further diluted to 10 times desired final concentration in the GPEM buffer solution described above by pipeting 5 μL of compound in DMSO into 95 μL of the GPEM buffer. The concentration range of the compounds was 0.1 to 30 μM.

Polymerization was initiated by the addition of 100 μL of the fresh tubulin solution at 4° C. to the plate at 37° C. and the change in turbidity of the solution monitored at 340 nm for extended periods up to 10 h using a SpectraMax plate reader (Molecular Devices Corp, CA) thermostated at 37° C. The turbidity at time zero was subtracted from the maximum turbidity reached during the polymerization, and replicate values for each compound concentration were averaged to provide the maximum turbidity value (A340max). For comparative purposes, the A340max for 10 μM of compound was compared to that for paclitaxel (A340p) at 10 μM and the ratio displayed in Table 1.

TABLE 1
Effect on tubulin polymerization by representative examples of the
invention relative to standard paclitaxel (p) as determined by change
in optical density (OD) at 340 nm.

| Compound | A340max/A340(p) |
|---|---|
| 272 | 1.79 |
| 324 | 1.54 |
| 3 | 2.21 |
| 240 | 3.21 |
| 53 | 2.67 |
| 263 | 2.67 |
| 415 | 2.54 |
| 155 | 2.42 |
| 460 | 3.29 |
| 477 | 2.54 |
| 479 | 3.13 |
| 483 | 4.58 |
| 482 | 4.17 |
| 481 | 3.75 |
| 478 | 2.42 |
| 480 | 2.08 |

### Cell Culture

Human rabdomyosarcoma (RD) and mouse neuroblastoma (N1E115) cell lines were obtained from the American Type Culture Collection (ATCC, Rockville, Md.). The RD cells were grown in Dulbecco's modified eagle medium (DMEM) supplemented with 4 mM glutamine, containing 10% fetal bovine serum (ATCC #30-2020) and supplemented with 1% penicillin and 1% streptomycin. When confluent, the cells were maintained by passage (about once per week) until needed. The N1E115 line was also cultured in DMEM with 4 mM glutamine containing 10% newborn calf serum (Gibco, Grand Island, N.Y.) and likewise maintained.

### Assay for Inhibition of Cell Proliferation

The proliferation of rhabdomyosarcoma cells was determined using a cell proliferation assay kit based on the formation of insoluble formazan crystals from 3-(4,5-dimethylthiazol-2-yl)-2,5-diphenyltetrazolium bromide (MTT). The rhabdomyosarcoma cells were cultured to a density of 10⁵ cells per mL. The cell culture (100 μL) was dispensed into wells of a 96-well plate to 10⁴ cells per well. The plate was incubated at 37° C. for 3 h until the cells firmly attached to the well surfaces.

A second 96-well round bottom plate was made up containing the test compounds, serially diluted to cover the concentration range of interest using the DMEM medium plus antibiotics. The final concentration of DMSO in which the compounds were initially dissolved was maintained at a constant 0.5% in each well and the volume of the compounds in the plate was 220 μL. Following the 3 h incubation the medium was removed from the plate containing the cells and replaced with 200 μL of the solutions containing the compounds. The cells were incubated for a further 96 h before the assessment of growth inhibition using MTT.

For the determination of the compound IC₅₀, the plate containing the cells was rinsed with saline solution composed of NaCl (120 mM), KCl (3 mM), MgCl₂ (2 mM), CaCl₂ (2 mM), D-Glucose (25 mM) and Herpes (10 mM) at pH 7.4. The cells were left bathing in 100 μL of the saline solution to which was added 100 μL of MTT in saline (12 mM). Incubation was continued for 4 h at 37° C. to produce the blue formazan color which was quantified by optical density measurements at 570 nm. Background correction of all test wells was performed by subtraction of the yellow MTT solution color measured at 570 nm in cell-free wells. The data were normalized to solvent only control wells. Table 2 shows the IC₅₀ of a representative set of the compounds compared to the average IC₅₀ for paclitaxel as determined from multiple experiments.

TABLE 2
Activities of representative examples of the invention relative to
standard paclitaxel against rhabdomyosarcoma (RD) and neuroblastoma
(N1E115) cells.

| | | RD Ratio | | N1E115 Ratio |
|---|---|---|---|---|
| | RD | (paclitaxel/ | N1E115 | (paclitaxel/ |
| Compound | IC₅₀ (nM) | compound) | IC₅₀ (nM) | compound) |
| 477 | 261.7* | 0.06 | 462.0 | 0.19 |
| 479 | 199.7* | 0.09 | 25.0 | 3.52 |
| 483 | 39.7* | 0.43 | 81.0 | 1.09 |
| 482 | 1.5* | 11.33 | 2.6 | 33.85 |
| 481 | 3.2* | 5.37 | 30.0 | 2.93 |
| 478 | 470.0** | 0.04 | 1090.0 | 0.08 |
| 480 | 463.0* | 0.04 | 393.0 | 0.22 |
| 498 | 3.8 | 4.47 | 14.0 | 6.29 |
| 494 | 0.4 | 42.50 | 1.3 | 67.69 |
| 493 | 0.6 | 28.33 | 4.5 | 19.56 |
| 495 | 35.0 | 0.49 | 353.0 | 0.25 |
| 497 | 77.0 | 0.22 | 180.0 | 0.49 |
| 496 | 43.0 | 0.40 | 90.0 | 0.98 |
| 457 | 1.1 | 15.45 | 39.0 | 2.26 |
| 503 | 114.0 | 0.15 | 256.0 | 0.34 |
| 504 | 16.3 | 1.04 | 102.0 | 0.86 |
| 499 | 10.0 | 1.70 | 12.0 | 7.33 |
| 500 | 2.0 | 8.50 | 16.0 | 5.50 |
| 501 | 57.0 | 0.30 | 62.0 | 1.42 |
| 485 | 27.5 | 0.62 | 151.0 | 0.58 |
| 484 | 10.0 | 1.70 | 285.0 | 0.31 |
| 491 | 28.5 | 0.60 | 22.0 | 4.00 |
| 492 | 3.3 | 5.15 | 16.0 | 5.50 |
| 324 | 50.0 | 0.34 | 147.0 | 0.60 |
| 155 | 20.0 | 0.85 | 75.0 | 1.17 |
| 263 | 50.0 | 0.34 | 80.0 | 1.10 |
| 415 | 40.0 | 0.43 | 60.0 | 1.47 |
| 1 | 500.0 | 0.03 | 110.0 | 0.80 |
| 3 | 3010.0 | 0.01 | 790.0 | 0.11 |
| 272 | 70.0 | 0.24 | 70.0 | 1.26 |
| 240 | 1670.0 | 0.01 | 59.0 | 1.49 |
| 53 | 230.0 | 0.07 | 700.0 | 0.13 |
| 460 | 90.0 | 0.19 | 170.0 | 0.52 |
| paclitaxel | 17.0 | — | 88.0 | — |

*Reported as the average of three replicated experiments.
**Reported as the average of two replicated experiments.

These results and observations confirm that compounds of Formula 1 are potent cytotoxins. In particular, the results of the assays conducted in relation to cancerous cell lines are predictive of anti-tumor efficacy in individuals.

1. A method of inhibiting undesired proliferation of an animal cell, said method comprising contacting said cell with a compound of Formula 1 and all pharmaceutically acceptable salts, N-oxides, hydrates, solvates, crystal forms or geometric and stereoisomers thereof: 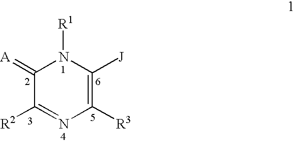
wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₅-C₁₀ alkylcycloalkylalkyl, C₇-C₁₄ alkylcycloalkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³, —NR⁸C(O)R²⁶, —NR⁸C(O)NR²⁷ or —NR⁸C(O)OR²⁸; or
R² is a 5- or 6-membered heteroaromatic ring or a 8-, 9- or 10-membered heteroaromatic bicyclic ring system, each ring or ring system optionally substituted with up to 5 substituents independently selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴; or
R² and R⁷ are taken together as —N═C(R¹⁶)—;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ is independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
each R⁸ is independently H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
R¹⁶ is H, halogen, cyano, C₁-C₆ alkyl, C₁-C₄ haloalkyl, C₃-C₆ cycloalkyl, C₃-C₆ halocycloalkyl, C₂-C₆ alkenyl, C₂-C₆ alkynyl, C₁-C₄ alkoxy, C₁-C₄ haloalkoxy, C₁-C₄ alkylthio, C₁-C₄ haloalkylthio or C₂-C₅ alkoxycarbonyl;
J is C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylsulfinyl, C₁-C₄ alkylsulfonyl, C₂-C₆ alkoxycarbonyl, C₂-C₆ alkylcarbonyl, C₁-C₄ alkylamino, C₂-C₆ dialkylamino and C₃-C₆ trialkylsilyl; or
J is a phenyl, benzyl, naphthalene, 5- or 6-membered heteroaromatic ring or 8-, 9- or 10-membered heteroaromatic bicyclic ring system, each ring or ring system optionally substituted with up to 5 substituents independently selected from R²⁹ and R³⁰;
R²⁹ is halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R³⁰ is —Y-X-Q;
Y is O, S(O)ₚ, NR³¹ or direct bond;
X is C₁-C₆ alkylene, C₂-C₆ alkenylene, C₃-C₆ alkynylene, C₃-C₆ cycloalkylene or C₃-C₆ cycloalkenylene, each optionally substituted with one or more substituents independently selected from the group consisting of halogen, cyano, nitro, hydroxy, (═O), C₁-C₆ alkoxy and C₁-C₆ haloalkoxy;
Q is NR³²R³³, OR³⁵ or S(O)ₚR³⁵;
R³¹ is H, C₁-C₆ alkyl, C₁-C₆ haloalkyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
each R³² and R³³ is independently H; or C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl; or R³² and R³³ when optionally taken together with the nitrogen atom to which each is attached form a heterocyclic ring of 3 to 6 ring atoms optionally substituted with R³⁴;
R³⁴ is halogen, C₁-C₆ alkyl, C₁-C₆ haloalkyl or C₁-C₆ alkoxy;
each R³⁵ is independently H, C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
p is 0, 1 or 2;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents independently selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ is independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or —CH₂CH(CH₃)OCH(CH₃)CH₂—;
each R²² and R²³ is independently H; or C₁-C₄ alkyl, C₁-C₄ alkoxy, C₃-C₈ cycloalkyl or C₄-C₈ cycloalkylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R²⁵ is H, C₁-C₄ alkyl or C₁-C₄ haloalkyl; and
R²⁶ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from R³⁶;
R³⁶ is C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy or C₁-C₄ haloalkoxy; and
each R²⁷ and R²⁸ is independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, halogen, cyano, nitro, C₁-C₄ alkoxy and C₁-C₄ haloalkoxy.

2. The method of claim 1 wherein
A is O or S;
R¹ is C₂-C₆ alkyl, C₂-C₆ haloalkyl, C₃-C₈ cycloalkyl, C₄-C₈ cycloalkylalkyl, NR⁴R⁵, G¹ or G²;
R² is cyano, —C(W)NR²²R²³ or —NR⁸C(═O)R²⁶; or a 5- or 6-membered heteroaromatic ring; or a 5- or 6-membered saturated or partially saturated heterocyclic ring, optionally including 1-3 ring members selected from the group consisting of C(═O);
W is O or S;
R³ is halogen, cyano or C₁-C₆ alkyl;
X is C₁-C₆ alkylene or C₂-C₆ alkenylene;
R⁴ and R⁵ are independently H, C₁-C₈ alkyl or C₁-C₈ haloalkyl; and J is phenyl optionally substituted with substituents independently selected from halogen, C₁-C₆ alkyl, C₁-C₆ haloalkyl and R³⁰.

3. The method of claim 2 wherein
A is O;
R¹ is C₂-C₆ alkyl, C₂-C₆ haloalkyl, C₄-C₈ cycloalkylalkyl, G¹ or G²;
R² is 5- or 6-membered heteroaromatic ring, cyano, —CONH₂ or —NHC(═O)CH₃;
R³ is halogen, cyano or C₁-C₃ alkyl;
X is C₃-C₄ alkylene or C₂-C₄ alkenylene; and
J is phenyl, optionally substituted at the 2, 3, 4 and 6 positions with substituents independently selected from halogen, C₁-C₆ alkyl, C₁-C₆ haloalkyl and R³⁰.

4. The method of claim 3 wherein
R¹ is C₃-C₆ alkyl, C₃-C₆ haloalkyl, C₄-C₈ cycloalkylalkyl, or phenyl, optionally substituted with from 1 to 4 substituents independently selected from R¹⁸;
R² is 5- or 6-membered heteroaromatic ring, each ring optionally substituted with up to 3 substituents independently selected from R²⁴; or —CONH₂ or —NHC(═O)CH₃;
R³ is fluoro, chloro, bromo or methyl;
X is C₃-C₄ alkylene; and
J is phenyl optionally substituted at the 2, 3, 4 and 6 positions with substituents independently selected from chloro and fluoro, methyl, and R³⁰.

5. The method of claim 4 wherein
R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₆ alkyl or C₁-C₄ haloalkyl; or —CONH₂;
Y is O or NR³¹; and
Q is NR³²R³³ or OR³⁵.

6. The method of claim 5 wherein
R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₄ alkyl or C₁-C₃ haloalkyl; or —CONH₂;
Y is O or NH; and
each R³², R³³ and R³⁵ is independently H or C₁-C₄ alkyl or C₁-C₃ haloalkyl.

7. The method according to claim 1 wherein the compound is selected from the group consisting of:
5-chloro-6-[4-[3-(dimethylamino)propoxy]-2,6-difluorophenyl]-1-[(2S)-2-methylbutyl]-3-(1H-pyrazol-1-yl)-2(1H)-pyrazinone,
5-chloro-1-cyclopropylmethyl-6-[4-[3-(dimethylamino)propoxy]-2,6-difluorophenyl]-3-(1H-pyrazol-1-yl)-2(1H)-pyrazinone,
5-chloro-6-[2,6-difluoro-4-[3-(methylamino)propoxy]phenyl]-1-[(2S)-2-methylbutyl]-3-(1H-pyrazol-1-yl)-2(1H)-pyrazinone,
6-chloro-5-[4-[3-(dimethylamino)propoxy]-2,6-difluorophenyl]-3,4-dihydro-4-[(2S)-2-methylbutyl]-3-oxopyrazinecarboxamide,
6-chloro-5-[2,6-difluoro-4-[3-(methylamino)propoxy]phenyl]-3,4-dihydro-4-[(2S)-2-methylbutyl]-3-oxopyrazinecarboxamide,
6-chloro-5-[4-[3-(dimethylamino)propoxy]-2,6-difluorophenyl]-3,4-dihydro-3-oxo-4-(3,3,3-trifluoro-2-methylpropyl)pyrazinecarboxamide,
6-chloro-5-[2,6-difluoro-4-[3-(methylamino)propoxy]phenyl]-3,4-dihydro-3-oxo-4-(3,3,3-trifluoro-2-methylpropyl)pyrazinecarboxamide,
5-chloro-6-[4-[3-(dimethylamino)propoxy]-2,6-difluorophenyl]-1-(3-fluorophenyl)-3-(1H-pyrazol-1-yl)-2(1H)-pyrazinone,
5-chloro-6-[2,6-difluoro-4-[3-(methylamino)propoxy]phenyl]-1-(3-fluorophenyl)-3-(1H-pyrazol-1-yl)-2(1H)-pyrazinone,
5-chloro-6-[4-[3-(dimethylamino)propoxy]-2,6-difluorophenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[4-[3-(dimethylamino)propoxy]-2,6-difluorophenyl]-1[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(1H-pyrazol-1-yl)-2(1H)-pyrazinone,
5-chloro-6-[2,6-difluoro-4-[3-(methylamino)propoxy]phenyl]-1-[(2S)-2-methylbutyl]-3-(1-methyl-1H-pyrazol-3-yl)-2(1H)-pyrazinone,
5-Chloro-1-[(2S)-2-methylbutyl)-3-(1H-pyrazol-1-yl)-6-(2,4,6-trifluorophenyl)-2(1H)-pyrazinone,
5-Chloro-1-[(2S)-2-methylbutyl)-3-(1H-pyrazol-1-yl)-6-(2,6-difluoro-4-methoxyphenyl)-2(1H)-pyrazinone, and
5-Chloro-1-[(2S)-2-methylbutyl)-3-(1H-3-methyl-pyrazol-1-yl)-6-(2,6-difluoro-4-methoxyphenyl)-2(1H)-pyrazinone.

8. The method according to claim 1 wherein said animal cell is comprised within a tissue or organ in which proliferation of said cell is not desired.

9-11. (canceled)

12. A compound of Formula 1 including all pharmaceutically acceptable salts, N-oxides, hydrates, solvates or geometric and stereoisomers thereof: 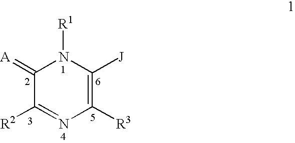
wherein
R¹ is NR⁴R⁵, —N═CR¹⁹R²¹, OR⁶, G¹ or G²; or C₁-C₈ alkyl, C₂-C₈ alkenyl, C₃-C₈ alkynyl, C₃-C₈ cycloalkyl, C₃-C₈ cycloalkenyl, C₄-C₈ cycloalkylalkyl, C₄-C₈ alkylcycloalkyl, C₅-C₁₀ alkylcycloalkylalkyl, C₇-C₁₄ alkylcycloalkylcycloalkyl, C₄-C₈ cycloalkenylalkyl or C₄-C₈ alkylcycloalkenyl, each optionally substituted with one or more substituents independently selected from the group consisting of halogen, cyano, nitro, hydroxy, C₁-C₄ alkoxy, C₁-C₄ haloalkoxy, C₁-C₄ alkylthio, C₁-C₄ alkylamino, C₁-C₄ alkylsulfinyl, C₁-C₄ alkylsulfonyl, C₂-C₆ alkoxycarbonyl, C₂-C₆ alkylcarbonyl, C₃-C₆ trialkylsilyl, G¹ and G²;
A is O, S or NR⁷;
R⁷ is H, C₁-C₄ alkyl, C₁-C₄ haloalkyl, C₂-C₆ alkylcarbonyl or C₂-C₆ alkoxycarbonyl;
R² is cyano, —NR⁸N═CR⁹R¹⁰, —ON═CR⁹R¹⁰, —NR⁸NR¹¹R¹², —ONR¹¹R¹², —CR¹³═NOR¹⁴, —CR¹³═NNR¹¹R¹², —C(W)NR²²R²³, —NR⁸C(O)R²⁶, —NR⁸C(O)NR²⁷ or —NR⁸C(O)OR²⁸; or
R² is a 5- or 6-membered heteroaromatic ring or a 8-, 9- or 10-membered heteroaromatic bicyclic ring system, each ring or ring system optionally substituted with up to 5 substituents independently selected from R²⁴; or 5- or 6-membered saturated or partially saturated heterocyclic ring, optionally including 1-3 ring members selected from the group consisting of C(═O), C(═S), S(O), or S(O)₂, optionally substituted with up to 5 substituents independently selected from R²⁴; or
R² and R⁷ are taken together as —N═C(R¹⁶)—;
W is O, S or ═NR²⁵;
R³ is H, halogen, cyano, C₁-C₆ alkyl, C₁-C₄ haloalkyl, C₃-C₆ cycloalkyl, C₃-C₆ halocycloalkyl, C₂-C₆ alkenyl, C₃-C₆ alkynyl, C₁-C₄ alkoxy, C₁-C₄ haloalkoxy, C₁-C₄ alkylthio, C₁-C₄ haloalkylthio, C₂-C₅ alkoxycarbonyl, hydroxycarbonyl, —SCN or —CHO;
each R⁴ and R⁵ is independently H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R⁴ and R⁵ are taken together as —(CH₂)₃—, —(CH₂)₄—, —(CH₂)₅—, —(CH₂)₆—, —CH₂CH₂OCH₂CH₂— or CH₂CH(CH₃)OCH(CH₃)CH₂—;
R⁶ is H; or C₁-C₈ alkyl, C₃-C₈ alkenyl, C₃-C₈ alkynyl, C₃-C₈ cycloalkyl, C₃-C₈ cycloalkenyl, C₄-C₈ cycloalkylalkyl or C₄-C₈ cycloalkenylalkyl, each optionally substituted with 1 to 4 substituents independently selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl;
each R⁸ is independently H, C₁-C₄ alkyl or C₁-C₄ haloalkyl;
R⁹ is C₁-C₄ alkyl or C₁-C₄ haloalkyl;
R¹⁰ is H, C₁-C₄ alkyl or C₁-C₄ haloalkyl; or
R⁹ and R¹⁰ are taken together as —(CH₂)₃—, —(CH₂)₄—, —(CH₂)₅— or —(CH₂)₆—;
R¹¹ is H, C₁-C₄ alkyl or C₁-C₄ haloalkyl;
R¹² is H, C₁-C₄ alkyl, C₁-C₄ haloalkyl, C₂-C₃ alkylcarbonyl or C₂-C₃ alkoxycarbonyl; or
R¹¹ and R¹² are taken together as —(CH₂)₄—, —(CH₂)₅, —CH₂CH₂OCH₂CH₂— or —CH₂CH(CH₃)OCH(CH₃)CH₂—;
R¹³ is H, NH₂, C₁-C₄ alkyl or C₁-C₄ haloalkyl;
R¹⁴ is H, C₁-C₄ alkyl or C₁-C₄ haloalkyl;
R¹⁶ is H, halogen, cyano, C₁-C₆ alkyl, C₁-C₄ haloalkyl, C₃-C₆ cycloalkyl, C₃-C₆ halocycloalkyl, C₂-C₆ alkenyl, C₂-C₆ alkynyl, C₁-C₄ alkoxy, C₁-C₄ haloalkoxy, C₁-C₄ alkylthio, C₁-C₄ haloalkylthio or C₂-C₅ alkoxycarbonyl;
J is a phenyl, benzyl, naphthalene, 5- or 6-membered heteroaromatic ring or 8-, 9- or 10-membered heteroaromatic bicyclic ring system, each ring or ring system substituted with 1 to 2 substituents independently selected from R³⁰ and optionally substituted up to 4 substituents independently selected from R²⁹;
R²⁹ is halogen, C₁-C₆ alkyl, C₂-C₆ alkenyl, C₂-C₆ alkynyl, C₃-C₆ cycloalkyl, C₁-C₆ haloalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R³⁰ is —Y-X-Q;
Y is O, S(O)ₚ, NR³¹ or direct bond;
X is C₁-C₆ alkylene, C₂-C₆ alkenylene, C₃-C₆ alkynylene, C₃-C₆ cycloalkylene or C₃-C₆ cycloalkenylene, each optionally substituted with one or more substituents independently selected from the group consisting of halogen, cyano, nitro, hydroxy, (═O), C₁-C₆ alkoxy and C₁-C₆ haloalkoxy;
Q is NR³²R³³, OR³⁵ or S(O)ₚR³⁵;
R³¹ is H or C₁-C₆ alkyl, C₁-C₆ haloalkyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
each R³² and R³³ is independently H; or C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl; or R³² and R³³ when optionally taken together with the nitrogen atom to which each is attached form a heterocyclic ring of 3 to 6 ring atoms optionally substituted with R³⁴;
R³⁴ is halogen, C₁-C₆ alkyl, C₁-C₆ haloalkyl or C₁-C₆ alkoxy;
each R³⁵ is independently H, C₁-C₆ alkyl, C₁-C₆ haloalkyl, C₃-C₆ cycloalkyl, C₃-C₆ halocycloalkyl, C₂-C₆ alkenyl, C₃-C₆ alkynyl, C₂-C₆ alkylcarbonyl, C₂-C₆ alkoxycarbonyl, C₂-C₆ alkylthiocarbonyl, C₂-C₆ alkoxythiocarbonyl, C₄-C₈ cycloalkylcarbonyl, C₄-C₈ cycloalkoxycarbonyl, C₄-C₈ cycloalkylthiocarbonyl or C₄-C₈ cycloalkoxythiocarbonyl;
p is 0, 1 or 2;
G¹ is a 3- to 7-membered nonaromatic carbocyclic or heterocyclic ring, optionally including 1 or 2 ring members selected from the group consisting of C(═O), C(═S), S(O) and S(O)₂ and optionally substituted with from 1 to 4 substituents independently selected from R¹⁷;
G² is a phenyl ring, 5- or 6-membered heteroaromatic ring, each ring or ring system optionally substituted with from 1 to 4 substituents independently selected from R¹⁸;
each R¹⁷ is independently C₁-C₂ alkyl, C₁-C₂ haloalkyl, halogen, cyano, nitro or C₁-C₂ alkoxy;
each R¹⁸ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, (C₁-C₄ alkyl)(C₃-C₆ cycloalkyl)amino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
each R¹⁹ and R²¹ is independently H, C₁-C₄ alkyl, C₁-C₄ haloalkyl or C₃-C₈ cycloalkyl; or
R¹⁹ and R²¹ are taken together as —(CH₂)₄—, —(CH₂)₅, —CH₂CH₂OCH₂CH₂— or CH₂CH(CH₃)OCH(CH₃)CH₂—;
each R²² and R²³ is independently H; or C₁-C₄ alkyl, C₁-C₄ alkoxy, C₃-C₈ cycloalkyl or C₄-C₈ cycloalkylalkyl, each optionally substituted with 1 to 4 substituents selected from halogen, cyano, C₁-C₆ alkoxy, C₁-C₆ thioalkyl, C₂-C₆ alkylcarbonyl, C₂-C₆ alkoxycarbonyl, C₂-C₆ dialkylamino, —SCN and C₃-C₆ trialkylsilyl; or
R²² and R²³ are taken together as —(CH₂)₄—, —(CH₂)₅, —CH₂CH₂OCH₂CH₂— or —CH₂CH(CH₃)OCH(CH₃)CH₂—;
each R²⁴ is independently halogen, C₁-C₆ alkyl, C₂-C₆ alkenyl, C₂-C₆ alkynyl, C₃-C₆ cycloalkyl, C₁-C₆ haloalkyl, C₂-C₆ alkoxyalkyl, C₃-C₆ dialkoxyalkyl, C₂-C₆ haloalkenyl, cyano, nitro, C₁-C₆ alkoxy, C₁-C₆ haloalkoxy, C₁-C₆ alkylthio, C₁-C₆ alkylsulfinyl, C₁-C₆ alkylsulfonyl, C₁-C₆ haloalkylthio, C₁-C₆ haloalkylsulfinyl, C₁-C₆ haloalkylsulfonyl, C₁-C₆ alkylamino, C₂-C₆ dialkylamino, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R²⁵ is H, C₁-C₄ alkyl or C₁-C₄ haloalkyl; and
R²⁶ is H, C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, 5- or 6-membered heteroaromatic ring, each ring or ring system optionally substituted with from 1 to 4 substituents independently selected from R³⁶;
R³⁶ is C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, cyano, nitro, C₁-C₄ alkoxy or C₁-C₄ haloalkoxy; and
each R²⁷ and R²⁸ is independently C₁-C₆ alkyl, C₁-C₄ haloalkyl, C₃-C₆ cycloalkyl, C₃-C₆ halocycloalkyl, C₂-C₆ alkenyl or C₃-C₆ alkynyl; or phenyl ring, optionally substituted with from 1 to 4 substituents independently selected from C₁-C₄ alkyl, C₂-C₄ alkenyl, C₂-C₄ alkynyl, C₃-C₆ cycloalkyl, C₁-C₄ haloalkyl, halogen, cyano, nitro, C₁-C₄ alkoxy and C₁-C₄ haloalkoxy.

13. The compound of claim 12 wherein
A is O or S;
R¹ is C₂-C₆ alkyl, C₂-C₆ haloalkyl, C₃-C₈ cycloalkyl, C₄-C₈ cycloalkylalkyl, NR⁴R⁵, G¹ or G²;
R² is cyano, —C(W)NR²²R²³ or —NR⁸C(═O)R²⁶; or a 5- or 6-membered heteroaromatic ring; or a 5- or 6-membered saturated or partially saturated heterocyclic ring, optionally including 1-3 ring members selected from the group consisting of C(═O);
W is O or S;
R³ is halogen, cyano or C₁-C₆ alkyl;
X is C₁-C₆ alkylene or C₂-C₆ alkenylene;
R⁴ and R⁵ are independently H, C₁-C₈ alkyl or C₁-C₈ haloalkyl; and
J is phenyl substituted with R³⁰.

14. The compound of claim 13 wherein
A is O;
R¹ is C₂-C₆ alkyl, C₂-C₆ haloalkyl, C₄-C₈ cycloalkylalkyl, G¹ or G²;
R² is 5- or 6-membered heteroaromatic ring, cyano, —CONH₂ or —NHC(═O)CH₃;
R³ is halogen, cyano or C₁-C₃ alkyl;
X is C₃-C₄ alkylene or C₂-C₄ alkenylene; and
J is phenyl substituted at the 4 position with R³⁰.

15. The compound of claim 14 wherein
R¹ is C₃-C₆ alkyl, C₃-C₆ haloalkyl, C₄-C₈ cycloalkylalkyl, or phenyl, optionally substituted with from 1 to 4 substituents independently selected from R¹⁸;
R² is 5- or 6-membered heteroaromatic ring, each ring optionally substituted with up to 3 substituents independently selected from R²⁴; or —CONH₂ or —NHC(═O)CH₃;
R³ is fluoro, chloro, bromo or methyl;
Y is O or NH;
X is C₃-C₄ alkylene or C₃-C₄ alkenylene;
Q is NR³²R³³ or OR³⁵;
each R³² and R³³ is independently H or C₂-C₆ alkyl or C₂-C₆ haloalkyl; and
R³⁵ is H, C₁-C₆ alkyl or C₁-C₆ haloalkyl.

16. The compound of claim 15 wherein
R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₆ alkyl or C₁-C₄ haloalkyl; or —CONH₂;
Y is NH;
X is C₃-C₄ alkylene; and
Q is NR³²R³³.

17. The compound of claim 16 wherein
R² is 1H-pyrazol-1-yl, 1H-1,2,4-triazol-1-yl, 1H-pyrazol-3-yl or 2-pyridinyl, each optionally substituted with from 1 to 3 substituents independently selected from halogen, cyano, C₁-C₄ alkyl or C₁-C₃ haloalkyl; or —CONH₂; and
each R³², R³³ and R³⁵ is each independently H or C₁-C₄ alkyl or C₁-C₃ haloalkyl.

18. The compound of claim 17 wherein the compound is selected from the group consisting of:
5-chloro-6-[4-[3-(dimethylamino)propoxy]-2,6-difluorophenyl]-1-[(2S)-2-methylbutyl]-3-(1H-pyrazol-1-yl)-2(1H)-pyrazinone,
5-chloro-1-cyclopropylmethyl-6-[4-[3-(dimethylamino)propoxy]-2,6-difluorophenyl]-3-(1H-pyrazol-1-yl)-2(1H)-pyrazinone,
5-chloro-6-[2,6-difluoro-4-[3-(methylamino)propoxy]phenyl]-1-[(2S)-2-methylbutyl]-3-(1H-pyrazol-1-yl)-2(1H)-pyrazinone,
6-chloro-5-[4-[3-(dimethylamino)propoxy]-2,6-difluorophenyl]-3,4-dihydro-4-[(2S)-2-methylbutyl]-3-oxopyrazinecarboxamide,
6-chloro-5-[2,6-difluoro-4-[3-(methylamino)propoxy]phenyl]-3,4-dihydro-4-[(2S)-2-methylbutyl]-3-oxopyrazinecarboxamide,
6-chloro-5-[4-[3-(dimethylamino)propoxy]-2,6-difluorophenyl]-3,4-dihydro-3-oxo-4-(3,3,3-trifluoro-2-methylpropyl)pyrazinecarboxamide,
6-chloro-5-[2,6-difluoro-4-[3-(methylamino)propoxy]phenyl]-3,4-dihydro-3-oxo-4-(3,3,3-trifluoro-2-methylpropyl)pyrazinecarboxamide,
5-chloro-6-[4-[3-(dimethylamino)propoxy]-2,6-difluorophenyl]-1-(3-fluorophenyl)-3-(1H-pyrazol-1-yl)-2(1H)-pyrazinone,
5-chloro-6-[2,6-difluoro-4-[3-(methylamino)propoxy]phenyl]-1-(3-fluorophenyl)-3-(1H-pyrazol-1-yl)-2(1H)-pyrazinone,
5-chloro-6-[4-[3-(dimethylamino)propoxy]-2,6-difluorophenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-3-(1H-pyrazol-1-yl)-1-(3,3,3-trifluoro-2-methylpropyl)-2(1H)-pyrazinone,
5-chloro-6-[2,6-difluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[4-[3-(dimethylamino)propoxy]-2,6-difluorophenyl]-1[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(3-methyl-1H-pyrazol-1-yl)-2(1H)-pyrazinone,
5-chloro-6-[2-chloro-6-fluoro-4-[3-(methylamino)propoxy]phenyl]-1-[(2S)-2-methylbutyl]-3-(1H-pyrazol-1-yl)-2(1H)-pyrazinone,
5-chloro-6-[2,6-difluoro-4-[3-(methylamino)propoxy]phenyl]-1-[(2S)-2-methylbutyl]-3-(1-methyl-1H-pyrazol-3-yl)-2(1H)-pyrazinone,
5-Chloro-1-[(2S)-2-methylbutyl)-3-(1H-pyrazol-1-yl)-6-(2,4,6-trifluorophenyl)-2(1H)-pyrazinone,
5-Chloro-1-[(2S)-2-methylbutyl)-3-(1H-pyrazol-1-yl)-6-(2,6-difluoro-4-methoxyphenyl)-2(1H)-pyrazinone, and
5-Chloro-1-[(2S)-2-methylbutyl)-3-(1H-3-methyl-pyrazol-1-yl)-6-(2,6-difluoro-4-methoxyphenyl)-2(1H)-pyrazinone.

19. A composition which comprises a compound of claim 12 or a pharmaceutically acceptable salt thereof together with a physiologically acceptable carrier.

20. A method of inhibiting undesired animal cellular proliferation said method comprising contacting an animal cell with a compound or composition of claim 12.

21-23. (canceled)

24. The method of claim 20 wherein said animal cell is comprised within a tissue or organ in which proliferation of said cell is not desired
